(19) United States
(12) Patent Application Publication
(10) Pub. No.: US 2024/0415866 A1
(43) Pub. Date: Dec. 19, 2024

(54) COMPOSITION AND METHODS FOR TREATMENT OF PRIMARY CILIARY DYSKINESIA

(71) Applicant: Translate Bio, Inc., Waltham, MA (US)

(72) Inventors: Caroline J. WOO, Cambridge, MA (US); Ayed ALLAWZI, Cambridge, MA (US); James SULLIVAN, Cambridge, MA (US); Sherry HE, Cambridge, MA (US); Neha KAUSHAL, Cambridge, MA (US); Shrirang KARVE, Cambridge, MA (US); Haiyan ZHANG, Cambridge, MA (US); Sadaf ATAROD, Cambridge, MA (US)

(21) Appl. No.: 18/661,393

(22) Filed: May 10, 2024

Related U.S. Application Data
(63) Continuation of application No. PCT/US2022/079650, filed on Nov. 10, 2022.
(60) Provisional application No. 63/278,007, filed on Nov. 10, 2021.

(51) Int. Cl. A61K 31/7105; A61K 9/127; A61K 38/17; A61P 11/00

(52) U.S. Cl. CPC A61K 31/7105; A61K 9/1272; A61K 38/1709; A61P 11/00

(57) ABSTRACT

The present invention provides, among other things, methods and compositions for treating primary ciliary dyskinesia (PCD) based on mRNA therapy. The present invention is based, in part, on the surprising discovery that administration of the mRNA encoding a DNAI1 protein at a dose ranging from 1 mg to 36 mg daily for five consecutive days resulted in restoration of ciliary function in PCD. Additionally, the methods of the present invention resulted in expression of the DNAI1 protein in airway epithelium at a level at least 10% of the wild-type level, an increase ciliary beat frequency (CBF), and DNAI1-positive transfection efficiency of greater than 5% in ciliated cells.

### RELATED APPLICATIONS

This application is a continuation of International Patent Application No. PCT/US2022/079650, filed Nov. 10, 2022, which claims the benefit of, U.S. Provisional Patent Application Ser. No. 63/278,007, filed Nov. 10, 2021, the entire contents of which are hereby incorporated herein by reference.

### SEQUENCE LISTING

The instant application contains a Sequence Listing which has been submitted electronically in XML format and is hereby incorporated by reference in its entirety. Said XML file, created on May 10, 2024, is named 754308_SA9-823PCCON_ST26.xml and is 39,280 bytes in size.

### BACKGROUND

Primary ciliary dyskinesia (PCD) is an auto recessive disorder characterized by abnormal cilia and flagella that are found in the linings of the airway, the reproductive system, and other organs and tissues. PCD occurs in approximately 1 in 16,000. Symptoms are present as early as at birth, with breathing problems, and the affected individuals develop frequent respiratory tract infections beginning in early childhood. In patients with PCD, cilia in the respiratory system have defective function, which prevents the clearance of mucous from the lungs, paranasal sinuses and middle ears. People with PCD also have year-round nasal congestion and chronic cough. Chronic respiratory tract infections can result in condition called bronchiectasis, which damages the passages, called bronchi, and can cause life-threatening breathing problems. Some individuals with PCD also have infertility, recurrent ear infections, abnormally placed organs within their chest and abdomen.

Among several genes confirmed to be directly involved in PCD pathogenesis, a significant number of mutations are found in two genes: DNAI1 and DNAH5, encoding intermediate and heavy chains of the axonemal dynein, respectively. Mutations in other genes, coding for proteins involved in the axonemal ultrastructure (DNAH11, DNAI2, TXNDC3, RSPH9, RSPH4A) or assembly (KTU, CRRC50), also have been reported, as well as mutations in the RPGR gene in certain cases of PCD. Mutations in DNAI1 and DNAH5, both associated with a ciliary outer dynein arm (ODA) defect phenotype, are collectively estimated to account for almost 40% of PCD cases.

There is currently no cure for PCD. Current standard of care includes aggressive measures to enhance clearance of mucus and with antibiotic therapy for bacterial infections of the airways. Routine immunizations are administered to prevent respiratory infections and other secondary complications. For some patients, lobectomy, lung transplantation, and sinus surgery are considered. Gene therapy has been studied to address the urgent need for new, more effective treatments of PCD. However, conventional gene therapy methods to deliver the therapeutic agent to the lungs, and specifically to cilia, still remain challenging.

### SUMMARY OF THE INVENTION

The present invention provides, among other things, methods and compositions for use in the treatment of primary ciliary dyskinesia (PCD). The present invention is based, in part, on the surprising discovery that administration of the mRNA encoding a DNAI1 protein at a dose ranging from 1 mg to 36 mg daily for five consecutive days resulted in restoration of ciliary function in PCD. Additionally, the methods of the present invention resulted in expression of the DNAI1 protein in airway epithelium at a level at least 10% of the wild-type level, an increase ciliary beat frequency (CBF), and DNAI1-positive transfection efficiency of greater than 5% in ciliated cells.

In one aspect, the present invention provides, among other things, a treating primary ciliary dyskinesia (PCD) comprising administering to a subject in need of treatment an mRNA encoding a dynein axonemal intermediate chain 1 (DNAI1) protein, wherein the mRNA is administered at a dose ranging from 1 mg to 36 mg daily for 5 consecutive days.

In some embodiments, the mRNA is administered at a dose ranging from 0.01 mg to 100 mg. In some embodiments, the mRNA is administered at a dose ranging from 1 mg to 50 mg. In some embodiments, the mRNA is administered at a dose ranging from 1.5 mg to 40 mg. In some embodiments, the mRNA is administered at a dose ranging from 2 mg to 36 mg. In some embodiments, the mRNA is administered at a dose ranging from 5 mg to 30 mg. In some embodiments, the mRNA is administered at a dose ranging from 10 mg to 20 mg. In some embodiments, the mRNA is administered at a dose ranging from 6 mg to 24 mg. In some embodiments, the mRNA is administered at a dose ranging from 8 mg to 16 mg. In some embodiments, the mRNA is administered at a dose of 0.5 mg. In some embodiments, the mRNA is administered at a dose of 1 mg. In some embodiments, the mRNA is administered at a dose of 1.5 mg. In some embodiments, the mRNA is administered at a dose of 2 mg. In some embodiments, the mRNA is administered at a dose of 2.5 mg. In some embodiments, the mRNA is administered at a dose of 3 mg. In some embodiments, the mRNA is administered at a dose of 4 mg. In some embodiments, the mRNA is administered at a dose of 5 mg. In some embodiments, the mRNA is administered at a dose of 6 mg. In some embodiments, the mRNA is administered at a dose of 7 mg. In some embodiments, the mRNA is administered at a dose of 8 mg. In some embodiments, the mRNA is administered at a dose of 9 mg. In some embodiments, the mRNA is administered at a dose of 10 mg. In some embodiments, the mRNA is administered at a dose of 11 mg. In some embodiments, the mRNA is administered at a dose of 12 mg. In some embodiments, the mRNA is administered at a dose of 13 mg. In some embodiments, the mRNA is administered at a dose of 14 mg. In some embodiments, the mRNA is administered at a dose of 15 mg. In some embodiments, the mRNA is administered at a dose of 16 mg. In some embodiments, the mRNA is administered at a dose of 17 mg. In some embodiments, the mRNA is administered at a dose of 18 mg. In some embodiments, the mRNA is administered at a dose of 20 mg. In some embodiments, the mRNA is administered at a dose of 21 mg. In some embodiments, the mRNA is administered at a dose of 22 mg. In some embodiments, the mRNA is administered at a dose of 23 mg. In some embodiments, the mRNA is administered at a dose of 24 mg. In some embodiments, the mRNA is administered at a dose of 25 mg. In some embodiments, the mRNA is administered at a dose of 26 mg. In some embodiments, the mRNA is administered at a dose of 27 mg. In some embodiments, the mRNA is administered at a dose of 28 mg. In some embodiments, the mRNA is administered at a dose of 29 mg. In some embodiments, the mRNA is administered at a dose of 30 mg. In some embodiments, the mRNA is administered at a dose of 31 mg. In some embodiments, the mRNA is administered at a dose of 32 mg. In some embodiments, the mRNA is administered at a dose of 33 mg. In some embodiments, the mRNA is administered at a dose of 34 mg. In some embodiments, the mRNA is administered at a dose of 35 mg. In some embodiments, the mRNA is administered at a dose of 36 mg. In some embodiments, the mRNA is administered at a dose of 37 mg. In some embodiments, the mRNA is administered at a dose of 38 mg. In some embodiments, the mRNA is administered at a dose of 39 mg. In some embodiments, the mRNA is administered at a dose of 40 mg.

In some embodiments, the mRNA is administered for 1 day. In some embodiments, the mRNA is administered for 2 consecutive days. In some embodiments, the mRNA is administered for 3 consecutive days. In some embodiments, the mRNA is administered for 4 consecutive days. In some embodiments, the mRNA is administered for at least 1 day. In some embodiments, the mRNA is administered for at least 2 days. In some embodiments, the mRNA is administered for at least 3 days. In some embodiments, the mRNA is administered for at least 4 days. In some embodiments, the administration is for at least for 5 consecutive days. In some embodiments, the mRNA is administered for 5 days. In some embodiments, the mRNA is administered for 6 days. In some embodiments, the mRNA is administered for 7 days. In some embodiments, the mRNA is administered for 8 days. In some embodiments, the mRNA is administered for 9 days. In some embodiments, the mRNA is administered for 10 days. In some embodiments, the mRNA is administered for 12 days. In some embodiments, the mRNA is administered for 14 days. In some embodiments, the mRNA is administered for 15 days. In some embodiments, the mRNA is administered for 16 days. In some embodiments, the mRNA is administered for 17 days. In some embodiments, the mRNA is administered for 18 days. In some embodiments, the mRNA is administered for 20 days. In some embodiments, the mRNA is administered for a week. In some embodiments, the mRNA is administered for two weeks. In some embodiments, the mRNA is administered for three weeks. In some embodiments, the mRNA is administered for four weeks. In some embodiments, the mRNA is administered for five weeks. In some embodiments, the mRNA is administered for six weeks. In some embodiments, the mRNA is administered for seven weeks. In some embodiments, the mRNA is administered for eight weeks. In some embodiments, the mRNA is administered for ten weeks. In some embodiments, the mRNA is administered for a month. In some embodiments, the mRNA is administered for two months. In some embodiments, the mRNA is administered for three months. In some embodiments, the mRNA is administered for four months. In some embodiments, the mRNA is administered for five months. In some embodiments, the mRNA is administered for six months. In some embodiments, the mRNA is administered for seven months. In some embodiments, the mRNA is administered for eight months. In some embodiments, the mRNA is administered for nine months. In some embodiments, the mRNA is administered for ten months. In some embodiments, the mRNA is administered for eleven months. In some embodiments, the mRNA is administered for twelve months. In some embodiments, the mRNA is administered for a year.

In some embodiments, the administration for 5 consecutive days is for at least for 5 consecutive days. In some embodiments, the mRNA is administered for 5 days. In some embodiments, the mRNA is administered for 6 days. In some embodiments, the mRNA is administered for 7 days. In some embodiments, the mRNA is administered for 8 days. In some embodiments, the mRNA is administered for 9 days. In some embodiments, the mRNA is administered for 10 days. In some embodiments, the mRNA is administered for 12 days. In some embodiments, the mRNA is administered for 14 days. In some embodiments, the mRNA is administered for 15 days. In some embodiments, the mRNA is administered for 16 days. In some embodiments, the mRNA is administered for 17 days. In some embodiments, the mRNA is administered for 18 days. In some embodiments, the mRNA is administered for 20 days. In some embodiments, the mRNA is administered for a week. In some embodiments, the mRNA is administered for two weeks. In some embodiments, the mRNA is administered for three weeks. In some embodiments, the mRNA is administered for four weeks. In some embodiments, the mRNA is administered for five weeks. In some embodiments, the mRNA is administered for six weeks. In some embodiments, the mRNA is administered for seven weeks. In some embodiments, the mRNA is administered for eight weeks. In some embodiments, the mRNA is administered for ten weeks. In some embodiments, the mRNA is administered for a month. In some embodiments, the mRNA is administered for two months. In some embodiments, the mRNA is administered for three months. In some embodiments, the mRNA is administered for four months. In some embodiments, the mRNA is administered for five months. In some embodiments, the mRNA is administered for six months. In some embodiments, the mRNA is administered for seven months. In some embodiments, the mRNA is administered for eight months. In some embodiments, the mRNA is administered for nine months. In some embodiments, the mRNA is administered for ten months. In some embodiments, the mRNA is administered for eleven months. In some embodiments, the mRNA is administered for twelve months. In some embodiments, the mRNA is administered for a year.

In one aspect, the present invention provides, among other things, a method of treating primary ciliary dyskinesia (PCD) comprising administering to a subject in need of treatment an mRNA encoding a dynein axonemal intermediate chain 1 (DNAI1) protein, wherein the mRNA is administered at a therapeutically effective dose and interval such that the subject achieves an increase in ciliary beat frequency (CBF) compared to a control.

In one aspect, the invention provides, among other things, a method of treating primary ciliary dyskinesia (PCD) comprising administering to a subject in need of treatment an mRNA encoding a dynein axonemal intermediate chain 1 (DNAI1) protein, wherein the mRNA is administered at a therapeutically effective dose and interval such that the subject maintains an expression of the DNAI1 protein in airway epithelium at a level at least 10% of the wild type level.

In some embodiments, the airway epithelium comprises lung epithelium. In some embodiments, the airway epithelium comprises basal cell. In some embodiments, the airway epithelium comprises club cell. In some embodiments, the airway epithelium comprises ciliated cell. In some embodiments, the airway epithelium comprises goblet cell. In some embodiments, the airway epithelium comprises tuft cell. In some embodiments, the airway epithelium comprises pulmonary neuroendocrine cell (PNEC). In some embodiments, the airway epithelium comprises pulmonary ionocyte. In some embodiments, the airway epithelium comprises microfold cell. In some embodiments, the airway epithelium comprises hillock. In some embodiments, the airway epithelium comprises cilia.

In some embodiments, the subject maintains an expression of the DNAI1 protein in airway epithelium at a level at least 12% of the wild type level. In some embodiments, the subject maintains an expression of the DNAI1 protein in airway epithelium at a level at least 15% of the wild type level. In some embodiments, the subject maintains an expression of the DNAI1 protein in airway epithelium at a level at least 18% of the wild type level. In some embodiments, the subject maintains an expression of the DNAI1 protein in airway epithelium at a level at least 20% of the wild type level. In some embodiments, the subject maintains an expression of the DNAI1 protein in airway epithelium at a level at least 22% of the wild type level. In some embodiments, the subject maintains an expression of the DNAI1 protein in airway epithelium at a level at least 25% of the wild type level. In some embodiments, the subject maintains an expression of the DNAI1 protein in airway epithelium at a level at least 28% of the wild type level. In some embodiments, the subject maintains an expression of the DNAI1 protein in airway epithelium at a level at least 30% of the wild type level. In some embodiments, the subject maintains an expression of the DNAI1 protein in airway epithelium at a level at least 35% of the wild type level. In some embodiments, the subject maintains an expression of the DNAI1 protein in airway epithelium at a level at least 38% of the wild type level. In some embodiments, the subject maintains an expression of the DNAI1 protein in airway epithelium at a level at least 40% of the wild type level. In some embodiments, the subject maintains an expression of the DNAI1 protein in airway epithelium at a level at least 42% of the wild type level. In some embodiments, the subject maintains an expression of the DNAI1 protein in airway epithelium at a level at least 45% of the wild type level. In some embodiments, the subject maintains an expression of the DNAI1 protein in airway epithelium at a level at least 50% of the wild type level. In some embodiments, the subject maintains an expression of the DNAI1 protein in airway epithelium at a level at least 60% of the wild type level. In some embodiments, the subject maintains an expression of the DNAI1 protein in airway epithelium at a level at least 70% of the wild type level. In some embodiments, the subject maintains an expression of the DNAI1 protein in airway epithelium at a level at least 80% of the wild type level. In some embodiments, the subject maintains an expression of the DNAI1 protein in airway epithelium at a level at least 90% of the wild type level. In some embodiments, the subject maintains an expression of the DNAI1 protein in airway epithelium at a level at least 95% of the wild type level. In some embodiments, the subject maintains an expression of the DNAI1 protein in airway epithelium at a level at least 99% of the wild type level.

In some embodiments, the CBF is increased by at least 3% as compared to a control. In some embodiments, the CBF is increased by at least 4% as compared to a control. In some embodiments, the CBF is increased by at least 5% as compared to a control. In some embodiments, the CBF is increased by at least 6% as compared to a control. In some embodiments, the CBF is increased by at least 7% as compared to a control. In some embodiments, the CBF is increased by at least 8% as compared to a control. In some embodiments, the CBF is increased by at least 9% as compared to a control. In some embodiments, the CBF is increased by at least 10% as compared to a control. In some embodiments, the CBF is increased by at least 11% as compared to a control. In some embodiments, the CBF is increased by at least 12% as compared to a control. In some embodiments, the CBF is increased by at least 13% as compared to a control. In some embodiments, the CBF is increased by at least 14% as compared to a control. In some embodiments, the CBF is increased by at least 15% as compared to a control. In some embodiments, the CBF is increased by at least 16% as compared to a control. In some embodiments, the CBF is increased by at least 17% as compared to a control. In some embodiments, the CBF is increased by at least 18% as compared to a control. In some embodiments, the CBF is increased by at least 19% as compared to a control. In some embodiments, the CBF is increased by at least 20% as compared to a control. In some embodiments, the CBF is increased by at least 22% as compared to a control. In some embodiments, the CBF is increased by at least 25% as compared to a control. In some embodiments, the CBF is increased by at least 28% as compared to a control. In some embodiments, the CBF is increased by at least 30% as compared to a control. In some embodiments, the CBF is increased by at least 35% as compared to a control. In some embodiments, the CBF is increased by at least 40% as compared to a control. In some embodiments, the CBF is increased by at least 45% as compared to a control. In some embodiments, the CBF is increased by at least 50% as compared to a control. In some embodiments, the CBF is increased by at least 55% as compared to a control. In some embodiments, the CBF is increased by at least 60% as compared to a control. In some embodiments, the CBF is increased by at least 65% as compared to a control. In some embodiments, the CBF is increased by at least 70% as compared to a control. In some embodiments, the CBF is increased by at least 75% as compared to a control. In some embodiments, the CBF is increased by at least 80% as compared to a control. In some embodiments, the CBF is increased by at least 85% as compared to a control. In some embodiments, the CBF is increased by at least 90% as compared to a control. In some embodiments, the CBF is increased by at least 95% as compared to a control. In some embodiments, the CBF is increased by at least 97% as compared to a control. In some embodiments, the CBF is increased by at least 98% as compared to a control. In some embodiments, the CBF is increased by at least 99% as compared to a control. In some embodiments, the CBF is increased by at least 100% as compared to a control. In some embodiments, the administration of the mRNA restores the CBF level as compared to a control.

In some embodiments, a control is a CBF in the subject before administration of the mRNA encoding a DNAI1 protein. In some embodiments, a control is a CBF in an untreated. In some embodiments, a control is a CBF in a subject administered with a placebo.

In some embodiments, the administration of the mRNA rescues the CBF in the subject to a normal CBF level.

In some embodiments, a normal CBF level is 6-16 Hz. In some embodiments, a normal CBF level is 6-10 Hz. In some embodiments, a normal CBF level is 6-9 Hz. In some embodiments, a normal CBF level is 6 Hz. In some embodiments, a normal CBF level is 7 Hz. In some embodiments, a normal CBF level is 8 Hz. In some embodiments, a normal CBF level is 9 Hz. In some embodiments, a normal CBF level is 10 Hz. In some embodiments, a normal CBF level is 11 Hz. In some embodiments, a normal CBF level is 12 Hz. In some embodiments, a normal CBF level is the level sufficient for proper ciliary function. In some embodiments, a normal CBF level is the level sufficient for mucus clearance.

In some embodiments, the CBF is measured by a high-speed video-microscopy (HSVM). In some embodiments, the CBF is measured by a high-speed digital video camera.

In some embodiments, the CBF is measured at a temperature of 25° C. In some embodiments, the CBF is measured at a temperature of 32° C. In some embodiments, the CBF is measured at a temperature of 37° C.

In some embodiments, the administration of the mRNA results in DNAI1-positive transfection efficiency of greater than 3% in ciliated cells. In some embodiments, the administration of the mRNA results in DNAI1-positive transfection efficiency of greater than 5% in ciliated cells. In some embodiments, the administration of the mRNA results in DNAI1-positive transfection efficiency of greater than 7% in ciliated cells. In some embodiments, the administration of the mRNA results in DNAI1-positive transfection efficiency of greater than 10% in ciliated cells. In some embodiments, the administration of the mRNA results in DNAI1-positive transfection efficiency of greater than 12% in ciliated cells. In some embodiments, the administration of the mRNA results in DNAI1-positive transfection efficiency of greater than 15% in ciliated cells. In some embodiments, the administration of the mRNA results in DNAI1-positive transfection efficiency of greater than 18% in ciliated cells. In some embodiments, the administration of the mRNA results in DNAI1-positive transfection efficiency of greater than 20% in ciliated cells. In some embodiments, the administration of the mRNA results in DNAI1-positive transfection efficiency of greater than 22% in ciliated cells. In some embodiments, the administration of the mRNA results in DNAI1-positive transfection efficiency of greater than 25% in ciliated cells. In some embodiments, the administration of the mRNA results in DNAI1-positive transfection efficiency of greater than 28% in ciliated cells. In some embodiments, the administration of the mRNA results in DNAI1-positive transfection efficiency of greater than 30% in ciliated cells. In some embodiments, the administration of the mRNA results in DNAI1-positive transfection efficiency of greater than 35% in ciliated cells. In some embodiments, the administration of the mRNA results in DNAI1-positive transfection efficiency of greater than 40% in ciliated cells. In some embodiments, the administration of the mRNA results in DNAI1-positive transfection efficiency of greater than 45% in ciliated cells. In some embodiments, the administration of the mRNA results in DNAI1-positive transfection efficiency of greater than 50% in ciliated cells. In some embodiments, the administration of the mRNA results in DNAI1-positive transfection efficiency of greater than 55% in ciliated cells. In some embodiments, the administration of the mRNA results in DNAI1-positive transfection efficiency of greater than 60% in ciliated cells. In some embodiments, the administration of the mRNA results in DNAI1-positive transfection efficiency of greater than 65% in ciliated cells. In some embodiments, the administration of the mRNA results in DNAI1-positive transfection efficiency of greater than 70% in ciliated cells. In some embodiments, the administration of the mRNA results in DNAI1-positive transfection efficiency of greater than 75% in ciliated cells. In some embodiments, the administration of the mRNA results in DNAI1-positive transfection efficiency of greater than 80% in ciliated cells. In some embodiments, the administration of the mRNA results in DNAI1-positive transfection efficiency of greater than 85% in ciliated cells. In some embodiments, the administration of the mRNA results in DNAI1-positive transfection efficiency of greater than 90% in ciliated cells. In some embodiments, the administration of the mRNA results in DNAI1-positive transfection efficiency of greater than 95% in ciliated cells. In some embodiments, the administration of the mRNA results in DNAI1-positive transfection efficiency of greater than 98% in ciliated cells. In some embodiments, the administration of the mRNA results in DNAI1-positive transfection efficiency of greater than 99% in ciliated cells.

In some embodiments, the DNAI1 mRNA is encapsulated in a liposome.

In some embodiments, the liposome comprises one or more cationic lipids, one or more non-cationic lipids and one or more PEG-modified lipids. In some embodiments, the liposome comprises no more than three distinct lipid components. In some embodiments, the liposome comprises no more than four distinct lipid components.

In some embodiments, the one or more cationic lipids are selected from the group consisting of TLT-01D-DMA, TL1-10D-DMA, GL-TES-SA-DMP-E18-2, HEP-E4-E10, HEP-E3-E10, TL1-04D-DMA, GL-TES-SA-DME-E18-2, Guan-SS-Chol, SY-3-E14-DMAPr, RL3-07D-DMA, cKK-E12, OF-02, ICE (Imidazol-based ester) and combinations thereof.

In some embodiments, the cationic lipid is TL1-01D-DMA. In some embodiments, the cationic lipid is TL1-10D-DMA. In some embodiments, the cationic lipid is GL-TES-SA-DMP-E18-2. In some embodiments, the cationic lipid is HEP-E4-E10. In some embodiments, the cationic lipid is HEP-E3-E10. In some embodiments, the cationic lipid is TL1-04D-DMA. In some embodiments, the cationic lipid is GL-TES-SA-DME-E18-2. In some embodiments, the cationic lipid is Guan-SS-Chol. In some embodiments, the cationic lipid is SY-3E14-DMAPr. In some embodiments, the cationic lipid is RL3-07D-DMA. In some embodiments, the cationic lipid is ICE.

In some embodiments, the one or more non-cationic lipids are selected from DSPC (1,2-distearoyl-sn-glycero-3-phosphocholine), DPPC (1,2-dipalmitoyl-sn-glycero-3-phosphocholine), DOPE (1,2-dioleyl-sn-glycero-3-phosphoethanolamine), DOPC (1,2-dioleyl-sn-glycero-3-phosphotidylcholine) DPPE (1,2-dipalmitoyl-sn-glycero-3-phosphoethanolamine), DMPE (1,2-dimyristoyl-sn-glycero-3-phosphoethanolamine), DOPG (1,2-dioleoyl-sn-glycero-3-phospho-(1′-rac-glycerol)) or combinations thereof. In some embodiments, the non-cationic lipid is DOPE.

In some embodiments, the one or more PEG-modified lipids comprise a poly(ethylene) glycol chain of up to 5 kDa in length covalently attached to a lipid with alkyl chain(s) of C6-C20 length. In some embodiments, the PEG-modified lipid is DMG-PEG2K.

In some embodiments, the cationic lipid constitutes about 30-60% of the liposome by molar ratio.

In some embodiments, the cationic lipid constitutes about 30%, 40%, 50%, or 60% of the liposome by molar ratio.

In some embodiments, the liposomes comprises four distinct lipid components, namely a cationic lipid, a non-cationic lipid, cholesterol and a PEG-modified lipid. In some embodiments, the molar ratio of cationic lipid to non-cationic lipid to cholesterol to PEG-modified lipid is between about 30-60:25-35:20-30:1-15, respectively.

In some embodiments, the liposomes comprises three distinct lipid components, namely a cationic lipid (typically a sterol-based cationic lipid), a non-cationic lipid, and a PEG-modified lipid. In some embodiments, the molar ratio of cationic lipid to non-cationic lipid to PEG-modified lipid is approximately 60:35:5, respectively.

In some embodiments, the liposome has a diameter of about 30 nm to 200 nm, optionally wherein the liposome has a diameter of about 100 nm or less than 100 nm. In some embodiments, the liposome has a size of less than 200 nm. In some embodiments, the liposome has a size of less than 180 nm. In some embodiments, the liposome has a size of less than 150 nm. In some embodiments, the liposome has a size of less than 125 nm. In some embodiments, the liposome has a size of less than 110 nm. In some embodiments, the liposome has a size of less than 200 nm. In some embodiments, the liposome has a size of less than 100 nm. In some embodiments, the liposome has a size of less than 90 nm. In some embodiments, the liposome has a size of less than 80 nm. In some embodiments, the liposome has a size of less than 75 nm. In some embodiments, the liposome has a size of less than 70 nm. In some embodiments, the liposome has a size of less than 65 nm. In some embodiments, the liposome has a size of less than 60 nm. In some embodiments, the liposome has a size of 30 nm to 180 nm. In some embodiments, the liposome has a size of 50 nm to 160 nm. In some embodiments, the liposome has a size of 60 nm to 120 nm. In some embodiments, the liposome has a size of 80 nm to 100 nm. In some embodiments, the liposome has a size of 60 nm to 100 nm. In some embodiments, the liposome has a size of 60 nm to 80 nm.

In some embodiments, the DNAI1 mRNA is codon optimized. In some embodiments, the codon-optimized mRNA produces at least 10% more, 15% more, 20% more, 25% more, or at least 30% more DNAI1 protein in comparison to a non-codon-optimized mRNA sequence. In some embodiments, the codon-optimized mRNA produces at least 10% more DNAI1 protein in comparison to a non-codon-optimized mRNA sequence. In some embodiments, the codon-optimized mRNA produces at least 15% more DNAI1 protein in comparison to a non-codon-optimized mRNA sequence. In some embodiments, the codon-optimized mRNA produces at least 20% more DNAI1 protein in comparison to a non-codon-optimized mRNA sequence. In some embodiments, the codon-optimized mRNA produces at least 25% more DNAI1 protein in comparison to a non-codon-optimized mRNA sequence. In some embodiments, the codon-optimized mRNA produces at least 30% more DNAI1 protein in comparison to a non-codon-optimized mRNA sequence. In some embodiments, the codon-optimized mRNA produces greater than 30% more DNAI1 protein in comparison to a non-codon-optimized mRNA sequence.

In some embodiments, the DNAI1 mRNA is codon optimized to include a high-producing codon optimized mRNA sequence that provides at least 10% more, 15% more, 20% more, 25% more, or at least 30% more DNAI1 protein in comparison to a second DNAI1 mRNA sequence that is codon optimized with a different, reference codon optimized sequence and delivered at the same dose. In some embodiments, the high-producing codon-optimized mRNA sequence provides at least 10% more DNAI1 protein in comparison to a second DNAI1 mRNA sequence that is codon optimized with a different, reference codon optimized sequence and delivered at the same dose. In some embodiments, the high-producing codon-optimized mRNA sequence provides at least 15% more DNAI1 protein in comparison to a second DNAI1 mRNA sequence that is codon optimized with a different, reference codon optimized sequence and delivered at the same dose. In some embodiments, the high-producing codon-optimized mRNA sequence provides at least 20% more DNAI1 protein in comparison to a second DNAI1 mRNA sequence that is codon optimized with a different, reference codon optimized sequence and delivered at the same dose. In some embodiments, the high-producing codon-optimized mRNA sequence provides at least 25% more DNAI1 protein in comparison to a second DNAI1 mRNA sequence that is codon optimized with a different, reference codon optimized sequence and delivered at the same dose. In some embodiments, the high-producing codon-optimized mRNA sequence provides at least 30% more DNAI1 protein in comparison to a second DNAI1 mRNA sequence that is codon optimized with a different, reference codon optimized sequence and delivered at the same dose. In some embodiments, the high-producing codon-optimized mRNA sequence provides greater than 30% more DNAI1 protein in comparison to a second DNAI1 mRNA sequence that is codon optimized with a different, reference codon optimized sequence and delivered at the same dose.

In some embodiments, the DNAI1 mRNA comprises one or more modified nucleotides.

In some embodiments, the one or more modified nucleotides are selected from pseudouridine, N-1-methyl-pseudouridine, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and/or 2-thiocytidine.

In some embodiments, one or more modified nucleotides comprises pseudouridine. In some embodiments, one or more modified nucleotides comprises N-1-methyl-pseudouridine. In some embodiments, one or more modified nucleotides comprises 2-aminoadenosine. In some embodiments, one or more modified nucleotides comprises 2-thiothymidine. In some embodiments, one or more modified nucleotides comprises inosine. In some embodiments, one or more modified nucleotides comprises pyrrolo-pyrimidine. In some embodiments, one or more modified nucleotides comprises 3-methyl adenosine. In some embodiments, one or more modified nucleotides comprises 5-methylcytidine. In some embodiments, one or more modified nucleotides comprises C-5 propynyl-cytidine. In some embodiments, one or more modified nucleotides comprises C-5 propynyl-uridine. In some embodiments, one or more modified nucleotides comprises 2-aminoadenosine. In some embodiments, one or more modified nucleotides comprises C5-bromouridine. In some embodiments, one or more modified nucleotides comprises C5-fluorouridine. In some embodiments, one or more modified nucleotides comprises C5-iodouridine. In some embodiments, one or more modified nucleotides comprises C5-propynyl-uridine. In some embodiments, one or more modified nucleotides comprises C5-propynyl-cytidine. In some embodiments, one or more modified nucleotides comprises C5-methylcytidine. In some embodiments, one or more modified nucleotides comprises 2-aminoadenosine. In some embodiments, one or more modified nucleotides comprises 7-deazaadenosine. In some embodiments, one or more modified nucleotides comprises 7-deazaguanosine. In some embodiments, one or more modified nucleotides comprises 8-oxoadenosine. In some embodiments, one or more modified nucleotides comprises 8-oxoguanosine. In some embodiments, one or more modified nucleotides comprises O(6)-methylguanine. In some embodiments, one or more modified nucleotides comprises 2-thiocytidine.

In some embodiments, the mRNA is unmodified.

In some embodiments, the mRNA comprises a 5′-untranslated region (5′-UTR) that has a sequence set forth in SEQ ID NO: 2 or SEQ ID NO: 3.

In some embodiments, the mRNA comprises a 3′-untranslated region (3′-UTR) that has a sequence set forth in SEQ ID NO: 4 or SEQ ID NO: 5.

In some embodiments, the mRNA comprises a coding sequence at least 70%, 75%, 80%, 85%, 90%, or 95% identical to any one of SEQ ID NO: 6 to SEQ ID NO: 10. In some embodiments, the mRNA comprises a coding sequence at least 70% identical to any one of SEQ ID NO: 6 to SEQ ID NO: 10. In some embodiments, the mRNA comprises a coding sequence at least 80% identical to any one of SEQ ID NO: 6 to SEQ ID NO: 10. In some embodiments, the mRNA comprises a coding sequence at least 90% identical to any one of SEQ ID NO: 6 to SEQ ID NO: 10. In some embodiments, the mRNA comprises a coding sequence at least 95% identical to any one of SEQ ID NO: 6 to SEQ ID NO: 10. In some embodiments, the mRNA comprises a coding sequence at least 96%, at least 97%, at least 98%, or at least 99% identical to SEQ ID NO: 6 to SEQ ID NO: 10. In some embodiments, the mRNA comprises a coding sequence set forth in SEQ ID NO: 6 to SEQ ID NO: 10.

In some embodiments, the mRNA comprises a coding sequence at least 70%, 75%, 80%, 85%, 90%, or 95% identical to SEQ ID NO: 6. In some embodiments, the mRNA comprises a coding sequence at least 70% identical to SEQ ID NO: 6. In some embodiments, the mRNA comprises a coding sequence at least 80% identical to SEQ ID NO: 6. In some embodiments, the mRNA comprises a coding sequence at least 90% identical to SEQ ID NO: 6. In some embodiments, the mRNA comprises a coding sequence at least 95% identical to SEQ ID NO: 6. In some embodiments, the mRNA comprises a coding sequence at least 96%, at least 97%, at least 98%, or at least 99% identical to SEQ ID NO: 6. In some embodiments, the mRNA comprises a coding sequence set forth in SEQ ID NO: 6.

In some embodiments, the mRNA comprises a coding sequence at least 70%, 75%, 80%, 85%, 90%, or 95% identical to SEQ ID NO: 7. In some embodiments, the mRNA comprises a coding sequence at least 70% identical to SEQ ID NO: 7. In some embodiments, the mRNA comprises a coding sequence at least 80% identical to SEQ ID NO: 7. In some embodiments, the mRNA comprises a coding sequence at least 90% identical to SEQ ID NO: 7. In some embodiments, the mRNA comprises a coding sequence at least 95% identical to SEQ ID NO: 7. In some embodiments, the mRNA comprises a coding sequence at least 96%, at least 97%, at least 98%, or at least 99% identical to SEQ ID NO: 7. In some embodiments, the mRNA comprises a coding sequence set forth in SEQ ID NO: 7.

In some embodiments, the mRNA comprises a coding sequence at least 70%, 75%, 80%, 85%, 90%, or 95% identical to SEQ ID NO: 8. In some embodiments, the mRNA comprises a coding sequence at least 70% identical to SEQ ID NO: 8. In some embodiments, the mRNA comprises a coding sequence at least 80% identical to SEQ ID NO: 8. In some embodiments, the mRNA comprises a coding sequence at least 90% identical to SEQ ID NO: 8. In some embodiments, the mRNA comprises a coding sequence at least 95% identical to SEQ ID NO: 8. In some embodiments, the mRNA comprises a coding sequence at least 96%, at least 97%, at least 98%, or at least 99% identical to SEQ ID NO: 8. In some embodiments, the mRNA comprises a coding sequence set forth in SEQ ID NO: 8.

In some embodiments, the mRNA comprises a coding sequence at least 70%, 75%, 80%, 85%, 90%, or 95% identical to SEQ ID NO: 9. In some embodiments, the mRNA comprises a coding sequence at least 70% identical to SEQ ID NO: 9. In some embodiments, the mRNA comprises a coding sequence at least 80% identical to SEQ ID NO: 9. In some embodiments, the mRNA comprises a coding sequence at least 90% identical to SEQ ID NO: 9. In some embodiments, the mRNA comprises a coding sequence at least 95% identical to SEQ ID NO: 9. In some embodiments, the mRNA comprises a coding sequence at least 96%, at least 97%, at least 98%, or at least 99% identical to SEQ ID NO: 9. In some embodiments, the mRNA comprises a coding sequence set forth in SEQ ID NO: 9.

In some embodiments, the mRNA comprises a coding sequence at least 70%, 75%, 80%, 85%, 90%, or 95% identical to SEQ ID NO: 10. In some embodiments, the mRNA comprises a coding sequence at least 70% identical to SEQ ID NO: 10. In some embodiments, the mRNA comprises a coding sequence at least 80% identical to SEQ ID NO: 10. In some embodiments, the mRNA comprises a coding sequence at least 90% identical to SEQ ID NO: 10. In some embodiments, the mRNA comprises a coding sequence at least 95% identical to SEQ ID NO: 10. In some embodiments, the mRNA comprises a coding sequence at least 96%, at least 97%, at least 98%, or at least 99% identical to SEQ ID NO: 10. In some embodiments, the mRNA comprises a coding sequence set forth in SEQ ID NO: 10.

In some embodiments, administering the mRNA to the subject is performed by intratracheal, intranasal, intravenous, intramuscular or subcutaneous delivery.

In some embodiments, administering the mRNA to the subject is performed by intratracheal delivery. In some embodiments, administering the mRNA to the subject is performed by intranasal delivery. In some embodiments, administering the mRNA to the subject is performed by aerosol delivery. In some embodiments, administering the mRNA to the subject is performed by nebulized delivery. In some embodiments, administering the mRNA to the subject is performed by dry powder inhalation.

In some embodiments, the composition is administered once a week.

In some embodiments, the composition is administered once every two weeks.

In some embodiments, the composition is administered twice a month.

In some embodiments, the composition is administered once a month.

In some embodiments, the composition is administered at repeat intervals. In some embodiments, the repeat intervals occur every day, 3 days, 1 week, 2 weeks, 3 weeks, or four weeks. Accordingly, in some embodiments, the repeat intervals occur every day. In some embodiments, the repeat intervals occur every 3 days. In some embodiments, the repeat intervals occur every 1 week. In some embodiments, the repeat intervals occur every 2 weeks. In some embodiments, the repeat intervals occur every 3 weeks. In some embodiments, the repeat intervals occur every 4 weeks. In some embodiments, the repeat intervals occur every 5 weeks. In some embodiments, the repeat intervals occur every 6 weeks. In some embodiments, the repeat intervals occur every 7 weeks. In some embodiments, the repeat intervals occur every 8 weeks. In some embodiments, the repeat intervals occur every 9 weeks. In some embodiments, the repeat intervals occur every 10 weeks. In some embodiments, the repeat intervals occur every 11 weeks. In some embodiments, the repeat intervals occur every 12 weeks.

In some embodiments, the composition is administered daily for 5 consecutive days. In some embodiments, the administration of daily for 5 consecutive days is for at least 5 consecutive days. Accordingly, in some embodiments, the composition is administered for 6 consecutive days, for a week, for a month, or for a year.

In some embodiments, the composition is administered daily for 6 consecutive days. In some embodiments, the composition is administered daily for 7 consecutive days. In some embodiments, the composition is administered daily for 8 consecutive days. In some embodiments, the composition is administered daily for 9 consecutive days. In some embodiments, the composition is administered daily for 10 consecutive days. In some embodiments, the composition is administered daily for 11 consecutive days. In some embodiments, the composition is administered daily for 12 consecutive days. In some embodiments, the composition is administered daily for 13 consecutive days. In some embodiments, the composition is administered daily for 14 consecutive days. In some embodiments, the composition is administered daily for 15 consecutive days. In some embodiments, the composition is administered daily for a week. In some embodiments, the composition is administered daily for 2 weeks. In some embodiments, the composition is administered daily for 2 weeks. In some embodiments, the composition is administered daily for 3 weeks. In some embodiments, the composition is administered daily for 4 weeks. In some embodiments, the composition is administered daily for 5 weeks. In some embodiments, the composition is administered daily for 6 weeks. In some embodiments, the composition is administered daily for 7 weeks. In some embodiments, the composition is administered daily for 8 weeks. In some embodiments, the composition is administered daily for 10 weeks. In some embodiments, the composition is administered daily for 12 weeks. In some embodiments, the composition is administered daily for 15 weeks. In some embodiments, the composition is administered daily for a month. In some embodiments, the composition is administered daily for two months. In some embodiments, the composition is administered daily for three months. In some embodiments, the composition is administered daily for four months. In some embodiments, the composition is administered daily for five months. In some embodiments, the composition is administered daily for six months. In some embodiments, the composition is administered daily for seven months. In some embodiments, the composition is administered daily for eight months. In some embodiments, the composition is administered daily for nine months. In some embodiments, the composition is administered daily for ten months. In some embodiments, the composition is administered daily for twelve months. In some embodiments, the composition is administered daily for fifteen months. In some embodiments, the composition is administered daily for twenty months. In some embodiments, the composition is administered daily for a year. In some embodiments, the composition is administered daily for two years. In some embodiments, the composition is administered daily for three years. In some embodiments, the composition is administered daily for five years.

In some embodiments, the composition is administered weekly for 5 consecutive weeks. In some embodiments, the administration of weekly for 5 consecutive weeks is for at least 5 consecutive weeks. Accordingly, in some embodiments, the composition is administered for 6 consecutive weeks, for 10 consecutive weeks, for a month, for a year, or for 10 years or more.

In some embodiments, the composition is administered weekly for 2 weeks. In some embodiments, the composition is administered weekly for 3 weeks. In some embodiments, the composition is administered weekly for 4 weeks. In some embodiments, the composition is administered weekly for 5 weeks. In some embodiments, the composition is administered weekly for 6 weeks. In some embodiments, the composition is administered weekly for 7 weeks. In some embodiments, the composition is administered weekly for 8 weeks. In some embodiments, the composition is administered weekly for 10 weeks. In some embodiments, the composition is administered weekly for 12 weeks. In some embodiments, the composition is administered weekly for 15 weeks. In some embodiments, the composition is administered weekly for a month. In some embodiments, the composition is administered weekly for two months. In some embodiments, the composition is administered weekly for three months. In some embodiments, the composition is administered weekly for four months. In some embodiments, the composition is administered weekly for five months. In some embodiments, the composition is administered weekly for six months. In some embodiments, the composition is administered weekly for seven months. In some embodiments, the composition is administered weekly for eight months. In some embodiments, the composition is administered weekly for nine months. In some embodiments, the composition is administered weekly for ten months. In some embodiments, the composition is administered weekly for twelve months. In some embodiments, the composition is administered weekly for fifteen months. In some embodiments, the composition is administered weekly for twenty months. In some embodiments, the composition is administered weekly for a year. In some embodiments, the composition is administered weekly for two years. In some embodiments, the composition is administered weekly for three years. In some embodiments, the composition is administered weekly for five years. In some embodiments, the composition is administered weekly for ten years or more.

In some embodiments, the administering the mRNA results in DNAI1 protein expression detectable in one or more internal organs selected from lung, heart, liver, spleen, kidney, brain, stomach, intestines, ovary and testis.

In some embodiments, the administering the mRNA results in DNAI1 protein expression detectable in the lung.

In some embodiments, the administering the mRNA results in DNAI1 protein expression detectable in the lung epithelium.

In some embodiments, the DNAI1 protein expression is detectable throughout the length of cilia. In some embodiments, the DNAI1 mRNA is detectable throughout the length of cilia.

In some aspects, the invention provides a composition for use in the treatment of primary ciliary dyskinesia (PCD), the composition comprising a therapeutically effective amount of an mRNA encoding human axonemal dynein intermediate chain 1 (DNAI1) encapsulated in a liposome, wherein the liposome comprises one or more cationic lipids, one or more non-cationic lipids and one or more PEG-modified lipids.

In some embodiments, the mRNA comprises a DNAI1 coding sequence at least 70%, 75%, 80%, 85%, 90%, or 95% identical to any one of SEQ ID NO: 6 to SEQ ID NO: 10.

In some embodiments, the mRNA comprises a coding sequence set forth in SEQ ID NO: 6. In some embodiments, the mRNA comprises a coding sequence set forth in SEQ ID NO: 7. In some embodiments, the mRNA comprises a coding sequence set forth in SEQ ID NO: 8. In some embodiments, the mRNA comprises a coding sequence set forth in SEQ ID NO: 9. In some embodiments, the mRNA comprises a coding sequence set forth in SEQ ID NO: 10.

In some embodiments, the mRNA has a 5′-untranslated region (5′-UTR) that has a sequence set forth in SEQ ID NO: 2 or SEQ ID NO: 3, and a 3′-untranslated region (3′-UTR) that has a sequence set forth in SEQ ID NO: 4 or SEQ ID NO: 5.

In some embodiments, wherein the mRNA has one or more modified nucleotides.

In some embodiments, the invention provides a pharmaceutical composition comprising the composition described above and a suitable excipient.

In some aspects, the present invention provides a method of delivery of a mRNA encoding for a protein or peptide in vivo comprising administering to a subject in need of delivery a mRNA encoding a protein or peptide and having a 5′-untranslated region (5′-UTR) that has a sequence at least 70%, 75%, 80%, 85%, 90%, or 95% identical to SEQ ID NO: 2 and that is not SEQ ID NO: 3. In some embodiments, the mRNA comprises a 5′-untranslated region (5′-UTR) that has a sequence at least 70% identical to SEQ ID NO: 2 that is not SEQ ID NO: 3. In some embodiments, the mRNA comprises a 5′-untranslated region (5′-UTR) that has a sequence at least 75% identical to SEQ ID NO: 2 that is not SEQ ID NO: 3. In some embodiments, the mRNA comprises a 5′-untranslated region (5′-UTR) that has a sequence at least 80% identical to SEQ ID NO: 2 that is not SEQ ID NO: 3. In some embodiments, the mRNA comprises a 5′-untranslated region (5′-UTR) that has a sequence at least 85% identical to SEQ ID NO: 2 that is not SEQ ID NO: 3. In some embodiments, the mRNA comprises a 5′-untranslated region (5′-UTR) that has a sequence at least 90% identical to SEQ ID NO: 2 that is not SEQ ID NO: 3. In some embodiments, the mRNA comprises a 5′-untranslated region (5′-UTR) that has a sequence at least 95% identical to SEQ ID NO: 2 that is not SEQ ID NO: 3. In some embodiments, the mRNA comprises a 5′-untranslated region (5′-UTR) that is at least 96%, at least 97%, at least 98%, or at least 99% identical to SEQ ID NO: 2 that is not SEQ ID NO: 3. In some embodiments, the mRNA comprises a 5′-untranslated region (5′-UTR) set forth in SEQ ID NO: 2.

Other features, objects, and advantages of the present invention are apparent in the detailed description, drawings and claims that follow. It should be understood, however, that the detailed description, the drawings, and the claims, while indicating embodiments of the present invention, are given by way of illustration only, not limitation. Various changes and modifications within the scope of the invention will become apparent to those skilled in the art.

### BRIEF DESCRIPTION OF THE DRAWING

The drawings are for illustration purposes only, not for limitation.

FIG. 1 is an exemplary schematic of airway basal cell directed-differentiation timeline to generate a pseudo-stratified epithelium.

FIG. 2A is an exemplary immunofluorescence imaging of differentiated cells of pseudo-stratified epithelium, illustrating that Iso-wt cultures contain ciliated cells (ACT staining). FIG. 2B is an exemplary immunofluorescence imaging of differentiated cells of pseudo-stratified epithelium, illustrating that Iso-wt cultures contain distinct goblet cell population and MUC5AC.

FIG. 3A is an exemplary imaging of the differentiated epithelium in Iso-WT, disease model, and HBEC-ALI model cultures after staining with hematoxylin and eosin (H&E). Both Iso-WT and HBEC cultures contained proper cilia, whereas the disease model had reduced amount of cilia as well as shorter cilia. FIG. 3B is an exemplary bar graphs showing cilia length in ALI cultures of the disease line, Iso-wt model, or HBEC. Cilia in the disease model is statistically shorter than the cilia in the Iso-wt or HBEC models.

FIG. 4A is an exemplary bar graphs showing ciliary beat frequency (CBF) in ALI cultures of the untreated disease line, treated disease line, Iso-wt model, or HBEC model. CBF in the disease line is rescued to a normal level by administration of mRNA of the present invention.

FIG. 5A is an exemplary graph and a table showing the transfected amount of mRNA over time and a dose-dependent response. mRNAs were administered by LNP comprising either SY-3-E14-DMAPr or TL1-01D-DMA as a cationic lipid. FIG. 5B is an exemplary bar graph showing the % of ciliated cells that are DNAI1-positive by a single administration of DNAI1 mRNA encapsulated in LNPs comprising TL1-01D-DMA. FIG. 5C is an exemplary immunohistochemical (IHC) staining of airway epithelium post administration of DNAI1 mRNA. Arrows indicate exemplary stained sites illustrating expression of DNAI1 protein.

FIG. 6A is an exemplary density plot of TUBA+DNAI1+lung epithelial cells from Cd-1 mice. Flow cytometry results were from lungs of mice that were either untreated or treated via nebulization with test article MRT-DNAI1-LNP1 for six hours (group 2, N=4). Lungs were collected 48 hours post-exposure, enzymatically digested for single cells, and stained before flow cytometry analysis. TUBA+/DNAI1+ gates were based on untreated samples. Results are representative of group results for 6-hour nebulized treatment. FIG. 6B is an exemplary bar graph illustrating exposure-expression relationship in multi-ciliated cells for DNAI1 in CDT mice with MRT-DNAI1-LNP. Flow cytometry results of multi-ciliated (TUBA+) cells that were DNAI1-positive from animals that were exposed to nebulized test article MRT-DNAI1/LNP. Lungs were collected 48 hours post-exposure, enzymatically digested for single cells, and stained before flow cytometry analysis. TUBA+/DNAI1+ gates were based on untreated samples. FIG. 6C is an exemplary bar graph illustrating exposure-expression relationship assessing mRNA levels immediately after exposure. Right lungs from animals immediately post-exposure (all groups, N=4 per group) were analyzed for the presence of mRNA levels by RTqPCR. A standard curve was used to assess mRNA copies per mg lung tissue.

FIG. 7 is a series of exemplary bar graphs illustrating duration of expression for DNAI1 after a single 6-hours exposure. Flow cytometry results of multi-ciliated (TUBA+) cells that were DNAI1-positive from animals that were exposed to nebulized test article MRT-DNAI1-LNP1 or LNP2. Lungs were collected 4, 8, and 15 days post-exposure, enzymatically digested for single cells, and stained before flow cytometry analysis. TUBA+/DNAI1+ gates were based on paired untreated samples. Analysis with 1-way ANOVA *p<0.05, Fisher's LSD Test compared to untreated.

FIG. 8A is an exemplary flow cytometry density plots showing the population of cells that are both ciliated and DNAI1-positive. The population of cells that are both ciliated and DNAI1-positive are located on upper right corner of each panel. Representative flow cytometry density plots are from lungs of CD-1 mice untreated or treated with either MRT-DNAI1-LNP1 (n=4 per time point) or MRT-DNAI1-LNP2 (n=4 per time point). Animals evaluated at day 4 had a single exposure. Animals evaluated on day 8 had 5 daily exposures on days 1-5 (groups 2 and 3) and were evaluated 3 days after the last exposure. Animals evaluated on day 12 had 5 daily exposures on days 1-5 (groups 2 and 3) and were evaluated 7 days after the last exposure. FIG. 8B is an exemplary bar graph showing the % of ciliated cells that are DNAI1-positive by a repeated administration of DNAI1 mRNA encapsulated in LNPs comprising TL1-01D-DMA or SY-3-E14-DMAPr. Flow cytometry results of multi-ciliated (TUBA+) cells that were DNAI1-positive from animals that were exposed to nebulized test articles with a single dose (Day 4) or after five consecutive daily doses (Day 8 and Day 12). Lungs were collected, enzymatically digested for single cells, and stained before flow cytometry analysis. TUBA+/DNAI1+ gates were based on untreated samples. *p<0.05, Fisher's LSD Test compared to Untreated, 1-way ANOVA. FIG. 8C is an exemplary histogram representation of geometric mean fluorescence intensity of DNAI1 expression. Flow cytometry data measuring geometric mean fluorescence intensity (gMFI) of DNAI1 from the TUBA-positive/DNAI1-positive multi-ciliated epithelial cells. Blue plots represent the gMFI of the untreated cells, red plots represent day 4 (72 hours after a single-exposure), orange plots represent day 8 (3 days after a five-day exposure), and green plots represent day 12 (7 days after a five-day exposure). FIG. 8D is an exemplary bar graph showing the expression level of DNAI1 from multi-ciliated airway epithelium after a repeated administration of DNAI1 mRNA encapsulated in LNPs comprising TL1-01D-DMA or SY-3-E14-DMAPr. In this experiment, mice were nebulized with mRNA-LNPs daily for five consecutive days. Analysis with 1-way ANOVA *p<0.05, Fisher's LSD Test compared to untreated. FIG. 8E is an exemplary bar graph showing ciliary beat frequency (CBF) in untreated WT, untreated heterozygote, and treated knockout mice. CBF in the knockout mice was rescued to a normal level by administration of mRNA of the present invention.

FIG. 9A is an exemplary bar graph showing % of ciliated cells that are DNAI1-positive by administration of DNAI1 mRNA encapsulated in LNPs comprising TL1-01D-DMA or SY-3-E14-DMAPr over 15 days. Protein expression peaks at day 8 and is still present at day 15. Approximate half-life of DNAI1 protein is 15 days. FIG. 9B is an exemplary graph illustrating the % of ciliated and DNAI1-positive cells over time based on data modeling of weekly dosing assuming half-life of DNAI1 protein as two weeks. The predicted level of eMax is achieved at least 40% by 5 weeks regardless of the cationic lipids used.

FIG. 10A is an exemplary Western blot analysis of DNAI1 protein from lung homogenates after weekly intratracheal instillation of MRT-DNA1-LNP. Western blot results of DNAI1 expression detected in lung homogenates of treated CD-1 mice. Mice were dosed weekly for up to 6 weeks with either saline or MRT-DNAI1-LNP at 15 μg per animal. A cohort of animals from all groups were taken down 7 days after a dose (doses 1-5) and left lungs were analyzed for expression. The western blot antibody detects both human DNAI1 (exogenous) and mouse Dnaic1 (endogenous), with the human DNAI1 protein migrating slightly higher in the SDS-PAGE gel (green bands). DNAI1 band intensities are graphed as DNAI1 RFUs representing the day in which the mice were taken down, seven days after the last dose. Triangles below the x-axis represent the days animals were dosed. (A-C) Correspond to representative IHC results that are shown in FIG. 10B. FIG. 10B is a series of representative images of DNAI1 IHC performed on lungs of either saline-treated or MRT-DNAI1-LNP-treated animals via intratracheal instillation. Animals treated with test articles were dosed at 15 μg per animal on a weekly basis (Group 2). Animals were taken down weekly, seven days after their last dose (n=4 per time point). Images demonstrate staining in test article treated animals in the pseudostratified columnar epithelial cells of the airways on the apical surface facing the airways. With increased number of doses, the signal intensity and the number of cells increases.

FIG. 11 is an exemplary bar graph illustrating ciliary beat frequency from mice that were untreated or treated with MRT-DNAI1-LNP. Tracheas from the mice were cleaned in situ to prevent any damage by cleaning ex vivo. Tracheas were accessed by generating a longitudinal cut and cilia were imaged using a 40× water dipping objective by acquiring a 5 second video at 500 frames per second. Statistical analysis was performed using One-way ANOVA/Tukey's multiple comparison test.

### DEFINITIONS

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

Alkyl: As used herein, “alkyl” refers to a radical of a straight-chain or branched saturated hydrocarbon group having from 1 to 15 carbon atoms (“C1-15 alkyl”). In some embodiments, an alkyl group has 1 to 3 carbon atoms (“C1-3 alkyl”). Examples of C1-3 alkyl groups include methyl (C1), ethyl (C2), n-propyl (C3), and isopropyl (C3). In some embodiments, an alkyl group has 8 to 12 carbon atoms (“C8-12 alkyl”). Examples of C8-12 alkyl groups include, without limitation, n-octyl (C8), n-nonyl (C9), n-decyl (C10), n-undecyl (C11), n-dodecyl (C12) and the like. The prefix “n-” (normal) refers to unbranched alkyl groups. For example, n-C8 alkyl refers to (CH2)7CH3, n-C10 alkyl refers to (CH2)9CH3, etc.

Amino acid: As used herein, term “amino acid,” in its broadest sense, refers to any compound and/or substance that can be incorporated into a polypeptide chain. In some embodiments, an amino acid has the general structure H2N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an 1-amino acid. “Standard amino acid” refers to any of the twenty standard 1-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Typically, the term “approximately” or “about” refers to a range of values that within 10%, or more typically 1%, of the stated reference value.

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active.

Codon-optimized: As used herein, the term describes a nucleic acid in which one or more of the nucleotides present in a naturally occurring nucleic acid sequence (also referred to as ‘wild-type’sequence) has been substituted with an alternative nucleotide to optimize protein expression without changing the amino acid sequence of the polypeptide encoded by the naturally occurring nucleic acid sequence. For example, the codon AAA may be altered to become AAG without changing the identity of the encoded amino acid (lysine). In some embodiments, the nucleic acids of the invention are codon optimized to increase protein expression of the protein encoded by the nucleic acid. For the purpose of this application, nucleobase thymidine (T) and uracil (U) are used interchangeably in narration of mRNA sequences.

Delivery: As used herein, the term “delivery” encompasses both local and systemic delivery. For example, delivery of mRNA encompasses situations in which an mRNA is delivered to a target tissue and the encoded protein is expressed and retained within the target tissue (also referred to as “local distribution” or “local delivery”), and situations in which an mRNA is delivered to a target tissue and the encoded protein is expressed and secreted into patient's circulation system (e.g., serum) and systematically distributed and taken up by other tissues (also referred to as “systemic distribution” or “systemic delivery).

Dosing interval: As used herein dosing interval in the context of a method for treating a disease is the frequency of administering a therapeutic composition in a subject (mammal) in need thereof, for example an mRNA composition, at an effective dose of the mRNA, such that one or more symptoms associated with the disease is reduced; or one or more biomarkers associated with the disease is reduced, at least for the period of the dosing interval. Dosing frequency and dosing interval may be used interchangeably in the current disclosure.

Expression: As used herein, “expression” of a nucleic acid sequence refers to translation of an mRNA into a polypeptide, assemble multiple polypeptides into an intact protein (e.g., enzyme) and/or post-translational modification of a polypeptide or fully assembled protein (e.g., enzyme). In this application, the terms “expression” and “production,” and grammatical equivalent, are used inter-changeably.

Effective dose: As used herein, an effective dose is a dose of the mRNA in the pharmaceutical composition which when administered to the subject in need thereof, hereby a mammalian subject, according to the methods of the invention, is effective to bring about an expected outcome in the subject, for example reduce a symptom associated with the disease.

Functional: As used herein, a “functional” biological molecule is a biological molecule in a form in which it exhibits a property and/or activity by which it is characterized.

Half-life: As used herein, the term “half-life” is the time required for a quantity such as nucleic acid or protein concentration or activity to fall to half of its value as measured at the beginning of a time period.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. As used herein, calculation of percent purity of isolated substances and/or entities should not include excipients (e.g., buffer, solvent, water, etc.).

Local distribution or delivery: As used herein, the terms “local distribution,” “local delivery,” or grammatical equivalent, refer to tissue specific delivery or distribution. Typically, local distribution or delivery requires a protein (e.g., enzyme) encoded by mRNAs be translated and expressed intracellularly or with limited secretion that avoids entering the patient's circulation system.

messenger RNA (mRNA): As used herein, the term “messenger RNA (mRNA)” refers to a polyribonucleotide that encodes at least one polypeptide. mRNA may contain one or more coding and non-coding regions. mRNA can be purified from natural sources, produced using recombinant expression systems and optionally purified, in vitro transcribed, chemically synthesized, etc. An mRNA sequence is presented in the 5′ to 3′ direction unless otherwise indicated. Typically, the mRNA of the present invention is synthesized from adenosine, guanosine, cytidine and uridine nucleotides that bear no modifications. Such mRNA is referred to herein as mRNA with unmodified nucleotides or ‘unmodified mRNA’ for short. Typically, this means that the mRNA of the present invention does not comprise any of the following nucleoside analogs: 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine. An mRNA suitable for practising the claimed invention commonly does not comprise nucleosides comprising chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

NIP Ratio: As used herein, the term “N/P ratio” refers to a molar ratio of positively charged molecular units in the cationic lipids in a lipid nanoparticle relative to negatively charged molecular units in the mRNA encapsulated within that lipid nanoparticle. As such, N/P ratio is typically calculated as the ratio of moles of amine groups in cationic lipids in a lipid nanoparticle relative to moles of phosphate groups in mRNA encapsulated within that lipid nanoparticle.

Nucleic acid: As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA.

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre- and post-natal forms.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Pharmaceutically acceptable salt: Pharmaceutically acceptable salts are well known in the art. For example, S. M. Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N+(C1-4 alkyl)4 salts. Representative alkali or alkaline earth metal salts include sodium, lithium, potassium, calcium, magnesium, and the like. Further pharmaceutically acceptable salts include, when appropriate, nontoxic ammonium. quaternary ammonium, and amine cations formed using counterions such as halide, hydroxide, carboxylate, sulfate, phosphate, nitrate, sulfonate and aryl sulfonate. Further pharmaceutically acceptable salts include salts formed from the quaternization of an amine using an appropriate electrophile, e.g., an alkyl halide, to form a quarternized alkylated amino salt.

Systemic distribution or delivery: As used herein, the terms “systemic distribution,” “systemic delivery,” or grammatical equivalent, refer to a delivery or distribution mechanism or approach that affect the entire body or an entire organism. Typically, systemic distribution or delivery is accomplished via body's circulation system, e.g., blood stream. Compared to the definition of “local distribution or delivery.”

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Target tissues: As used herein, the term “target tissues” refers to any tissue that is affected by a disease to be treated. In some embodiments, target tissues include those tissues that display disease-associated pathology, symptom, or feature.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

Various aspects of the invention are described in detail in the following sections. The use of sections is not meant to limit the invention. Each section can apply to any aspect of the invention. In this application, the use of “or” means “and/or” unless stated otherwise.

### DETAILED DESCRIPTION

### Primary Ciliary Dyskinesia (PCD)

Primary ciliary dyskinesia (PCD) is an autosomal recessive disorder characterized by abnormal cilia and flagella that are found in the linings of the airway, the reproductive system, and other organs and tissues. The main ciliary defect found in PCD is an absence of dynein arms, affecting almost all cilia. Ciliary dysfunction prevents the clearance of mucous from the lungs, paranasal sinuses and middle ears. Bacteria and other irritants in the mucous lead to frequent respiratory infections.

Dyneins consist of a large family of proteins involved in many types of microtubule-dependent cell motility in both lower and higher eukaryotes. Two major classes of dyneins have been described: cytoplasmic and axonemal dyneins. Cytoplasmic dyneins are involved in a wide range of intracellular functions, including chromosome movements on the mitotic spindle and the transport of membranous organelles toward the minus ends of microtubules. Axonemal dyneins are found in ciliary and flagellar axonemes. Two dynein arms—outer and inner—are bound to each peripheral microtubule doublet; these dynein arms are essential for ciliary and flagellar beating, which they generate through ATP-dependent cycles of attachment/detachment to the adjacent microtubule doublet.

Among several genes confirmed to be directly involved in PCD pathogenesis, a significant number of mutations were found in two genes: DNAI1 and DNAH5, encoding intermediate and heavy chains of the axonemal dynein, respectively. Mutations in other genes, coding for proteins involved in the axonemal ultrastructure (DNAH11, DNAI2, TXNDC3, RSPH9, RSPH4A) or assembly (KTU, CRRC50), also have been reported, as well as mutations in the RPGR gene, in some cases of PCD. Mutations in DNAI1 and DNAH5, both associated with a ciliary outer dynein arm (ODA) defect phenotype, are combined estimated to account for almost 40% of PCD cases.

Mutations in the DNAI1 gene result in an absent or abnormal dynein axonemal intermediate chain 1, which is required for the proper functioning of cilia. Without a normal version of DNAI1, defective cilia cannot produce the force and movement needed to eliminate fluid, bacteria, and particles from the lungs, to establish the left-right axis during embryonic development, and to propel the sperm cells. PCD can lead to chronic respiratory tract infections, bronchiectasis, year-round nasal congestion, abnormally placed organs within their chest and abdomen, and infertility.

Polyribonucleotides of the disclosure can be used, for example, to treat a subject having or at risk of having primary ciliary dyskinesia or any other condition associated with a defect or malfunction of a gene whose function is linked to cilia maintenance and function. Non limiting examples of genes that have been associated with primary ciliary dyskinesia include: armadillo repeat containing 4 (ARMC4), chromosome 21 open reading frame 59 (C21orf59), coiled-coil domain containing 103 (CCDC103), coiled-coil domain containing 114 (CCDC114), coiled-coil domain containing 39 (CCDC39), coiled-coil domain containing 40 (CCDC40), coiled-coil domain containing 65 (CCDC65), cyclin 0 (CCNO), dynein (axonemal) assembly factor 1 (DNAAF1), dynein (axonemal) assembly factor 2 (DNAAF2), dynein (axonemal) assembly factor 3 (DNAAF3), dynein (axonemal) assembly factor 5 (DNAAF5), dynein axonemal heavy chain 11 (DNAHI 1), dynein axonemal heavy chain 5 (DNAH5), dynein axonemal heavy chain 6 (DNAH6), dynein axonemal heavy chain 8 (DNAH8), dynein axonemal intermediate chain 1 (DNAI1), dynein axonemal intermediate chain 2 (DNAI2), dynein axonemal light chain 1 (DNAL1), dynein regulatory complex subunit 1 (DRC1), dyslexia susceptibility 1 candidate 1 (DYX1C1), growth arrest specific 8 (GAS8), axonemal central pair apparatus protein (HYDIN), leucine rich repeat containing 6 (LRRC6), ME/M23 family member 8 (NME8), oral-facial-digital syndrome 1 (OFD1), retinitis pigmentosa GTPase regulator (RPGR), radial spoke head 1 homolog (Chlamydomonas) (RSPH1), radial spoke head 4 homolog A (Chlamydomonas) (RSPH4A), radial spoke head 9 homolog (Chlamydomonas) (RSPH9), sperm associated antigen 1 (SPAG1), and zinc finger MY D-type containing 10 (ZMYND10).

### Treatment of PCD

According to the present invention, a DNAI1 mRNA is delivered to a PCD patient in need of treatment at a therapeutically effective dose and an administration interval for a treatment period sufficient to improve, stabilize or reduce one or more symptoms of PCD relative to a control. The terms “treat” or “treatment”, as used in the context of PCD herein, refers to amelioration of one or more symptoms associated with PCD, prevention or delay of the onset of one or more symptoms of PCD, and/or lessening of the severity or frequency of one or more symptoms of PCD. Particularly, the administration of the composition of the present invention by repeat dosing to the PCD patient results in improved ciliary function, as measured by ciliary beat frequency (CBF). Additionally, the methods of the present invention resulted in expression of the DNAI1 protein in airway epithelium at a level at least 10% of the wild-type level, an increase ciliary beat frequency (CBF), and DNAI1-positive transfection efficiency of greater than 5% in ciliated cells.

In some embodiments, a suitable administration interval of the treatment is daily, twice a week, weekly, once every two weeks, once every three weeks, once every four weeks, monthly, once every two months, once every three months, once every 6 months, yearly, once every two years, or once every five years. Typically, daily or weekly administration of a therapeutically effective dose of a DNAI1 mRNA in accordance with the invention is sufficient to effectively reduce the severity of one or more symptoms in a PCD patient. For example, a nominal dose of 1-36 mg of a DNAI1 mRNA (e.g., a nominal dose of 6-30 mg, e.g., 8 mg, 16 mg, 20 mg or 24 mg) administered daily or weekly by nebulization is effective in providing the subject with a at least 3% DNAI1-positive transfection efficiency in ciliated cells, expression of the DNAI1 protein in airway epithelium at a level at least 10% of the wild-type level, or increase in CBF from a control or baseline CBF.

In some embodiments, the duration of nebulization is at least 10 minute, 20 minutes, 30 minutes, 40 minutes, 50 minutes, 60 minutes, at least 65 minutes, at least 70 minutes, at least 75 minutes, at least 80 minutes, at least 85 minutes, at least 90 minutes, at least 95 minutes, at least 100 minutes, at least 105 minutes, at least 110 minutes, at least 115 minutes, or at least 120 minutes. For example, the duration of nebulization may be between 45 minutes and 135 minutes, between 65 minutes and 115 minutes, or between 70 minutes and 90 minutes.

In some embodiments, the present invention provides a method of treating primary ciliary dyskinesia (PCD) comprising administering to a subject in need of treatment an mRNA encoding a dynein axonemal intermediate chain 1 (DNAI1) protein, wherein the mRNA is administered at a dose ranging from 1 mg to 36 mg daily for at least 5 consecutive days. In some embodiments, mRNA is administered weekly. In a suitable composition, the DNAI1 mRNA is encapsulated in a liposome.

In some embodiments, the treatment period or how long the patient is administered a therapeutically effective dose of a DNAI1mRNA is for the life of the patient. In some embodiments, a suitable treatment period is at least two weeks, three weeks, four weeks, a month, two months, three months, four months, five months, six months, seven months, eight months, nine months, ten months, eleven months, twelve months, 1 year, 2 years, 3 years, 4 years, 5 years, 10 years, 20 year, 30 years or 50 years.

Typically, the therapeutic effect of administration of a DNAI1 mRNA on a PCD patient is measured relative to a control. In some embodiments, a control is the severity of one or more symptoms in the same patient before the treatment. In some embodiments, a control is indicative of a historical reference level of one or more symptoms in PCD patients. In some embodiments, a control is indicative of a normal level of ability, physical conditions or biomarker corresponding to the one or more symptoms being measured. In some embodiments, a control is ciliary beat frequency (CBF). In some embodiments, a control is a transfection efficiency of cells that are both ciliated and DNAI1-positive.

In some embodiments, the therapeutic effect of administration of a composition comprising an mRNA encoding DNAI1 protein to a subject by nebulization at an effective dose is measured by an increase in CBF. Accordingly, a suitable dose for use in the methods of the invention is selected on the basis that it provides the human subject with at least a 3% increase in CBF from baseline CBF at two days following the administration. In some embodiments, the dose is selected to provide the human subject with at least a 10% increase in CBF from baseline CBF at two days following the administration.

An additional or alternative consideration is selecting a dose for use in the method of the invention is whether it provides an increase in CBF from baseline CBF at one week following the administration. In some embodiments, the dose is selected to provide the human subject with at least a 2% increase in CBF from baseline CBF at one week following the administration. For instance, the dose may be selected to provide the human subject with at least a 7% increase in CBF from baseline CBF through one week following administration. In some embodiments, the dose is selected to provide the human subject with at least an 8% increase in CBF from baseline CBF at one week following the administration. In a specific embodiment, the dose is selected to provide the human subject with at least a 12% increase in CBF from baseline CBF through one week following administration.

In some embodiments, the mRNA is administered at a dose ranging from 0.01 mg to 100 mg. In some embodiments, the mRNA is administered at a dose ranging from 0.1 mg to 50 mg. In some embodiments, the mRNA is administered at a dose ranging from 1 mg to 100 mg. In some embodiments, the mRNA is administered at a dose ranging from 0.05 mg to 0.1 mg. In some embodiments, the mRNA is administered at a dose ranging from 0.5 mg to 1 mg. In some embodiments, the mRNA is administered at a dose ranging from 5 mg to 10 mg. In some embodiments, the mRNA is administered at a dose ranging from 0.05 mg to 50 mg. In some embodiments, the mRNA is administered at a dose ranging from 0.5 mg to 50 mg. In some embodiments, the mRNA is administered at a dose ranging from 5 mg to 50 mg. In some embodiments, the mRNA is administered at a dose ranging from 0.01 mg to 50 mg. In some embodiments, the mRNA is administered at a dose ranging from 1 mg to 50 mg. In some embodiments, the mRNA is administered at a dose ranging from 1.5 mg to 40 mg. In some embodiments, the mRNA is administered at a dose ranging from 2 mg to 36 mg. In some embodiments, the mRNA is administered at a dose ranging from 5 mg to 30 mg. In some embodiments, the mRNA is administered at a dose ranging from 10 mg to 20 mg. In some embodiments, the mRNA is administered at a dose ranging from 6 mg to 24 mg. In some embodiments, the mRNA is administered at a dose ranging from 8 mg to 16 mg. In some embodiments, the mRNA is administered at a dose of 0.5 mg. In some embodiments, the mRNA is administered at a dose of 0.01 mg. In some embodiments, the mRNA is administered at a dose of 1 mg. In some embodiments, the mRNA is administered at a dose of 0.1 mg. In some embodiments, the mRNA is administered at a dose of 0.5 mg. In some embodiments, the mRNA is administered at a dose of 1 mg. In some embodiments, the mRNA is administered at a dose of 0.05 mg. In some embodiments, the mRNA is administered at a dose of 1 mg. In some embodiments, the mRNA is administered at a dose of 1.5 mg. In some embodiments, the mRNA is administered at a dose of 2 mg. In some embodiments, the mRNA is administered at a dose of 2.5 mg. In some embodiments, the mRNA is administered at a dose of 3 mg. In some embodiments, the mRNA is administered at a dose of 4 mg. In some embodiments, the mRNA is administered at a dose of 5 mg. In some embodiments, the mRNA is administered at a dose of 6 mg. In some embodiments, the mRNA is administered at a dose of 7 mg. In some embodiments, the mRNA is administered at a dose of 8 mg. In some embodiments, the mRNA is administered at a dose of 9 mg. In some embodiments, the mRNA is administered at a dose of 10 mg. In some embodiments, the mRNA is administered at a dose of 11 mg. In some embodiments, the mRNA is administered at a dose of 12 mg. In some embodiments, the mRNA is administered at a dose of 13 mg. In some embodiments, the mRNA is administered at a dose of 14 mg. In some embodiments, the mRNA is administered at a dose of 15 mg. In some embodiments, the mRNA is administered at a dose of 16 mg. In some embodiments, the mRNA is administered at a dose of 17 mg. In some embodiments, the mRNA is administered at a dose of 18 mg. In some embodiments, the mRNA is administered at a dose of 20 mg. In some embodiments, the mRNA is administered at a dose of 21 mg. In some embodiments, the mRNA is administered at a dose of 22 mg. In some embodiments, the mRNA is administered at a dose of 23 mg. In some embodiments, the mRNA is administered at a dose of 24 mg. In some embodiments, the mRNA is administered at a dose of 25 mg. In some embodiments, the mRNA is administered at a dose of 26 mg. In some embodiments, the mRNA is administered at a dose of 27 mg. In some embodiments, the mRNA is administered at a dose of 28 mg. In some embodiments, the mRNA is administered at a dose of 29 mg. In some embodiments, the mRNA is administered at a dose of 30 mg. In some embodiments, the mRNA is administered at a dose of 31 mg. In some embodiments, the mRNA is administered at a dose of 32 mg. In some embodiments, the mRNA is administered at a dose of 33 mg. In some embodiments, the mRNA is administered at a dose of 34 mg. In some embodiments, the mRNA is administered at a dose of 35 mg. In some embodiments, the mRNA is administered at a dose of 36 mg. In some embodiments, the mRNA is administered at a dose of 37 mg. In some embodiments, the mRNA is administered at a dose of 38 mg. In some embodiments, the mRNA is administered at a dose of 39 mg. In some embodiments, the mRNA is administered at a dose of 40 mg.

In some embodiments, the mRNA is administered for 1 day. In some embodiments, the mRNA is administered for 2 days. In some embodiments, the mRNA is administered for 3 days. In some embodiments, the mRNA is administered for 4 days. In some embodiments, the mRNA is administered for more than 1 day. In some embodiments, the mRNA is administered for more than 2 days. In some embodiments, the mRNA is administered for more than 3 days. In some embodiments, the mRNA is administered for more than 4 days. In some embodiments, the administration for 5 consecutive days is for at least for 5 consecutive days. In some embodiments, the mRNA is administered for 5 days. In some embodiments, the mRNA is administered for 6 days. In some embodiments, the mRNA is administered for 7 days. In some embodiments, the mRNA is administered for 8 days. In some embodiments, the mRNA is administered for 9 days. In some embodiments, the mRNA is administered for 10 days. In some embodiments, the mRNA is administered for 12 days. In some embodiments, the mRNA is administered for 14 days. In some embodiments, the mRNA is administered for 15 days. In some embodiments, the mRNA is administered for 16 days. In some embodiments, the mRNA is administered for 17 days. In some embodiments, the mRNA is administered for 18 days. In some embodiments, the mRNA is administered for 20 days. In some embodiments, the mRNA is administered for a week. In some embodiments, the mRNA is administered for two weeks. In some embodiments, the mRNA is administered for three weeks. In some embodiments, the mRNA is administered for four weeks. In some embodiments, the mRNA is administered for five weeks. In some embodiments, the mRNA is administered for six weeks. In some embodiments, the mRNA is administered for seven weeks. In some embodiments, the mRNA is administered for eight weeks. In some embodiments, the mRNA is administered for ten weeks. In some embodiments, the mRNA is administered for a month. In some embodiments, the mRNA is administered for two months. In some embodiments, the mRNA is administered for three months. In some embodiments, the mRNA is administered for four months. In some embodiments, the mRNA is administered for five months. In some embodiments, the mRNA is administered for six months. In some embodiments, the mRNA is administered for seven months. In some embodiments, the mRNA is administered for eight months. In some embodiments, the mRNA is administered for nine months. In some embodiments, the mRNA is administered for ten months. In some embodiments, the mRNA is administered for eleven months. In some embodiments, the mRNA is administered for twelve months. In some embodiments, the mRNA is administered for a year.

In some embodiments, the mRNA is administered for 1 day. In some embodiments, the mRNA is administered for 2 days. In some embodiments, the mRNA is administered for 3 days. In some embodiments, the mRNA is administered for 4 days. In some embodiments, the mRNA is administered for more than 1 day. In some embodiments, the mRNA is administered for more than 2 days. In some embodiments, the mRNA is administered for more than 3 days. In some embodiments, the mRNA is administered for more than 4 days. In some embodiments, the administration for 5 consecutive days is for at least for 5 consecutive days. In some embodiments, the mRNA is administered for 5 days. In some embodiments, the mRNA is administered for 6 days. In some embodiments, the mRNA is administered for 7 days. In some embodiments, the mRNA is administered for 8 days. In some embodiments, the mRNA is administered for 9 days. In some embodiments, the mRNA is administered for 10 days. In some embodiments, the mRNA is administered for 12 days. In some embodiments, the mRNA is administered for 14 days. In some embodiments, the mRNA is administered for 15 days. In some embodiments, the mRNA is administered for 16 days. In some embodiments, the mRNA is administered for 17 days. In some embodiments, the mRNA is administered for 18 days. In some embodiments, the mRNA is administered for 20 days. In some embodiments, the mRNA is administered for a week. In some embodiments, the mRNA is administered for two weeks. In some embodiments, the mRNA is administered for three weeks. In some embodiments, the mRNA is administered for four weeks. In some embodiments, the mRNA is administered for five weeks. In some embodiments, the mRNA is administered for six weeks. In some embodiments, the mRNA is administered for seven weeks. In some embodiments, the mRNA is administered for eight weeks. In some embodiments, the mRNA is administered for ten weeks. In some embodiments, the mRNA is administered for a month. In some embodiments, the mRNA is administered for two months. In some embodiments, the mRNA is administered for three months. In some embodiments, the mRNA is administered for four months. In some embodiments, the mRNA is administered for five months. In some embodiments, the mRNA is administered for six months. In some embodiments, the mRNA is administered for seven months. In some embodiments, the mRNA is administered for eight months. In some embodiments, the mRNA is administered for nine months. In some embodiments, the mRNA is administered for ten months. In some embodiments, the mRNA is administered for eleven months. In some embodiments, the mRNA is administered for twelve months. In some embodiments, the mRNA is administered for a year or more.

In some embodiments, the mRNA is administered at a dose ranging from 1 mg to 50 mg for at least 2 days. In some embodiments, the mRNA is administered at a dose ranging from 1.5 mg to 40 mg for at least 2 days. In some embodiments, the mRNA is administered at a dose ranging from 2 mg to 36 mg for at least 2 days. In some embodiments, the mRNA is administered at a dose ranging from 2 mg to 36 mg for at least 2 days. In some embodiments, the mRNA is administered at a dose ranging from 5 mg to 30 mg for at least 2 days. In some embodiments, the mRNA is administered at a dose ranging from 10 mg to 20 mg for at least 2 days. In some embodiments, the mRNA is administered at a dose ranging from 6 mg to 24 mg for at least 2 days. In some embodiments, the mRNA is administered at a dose ranging from 8 mg to 16 mg for at least 2 days.

In some embodiments, the mRNA is administered at a dose ranging from 1 mg to 50 mg for at least 3 days. In some embodiments, the mRNA is administered at a dose ranging from 1.5 mg to 40 mg for at least 3 days. In some embodiments, the mRNA is administered at a dose ranging from 2 mg to 36 mg for at least 3 days. In some embodiments, the mRNA is administered at a dose ranging from 2 mg to 36 mg for at least 3 days. In some embodiments, the mRNA is administered at a dose ranging from 5 mg to 30 mg for at least 3 days. In some embodiments, the mRNA is administered at a dose ranging from 10 mg to 20 mg for at least 3 days. In some embodiments, the mRNA is administered at a dose ranging from 6 mg to 24 mg for at least 3 days. In some embodiments, the mRNA is administered at a dose ranging from 8 mg to 16 mg for at least 3 days.

In some embodiments, the mRNA is administered at a dose ranging from 1 mg to 50 mg for at least 4 days. In some embodiments, the mRNA is administered at a dose ranging from 1.5 mg to 40 mg for at least 4 days. In some embodiments, the mRNA is administered at a dose ranging from 2 mg to 36 mg for at least 4 days. In some embodiments, the mRNA is administered at a dose ranging from 2 mg to 36 mg for at least 4 days. In some embodiments, the mRNA is administered at a dose ranging from 5 mg to 30 mg for at least 4 days. In some embodiments, the mRNA is administered at a dose ranging from 10 mg to 20 mg for at least 4 days. In some embodiments, the mRNA is administered at a dose ranging from 6 mg to 24 mg for at least 4 days. In some embodiments, the mRNA is administered at a dose ranging from 8 mg to 16 mg for at least 4 days.

In some embodiments, the mRNA is administered at a dose ranging from 1 mg to 50 mg for at least 5 days. In some embodiments, the mRNA is administered at a dose ranging from 1.5 mg to 40 mg for at least 5 days. In some embodiments, the mRNA is administered at a dose ranging from 2 mg to 36 mg for at least 5 days. In some embodiments, the mRNA is administered at a dose ranging from 2 mg to 36 mg for at least 5 days. In some embodiments, the mRNA is administered at a dose ranging from 5 mg to 30 mg for at least 5 days. In some embodiments, the mRNA is administered at a dose ranging from 10 mg to 20 mg for at least 5 days. In some embodiments, the mRNA is administered at a dose ranging from 6 mg to 24 mg for at least 5 days. In some embodiments, the mRNA is administered at a dose ranging from 8 mg to 16 mg for at least 5 days.

In some embodiments, the mRNA is administered at a dose ranging from 1 mg to 50 mg for at least 7 days. In some embodiments, the mRNA is administered at a dose ranging from 1.5 mg to 40 mg for at least 7 days. In some embodiments, the mRNA is administered at a dose ranging from 2 mg to 36 mg for at least 7 days. In some embodiments, the mRNA is administered at a dose ranging from 2 mg to 36 mg for at least 7 days. In some embodiments, the mRNA is administered at a dose ranging from 5 mg to 30 mg for at least 7 days. In some embodiments, the mRNA is administered at a dose ranging from 10 mg to 20 mg for at least 7 days. In some embodiments, the mRNA is administered at a dose ranging from 6 mg to 24 mg for at least 7 days. In some embodiments, the mRNA is administered at a dose ranging from 8 mg to 16 mg for at least 7 days.

In some embodiments, the mRNA is administered at a dose ranging from 1 mg to 50 mg for at least 10 days. In some embodiments, the mRNA is administered at a dose ranging from 1.5 mg to 40 mg for at least 10 days. In some embodiments, the mRNA is administered at a dose ranging from 2 mg to 36 mg for at least 10 days. In some embodiments, the mRNA is administered at a dose ranging from 2 mg to 36 mg for at least 10 days. In some embodiments, the mRNA is administered at a dose ranging from 5 mg to 30 mg for at least 10 days. In some embodiments, the mRNA is administered at a dose ranging from 10 mg to 20 mg for at least 10 days. In some embodiments, the mRNA is administered at a dose ranging from 6 mg to 24 mg for at least 10 days. In some embodiments, the mRNA is administered at a dose ranging from 8 mg to 16 mg for at least 10 days.

In some embodiments, the mRNA is administered at a dose ranging from 1 mg to 50 mg for at least 14 days. In some embodiments, the mRNA is administered at a dose ranging from 1.5 mg to 40 mg for at least 14 days. In some embodiments, the mRNA is administered at a dose ranging from 2 mg to 36 mg for at least 14 days. In some embodiments, the mRNA is administered at a dose ranging from 2 mg to 36 mg for at least 14 days. In some embodiments, the mRNA is administered at a dose ranging from 5 mg to 30 mg for at least 14 days. In some embodiments, the mRNA is administered at a dose ranging from 10 mg to 20 mg for at least 14 days. In some embodiments, the mRNA is administered at a dose ranging from 6 mg to 24 mg for at least 14 days. In some embodiments, the mRNA is administered at a dose ranging from 8 mg to 16 mg for at least 14 days.

### Dynein Axonemal Intermediate Chain 1 (DNAI1) Gene and Protein Sequence

In some embodiments, the present invention provides methods and compositions for delivering mRNA encoding to a subject for the treatment of PCD. A suitable DNAI1 mRNA encodes any full length, fragment or portion of a DNAI1 protein which can be substituted for naturally-occurring DNAI1 protein activity and/or reduce the intensity, severity, and/or frequency of one or more symptoms associated with PCD.

In some embodiments, a suitable mRNA sequence is an mRNA sequence encoding a human DNAI1 protein. The naturally-occurring human DNAI1 mRNA coding sequence and the corresponding amino acid sequence are shown in Table 1:

TABLE 1
Human DNAI1 Amino Acid Sequence

| | |
|---|---|
| Human | MIPASAKAPHKQPHKQSISIGRGTRKRDEDSGTEVGEGTDEWAQSKATVRP |
| DNAI1 | PDQLELTDAELKEEFTRILTANNPHAPQNIVRYSFKEGTYKPIGFVNQLAVH |
| Protein | YTQVGNLIPKDSDEGRRQHYRDELVAGSQESVKVISETGNLEEDEEPKELET |
| Sequence | EPGSQTDVPAAGAAEKVTEEELMTPKQPKERKLTNQFNFSERASQTYNNPV |
| | RDRECQTEPPPRTNFSATANQWEIYDAYVEELEKQEKTKEKEKAKTPVAKK |
| | SGKMAMRKLTSMESQTDDLIKLSQAAKIMERMVNQNTYDDIAQDFKYYDD |
| | AADEYRDQVGTLLPLWKFQNDKAKRLSVTALCWNPKYRDLFAVGYGSYD |
| | FMKQSRGMLLLYSLKNPSFPEYMFSSNSGVMCLDIHVDHPYLVAVGHYDG |
| | NVAIYNLKKPHSQPSFCSSAKSGKHSDPVWQVKWQKDDMDQNLNFFSVSS |
| | DGRIVSWTLVKRKLVHIDVIKLKVEGSTTEVPEGLQLHPVGCGTAFDFHKEI |
| | DYMFLVGTEEGKIYKCSKSYSSQFLDTYDAHNMSVDTVSWNPYHTKVFMS |
| | CSSDWTVKIWDHTIKTPMFIYDLNSAVGDVAWAPYSSTVFAAVTTDGKAHI |
| | FDLAINKYEAICNQPVAAKKNRLTHVQFNLIHPIIIVGDDRGHIISLKLSPNLR |
| | KMPKEKKGQEVQKGPAVEIAKLDKLLNLVREVKIKT (SEQ ID NO: 1) |

Various mRNA sequences used in the Example section or useful for the present invention are shown in Table 2:

TABLE 2
Various DNAI1 Nucleic Acid Sequences

| | |
|---|---|
| DNAI1- | ATGATCCCCGCCTCCGCCAAAGCCCCTCACAAGCAACCGCACAAGCAAA |
| FLAG | GCATTAGCATTGGGCGGGGTACTCGGAAGCGCGACGAGGACTCGGGAA |
| (Human) | CTGAAGTCGGAGAGGGGACCGACGAATGGGCGCAGTCAAAGGCCACCG |
| | TGCGCCCACCGGACCAGCTCGAGCTGACCGATGCTGAGCTGAAGGAGGA |
| | GTTTACCCGGATCCTGACAGCCAACAACCCACATGCACCGCAGAACATC |
| | GTGCGGTACAGCTTCAAAGAGGGAACTTATAAGCCCATTGGCTTCGTGA |
| | ACCAACTCGCGGTGCATTACACCCAAGTCGGAAACCTTATTCCGAAGGA |
| | CTCGGACGAAGGCAGACGCCAGCACTACCGGGACGAGCTCGTGGCAGG |
| | ATCCCAGGAAAGCGTCAAGGTCATTTCCGAGACTGGCAACCTCGAGGAG |
| | GACGAAGAACCTAAGGAGCTGGAAACCGAACCCGGATCCCAGACCGAC |
| | GTGCCGGCCGCTGGGGCTGCCGAGAAAGTCACTGAAGAGGAACTCATGA |
| | CCCCGAAGCAGCCGAAAGAGAGAAAGCTCACCAACCAATTCAACTTCAG |
| | CGAGCGCGCCAGCCAAACCTACAACAACCCAGTCAGGGATCGGGAATGT |
| | CAGACCGAACCGCCTCCGAGAACGAACTTCTCGGCGACCGCGAACCAAT |
| | GGGAGATCTACGACGCCTACGTGGAAGAACTGGAAAAGCAGGAAAAGA |
| | CTAAGGAAAAGGAAAAGGCCAAGACTCCCGTCGCCAAGAAGTCGGGCA |
| | AAATGGCCATGCGGAAGCTCACCTCCATGGAATCACAGACTGACGACTT |
| | GATCAAGTTGAGCCAGGCCGCAAAGATCATGGAGCGCATGGTCAACCAA |
| | AATACTTACGACGATATCGCCCAAGACTTCAAGTACTACGACGACGCTG |
| | CCGATGAATACCGAGATCAAGTCGGCACCCTACTGCCGCTTTGGAAGTT |
| | CCAGAATGACAAGGCCAAGAGGCTGAGCGTGACCGCGCTGTGCTGGAA |
| | CCCCAAATACCGCGACCTCTTCGCCGTGGGATACGGCTCCTACGATTTCA |
| | TGAAGCAGAGCCGGGGAATGTTGCTCCTTTACTCCCTGAAGAACCCCTC |
| | CTTCCCTGAGTACATGTTCAGCTCAAACAGCGGCGTGATGTGCCTCGACA |
| | TTCACGTGGACCACCCTTACCTCGTGGCCGTGGGTCACTACGACGGCAA |
| | CGTCGCGATCTACAACTTGAAGAAGCCGCATTCACAGCCCTCGTTTTGCT |
| | CCTCGGCCAAGTCCGGCAAACATTCGGACCCAGTGTGGCAAGTCAAGTG |
| | GCAGAAAGATGACATGGACCAAAACTTGAACTTCTTCAGCGTGTCCTCC |
| | GACGGACGGATCGTGTCCTGGACCCTCGTGAAGCGGAAGTTGGTGCATA |
| | TCGACGTGATCAAATTGAAGGTCGAGGGTTCGACCACCGAAGTGCCTGA |
| | AGGCCTGCAGCTTCACCCCGTGGGATGCGGCACTGCCTTCGACTTCCACA |
| | AGGAGATCGACTACATGTTCCTCGTGGGAACCGAGGAAGGGAAGATCTA |
| | CAAATGCAGCAAGTCCTACTCATCACAATTCCTGGATACCTACGATGCCC |
| | ACAACATGAGCGTGGATACCGTGTCGTGGAACCCCTATCACACCAAGGT |
| | ATTCATGTCCTGCTCCTCCGACTGGACCGTCAAGATTTGGGACCACACCA |
| | TCAAGACCCCCATGTTCATCTACGACCTGAACTCCGCCGTGGGGGATGT |
| | GGCCTGGGCCCCCTACTCGTCGACCGTGTTTGCCGCGGTCACCACGGAC |
| | GGAAAGGCACACATTTTCGACCTTGCGATTAACAAATACGAGGCGATTT |
| | GCAACCAGCCCGTGGCCGCCAAAAAGAACCGCCTGACCCACGTTCAATT |
| | CAACTTAATCCACCCAATCATCATCGTCGGCGATGACAGAGGACACATT |
| | ATTAGCCTGAAACTTAGCCCCAACCTCCGCAAGATGCCCAAGGAGAAGA |
| | AGGGACAGGAAGTCCAGAAGGGCCCTGCCGTGGAGATTGCAAAGCTCG |
| | ATAAGCTCCTGAACTTAGTCCGGGAAGTGAAGATCAAGACTGGGGGCGG |
| | ATCCGACTATAAGGATGATGACGATAAGTAA (SEQ ID NO: 12) |
| DNAI1- | ATGATCCCCGCCTCCGCCAAAGCCCCTCACAAGCAACCGCACAAGCAAA |
| HA | GCATTAGCATTGGGCGGGGTACTCGGAAGCGCGACGAGGACTCGGGAA |
| (Human) | CTGAAGTCGGAGAGGGGACCGACGAATGGGCGCAGTCAAAGGCCACCG |
| | TGCGCCCACCGGACCAGCTCGAGCTGACCGATGCTGAGCTGAAGGAGGA |
| | GTTTACCCGGATCCTGACAGCCAACAACCCACATGCACCGCAGAACATC |
| | GTGCGGTACAGCTTCAAAGAGGGAACTTATAAGCCCATTGGCTTCGTGA |
| | ACCAACTCGCGGTGCATTACACCCAAGTCGGAAACCTTATTCCGAAGGA |
| | CTCGGACGAAGGCAGACGCCAGCACTACCGGGACGAGCTCGTGGCAGG |
| | ATCCCAGGAAAGCGTCAAGGTCATTTCCGAGACTGGCAACCTCGAGGAG |
| | GACGAAGAACCTAAGGAGCTGGAAACCGAACCCGGATCCCAGACCGAC |
| | GTGCCGGCCGCTGGGGCTGCCGAGAAAGTCACTGAAGAGGAACTCATGA |
| | CCCCGAAGCAGCCGAAAGAGAGAAAGCTCACCAACCAATTCAACTTCAG |
| | CGAGCGCGCCAGCCAAACCTACAACAACCCAGTCAGGGATCGGGAATGT |
| | CAGACCGAACCGCCTCCGAGAACGAACTTCTCGGCGACCGCGAACCAAT |
| | GGGAGATCTACGACGCCTACGTGGAAGAACTGGAAAAGCAGGAAAAGA |
| | CTAAGGAAAAGGAAAAGGCCAAGACTCCCGTCGCCAAGAAGTCGGGCA |
| | AAATGGCCATGCGGAAGCTCACCTCCATGGAATCACAGACTGACGACTT |
| | GATCAAGTTGAGCCAGGCCGCAAAGATCATGGAGCGCATGGTCAACCAA |
| | AATACTTACGACGATATCGCCCAAGACTTCAAGTACTACGACGACGCTG |
| | CCGATGAATACCGAGATCAAGTCGGCACCCTACTGCCGCTTTGGAAGTT |
| | CCAGAATGACAAGGCCAAGAGGCTGAGCGTGACCGCGCTGTGCTGGAA |
| | CCCCAAATACCGCGACCTCTTCGCCGTGGGATACGGCTCCTACGATTTCA |
| | TGAAGCAGAGCCGGGGAATGTTGCTCCTTTACTCCCTGAAGAACCCCTC |
| | CTTCCCTGAGTACATGTTCAGCTCAAACAGCGGCGTGATGTGCCTCGACA |
| | TTCACGTGGACCACCCTTACCTCGTGGCCGTGGGTCACTACGACGGCAA |
| | CGTCGCGATCTACAACTTGAAGAAGCCGCATTCACAGCCCTCGTTTTGCT |
| | CCTCGGCCAAGTCCGGCAAACATTCGGACCCAGTGTGGCAAGTCAAGTG |
| | GCAGAAAGATGACATGGACCAAAACTTGAACTTCTTCAGCGTGTCCTCC |
| | GACGGACGGATCGTGTCCTGGACCCTCGTGAAGCGGAAGTTGGTGCATA |
| | TCGACGTGATCAAATTGAAGGTCGAGGGTTCGACCACCGAAGTGCCTGA |
| | AGGCCTGCAGCTTCACCCCGTGGGATGCGGCACTGCCTTCGACTTCCACA |
| | AGGAGATCGACTACATGTTCCTCGTGGGAACCGAGGAAGGGAAGATCTA |
| | CAAATGCAGCAAGTCCTACTCATCACAATTCCTGGATACCTACGATGCCC |
| | ACAACATGAGCGTGGATACCGTGTCGTGGAACCCCTATCACACCAAGGT |
| | ATTCATGTCCTGCTCCTCCGACTGGACCGTCAAGATTTGGGACCACACCA |
| | TCAAGACCCCCATGTTCATCTACGACCTGAACTCCGCCGTGGGGGATGT |
| | GGCCTGGGCCCCCTACTCGTCGACCGTGTTTGCCGCGGTCACCACGGAC |
| | GGAAAGGCACACATTTTCGACCTTGCGATTAACAAATACGAGGCGATTT |
| | GCAACCAGCCCGTGGCCGCCAAAAAGAACCGCCTGACCCACGTTCAATT |
| | CAACTTAATCCACCCAATCATCATCGTCGGCGATGACAGAGGACACATT |
| | ATTAGCCTGAAACTTAGCCCCAACCTCCGCAAGATGCCCAAGGAGAAGA |
| | AGGGACAGGAAGTCCAGAAGGGCCCTGCCGTGGAGATTGCAAAGCTCG |
| | ATAAGCTCCTGAACTTAGTCCGGGAAGTGAAGATCAAGACTGGGGGCGG |
| | ATCCTACCCCTACGACGTGCCAGACTACGCATAA (SEQ ID NO: 13) |
| DNAI1- | ATGATCCCCGCCTAAGCCAAAGCCCCTCATAAGCAACCGCACAAGCAAA |
| stop | GCATAAGCTGAGGGCGGGGTACTCGGAAGCGTGACGAGGACTCGGGAA |
| (Human) | CTGAAGTAGGAGAGGGGACCGACGAATGGGCGCAGTCAAAGGCCACCG |
| | TGCGCCCACCGGACCAGCTCGAGCTGACCGATGCTGAGCTGAAGGAGGA |
| | GTTTACCCGGATCCTGACAGCCAACAACCCACATGCACCGCAGAACATC |
| | GTGCGGTACAGCTTCAAAGAGGGAACTTATAAGCCCATTGGCTTCGTGA |
| | ACCAACTCGCGGTGCATTACACCCAAGTCGGAAACCTTATTCCGAAGGA |
| | CTCGGACGAAGGCAGACGCCAGCACTACCGGGACGAGCTCGTGGCAGG |
| | ATCCCAGGAAAGCGTCAAGGTCATTTCCGAGACTGGCAACCTCGAGGAG |
| | GACGAAGAACCTAAGGAGCTGGAAACCGAACCCGGATCCCAGACCGAC |
| | GTGCCGGCCGCTGGGGCTGCCGAGAAAGTCACTGAAGAGGAACTCATGA |
| | CCCCGAAGCAGCCGAAAGAGAGAAAGCTCACCAACCAATTCAACTTCAG |
| | CGAGCGCGCCAGCCAAACCTACAACAACCCAGTCAGGGATCGGGAATGT |
| | CAGACCGAACCGCCTCCGAGAACGAACTTCTCGGCGACCGCGAACCAAT |
| | GGGAGATCTACGACGCCTACGTGGAAGAACTGGAAAAGCAGGAAAAGA |
| | CTAAGGAAAAGGAAAAGGCCAAGACTCCCGTCGCCAAGAAGTCGGGCA |
| | AAATGGCCATGCGGAAGCTCACCTCCATGGAATCACAGACTGACGACTT |
| | GATCAAGTTGAGCCAGGCCGCAAAGATCATGGAGCGCATGGTCAACCAA |
| | AATACTTACGACGATATCGCCCAAGACTTCAAGTACTACGACGACGCTG |
| | CCGATGAATACCGAGATCAAGTCGGCACCCTACTGCCGCTTTGGAAGTT |
| | CCAGAATGACAAGGCCAAGAGGCTGAGCGTGACCGCGCTGTGCTGGAA |
| | CCCCAAATACCGCGACCTCTTCGCCGTGGGATACGGCTCCTACGATTTCA |
| | TGAAGCAGAGCCGGGGAATGTTGCTCCTTTACTCCCTGAAGAACCCCTC |
| | CTTCCCTGAGTACATGTTCAGCTCAAACAGCGGCGTGATGTGCCTCGACA |
| | TTCACGTGGACCACCCTTACCTCGTGGCCGTGGGTCACTACGACGGCAA |
| | CGTCGCGATCTACAACTTGAAGAAGCCGCATTCACAGCCCTCGTTTTGCT |
| | CCTCGGCCAAGTCCGGCAAACATTCGGACCCAGTGTGGCAAGTCAAGTG |
| | GCAGAAAGATGACATGGACCAAAACTTGAACTTCTTCAGCGTGTCCTCC |
| | GACGGACGGATCGTGTCCTGGACCCTCGTGAAGCGGAAGTTGGTGCATA |
| | TCGACGTGATCAAATTGAAGGTCGAGGGTTCGACCACCGAAGTGCCTGA |
| | AGGCCTGCAGCTTCACCCCGTGGGATGCGGCACTGCCTTCGACTTCCACA |
| | AGGAGATCGACTACATGTTCCTCGTGGGAACCGAGGAAGGGAAGATCTA |
| | CAAATGCAGCAAGTCCTACTCATCACAATTCCTGGATACCTACGATGCCC |
| | ACAACATGAGCGTGGATACCGTGTCGTGGAACCCCTATCACACCAAGGT |
| | ATTCATGTCCTGCTCCTCCGACTGGACCGTCAAGATTTGGGACCACACCA |
| | TCAAGACCCCCATGTTCATCTACGACCTGAACTCCGCCGTGGGGGATGT |
| | GGCCTGGGCCCCCTACTCGTCGACCGTGTTTGCCGCGGTCACCACGGAC |
| | GGAAAGGCACACATTTTCGACCTTGCGATTAACAAATACGAGGCGATTT |
| | GCAACCAGCCCGTGGCCGCCAAAAAGAACCGCCTGACCCACGTTCAATT |
| | CAACTTAATCCACCCAATCATCATCGTCGGCGATGACAGAGGACACATT |
| | ATTAGCCTGAAACTTAGCCCCAACCTCCGCAAGATGCCCAAGGAGAAGA |
| | AGGGACAGGAAGTCCAGAAGGGCCCTGCCGTGGAGATTGCAAAGCTCG |
| | ATAAGCTCCTGAACTTAGTCCGGGAAGTGAAGATCAAGACTTAA (SEQ |
| | ID NO: 14) |
| Dnaic1 | ATGCCATCCAAACAAATTAGAAAGCAGAGCATTAGCGTGACCCGCGGAG |
| (Mouse) | CCCGGAGAAGAGATGAGGACTCGGGCACCGATGTCGGCGAAGGAACCG |
| | ACGAGTGGTCGCAGTCAAAGGCTACCGTGCGGCCGCCTGACCAGCTTGA |
| | ACTTACCGATGCCGAGCTGAAGGAAGAGTTCACTCGCATCCTGACCGCC |
| | AACAATCCCCACGCGCCGCAGAACATCGTGCGCTACTCCTTCAAAGAAG |
| | GAACGTACAAGCTCATTGGATTCGTGAACCAAATGGCCGTGCACTTTTCC |
| | CAAGTCGGAAACCTCATTCCGAAGGATTCCGACGAGGGCCGGCGGCAGC |
| | ATTACCGCGACGAAATGGTGGCCGGATCGCAAGAAAGCATCAAGGTCGT |
| | GACTTCCGAGGCCGAGAACCTCGAGGAAGAAGAAGAACCGAAAGAGGG |
| | CGAAGGAGAAGCCGAAGCCGAGGCTGAGGCCGGAAGCCAGACCGATAT |
| | CCCCGCTGCCGCAGAAACTACCGAAAAGGTCATCGAAGAGGAGTTGATG |
| | GCCCCGGTGCAGCCTAAGGAACGGAAGTTGACTAACCAATTCAACTTCA |
| | GCGAGAGAGCCTCACAGACCTTCAACAACCCACTGAGAGATAGGGAAT |
| | GTCAAATGGAGCCTCCGCCCCGGACGAACTTCTCGGCCACTGCAAACCA |
| | ATGGGAGATCTACGACGCTTACGTGGATGAACTGGAGAAGCAGGAAAA |
| | GACCAAGGAAAAGGAGAAAGCCAAGACCCCGGTCGCCAAAAAGACTGA |
| | GAAAATGGCCATGCGGAAGCTCACTTCCATGGAGAGCCAGTCCGACGAT |
| | ATTACTAAGGTTACCCAGGCCGCCAAGATCGTGGAAAGAATGGTGAACC |
| | AGAATACCTACGACGACGTGGCACAGGATTTTAAGTACTATGAGGACAC |
| | CGCTGACGAATACCGGGATCAAGAAGGCACCCTCCTGCCCTTGTGGAAG |
| | TTCCAGAATGACAAGGCCAAGAGGCTGGCCGTGACTGCGCTGTGCTGGA |
| | ACCCGAAGTATAAGGACCTCTTCGCCGTGGGTCATGGCTCATACGACTTT |
| | ATGAAGCAATCGCGCGGGATGCTCCTGCTCTACTCGATGAAGAACCCTT |
| | CCTTCCCCGAGTACATGTTCAGCTCCGAGTCAGGGATCATGTGCCTGGAT |
| | GTCCATGTCGACCACCCTTACCTCGTGGTCGTGGGATATTACGACGGCAA |
| | CGTGGCGATCTACAACCTTAAGAAACCGCATAGCCAGCCCTGCTTCCGG |
| | AGCACCTCAAAGAGCGGAAAGCACACCGACCCCGTGTGGCAAGTCAAG |
| | TGGCAAAAGGACGACATGGACCACAACTTGAACTTCTTCAGCGTGTCGT |
| | CCGACGGACGGATCGTGTCCTGGACCCTCGTCAAGTCCGAGCTCGTGCA |
| | CATTGACATTATCAAGCTCAAAACCGAGGGTTCCACCACTGAAATCCCC |
| | GAGGGCCTGCAGCTCCATACCGTGGGATGCGGCACCGCGTTCGACTTCC |
| | ATAAAGAGATCGACTACATGTTCCTCGTGGGGACTGAAGAGGGGAAGAT |
| | CTACAAGTGCAGCAAGTCGTACTCTTCCCAATTCCTGGACACCTACGACG |
| | CTCATAACATGGCAGTGGACGCCGTGCTGTGGAACCCATACCACACTAA |
| | GGTCTTTATGTCCTGCTCCTCGGACTGGACTGTGAAGATTTGGGACCACA |
| | CCATCAAAACCCCAATGTTCATTTACGACCTGAACTCCGCCGTGGGGGA |
| | TGTGGCCTGGGCCCCGTATTCCTCCACCGTATTCGCCGCCGTGACCACCG |
| | ACGGAAAGGCACACGTCTTTGACCTGGCCGTGAACAAATACGAGGCGAT |
| | TTGCAACCAGCCAGTGGTGGCCAAGAAGAAAAACAAGATCACTCACGTG |
| | CAATTCAACCCCATCCACCCCATTATTATCGTCGGCGACGATAGGGGTCA |
| | CATTATATGCCTGAAGCTCAGCCCGAACCTTCGCAAGATGCCTAAGGAA |
| | AAGAAGGGCCAGGAGGTGCAGAAGGGTCCTGCCGTCGAAATTGCGAAG |
| | TTGGACAAGCTCCTGAACCTCGTGCGCGAGGTCAAGACTAAGACCTAA |
| | (SEQ ID NO: 11) |
| Dnaic1- | ATGCCATCCAAACAAATTAGAAAGCAGAGCATTAGCGTGACCCGCGGAG |
| FLAG | CCCGGAGAAGAGATGAGGACTCGGGCACCGATGTCGGCGAAGGAACCG |
| (Mouse) | ACGAGTGGTCGCAGTCAAAGGCTACCGTGCGGCCGCCTGACCAGCTTGA |
| | ACTTACCGATGCCGAGCTGAAGGAAGAGTTCACTCGCATCCTGACCGCC |
| | AACAATCCCCACGCGCCGCAGAACATCGTGCGCTACTCCTTCAAAGAAG |
| | GAACGTACAAGCTCATTGGATTCGTGAACCAAATGGCCGTGCACTTTTCC |
| | CAAGTCGGAAACCTCATTCCGAAGGATTCCGACGAGGGCCGGCGGCAGC |
| | ATTACCGCGACGAAATGGTGGCCGGATCGCAAGAAAGCATCAAGGTCGT |
| | GACTTCCGAGGCCGAGAACCTCGAGGAAGAAGAAGAACCGAAAGAGGG |
| | CGAAGGAGAAGCCGAAGCCGAGGCTGAGGCCGGAAGCCAGACCGATAT |
| | CCCCGCTGCCGCAGAAACTACCGAAAAGGTCATCGAAGAGGAGTTGATG |
| | GCCCCGGTGCAGCCTAAGGAACGGAAGTTGACTAACCAATTCAACTTCA |
| | GCGAGAGAGCCTCACAGACCTTCAACAACCCACTGAGAGATAGGGAAT |
| | GTCAAATGGAGCCTCCGCCCCGGACGAACTTCTCGGCCACTGCAAACCA |
| | ATGGGAGATCTACGACGCTTACGTGGATGAACTGGAGAAGCAGGAAAA |
| | GACCAAGGAAAAGGAGAAAGCCAAGACCCCGGTCGCCAAAAAGACTGA |
| | GAAAATGGCCATGCGGAAGCTCACTTCCATGGAGAGCCAGTCCGACGAT |
| | ATTACTAAGGTTACCCAGGCCGCCAAGATCGTGGAAAGAATGGTGAACC |
| | AGAATACCTACGACGACGTGGCACAGGATTTTAAGTACTATGAGGACAC |
| | CGCTGACGAATACCGGGATCAAGAAGGCACCCTCCTGCCCTTGTGGAAG |
| | TTCCAGAATGACAAGGCCAAGAGGCTGGCCGTGACTGCGCTGTGCTGGA |
| | ACCCGAAGTATAAGGACCTCTTCGCCGTGGGTCATGGCTCATACGACTTT |
| | ATGAAGCAATCGCGCGGGATGCTCCTGCTCTACTCGATGAAGAACCCTT |
| | CCTTCCCCGAGTACATGTTCAGCTCCGAGTCAGGGATCATGTGCCTGGAT |
| | GTCCATGTCGACCACCCTTACCTCGTGGTCGTGGGATATTACGACGGCAA |
| | CGTGGCGATCTACAACCTTAAGAAACCGCATAGCCAGCCCTGCTTCCGG |
| | AGCACCTCAAAGAGCGGAAAGCACACCGACCCCGTGTGGCAAGTCAAG |
| | TGGCAAAAGGACGACATGGACCACAACTTGAACTTCTTCAGCGTGTCGT |
| | CCGACGGACGGATCGTGTCCTGGACCCTCGTCAAGTCCGAGCTCGTGCA |
| | CATTGACATTATCAAGCTCAAAACCGAGGGTTCCACCACTGAAATCCCC |
| | GAGGGCCTGCAGCTCCATACCGTGGGATGCGGCACCGCGTTCGACTTCC |
| | ATAAAGAGATCGACTACATGTTCCTCGTGGGGACTGAAGAGGGGAAGAT |
| | CTACAAGTGCAGCAAGTCGTACTCTTCCCAATTCCTGGACACCTACGACG |
| | CTCATAACATGGCAGTGGACGCCGTGCTGTGGAACCCATACCACACTAA |
| | GGTCTTTATGTCCTGCTCCTCGGACTGGACTGTGAAGATTTGGGACCACA |
| | CCATCAAAACCCCAATGTTCATTTACGACCTGAACTCCGCCGTGGGGGA |
| | TGTGGCCTGGGCCCCGTATTCCTCCACCGTATTCGCCGCCGTGACCACCG |
| | ACGGAAAGGCACACGTCTTTGACCTGGCCGTGAACAAATACGAGGCGAT |
| | TTGCAACCAGCCAGTGGTGGCCAAGAAGAAAAACAAGATCACTCACGTG |
| | CAATTCAACCCCATCCACCCCATTATTATCGTCGGCGACGATAGGGGTCA |
| | CATTATATGCCTGAAGCTCAGCCCGAACCTTCGCAAGATGCCTAAGGAA |
| | AAGAAGGGCCAGGAGGTGCAGAAGGGTCCTGCCGTCGAAATTGCGAAG |
| | TTGGACAAGCTCCTGAACCTCGTGCGCGAGGTCAAGACTAAGACCGGGG |
| | GTGGTTCCGACTACAAGGATGATGACGATAA (SEQ ID NO: 15) |
| Dnaic1- | ATGCCATCCAAACAAATTAGAAAGCAGAGCATTAGCGTGACCCGCGGAG |
| HA | CCCGGAGAAGAGATGAGGACTCGGGCACCGATGTCGGCGAAGGAACCG |
| (Mouse) | ACGAGTGGTCGCAGTCAAAGGCTACCGTGCGGCCGCCTGACCAGCTTGA |
| | ACTTACCGATGCCGAGCTGAAGGAAGAGTTCACTCGCATCCTGACCGCC |
| | AACAATCCCCACGCGCCGCAGAACATCGTGCGCTACTCCTTCAAAGAAG |
| | GAACGTACAAGCTCATTGGATTCGTGAACCAAATGGCCGTGCACTTTTCC |
| | CAAGTCGGAAACCTCATTCCGAAGGATTCCGACGAGGGCCGGCGGCAGC |
| | ATTACCGCGACGAAATGGTGGCCGGATCGCAAGAAAGCATCAAGGTCGT |
| | GACTTCCGAGGCCGAGAACCTCGAGGAAGAAGAAGAACCGAAAGAGGG |
| | CGAAGGAGAAGCCGAAGCCGAGGCTGAGGCCGGAAGCCAGACCGATAT |
| | CCCCGCTGCCGCAGAAACTACCGAAAAGGTCATCGAAGAGGAGTTGATG |
| | GCCCCGGTGCAGCCTAAGGAACGGAAGTTGACTAACCAATTCAACTTCA |
| | GCGAGAGAGCCTCACAGACCTTCAACAACCCACTGAGAGATAGGGAAT |
| | GTCAAATGGAGCCTCCGCCCCGGACGAACTTCTCGGCCACTGCAAACCA |
| | ATGGGAGATCTACGACGCTTACGTGGATGAACTGGAGAAGCAGGAAAA |
| | GACCAAGGAAAAGGAGAAAGCCAAGACCCCGGTCGCCAAAAAGACTGA |
| | GAAAATGGCCATGCGGAAGCTCACTTCCATGGAGAGCCAGTCCGACGAT |
| | ATTACTAAGGTTACCCAGGCCGCCAAGATCGTGGAAAGAATGGTGAACC |
| | AGAATACCTACGACGACGTGGCACAGGATTTTAAGTACTATGAGGACAC |
| | CGCTGACGAATACCGGGATCAAGAAGGCACCCTCCTGCCCTTGTGGAAG |
| | TTCCAGAATGACAAGGCCAAGAGGCTGGCCGTGACTGCGCTGTGCTGGA |
| | ACCCGAAGTATAAGGACCTCTTCGCCGTGGGTCATGGCTCATACGACTTT |
| | ATGAAGCAATCGCGCGGGATGCTCCTGCTCTACTCGATGAAGAACCCTT |
| | CCTTCCCCGAGTACATGTTCAGCTCCGAGTCAGGGATCATGTGCCTGGAT |
| | GTCCATGTCGACCACCCTTACCTCGTGGTCGTGGGATATTACGACGGCAA |
| | CGTGGCGATCTACAACCTTAAGAAACCGCATAGCCAGCCCTGCTTCCGG |
| | AGCACCTCAAAGAGCGGAAAGCACACCGACCCCGTGTGGCAAGTCAAG |
| | TGGCAAAAGGACGACATGGACCACAACTTGAACTTCTTCAGCGTGTCGT |
| | CCGACGGACGGATCGTGTCCTGGACCCTCGTCAAGTCCGAGCTCGTGCA |
| | CATTGACATTATCAAGCTCAAAACCGAGGGTTCCACCACTGAAATCCCC |
| | GAGGGCCTGCAGCTCCATACCGTGGGATGCGGCACCGCGTTCGACTTCC |
| | ATAAAGAGATCGACTACATGTTCCTCGTGGGGACTGAAGAGGGGAAGAT |
| | CTACAAGTGCAGCAAGTCGTACTCTTCCCAATTCCTGGACACCTACGACG |
| | CTCATAACATGGCAGTGGACGCCGTGCTGTGGAACCCATACCACACTAA |
| | GGTCTTTATGTCCTGCTCCTCGGACTGGACTGTGAAGATTTGGGACCACA |
| | CCATCAAAACCCCAATGTTCATTTACGACCTGAACTCCGCCGTGGGGGA |
| | TGTGGCCTGGGCCCCGTATTCCTCCACCGTATTCGCCGCCGTGACCACCG |
| | ACGGAAAGGCACACGTCTTTGACCTGGCCGTGAACAAATACGAGGCGAT |
| | TTGCAACCAGCCAGTGGTGGCCAAGAAGAAAAACAAGATCACTCACGTG |
| | CAATTCAACCCCATCCACCCCATTATTATCGTCGGCGACGATAGGGGTCA |
| | CATTATATGCCTGAAGCTCAGCCCGAACCTTCGCAAGATGCCTAAGGAA |
| | AAGAAGGGCCAGGAGGTGCAGAAGGGTCCTGCCGTCGAAATTGCGAAG |
| | TTGGACAAGCTCCTGAACCTCGTGCGCGAGGTCAAGACTAAGACCGGGG |
| | GTGGTTCCTACCCATACGATGTGCCTGACTACGCGTA (SEQ ID NO: 16) |

In some embodiments, a suitable mRNA is a wild-type human DNAI1 mRNA of sequence. In some embodiments, a suitable therapeutic candidate mRNA is a codon-optimized DNAI1 sequence that can encodes a DNAI1 amino acid sequence shown in Table 1 as SEQ ID NO: 1 or an amino acid sequence at least 70%, 75%, 80%, 85%, 90%, 95%, 96%, 97%, 98%, or 99% identical to SEQ ID NO: 1. In some embodiments, an mRNA according to the present invention encodes a DNAI1 protein with an amino acid sequence that is identical to SEQ ID NO: 1.

### Codon Optimization

According to an increasing amount of research, mRNAs contain numerous layers of information that overlap the amino acid code. Traditionally, codon optimization has been used to remove rare codons which were thought to be rate-limiting for protein expression. While fast growing bacteria and yeast both exhibit strong codon bias in highly expressed genes, higher eukaryotes exhibit much less codon bias, making it more difficult to discern codons that may be rate-limiting. In addition, it has been found that codon bias per se does not necessarily yield high expression but requires other features.

For example, rare codons have been implicated in slowing translation and forming pause sites, which may be required for correct protein folding. Therefore, variations in codon usage may provide a mechanism to fine-tune the temporal pattern of elongation and thus increase the time available for a protein to take on its correct confirmation. Codon optimization can interfere with this fine-tuning mechanism, resulting in less efficient protein translation or an increased amount of incorrectly folded proteins. Similarly, codon optimization may disrupt the normal patterns of cognate and wobble tRNA usage, thereby affecting protein structure and function because wobble-dependent slowing of elongation may likewise have been selected as a mechanism for achieving correct protein folding.

Various methods of performing codon optimization are known in the art, however, each has significant drawbacks and limitations from a computational and/or therapeutic point of view. In particular, known methods of codon optimization often involve, for each amino acid, replacing every codon with the codon having the highest usage for that amino acid, such that the “optimized” sequence contains only one codon encoding each amino acid (so may be referred to as a one-to-one sequence).

Despite these obstacles, the inventors have arrived at a codon-optimized hDNAI1 sequence that improves expression of the DNAI1 protein at least threefold over the coding sequence of the wild type gene. The increase in expression is not limited to cell cultures of mammalian cells but was also observed in vivo in a mouse model. It is expected that the observed improvement in expression of the codon-optimised DNAI1 coding sequence will result in an improved, more cost-effective mRNA replacement therapy for patients suffering from PCD, because it does not require the use of modified nucleotides for the preparation of the mRNA and allows treatment with a reduced dose and/or at extended dosing intervals.

In some embodiments, codon-optimized mRNA sequences according to the present invention were further codon-optimized by a new process: the process first generates a list of codon-optimized sequences and then applies three filters to the list. Specifically, it applies a motif screen filter, guanine-cytosine (GC) content analysis filter, and codon adaptation index (CAI) analysis filter to produce an updated list of optimized nucleotide sequences. The updated list no longer includes nucleotide sequences containing features that are expected to interfere with effective transcription and/or translation of the encoded protein antigen.

Exemplary Codon Optimized DNAI1 mRNA Sequences

The sequences that follow recite select, exemplary codon-optimized DNAI1 mRNA sequences.

In some embodiments, a suitable mRNA may be a codon-optimized sequence, as shown in SEQ ID NO: 6-10.

In some embodiments, a suitable mRNA sequence may be an mRNA sequence a homolog or an analog of human DNAI1 protein. For example, a homolog or an analog of human DNAI1 protein may be a modified human DNAI1 protein containing one or more amino acid substitutions, deletions, and/or insertions as compared to a wild-type or naturally-occurring human DNAI1 protein while retaining substantial DNAI1 protein activity. In some embodiments, an mRNA suitable for the present invention encodes an amino acid sequence at least 70%, 75%, 80%, 85%, 90%, 91%, 92%, 93%, 94%, 95%, 96%, 97%, 98%, 99% or more homologous to SEQ ID NO: 1. In some embodiments, an mRNA suitable for the present invention encodes a protein substantially identical to human DNAI1 protein. In some embodiments, an mRNA suitable for the present invention encodes an amino acid sequence at least 70%, 75%, 80%, 85%, 90%, 91%, 92%, 93%, 94%, 95%, 96%, 97%, 98%, 99% or more identical to SEQ ID NO: 1. Typically, an mRNA according to the present invention encodes a DNAI1 protein with an amino acid sequence that is identical to SEQ ID NO: 1.

In some embodiments, an mRNA suitable for the present invention encodes a fragment or a portion of human DNAI1 protein. In some embodiments, an mRNA suitable for the present invention encodes a fragment or a portion of human DNAI1 protein, wherein the fragment or portion of the protein still maintains DNAI1 activity similar to that of the wild-type protein.

In some embodiments, a suitable mRNA encodes a fusion protein comprising a full length, fragment or portion of a DNAI1 protein fused to another protein (e.g., an N or C terminal fusion). In some embodiments, the protein fused to the mRNA encoding a full length, fragment or portion of a DNAI1 protein encodes a signal or a cellular targeting sequence.

In some embodiments, an mRNA suitable for the present invention comprises a nucleotide sequence at least 85%, 90%, 91%, 92%, 93%, 94%, 95%, 96%, 97%, 98%, 99% or more identical to SEQ ID NO: 6, SEQ ID NO: 7, SEQ ID NO: 8, SEQ ID NO: 9, or SEQ ID NO: 10. In some embodiments, an mRNA in accordance with the present invention comprises a nucleotide sequence at least 95% identical to SEQ ID NO: 6. In some embodiments, an mRNA according to the present invention comprises a nucleotide sequence at least 99% identical to SEQ ID NO: 6. For example, an mRNA according to the present invention comprises the nucleotide sequence of SEQ ID NO: 6. In some embodiments, an mRNA in accordance with the present invention comprises a nucleotide sequence at least 95% identical to SEQ ID NO: 7. In some embodiments, an mRNA according to the present invention comprises a nucleotide sequence at least 99% identical to SEQ ID NO: 7. For example, an mRNA according to the present invention comprises the nucleotide sequence of SEQ ID NO: 7. In some embodiments, an mRNA in accordance with the present invention comprises a nucleotide sequence at least 95% identical to SEQ ID NO: 8. In some embodiments, an mRNA according to the present invention comprises a nucleotide sequence at least 99% identical to SEQ ID NO: 8. For example, an mRNA according to the present invention comprises the nucleotide sequence of SEQ ID NO: 8. In some embodiments, an mRNA in accordance with the present invention comprises a nucleotide sequence at least 95% identical to SEQ ID NO: 9. In some embodiments, an mRNA according to the present invention comprises a nucleotide sequence at least 99% identical to SEQ ID NO: 9. For example, an mRNA according to the present invention comprises the nucleotide sequence of SEQ ID NO: 9. In some embodiments, an mRNA in accordance with the present invention comprises a nucleotide sequence at least 95% identical to SEQ ID NO: 10. In some embodiments, an mRNA according to the present invention comprises a nucleotide sequence at least 99% identical to SEQ ID NO: 10. For example, an mRNA according to the present invention comprises the nucleotide sequence of SEQ ID NO: 10.

Exemplary codon-optimized DNAI1 mRNA sequences are shown in Table 3:

TABLE 3
Exemplary DNAI1 Codon-Optimized Sequence

| | |
|---|---|
| SEQ ID | ATGATCCCCGCCTCCGCCAAAGCCCCTCACAAGCAACCGCACAAGCAAA |
| NO: 6 | GCATTAGCATTGGGCGGGGTACTCGGAAGCGCGACGAGGACTCGGGAA |
| | CTGAAGTCGGAGAGGGGACCGACGAATGGGCGCAGTCAAAGGCCACCG |
| | TGCGCCCACCGGACCAGCTCGAGCTGACCGATGCTGAGCTGAAGGAGGA |
| | GTTTACCCGGATCCTGACAGCCAACAACCCACATGCACCGCAGAACATC |
| | GTGCGGTACAGCTTCAAAGAGGGAACTTATAAGCCCATTGGCTTCGTGA |
| | ACCAACTCGCGGTGCATTACACCCAAGTCGGAAACCTTATTCCGAAGGA |
| | CTCGGACGAAGGCAGACGCCAGCACTACCGGGACGAGCTCGTGGCAGG |
| | ATCCCAGGAAAGCGTCAAGGTCATTTCCGAGACTGGCAACCTCGAGGAG |
| | GACGAAGAACCTAAGGAGCTGGAAACCGAACCCGGATCCCAGACCGAC |
| | GTGCCGGCCGCTGGGGCTGCCGAGAAAGTCACTGAAGAGGAACTCATGA |
| | CCCCGAAGCAGCCGAAAGAGAGAAAGCTCACCAACCAATTCAACTTCAG |
| | CGAGCGCGCCAGCCAAACCTACAACAACCCAGTCAGGGATCGGGAATGT |
| | CAGACCGAACCGCCTCCGAGAACGAACTTCTCGGCGACCGCGAACCAAT |
| | GGGAGATCTACGACGCCTACGTGGAAGAACTGGAAAAGCAGGAAAAGA |
| | CTAAGGAAAAGGAAAAGGCCAAGACTCCCGTCGCCAAGAAGTCGGGCA |
| | AAATGGCCATGCGGAAGCTCACCTCCATGGAATCACAGACTGACGACTT |
| | GATCAAGTTGAGCCAGGCCGCAAAGATCATGGAGCGCATGGTCAACCAA |
| | AATACTTACGACGATATCGCCCAAGACTTCAAGTACTACGACGACGCTG |
| | CCGATGAATACCGAGATCAAGTCGGCACCCTACTGCCGCTTTGGAAGTT |
| | CCAGAATGACAAGGCCAAGAGGCTGAGCGTGACCGCGCTGTGCTGGAA |
| | CCCCAAATACCGCGACCTCTTCGCCGTGGGATACGGCTCCTACGATTTCA |
| | TGAAGCAGAGCCGGGGAATGTTGCTCCTTTACTCCCTGAAGAACCCCTC |
| | CTTCCCTGAGTACATGTTCAGCTCAAACAGCGGCGTGATGTGCCTCGACA |
| | TTCACGTGGACCACCCTTACCTCGTGGCCGTGGGTCACTACGACGGCAA |
| | CGTCGCGATCTACAACTTGAAGAAGCCGCATTCACAGCCCTCGTTTTGCT |
| | CCTCGGCCAAGTCCGGCAAACATTCGGACCCAGTGTGGCAAGTCAAGTG |
| | GCAGAAAGATGACATGGACCAAAACTTGAACTTCTTCAGCGTGTCCTCC |
| | GACGGACGGATCGTGTCCTGGACCCTCGTGAAGCGGAAGTTGGTGCATA |
| | TCGACGTGATCAAATTGAAGGTCGAGGGTTCGACCACCGAAGTGCCTGA |
| | AGGCCTGCAGCTTCACCCCGTGGGATGCGGCACTGCCTTCGACTTCCACA |
| | AGGAGATCGACTACATGTTCCTCGTGGGAACCGAGGAAGGGAAGATCTA |
| | CAAATGCAGCAAGTCCTACTCATCACAATTCCTGGATACCTACGATGCCC |
| | ACAACATGAGCGTGGATACCGTGTCGTGGAACCCCTATCACACCAAGGT |
| | ATTCATGTCCTGCTCCTCCGACTGGACCGTCAAGATTTGGGACCACACCA |
| | TCAAGACCCCCATGTTCATCTACGACCTGAACTCCGCCGTGGGGGATGT |
| | GGCCTGGGCCCCCTACTCGTCGACCGTGTTTGCCGCGGTCACCACGGAC |
| | GGAAAGGCACACATTTTCGACCTTGCGATTAACAAATACGAGGCGATTT |
| | GCAACCAGCCCGTGGCCGCCAAAAAGAACCGCCTGACCCACGTTCAATT |
| | CAACTTAATCCACCCAATCATCATCGTCGGCGATGACAGAGGACACATT |
| | ATTAGCCTGAAACTTAGCCCCAACCTCCGCAAGATGCCCAAGGAGAAGA |
| | AGGGACAGGAAGTCCAGAAGGGCCCTGCCGTGGAGATTGCAAAGCTCG |
| | ATAAGCTCCTGAACTTAGTCCGGGAAGTGAAGATCAAGACTTAA |
| SEQ ID | ATGATCCCCGCTAGCGCTAAGGCCCCTCACAAACAGCCCCACAAGCAGA |
| NO: 7 | GCATTAGCATTGGACGCGGCACACGGAAACGGGACGAAGATTCCGGAA |
| | CCGAAGTCGGGGAAGGCACCGATGAATGGGCCCAGTCCAAGGCCACCG |
| | TGCGGCCTCCTGACCAATTGGAACTGACTGACGCGGAACTGAAGGAAGA |
| | ATTCACCAGAATTCTAACCGCCAACAATCCTCATGCCCCGCAAAACATC |
| | GTGCGCTACTCATTCAAGGAAGGAACCTACAAGCCCATTGGATTCGTGA |
| | ACCAACTCGCCGTGCACTACACTCAAGTCGGCAACTTAATTCCCAAGGA |
| | TTCGGACGAGGGCCGCCGGCAGCACTACAGGGATGAGCTTGTGGCAGGC |
| | TCACAGGAAAGCGTCAAAGTCATTAGCGAAACCGGCAACCTTGAAGAG |
| | GATGAGGAGCCTAAGGAGCTGGAAACCGAGCCGGGTTCGCAAACCGAC |
| | GTGCCTGCCGCCGGAGCCGCAGAGAAGGTCACCGAAGAGGAACTCATG |
| | ACCCCCAAGCAGCCGAAGGAGCGCAAACTCACTAACCAATTCAACTTCA |
| | GCGAGAGAGCGAGCCAGACCTACAACAACCCAGTGCGCGACCGGGAAT |
| | GCCAGACTGAGCCGCCCCCGAGAACTAACTTCAGCGCGACTGCCAACCA |
| | ATGGGAGATCTACGACGCCTACGTGGAAGAGTTGGAGAAGCAGGAAAA |
| | GACCAAGGAGAAGGAAAAGGCCAAGACCCCGGTGGCGAAGAAGTCCGG |
| | AAAGATGGCGATGCGGAAGTTGACGTCCATGGAATCCCAGACCGATGAC |
| | CTCATCAAGCTCAGCCAGGCAGCAAAGATCATGGAAAGGATGGTGAACC |
| | AGAACACCTACGATGACATTGCTCAGGACTTTAAGTACTACGACGATGC |
| | CGCCGACGAATACCGCGACCAGGTCGGAACCCTCCTCCCGCTTTGGAAA |
| | TTCCAGAACGATAAAGCCAAGCGCCTCAGCGTGACTGCGCTGTGCTGGA |
| | ACCCGAAGTACCGGGACCTTTTCGCCGTGGGCTATGGATCCTACGACTTC |
| | ATGAAGCAATCGCGGGGGATGCTCCTCCTGTATAGCCTGAAGAACCCGA |
| | GCTTCCCCGAGTACATGTTCAGCTCAAACTCCGGAGTGATGTGCCTCGAC |
| | ATCCACGTGGACCACCCATACTTGGTGGCCGTGGGCCACTACGATGGCA |
| | ATGTCGCGATCTACAACCTCAAGAAGCCCCACTCCCAACCCTCGTTCTGC |
| | AGCAGCGCCAAGAGCGGGAAACACTCCGACCCAGTGTGGCAAGTCAAA |
| | TGGCAGAAGGACGACATGGATCAGAACTTGAACTTCTTCAGCGTGTCCT |
| | CAGACGGACGGATCGTGTCCTGGACCCTCGTGAAGAGGAAGTTGGTCCA |
| | TATCGACGTGATCAAGCTCAAGGTCGAGGGTTCAACCACCGAGGTGCCC |
| | GAGGGCCTGCAGCTCCACCCCGTGGGCTGCGGCACGGCCTTCGACTTTC |
| | ATAAGGAAATTGACTACATGTTCCTCGTGGGGACTGAAGAGGGAAAGAT |
| | CTACAAGTGCAGCAAATCCTACTCGTCCCAATTCCTCGACACCTACGATG |
| | CGCACAACATGAGCGTCGACACGGTGTCATGGAACCCCTACCATACAAA |
| | GGTATTCATGTCGTGCTCCTCCGACTGGACCGTGAAGATTTGGGATCACA |
| | CTATCAAGACCCCGATGTTCATATACGACCTGAACTCCGCCGTGGGAGA |
| | TGTGGCTTGGGCGCCCTACTCATCGACCGTGTTTGCCGCCGTGACCACCG |
| | ACGGAAAGGCTCACATTTTCGATCTTGCCATCAACAAGTACGAGGCGAT |
| | TTGCAACCAGCCAGTGGCCGCCAAGAAGAACAGACTGACTCACGTGCAG |
| | TTTAACTTAATCCACCCCATTATTATCGTGGGGGACGACCGCGGACACAT |
| | TATTAGCCTGAAGCTCAGCCCGAACCTCCGGAAGATGCCTAAGGAGAAG |
| | AAGGGGCAGGAAGTGCAGAAAGGCCCGGCCGTGGAAATTGCCAAATTG |
| | GACAAGTTACTGAACCTTGTCCGCGAAGTCAAGATCAAAACTTAA |
| SEQ ID | ATGATCCCCGCATCCGCCAAAGCCCCTCATAAACAGCCCCACAAACAGT |
| NO: 8 | CCATCTCCATTGGACGGGGGACCCGGAAAAGGGATGAGGACTCTGGGAC |
| | GGAAGTTGGAGAAGGCACTGACGAATGGGCACAGAGTAAGGCTACCGT |
| | GAGACCTCCCGACCAGCTGGAGCTCACTGACGCAGAACTGAAGGAGGA |
| | GTTTACTAGGATCCTGACAGCAAATAACCCCCACGCCCCACAGAATATC |
| | GTCAGATATAGCTTCAAAGAGGGCACATACAAGCCTATTGGGTTCGTGA |
| | ACCAGCTGGCTGTGCATTACACACAGGTGGGGAACCTTATTCCTAAAGA |
| | CTCTGATGAAGGCCGCAGACAGCATTATAGAGATGAACTGGTTGCAGGA |
| | TCCCAAGAGTCTGTGAAAGTGATTAGCGAGACCGGCAACCTGGAAGAAG |
| | ATGAGGAACCAAAAGAACTGGAGACAGAGCCTGGGTCTCAGACAGACG |
| | TGCCAGCAGCTGGCGCTGCCGAGAAAGTGACAGAGGAGGAGCTGATGA |
| | CACCTAAACAGCCAAAAGAGAGGAAGCTGACAAACCAATTCAATTTTTC |
| | CGAACGGGCATCACAGACCTACAACAACCCAGTGCGCGACCGGGAGTGT |
| | CAAACCGAACCTCCTCCTAGAACAAACTTTTCTGCTACTGCAAATCAGTG |
| | GGAGATCTACGATGCCTACGTGGAGGAGCTGGAGAAGCAGGAAAAGAC |
| | TAAGGAGAAGGAGAAGGCAAAGACCCCCGTGGCCAAAAAATCCGGCAA |
| | AATGGCAATGCGGAAGCTGACTTCTATGGAAAGCCAGACTGATGACCTG |
| | ATCAAACTGTCCCAGGCAGCTAAGATTATGGAAAGGATGGTCAATCAGA |
| | ATACATATGACGACATTGCTCAGGACTTTAAGTATTATGATGATGCCGCT |
| | GACGAGTATCGGGACCAAGTGGGGACACTGCTGCCACTGTGGAAGTTTC |
| | AAAACGACAAGGCTAAAAGGCTGTCCGTGACAGCACTCTGCTGGAATCC |
| | CAAGTACCGGGACCTCTTTGCCGTGGGGTACGGATCTTACGACTTCATGA |
| | AACAGTCCAGAGGCATGCTGCTGCTGTACAGCTTGAAGAACCCCTCCTTT |
| | CCCGAGTACATGTTCAGCTCTAATTCTGGAGTGATGTGCCTGGACATCCA |
| | CGTGGATCACCCTTACCTCGTGGCCGTTGGACACTATGACGGCAATGTG |
| | GCCATCTACAACCTGAAAAAACCACACTCTCAGCCTTCCTTTTGTAGCTC |
| | TGCAAAGTCCGGAAAGCATTCCGACCCCGTGTGGCAAGTGAAATGGCAG |
| | AAAGACGACATGGACCAGAATCTGAACTTCTTCTCCGTCTCTTCAGACG |
| | GCAGAATCGTCTCATGGACTCTGGTCAAACGGAAGCTGGTTCACATCGA |
| | CGTGATCAAACTCAAGGTCGAAGGATCGACTACTGAGGTGCCAGAAGGA |
| | CTGCAGCTGCACCCAGTGGGATGTGGAACTGCATTTGATTTCCATAAAG |
| | AAATCGACTACATGTTTCTGGTGGGAACTGAAGAGGGGAAGATCTATAA |
| | GTGTAGCAAATCCTATTCTAGCCAGTTTCTGGATACATACGACGCTCACA |
| | ACATGTCCGTGGACACTGTAAGCTGGAACCCCTATCATACCAAGGTGTT |
| | CATGTCCTGCAGCTCCGATTGGACTGTTAAGATTTGGGATCACACAATCA |
| | AGACCCCTATGTTTATCTACGATCTGAACTCTGCCGTGGGGGATGTGGCC |
| | TGGGCACCATATAGCTCCACAGTCTTCGCAGCTGTCACTACCGATGGAA |
| | AGGCCCACATTTTTGACCTGGCTATCAACAAATACGAGGCCATCTGCAA |
| | TCAGCCTGTGGCAGCAAAGAAGAACCGCCTGACTCACGTGCAATTCAAC |
| | CTGATTCACCCTATCATCATTGTTGGGGATGATAGGGGCCACATTATTTC |
| | TCTAAAGCTGTCCCCAAATCTGCGGAAAATGCCCAAGGAGAAGAAAGGC |
| | CAGGAGGTGCAGAAAGGCCCAGCCGTTGAAATCGCAAAGCTGGACAAG |
| | CTGCTCAACCTCGTCCGGGAGGTTAAAATCAAAACCTGA |
| SEQ ID | ATGATCCCAGCAAGCGCCAAGGCCCCACACAAACAGCCCCACAAGCAGT |
| NO: 9 | CGATCAGCATTGGCAGGGGGACTCGCAAGAGAGACGAGGACTCCGGAA |
| | CAGAAGTGGGGGAGGGGACAGATGAATGGGCCCAGTCTAAGGCCACTG |
| | TTCGCCCTCCGGATCAGCTGGAACTGACAGATGCCGAGCTGAAGGAAGA |
| | GTTCACCAGGATTCTGACTGCAAATAATCCACACGCTCCACAGAACATT |
| | GTGAGATATTCTTTTAAGGAGGGCACTTACAAACCCATCGGGTTTGTGA |
| | ATCAGCTGGCAGTGCATTACACTCAAGTGGGCAACCTGATCCCCAAAGA |
| | CTCTGATGAAGGGAGGCGGCAGCACTATAGGGACGAGCTGGTCGCTGGG |
| | TCCCAAGAGAGCGTGAAAGTCATTTCTGAGACTGGCAACCTGGAAGAGG |
| | ATGAGGAGCCAAAGGAGCTGGAGACTGAACCAGGGTCTCAGACAGATG |
| | TGCCCGCCGCTGGAGCTGCTGAGAAGGTGACAGAGGAGGAACTGATGA |
| | CCCCTAAACAGCCTAAGGAACGGAAGCTCACCAACCAGTTCAACTTCAG |
| | CGAAAGAGCTAGCCAGACTTATAATAACCCTGTGCGCGACCGGGAGTGT |
| | CAGACTGAGCCCCCACCAAGAACCAATTTCTCCGCCACTGCCAACCAGT |
| | GGGAAATCTATGACGCTTACGTCGAGGAGCTGGAGAAACAGGAGAAAA |
| | CTAAGGAGAAAGAAAAGGCCAAAACACCCGTCGCCAAAAAGTCTGGCA |
| | AGATGGCCATGAGAAAACTGACCTCCATGGAGTCTCAGACCGACGACCT |
| | GATCAAACTGTCCCAGGCAGCCAAGATCATGGAGAGGATGGTGAACCA |
| | GAACACCTATGATGACATTGCCCAGGACTTTAAATACTACGATGATGCC |
| | GCTGACGAGTATCGGGACCAGGTGGGGACTCTGCTGCCTCTGTGGAAAT |
| | TCCAGAATGATAAGGCTAAACGCCTGTCCGTGACCGCCCTCTGCTGGAA |
| | CCCTAAGTACCGCGACCTCTTTGCTGTGGGGTACGGATCTTACGACTTCA |
| | TGAAACAGTCCAGAGGCATGCTGCTGCTGTACAGCTTGAAGAACCCCTC |
| | CTTTCCCGAGTACATGTTCAGCTCTAATTCTGGAGTGATGTGCCTGGACA |
| | TCCACGTGGATCACCCTTACCTCGTGGCCGTTGGACACTATGACGGCAAT |
| | GTGGCCATCTACAACCTGAAAAAACCACACTCTCAGCCTTCCTTTTGTAG |
| | CTCTGCAAAGTCCGGAAAGCATTCCGACCCCGTGTGGCAAGTGAAATGG |
| | CAGAAAGACGACATGGACCAGAATCTGAACTTCTTCTCCGTCTCTTCAG |
| | ACGGCAGAATCGTCTCATGGACTCTGGTCAAACGGAAGCTGGTTCACAT |
| | CGACGTGATCAAACTCAAGGTCGAAGGATCGACTACTGAGGTGCCAGAA |
| | GGACTGCAGCTGCACCCAGTGGGATGTGGAACTGCATTTGATTTCCATA |
| | AAGAAATCGACTACATGTTTCTGGTGGGAACTGAAGAGGGGAAGATCTA |
| | TAAGTGTAGCAAATCCTATTCTAGCCAGTTTCTGGATACATACGACGCTC |
| | ACAACATGTCCGTGGACACTGTAAGCTGGAACCCCTATCATACCAAGGT |
| | GTTCATGTCCTGCAGCTCCGATTGGACTGTTAAGATTTGGGATCACACAA |
| | TCAAGACCCCTATGTTTATCTACGATCTGAACTCTGCCGTGGGGGATGTG |
| | GCCTGGGCACCATATAGCTCCACAGTCTTCGCAGCTGTCACTACCGATGG |
| | AAAGGCCCACATTTTTGACCTGGCTATCAACAAATACGAGGCCATCTGC |
| | AATCAGCCTGTGGCAGCAAAGAAGAACCGCCTGACTCACGTGCAATTCA |
| | ACCTGATTCACCCTATCATCATTGTTGGGGATGATAGGGGCCACATTATT |
| | TCTCTAAAGCTGTCCCCAAATCTGCGGAAAATGCCCAAGGAGAAGAAAG |
| | GCCAGGAGGTGCAGAAAGGCCCAGCCGTTGAAATCGCAAAGCTGGACA |
| | AGCTGCTCAACCTCGTCCGGGAGGTTAAAATCAAAACCTGA |
| SEQ ID | ATGATCCCAGCTTCTGCCAAGGCCCCACACAAGCAGCCACACAAACAGA |
| NO: 10 | GCATTTCCATTGGGCGCGGCACAAGGAAGAGAGACGAGGACTCAGGCA |
| | CAGAGGTGGGCGAAGGAACCGACGAGTGGGCTCAGAGCAAAGCCACAG |
| | TGAGGCCCCCAGATCAGCTGGAGCTGACAGACGCCGAGCTGAAGGAGG |
| | AGTTTACCCGCATCCTGACTGCCAATAACCCACACGCACCCCAGAACAT |
| | CGTGCGCTATTCTTTTAAGGAAGGAACCTATAAGCCAATCGGCTTTGTCA |
| | ATCAGCTGGCTGTGCACTACACCCAGGTTGGGAACCTGATCCCCAAGGA |
| | TAGCGACGAGGGCAGGAGACAGCATTATAGAGACGAGCTCGTCGCCGG |
| | AAGCCAGGAGTCTGTCAAAGTGATCAGCGAAACAGGAAACCTGGAGGA |
| | GGATGAGGAGCCCAAGGAACTGGAAACCGAGCCTGGCAGCCAGACAGA |
| | TGTGCCAGCCGCAGGAGCCGCAGAGAAGGTGACAGAAGAGGAGCTCAT |
| | GACCCCCAAACAGCCAAAGGAGCGGAAACTGACAAACCAGTTCAACTTC |
| | AGCGAAAGAGCCAGCCAGACCTACAATAACCCCGTGCGGGACAGAGAA |
| | TGCCAGACAGAGCCTCCACCACGCACCAACTTCTCCGCAACAGCTAACC |
| | AGTGGGAGATCTATGATGCCTACGTGGAGGAGCTGGAAAAGCAGGAGA |
| | AGACCAAAGAAAAGGAGAAAGCCAAGACCCCTGTCGCCAAGAAGTCCG |
| | GCAAAATGGCTATGAGAAAGCTGACATCTATGGAATCCCAGACTGATGA |
| | CCTGATCAAGCTGTCTCAGGCAGCCAAGATTATGGAAAGAATGGTGAAT |
| | CAGAACACCTATGACGACATCGCCCAGGATTTTAAGTACTATGATGACG |
| | CTGCAGACGAGTATAGAGATCAGGTGGGGACCCTGCTGCCACTGTGGAA |
| | GTTCCAGAATGACAAGGCTAAGCGCCTGTCCGTGACAGCTCTGTGCTGG |
| | AATCCAAAATATAGGGACCTCTTCGCCGTGGGCTACGGCTCTTATGACTT |
| | CATGAAGCAGTCACGCGGGATGCTGCTGCTGTACAGCCTGAAAAATCCC |
| | TCCTTTCCCGAGTACATGTTCAGCTCTAACTCCGGGGTCATGTGTCTGGA |
| | TATTCATGTGGACCATCCATACCTGGTGGCTGTCGGGCACTACGATGGA |
| | AACGTGGCTATCTACAATCTGAAGAAGCCACACTCCCAGCCCTCCTTTTG |
| | CTCCTCCGCCAAGTCCGGCAAGCACTCCGACCCTGTGTGGCAGGTCAAG |
| | TGGCAGAAGGACGACATGGACCAGAACCTGAACTTCTTTTCTGTGTCTA |
| | GCGATGGCAGGATCGTGTCCTGGACCCTGGTGAAGAGAAAACTGGTGCA |
| | CATCGATGTTATCAAGCTCAAAGTCGAGGGAAGCACCACCGAGGTTCCT |
| | GAGGGCCTGCAGCTGCACCCAGTGGGCTGCGGCACAGCCTTCGACTTTC |
| | ATAAAGAGATTGACTACATGTTCCTGGTGGGCACAGAGGAGGGGAAGAT |
| | CTACAAGTGCTCCAAATCCTACTCCAGCCAGTTTCTGGACACTTACGACG |
| | CTCATAATATGAGCGTGGACACCGTGTCCTGGAACCCTTACCACACAAA |
| | GGTGTTCATGAGCTGCAGCAGCGACTGGACTGTGAAGATTTGGGACCAT |
| | ACTATCAAAACCCCAATGTTTATCTATGATCTCAATTCTGCCGTGGGCGA |
| | CGTGGCTTGGGCCCCCTATTCCTCCACAGTGTTCGCAGCCGTGACTACCG |
| | ACGGAAAAGCCCACATTTTCGACCTCGCTATTAACAAGTATGAGGCCAT |
| | TTGTAACCAGCCAGTGGCTGCCAAGAAGAACCGCCTGACCCACGTGCAG |
| | TTCAACCTGATTCACCCAATTATCATTGTGGGGGACGACAGAGGACACA |
| | TTATCTCACTGAAGCTGTCTCCTAATCTGAGAAAGATGCCTAAGGAGAA |
| | GAAAGGACAGGAGGTGCAGAAGGGCCCTGCCGTGGAAATTGCCAAACT |
| | CGACAAGCTGCTGAACCTGGTGAGGGAGGTGAAGATCAAGACATGA |

mRNA Synthesis

mRNAs according to the present invention may be synthesized according to any of a variety of known methods. Various methods are described in published U.S. Application No. US 2018/0258423, and can be used to practice the present invention, all of which are incorporated herein by reference. For example, mRNAs according to the present invention may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7, or SP6 RNA polymerase), DNAse I, pyrophosphatase, and/or RNAse inhibitor. The exact conditions will vary according to the specific application.

### 5′ and 3′ Untranslated Region

In some embodiments, mRNAs include a 5′ untranslated region (UTR). In some embodiments, mRNAs include a 3′ untranslated region. In some embodiments, mRNAs include both a 5′ untranslated region and a 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length.

In some embodiments, a 3′ untranslated region includes one or more of a polyadenylation signal, a binding site for proteins that affect an mRNA's stability of location in a cell, or one or more binding sites for miRNAs. In some embodiments, a 3′ untranslated region may be between 50 and 500 nucleotides in length or longer.

Exemplary 3′ and 5′ untranslated region sequences can be derived from mRNA molecules which are stable (e.g., globin, actin, GAPDH, tubulin, histone, or citric acid cycle enzymes) to increase the stability of the sense mRNA molecule. For example, a 5′ UTR sequence may include a partial sequence of a CMV immediate-early 1 (IE1) gene, or a fragment thereof to improve the nuclease resistance and/or improve the half-life of the polynucleotide. Also contemplated is the inclusion of a sequence encoding human growth hormone (hGH), or a fragment thereof to the 3′ end or untranslated region of the polynucleotide (e.g., mRNA) to further stabilize the polynucleotide. Generally, these modifications improve the stability and/or pharmacokinetic properties (e.g., half-life) of the polynucleotide relative to their unmodified counterparts, and include, for example modifications made to improve such polynucleotides' resistance to in vivo nuclease digestion.

In certain embodiments, the codon-optimized DNAI1 mRNA includes a coding region having a codon-optimized coding region flanked by 5′ and 3′ untranslated regions as represented as X and Y, respectively (vide infra)
X-Coding Region-Y

where the coding region sequence is SEQ ID NO: 6, or a sequence 70%, 75%, 80%, 85%, 90%, 91%, 92%, 93%, 94%, 95%, 96%, 97%, 98%, 99% or more identical to SEQ ID NO: 6; SEQ ID NO: 7 SEQ ID NO: 8; SEQ ID NO: 9; or SEQ ID NO: 10 or a sequence 70%, 75%, 80%, 85%, 90%, 91%, 92%, 93%, 94%, 95%, 96%, 97%, 98%, 99% or more identical to SEQ ID NO: 10; and where

X (5′ UTR Sequence) is

AGACAGAUCGCCUGGAGACGCCAUCCACGCUGUUUUGACCUCCAUAGA AGACACCGGGACCGAUCCAGCCUCCGCGGCCGGGAACGGUGCAUUGGAACGCGGAUU CCCCGUGCCAAGAGUGACUCACCGUCCUUGACACG [SEQ ID NO.: 2] or a sequence 70%, 75%, 80%, 85%, 90%, 91%, 92%, 93%, 94%, 95%, 96%, 97%, 98%, 99% or more identical to SEQ ID NO: 2, or

GGACAGAUCGCCUGGAGACGCCAUCCACGCUGUUUUGACCUCCAUAGAAGAC ACCGGGACCGAUCCAGCCUCCGCGGCCGGGAACGGUGCAUUGGAACGCGGAUUCCCC GUGCCAAGAGUGACUCACCGUCCUUGACACG (SEQ ID NO: 3) or a sequence 70%, 75%, 80%, 85%, 90%, 91%, 92%, 93%, 94%, 95%, 96%, 97%, 98%, 99% or more identical to SEQ ID NO: 3; and where Y (3′ UTR Sequence) is

CGGGUGGCAUCCCUGUGACCCCUCCCCAGUGCCUCUCCUGGCCCUGGAAGUUG CCACUCCAGUGCCCACCAGCCUUGUCCUAAUAAAAUUAAGUUGCAUCAAGCU (SEQ ID NO: 4) or a sequence 70%, 75%, 80%, 85%, 90%, 91%, 92%, 93%, 94%, 95%, 96%, 97%, 98%, 99% or more identical to SEQ ID NO: 4, or

GGGUGGCAUCCCUGUGACCCCUCCCCAGUGCCUCUCCUGGCCCUGGAAGUUGCCACU CCAGUGCCCACCAGCCUUGUCCUAAUAAAAUUAAGUUGCAUCAAAGCU (SEQ ID NO: 5) or a sequence 70%, 75%, 80%, 85%, 90%, 91%, 92%, 93%, 94%, 95%, 96%, 97%, 98%, 99% or more identical to SEQ ID NO: 5.

The present invention may be used to deliver mRNAs of a variety of lengths. In some embodiments, the present invention may be used to deliver in vitro synthesized mRNA of or greater than about 0.5 kb, 1 kb, 1.5 kb, 2 kb, 2.5 kb, 3 kb, 3.5 kb, 4 kb, 4.5 kb, 5 kb 6 kb, 7 kb, 8 kb, 9 kb, 10 kb, 11 kb, 12 kb, 13 kb, 14 kb, 15 kb, 20 kb, 30 kb, 40 kb, or 50 kb in length. In some embodiments, the present invention may be used to deliver in vitro synthesized mRNA ranging from about 1-20 kb, about 1-15 kb, about 1-10 kb, about 5-20 kb, about 5-15 kb, about 5-12 kb, about 5-10 kb, about 8-20 kb, or about 8-50 kb in length.

In some embodiments, for the preparation of mRNA according to the invention, a DNA template is transcribed in vitro. A suitable DNA template typically has a promoter, for example a T3, T7 or SP6 promoter, for in vitro transcription, followed by desired nucleotide sequence for desired mRNA and a termination signal.

### Nucleotides

Various naturally-occurring or modified nucleosides may be used to produce mRNA according to the present invention. In some embodiments, an mRNA is or comprises naturally-occurring nucleosides (or unmodified nucleotides; e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, pseudouridine, (e.g., N-1-methyl-pseudouridine), 2-thiouridine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

In some embodiments, a suitable mRNA may contain backbone modifications, sugar modifications and/or base modifications. For example, modified nucleotides may include, but not be limited to, modified purines (adenine (A), guanine (G)) or pyrimidines (thymine (T), cytosine (C), uracil (U)), and as modified nucleotides analogues or derivatives of purines and pyrimidines, such as e.g. 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxyacetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxy acetic acid (v), 1-methyl-pseudouracil, queosine, .beta.-D-mannosyl-queosine, wybutoxosine, and phosphoramidates, phosphorothioates, peptide nucleotides, methylphosphonates, 7-deazaguanosine, 5-methylcytosine and inosine. The preparation of such analogues is known to a person skilled in the art e.g., from the U.S. Pat. Nos. 4,373,071, 4,401,796, 4,415,732, 4,458,066, 4,500,707, 4,668,777, 4,973,679, 5,047,524, 5,132,418, 5,153,319, 5,262,530 and 5,700,642, the disclosures of which are incorporated by reference in their entirety.

In some embodiments, the mRNA comprises one or more nonstandard nucleotide residues. The nonstandard nucleotide residues may include, e.g., 5-methyl-cytidine (“5mC”), pseudouridine (“EU”), and/or 2-thio-uridine (“2sU”). See, e.g., U.S. Pat. No. 8,278,036 or WO 2011/012316 for a discussion of such residues and their incorporation into mRNA. The mRNA may be RNA, which is defined as RNA in which 25% of U residues are 2-thio-uridine and 25% of C residues are 5-methylcytidine. Teachings for the use of RNA are disclosed US Patent Publication US 2012/0195936 and international publication WO 2011/012316, both of which are hereby incorporated by reference in their entirety. The presence of nonstandard nucleotide residues may render an mRNA more stable and/or less immunogenic than a control mRNA with the same sequence but containing only standard residues. In further embodiments, the mRNA may comprise one or more nonstandard nucleotide residues chosen from isocytosine, pseudoisocytosine, 5-bromouracil, 5-propynyluracil, 6-aminopurine, 2-aminopurine, inosine, diaminopurine and 2-chloro-6-aminopurine cytosine, as well as combinations of these modifications and other nucleobase modifications. Some embodiments may further include additional modifications to the furanose ring or nucleobase. Additional modifications may include, for example, sugar modifications or substitutions (e.g., one or more of a 2′-O-alkyl modification, a locked nucleic acid (LNA)). In some embodiments, the RNAs may be complexed or hybridized with additional polynucleotides and/or peptide polynucleotides (PNA). In some embodiments where the sugar modification is a 2′-O-alkyl modification, such modification may include, but are not limited to a 2′-deoxy-2′-fluoro modification, a 2′-O-methyl modification, a 2′-O-methoxyethyl modification and a 2′-deoxy modification. In some embodiments, any of these modifications may be present in 0-100% of the nucleotides—for example, more than 0%, 1%, 10%, 25%, 50%, 75%, 85%, 90%, 95%, or 100% of the constituent nucleotides individually or in combination.

In some embodiments, mRNAs may contain RNA backbone modifications. Typically, a backbone modification is a modification in which the phosphates of the backbone of the nucleotides contained in the RNA are modified chemically. Exemplary backbone modifications typically include, but are not limited to, modifications from the group consisting of methylphosphonates, methylphosphoramidates, phosphoramidates, phosphorothioates (e.g., cytidine 5′-O-(1-thiophosphate)), boranophosphates, positively charged guanidinium groups etc., which means by replacing the phosphodiester linkage by other anionic, cationic or neutral groups.

In some embodiments, mRNAs may contain sugar modifications. A typical sugar modification is a chemical modification of the sugar of the nucleotides it contains including, but not limited to, sugar modifications chosen from the group consisting of 2′-deoxy-2′-fluoro-oligoribonucleotide (2′-fluoro-2′-deoxycytidine 5′-triphosphate, 2′-fluoro-2′-deoxyuridine 5′-triphosphate), 2′-deoxy-2′-deamine-oligoribonucleotide (2′-amino-2′-deoxycytidine 5′-triphosphate, 2′-amino-2′-deoxyuridine 5′-triphosphate), 2′-O-alkyloligoribonucleotide, 2′-deoxy-2′-C-alkyloligoribonucleotide (2′-O-methylcytidine 5′-triphosphate, 2′-methyluridine 5′-triphosphate), 2′-C-alkyloligoribonucleotide, and isomers thereof (2′-aracytidine 5′-triphosphate, 2′-arauridine 5′-triphosphate), or azidotriphosphates (2′-azido-2′-deoxycytidine 5′-triphosphate, 2′-azido-2′-deoxyuridine 5′-triphosphate).

### Post-Synthesis Processing

Typically, a 5′ cap and/or a 3′ tail may be added after the synthesis. The presence of the cap is important in providing resistance to nucleases found in most eukaryotic cells. The presence of a “tail” serves to protect the mRNA from exonuclease degradation.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp(5′(A,G(5′)ppp(5′)A and G(5′)ppp(5′)G. Additional cap structures are described in published U.S. Application No. US 2016/0032356 and published U.S. Application No. US 2018/0125989, which are incorporated herein by reference.

Typically, a tail structure includes a poly(A) and/or poly(C) tail. A poly-A or poly-C tail on the 3′ terminus of mRNA typically includes at least 50 adenosine or cytosine nucleotides, at least 150 adenosine or cytosine nucleotides, at least 200 adenosine or cytosine nucleotides, at least 250 adenosine or cytosine nucleotides, at least 300 adenosine or cytosine nucleotides, at least 350 adenosine or cytosine nucleotides, at least 400 adenosine or cytosine nucleotides, at least 450 adenosine or cytosine nucleotides, at least 500 adenosine or cytosine nucleotides, at least 550 adenosine or cytosine nucleotides, at least 600 adenosine or cytosine nucleotides, at least 650 adenosine or cytosine nucleotides, at least 700 adenosine or cytosine nucleotides, at least 750 adenosine or cytosine nucleotides, at least 800 adenosine or cytosine nucleotides, at least 850 adenosine or cytosine nucleotides, at least 900 adenosine or cytosine nucleotides, at least 950 adenosine or cytosine nucleotides, or at least 1 kb adenosine or cytosine nucleotides, respectively. In some embodiments, a poly A or poly C tail may be about 10 to 800 adenosine or cytosine nucleotides (e.g., about 10 to 200 adenosine or cytosine nucleotides, about 10 to 300 adenosine or cytosine nucleotides, about 10 to 400 adenosine or cytosine nucleotides, about 10 to 500 adenosine or cytosine nucleotides, about 10 to 550 adenosine or cytosine nucleotides, about 10 to 600 adenosine or cytosine nucleotides, about 50 to 600 adenosine or cytosine nucleotides, about 100 to 600 adenosine or cytosine nucleotides, about 150 to 600 adenosine or cytosine nucleotides, about 200 to 600 adenosine or cytosine nucleotides, about 250 to 600 adenosine or cytosine nucleotides, about 300 to 600 adenosine or cytosine nucleotides, about 350 to 600 adenosine or cytosine nucleotides, about 400 to 600 adenosine or cytosine nucleotides, about 450 to 600 adenosine or cytosine nucleotides, about 500 to 600 adenosine or cytosine nucleotides, about 10 to 150 adenosine or cytosine nucleotides, about 10 to 100 adenosine or cytosine nucleotides, about 20 to 70 adenosine or cytosine nucleotides, or about 20 to 60 adenosine or cytosine nucleotides) respectively. In some embodiments, a tail structure includes is a combination of poly (A) and poly (C) tails with various lengths described herein. In some embodiments, a tail structure includes at least 50%, 55%, 65%, 70%, 75%, 80%, 85%, 90%, 92%, 94%, 95%, 96%, 97%, 98%, or 99% adenosine nucleotides. In some embodiments, a tail structure includes at least 50%, 55%, 65%, 70%, 75%, 80%, 85%, 90%, 92%, 94%, 95%, 96%, 97%, 98%, or 99% cytosine nucleotides.

As described herein, the addition of the 5′ cap and/or the 3′ tail facilitates the detection of abortive transcripts generated during in vitro synthesis because without capping and/or tailing, the size of those prematurely aborted mRNA transcripts can be too small to be detected. Thus, in some embodiments, the 5′ cap and/or the 3′ tail are added to the synthesized mRNA before the mRNA is tested for purity (e.g., the level of abortive transcripts present in the mRNA). In some embodiments, the 5′ cap and/or the 3′ tail are added to the synthesized mRNA before the mRNA is purified as described herein. In other embodiments, the 5′ cap and/or the 3′ tail are added to the synthesized mRNA after the mRNA is purified as described herein.

mRNA synthesized according to the present invention may be used without further purification. In particular, mRNA synthesized according to the present invention may be used without a step of removing shortmers. In some embodiments, mRNA synthesized according to the present invention may be further purified. Various methods may be used to purify mRNA synthesized according to the present invention. For example, purification of mRNA can be performed using centrifugation, filtration and/or chromatographic methods. In some embodiments, the synthesized mRNA is purified by ethanol precipitation or filtration or chromatography, or gel purification or any other suitable means. In some embodiments, the mRNA is purified by HPLC. In some embodiments, the mRNA is extracted in a standard phenol:chloroform:isoamyl alcohol solution, well known to one of skill in the art. In some embodiments, the mRNA is purified using Tangential Flow Filtration. Suitable purification methods include those described in published U.S. Application No. US 2016/0040154, published U.S. Application No. US 2015/0376220, published U.S. Application No. US 2018/0251755, published U.S. Application No. US 2018/0251754, U.S. Provisional Application No. 62/757,612 filed on Nov. 8, 2018, and U.S. Provisional Application No. 62/891,781 filed on Aug. 26, 2019, all of which are incorporated by reference herein and may be used to practice the present invention.

In some embodiments, the mRNA is purified before capping and tailing. In some embodiments, the mRNA is purified after capping and tailing. In some embodiments, the mRNA is purified both before and after capping and tailing.

In some embodiments, the mRNA is purified either before or after or both before and after capping and tailing, by centrifugation.

In some embodiments, the mRNA is purified either before or after or both before and after capping and tailing, by filtration.

In some embodiments, the mRNA is purified either before or after or both before and after capping and tailing, by Tangential Flow Filtration (TFF).

In some embodiments, the mRNA is purified either before or after or both before and after capping and tailing by chromatography.

Characterization of Purified mRNA

The mRNA composition described herein is substantially free of contaminants comprising short abortive RNA species, long abortive RNA species, double-stranded RNA (dsRNA), residual plasmid DNA, residual in vitro transcription enzymes, residual solvent and/or residual salt.

The mRNA composition described herein has a purity of about between 60% and about 100%. Accordingly, in some embodiments, the purified mRNA has a purity of about 60%. In some embodiments, the purified mRNA has a purity of about 65%. In some embodiments, the purified mRNA has a purity of about 70%. In some embodiments, the purified mRNA has a purity of about 75%. In some embodiments, the purified mRNA has a purity of about 80%. In some embodiments, the purified mRNA has a purity of about 85%. In some embodiments, the purified mRNA has a purity of about 90%. In some embodiments, the purified mRNA has a purity of about 910%. In some embodiments, the purified mRNA has a purity of about 92%. In some embodiments, the purified mRNA has a purity of about 93%. In some embodiments, the purified mRNA has a purity of about 94%. In some embodiments, the purified mRNA has a purity of about 95%. In some embodiments, the purified mRNA has a purity of about 96%. In some embodiments, the purified mRNA has a purity of about 97%. In some embodiments, the purified mRNA has a purity of about 98%. In some embodiments, the purified mRNA has a purity of about 99%. In some embodiments, the purified mRNA has a purity of about 100%.

In some embodiments, the mRNA composition described herein has less than 10%, less than 9%, less than 8%, less than 7%, less than 6%, less than 5%, less than 4%, less than 3%, less than 2%, less than 1%, less than 0.5%, and/or less than 0.1% impurities other than full-length mRNA. The impurities include IVT contaminants, e.g., proteins, enzymes, DNA templates, free nucleotides, residual solvent, residual salt, double-stranded RNA (dsRNA), prematurely aborted RNA sequences (“shortmers” or “short abortive RNA species”), and/or long abortive RNA species. In some embodiments, the purified mRNA is substantially free of process enzymes.

In some embodiments, the residual plasmid DNA in the purified mRNA of the present invention is less than about 1 pg/mg, less than about 2 pg/mg, less than about 3 pg/mg, less than about 4 pg/mg, less than about 5 pg/mg, less than about 6 pg/mg, less than about 7 pg/mg, less than about 8 pg/mg, less than about 9 pg/mg, less than about 10 pg/mg, less than about 11 pg/mg, or less than about 12 pg/mg. Accordingly, the residual plasmid DNA in the purified mRNA is less than about 1 pg/mg. In some embodiments, the residual plasmid DNA in the purified mRNA is less than about 2 pg/mg. In some embodiments, the residual plasmid DNA in the purified mRNA is less than about 3 pg/mg. In some embodiments, the residual plasmid DNA in the purified mRNA is less than about 4 pg/mg. In some embodiments, the residual plasmid DNA in the purified mRNA is less than about 5 pg/mg. In some embodiments, the residual plasmid DNA in the purified mRNA is less than about 6 pg/mg. In some embodiments, the residual plasmid DNA in the purified mRNA is less than about 7 pg/mg. In some embodiments, the residual plasmid DNA in the purified mRNA is less than about 8 pg/mg. In some embodiments, the residual plasmid DNA in the purified mRNA is less than about 9 pg/mg. In some embodiments, the residual plasmid DNA in the purified mRNA is less than about 10 pg/mg. In some embodiments, the residual plasmid DNA in the purified mRNA is less than about 11 pg/mg. In some embodiments, the residual plasmid DNA in the purified mRNA is less than about 12 pg/mg.

In some embodiments, a method according to the invention removes more than about 90%, 95%, 96%, 97%, 98%, 99% or substantially all prematurely aborted RNA sequences (also known as “shortmers”). In some embodiments, mRNA composition is substantially free of prematurely aborted RNA sequences. In some embodiments, mRNA composition contains less than about 5% (e.g., less than about 4%, 3%, 2%, or 1%) of prematurely aborted RNA sequences. In some embodiments, mRNA composition contains less than about 1% (e.g., less than about 0.9%, 0.8%, 0.7%, 0.6%, 0.5%, 0.4%, 0.3%, 0.2%, or 0.1%) of prematurely aborted RNA sequences. In some embodiments, mRNA composition undetectable prematurely aborted RNA sequences as determined by, e.g., high-performance liquid chromatography (HPLC) (e.g., shoulders or separate peaks), ethidium bromide, Coomassie staining, capillary electrophoresis or Glyoxal gel electrophoresis (e.g., presence of separate lower band). As used herein, the term “shortmers”, “short abortive RNA species”, “prematurely aborted RNA sequences” or “long abortive RNA species” refers to any transcripts that are less than full-length. In some embodiments, “shortmers”, “short abortive RNA species”, or “prematurely aborted RNA sequences” are less than 100 nucleotides in length, less than 90, less than 80, less than 70, less than 60, less than 50, less than 40, less than 30, less than 20, or less than 10 nucleotides in length. In some embodiments, shortmers are detected or quantified after adding a 5′-cap, and/or a 3′-poly A tail. In some embodiments, prematurely aborted RNA transcripts comprise less than 15 bases (e.g., less than 14, 13, 12, 11, 10, 9, 8, 7, 6, 5, 4, or 3 bases). In some embodiments, the prematurely aborted RNA transcripts contain about 8-15, 8-14, 8-13, 8-12, 8-11, or 8-10 bases.

In some embodiments, a purified mRNA of the present invention is substantially free of enzyme reagents used in in vitro synthesis including, but not limited to, T7 RNA polymerase, DNAse I, pyrophosphatase, and/or RNAse inhibitor. In some embodiments, a purified mRNA according to the present invention contains less than about 5% (e.g., less than about 4%, 3%, 2%, or 1%) of enzyme reagents used in in vitro synthesis including. In some embodiments, a purified mRNA contains less than about 1% (e.g., less than about 0.9%, 0.8%, 0.7%, 0.6%, 0.5%, 0.4%, 0.3%, 0.2%, or 0.1%) of enzyme reagents used in in vitro synthesis including. In some embodiments, a purified mRNA contains undetectable enzyme reagents used in in vitro synthesis including as determined by, e.g., silver stain, gel electrophoresis, high-performance liquid chromatography (HPLC), ultra-performance liquid chromatography (UPLC), and/or capillary electrophoresis, ethidium bromide and/or Coomassie staining.

In various embodiments, a purified mRNA of the present invention maintains high degree of integrity. As used herein, the term “mRNA integrity” generally refers to the quality of mRNA after purification. mRNA integrity may be determined using methods well known in the art, for example, by RNA agarose gel electrophoresis. In some embodiments, mRNA integrity may be determined by banding patterns of RNA agarose gel electrophoresis. In some embodiments, a purified mRNA of the present invention shows little or no banding compared to reference band of RNA agarose gel electrophoresis. In some embodiments, a purified mRNA of the present invention has an integrity greater than about 95% (e.g., greater than about 96%, 97%, 98%, 99% or more). In some embodiments, a purified mRNA of the present invention has an integrity greater than 98%. In some embodiments, a purified mRNA of the present invention has an integrity greater than 99%. In some embodiments, a purified mRNA of the present invention has an integrity of approximately 100%.

In some embodiments, the purified mRNA is assessed for one or more of the following characteristics: appearance, identity, quantity, concentration, presence of impurities, microbiological assessment, pH level and activity. In some embodiments, acceptable appearance includes a clear, colorless solution, essentially free of visible particulates. In some embodiments, the identity of the mRNA is assessed by sequencing methods. In some embodiments, the concentration is assessed by a suitable method, such as UV spectrophotometry. In some embodiments, a suitable concentration is between about 90% and 110% nominal (0.9-1.1 mg/mL).

In some embodiments, assessing the purity of the mRNA includes assessment of mRNA integrity, assessment of residual plasmid DNA, and assessment of residual solvent. In some embodiments, acceptable levels of mRNA integrity are assessed by agarose gel electrophoresis. The gels are analyzed to determine whether the banding pattern and apparent nucleotide length is consistent with an analytical reference standard. Additional methods to assess RNA integrity include, for example, assessment of the purified mRNA using capillary gel electrophoresis (CGE). In some embodiments, acceptable purity of the purified mRNA as determined by CGE is that the purified mRNA composition has no greater than about 55% long abortive/degraded species. In some embodiments, residual plasmid DNA is assessed by methods in the art, for example by the use of qPCR. In some embodiments, less than 10 pg/mg (e.g., less than 10 pg/mg, less than 9 pg/mg, less than 8 pg/mg, less than 7 pg/mg, less than 6 pg/mg, less than 5 pg/mg, less than 4 pg/mg, less than 3 pg/mg, less than 2 pg/mg, or less than 1 pg/mg) is an acceptable level of residual plasmid DNA. In some embodiments, acceptable residual solvent levels are not more than 10,000 ppm, 9,000 ppm, 8,000 ppm, 7,000 ppm, 6,000 ppm, 5,000 ppm, 4,000 ppm, 3,000 ppm, 2,000 ppm, 1,000 ppm. Accordingly, in some embodiments, acceptable residual solvent levels are not more than 10,000 ppm. In some embodiments, acceptable residual solvent levels are not more than 9,000 ppm. In some embodiments, acceptable residual solvent levels are not more than 8,000 ppm. In some embodiments, acceptable residual solvent levels are not more than 7,000 ppm. In some embodiments, acceptable residual solvent levels are not more than 6,000 ppm. In some embodiments, acceptable residual solvent levels are not more than 5,000 ppm. In some embodiments, acceptable residual solvent levels are not more than 4,000 ppm. In some embodiments, acceptable residual solvent levels are not more than 3,000 ppm. In some embodiments, acceptable residual solvent levels are not more than 2,000 ppm. In some embodiments, acceptable residual solvent levels are not more than 1,000 ppm.

In some embodiments, microbiological tests are performed on the purified mRNA, which include, for example, assessment of bacterial endotoxins. In some embodiments, bacterial endotoxins are <0.5 EU/mL, <0.4 EU/mL, <0.3 EU/mL, <0.2 EU/mL or <0.1 EU/mL. Accordingly, in some embodiments, bacterial endotoxins in the purified mRNA are <0.5 EU/mL. In some embodiments, bacterial endotoxins in the purified mRNA are <0.4 EU/mL. In some embodiments, bacterial endotoxins in the purified mRNA are <0.3 EU/mL. In some embodiments, bacterial endotoxins in the purified mRNA are <0.2 EU/mL. In some embodiments, bacterial endotoxins in the purified mRNA are <0.2 EU/mL. In some embodiments, bacterial endotoxins in the purified mRNA are <0.1 EU/mL. In some embodiments, the purified mRNA has not more than 1 CFU/10 mL, 1 CFU/25 mL, 1 CFU/50 mL, 1 CFU/75 mL, or not more than 1 CFU/100 mL. Accordingly, in some embodiments, the purified mRNA has not more than 1 CFU/10 mL. In some embodiments, the purified mRNA has not more than 1 CFU/25 mL. In some embodiments, the purified mRNA has not more than 1 CFU/50 mL. In some embodiments, the purified mRNA has not more than 1 CFR/75 mL. In some embodiments, the purified mRNA has 1 CFU/100 mL.

In some embodiments, the pH of the purified mRNA is assessed. In some embodiments, acceptable pH of the purified mRNA is between 5 and 8. Accordingly, in some embodiments, the purified mRNA has a pH of about 5. In some embodiments, the purified mRNA has a pH of about 6. In some embodiments, the purified mRNA has a pH of about 7. In some embodiments, the purified mRNA has a pH of about 7. In some embodiments, the purified mRNA has a pH of about 8.

In some embodiments, the translational fidelity of the purified mRNA is assessed. The translational fidelity can be assessed by various methods and include, for example, transfection and Western blot analysis. Acceptable characteristics of the purified mRNA includes banding pattern on a Western blot that migrates at a similar molecular weight as a reference standard.

In some embodiments, the purified mRNA is assessed for conductance. In some embodiments, acceptable characteristics of the purified mRNA include a conductance of between about 50% and 150% of a reference standard.

The purified mRNA is also assessed for Cap percentage and for PolyA tail length. In some embodiments, an acceptable Cap percentage includes Cap1, % Area: NLT90. In some embodiments, an acceptable PolyA tail length is about 100-1500 nucleotides (e.g., 100, 150, 200, 250, 300, 350, 400, 450, 500, 550, 600, 650, 700, 750, 800, 850, 900, 950, and 1000, 1100, 1200, 1300, 1400, or 1500 nucleotides).

In some embodiments, the purified mRNA is also assessed for any residual PEG. In some embodiments, the purified mRNA has less than between 10 ng PEG/mg of purified mRNA and 1000 ng PEG/mg of mRNA. Accordingly, in some embodiments, the purified mRNA has less than about 10 ng PEG/mg of purified mRNA. In some embodiments, the purified mRNA has less than about 100 ng PEG/mg of purified mRNA. In some embodiments, the purified mRNA has less than about 250 ng PEG/mg of purified mRNA. In some embodiments, the purified mRNA has less than about 500 ng PEG/mg of purified mRNA. In some embodiments, the purified mRNA has less than about 750 ng PEG/mg of purified mRNA. In some embodiments, the purified mRNA has less than about 1000 ng PEG/mg of purified mRNA.

Various methods of detecting and quantifying mRNA purity are known in the art. For example, such methods include, blotting, capillary electrophoresis, chromatography, fluorescence, gel electrophoresis, HPLC, silver stain, spectroscopy, ultraviolet (UV), or UPLC, or a combination thereof. In some embodiments, mRNA is first denatured by a Glyoxal dye before gel electrophoresis (“Glyoxal gel electrophoresis”). In some embodiments, synthesized mRNA is characterized before capping or tailing. In some embodiments, synthesized mRNA is characterized after capping and tailing.

### Delivery Vehicles

According to the present invention, mRNA or MCNA encoding a protein or a peptide (e.g., a full length, fragment, or portion of a protein or a peptide) as described herein may be delivered as naked RNA (unpackaged) or via delivery vehicles. As used herein, the terms “delivery vehicle,” “transfer vehicle,” “nanoparticle” or grammatical equivalent, are used interchangeably.

Delivery vehicles can be formulated in combination with one or more additional nucleic acids, carriers, targeting ligands or stabilizing reagents, or in pharmacological compositions where it is mixed with suitable excipients. Techniques for formulation and administration of drugs may be found in “Remington's Pharmaceutical Sciences,” Mack Publishing Co., Easton, Pa., latest edition. A particular delivery vehicle is selected based upon its ability to facilitate the transfection of a nucleic acid to a target cell.

In some embodiments, mRNAs or MCNAs encoding at least one protein or peptide may be delivered via a single delivery vehicle. In some embodiments, mRNAs or MCNAs encoding at least one protein or peptide may be delivered via one or more delivery vehicles each of a different composition. In some embodiments, the one or more mRNAs and/or MCNAs are encapsulated within the same lipid nanoparticles. In some embodiments, the one or more mRNAs are encapsulated within separate lipid nanoparticles.

According to various embodiments, suitable delivery vehicles include, but are not limited to polymer based carriers, such as polyethyleneimine (PEI), lipid nanoparticles and liposomes, nanoliposomes, ceramide-containing nanoliposomes, proteoliposomes, both natural and synthetically-derived exosomes, natural, synthetic and semi-synthetic lamellar bodies, nanoparticulates, calcium phosphor-silicate nanoparticulates, calcium phosphate nanoparticulates, silicon dioxide nanoparticulates, nanocrystalline particulates, semiconductor nanoparticulates, poly(D-arginine), sol-gels, nanodendrimers, starch-based delivery systems, micelles, emulsions, niosomes, multi-domain-block polymers (vinyl polymers, polypropyl acrylic acid polymers, dynamic polyconjugates), dry powder formulations, plasmids, viruses, calcium phosphate nucleotides, aptamers, peptides and other vectorial tags. Also contemplated is the use of bionanocapsules and other viral capsid proteins assemblies as a suitable transfer vehicle. (Hum. Gene Ther. 2008 September; 19(9):887-95).

### Liposomal Delivery Vehicles

In some embodiments, a suitable delivery vehicle is a liposomal delivery vehicle, e.g., a lipid nanoparticle. As used herein, liposomal delivery vehicles, e.g., lipid nanoparticles, are usually characterized as microscopic vesicles having an interior aqua space sequestered from an outer medium by a membrane of one or more bilayers. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphiphilic polymers and surfactants (e.g., polymerosomes, niosomes, etc.). In the context of the present invention, a liposomal delivery vehicle typically serves to transport a desired nucleic acid (e.g., mRNA or MCNA) to a target cell or tissue. In some embodiments, a nanoparticle delivery vehicle is a liposome. In some embodiments, a liposome comprises one or more cationic lipids, one or more non-cationic lipids, one or more cholesterol-based lipids, or one or more PEG-modified lipids. In some embodiments, a liposome comprises no more than three distinct lipid components. In some embodiments, one distinct lipid component is a sterol-based cationic lipid.

### Cationic Lipids

As used herein, the phrase “cationic lipids” refers to any of a number of lipid species that have a net positive charge at a selected pH, such as physiological pH.

Suitable cationic lipids for use in the compositions and methods of the invention include the cationic lipids as described in International Patent Publication WO 2010/144740, which is incorporated herein by reference. In certain embodiments, the compositions and methods of the present invention include a cationic lipid, (6Z,9Z,28Z,31Z)-heptatriaconta-6,9,28,31-tetraen-19-yl 4-(dimethylamino) butanoate, having a compound structure of:

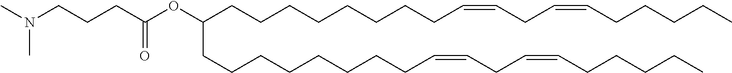 [SMILES: CCCCC/C=C\C/C=C\CCCCCCCCC(CCCCCCCC/C=C\C/C=C\CCCCC)OC(=O)CCCN(C)C]

and pharmaceutically acceptable salts thereof.

Other suitable cationic lipids for use in the compositions and methods of the present invention include ionizable cationic lipids as described in International Patent Publication WO 2013/149140, which is incorporated herein by reference. In some embodiments, the compositions and methods of the present invention include a cationic lipid of one of the following formulas:

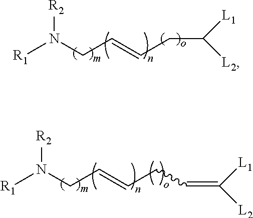

or a pharmaceutically acceptable salt thereof, wherein R₁ and R₂ are each independently selected from the group consisting of hydrogen, an optionally substituted, variably saturated or unsaturated C₁-C₂₀ alkyl and an optionally substituted, variably saturated or unsaturated C₆-C₂₀ acyl; wherein L₁ and L₂ are each independently selected from the group consisting of hydrogen, an optionally substituted C₁-C₃₀ alkyl, an optionally substituted variably unsaturated C₁-C₃ alkenyl, and an optionally substituted C₁-C₃₀ alkynyl; wherein m and o are each independently selected from the group consisting of zero and any positive integer (e.g., where m is three); and wherein n is zero or any positive integer (e.g., where n is one). In certain embodiments, the compositions and methods of the present invention include the cationic lipid (15Z,18Z)—N,N-dimethyl-6-(9Z,12Z)-octadeca-9,12-dien-1-yl)tetracosa-15,18-dien-1-amine (“HGT5000”), having a compound structure of:

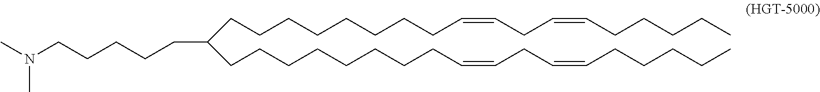 [SMILES: CCCCC/C=C\C/C=C\CCCCCCCCC(CCCCCCCC/C=C\C/C=C\CCCCC)CCCCCN(C)C]

and pharmaceutically acceptable salts thereof. In certain embodiments, the compositions and methods of the present invention include the cationic lipid (15Z,18Z)—N,N-dimethyl-6-((9Z,12Z)-octadeca-9,12-dien-1-yl)tetracosa-4,15,18-trien-1-amine (“HGT5001”), having a compound structure of

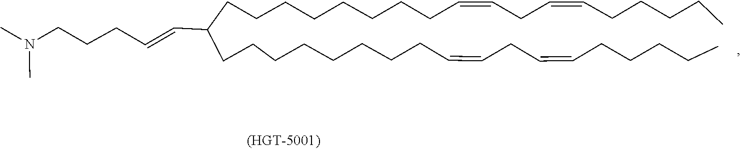 [SMILES: CCCCC/C=C\C/C=C\CCCCCCCCC(/C=C/CCCN(C)C)CCCCCCCC/C=C\C/C=C\CCCCC]

and pharmaceutically acceptable salts thereof. In certain embodiments, the compositions and methods of the present invention include the cationic lipid and (15Z,18Z)—N,N-dimethyl-6-((9Z,12Z)-octadeca-9,12-dien-1-yl)tetracosa-5,15,18-trien-1-amine (“HGT5002”), having a compound structure of

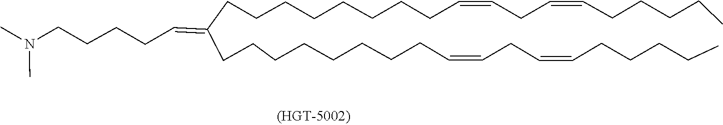 [SMILES: CCCCC/C=C\C/C=C\CCCCCCCCC(=CCCCCN(C)C)CCCCCCCC/C=C\C/C=C\CCCCC]

and pharmaceutically acceptable salts thereof.

Other suitable cationic lipids for use in the compositions and methods of the invention include cationic lipids described as aminoalcohol lipidoids in International Patent Publication WO 2010/053572, which is incorporated herein by reference. In certain embodiments, the compositions and methods of the present invention include a cationic lipid having a compound structure of:

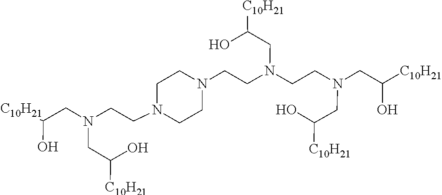

and pharmaceutically acceptable salts thereof.

Other suitable cationic lipids for use in the compositions and methods of the invention include the cationic lipids as described in International Patent Publication WO 2016/118725, which is incorporated herein by reference. In certain embodiments, the compositions and methods of the present invention include a cationic lipid having a compound structure of:

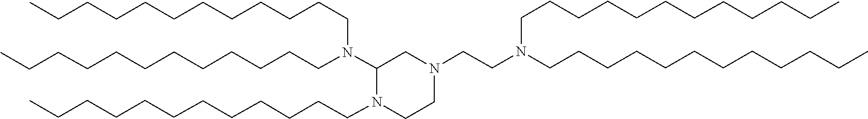 [SMILES: CCCCCCCCCCCCN(CCCCCCCCCCCC)CCN1CCN(CCCCCCCCCCCC)C(N(CCCCCCCCCCCC)CCCCCCCCCCCC)C1]

and pharmaceutically acceptable salts thereof.

Other suitable cationic lipids for use in the compositions and methods of the invention include the cationic lipids as described in International Patent Publication WO 2016/118724, which is incorporated herein by reference. In certain embodiments, the compositions and methods of the present invention include a cationic lipid having a compound structure of:

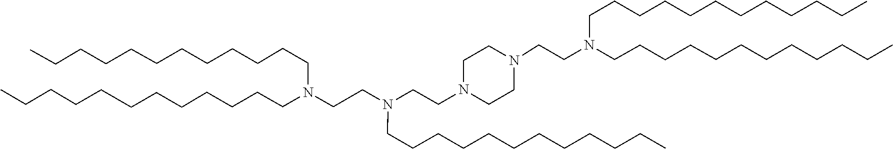 [SMILES: CCCCCCCCCCCCCN(CCCCCCCCCCCC)CCCN(CCCCCCCCCCCC)CCN1CCN(CCN(CCCCCCCCCCCC)CCCCCCCCCCCC)CC1]

and pharmaceutically acceptable salts thereof.

Other suitable cationic lipids for use in the compositions and methods of the invention include a cationic lipid having the formula of 14,25-ditridecyl 15,18,21,24-tetraaza-octatriacontane, and pharmaceutically acceptable salts thereof.

Other suitable cationic lipids for use in the compositions and methods of the invention include the cationic lipids as described in International Patent Publications WO 2013/063468 and WO 2016/205691, each of which are incorporated herein by reference. In some embodiments, the compositions and methods of the present invention include a cationic lipid of the following formula:

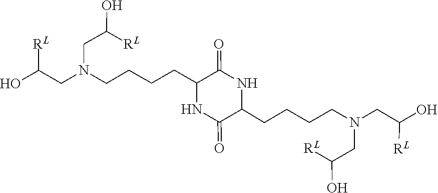

or pharmaceutically acceptable salts thereof, wherein each instance of R^{L} is independently optionally substituted C₆-C₄₀ alkenyl. In certain embodiments, the compositions and methods of the present invention include a cationic lipid having a compound structure of:

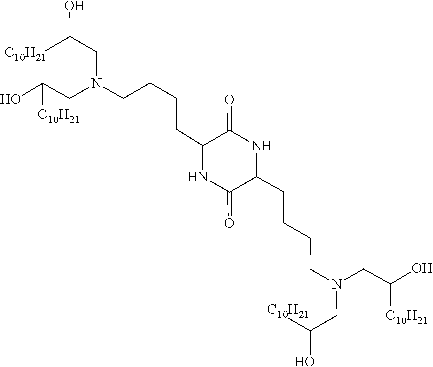

and pharmaceutically acceptable salts thereof. In certain embodiments, the compositions and methods of the present invention include a cationic lipid having a compound structure of:

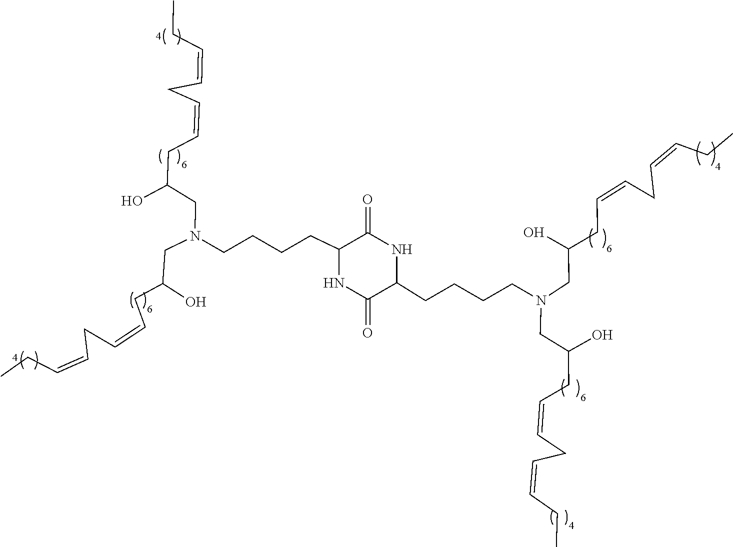

and pharmaceutically acceptable salts thereof. In certain embodiments, the compositions and methods of the present invention include a cationic lipid having a compound structure of:

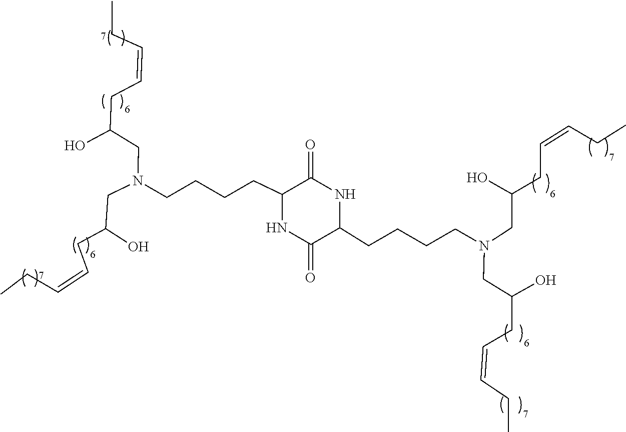

and pharmaceutically acceptable salts thereof. In certain embodiments, the compositions and methods of the present invention include a cationic lipid having a compound structure of:

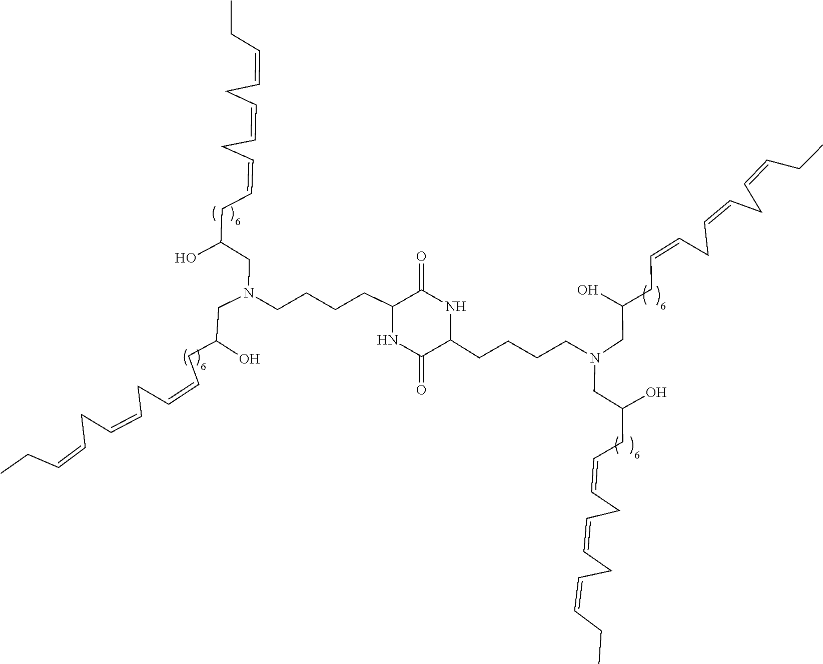

and pharmaceutically acceptable salts thereof.

Other suitable cationic lipids for use in the compositions and methods of the invention include the cationic lipids as described in International Patent Publication WO 2015/184256, which is incorporated herein by reference. In some embodiments, the compositions and methods of the present invention include a cationic lipid of the following formula:

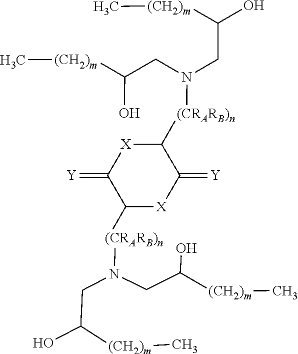

or a pharmaceutically acceptable salt thereof, wherein each X independently is O or S; each Y independently is O or S; each m independently is 0 to 20; each n independently is 1 to 6; each R_{A} is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen; and each R_{B} is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen. In certain embodiments, the compositions and methods of the present invention include a cationic lipid, “Target 23”, having a compound structure of

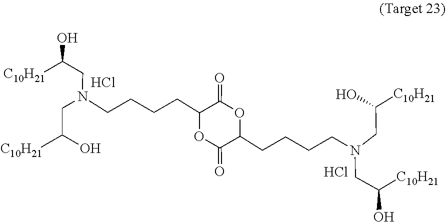

and pharmaceutically acceptable salts thereof.

Other suitable cationic lipids for use in the compositions and methods of the invention include the cationic lipids as described in International Patent Publication WO 2016/004202, which is incorporated herein by reference. In some embodiments, the compositions and methods of the present invention include a cationic lipid having the compound structure:

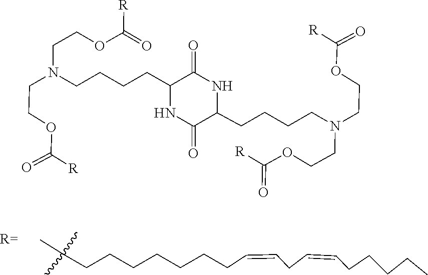

or a pharmaceutically acceptable salt thereof. In some embodiments, the compositions and methods of the present invention include a cationic lipid having the compound structure:

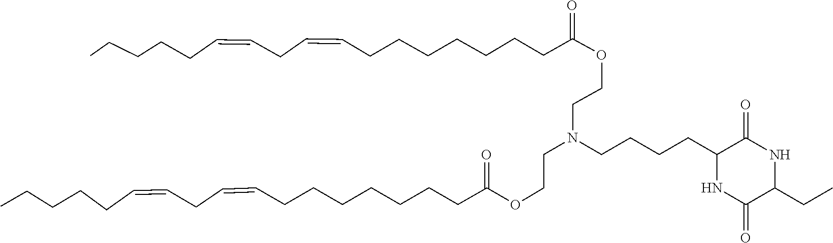 [SMILES: CCCCC/C=C\C/C=C\CCCCCCCC(=O)OCCN(CCCCC1NC(=O)C(CC)NC1=O)CCOC(=O)CCCCCCC/C=C\C/C=C\CCCCC]

or a pharmaceutically acceptable salt thereof. In some embodiments, the compositions and methods of the present invention include a cationic lipid having the compound structure:

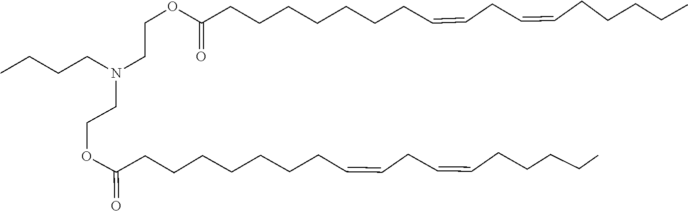 [SMILES: CCCCC/C=C\C/C=C\CCCCCCCC(=O)OCCN(CCCC)CCOC(=O)CCCCCCC/C=C\C/C=C\CCCCC]

or a pharmaceutically acceptable salt thereof.

Other suitable cationic lipids for use in the compositions and methods of the present invention include cationic lipids as described in U.S. Provisional Patent Application Ser. No. 62/758,179, which is incorporated herein by reference. In some embodiments, the compositions and methods of the present invention include a cationic lipid of the following formula:

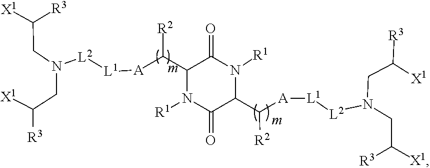

or a pharmaceutically acceptable salt thereof, wherein each R¹ and R² is independently H or C₁-C₆ aliphatic; each m is independently an integer having a value of 1 to 4; each A is independently a covalent bond or arylene; each L¹ is independently an ester, thioester, disulfide, or anhydride group; each L² is independently C₂-C₁₀ aliphatic; each X¹ is independently H or OH; and each R³ is independently C₆-C₂₀ aliphatic. In some embodiments, the compositions and methods of the present invention include a cationic lipid of the following formula:

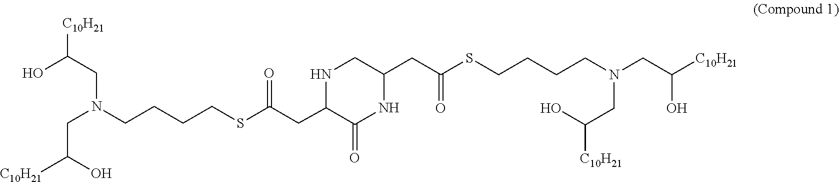

or a pharmaceutically acceptable salt thereof. In some embodiments, the compositions and methods of the present invention include a cationic lipid of the following formula:

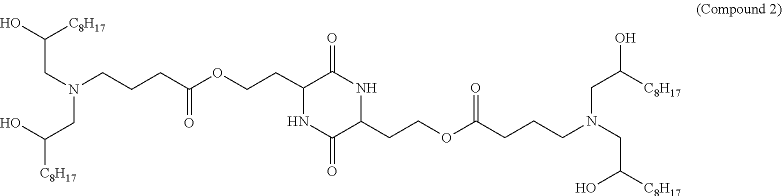

or a pharmaceutically acceptable salt thereof. In some embodiments, the compositions and methods of the present invention include a cationic lipid of the following formula:

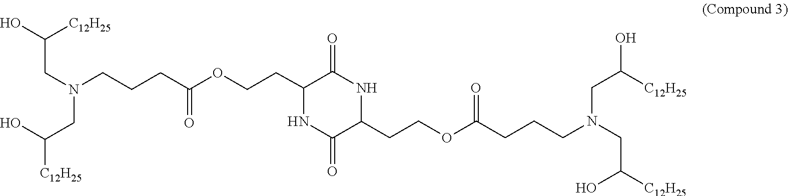

or a pharmaceutically acceptable salt thereof.

Other suitable cationic lipids for use in the compositions and methods of the present invention include the cationic lipids as described in J. McClellan, M. C. King, Cell 2010, 141, 210-217 and in Whitehead et al., Nature Communications (2014) 5:4277, which is incorporated herein by reference. In certain embodiments, the cationic lipids of the compositions and methods of the present invention include a cationic lipid having a compound structure of:

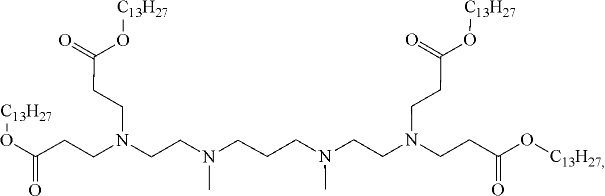

and pharmaceutically acceptable salts thereof.

Other suitable cationic lipids for use in the compositions and methods of the invention include the cationic lipids as described in International Patent Publication WO 2015/199952, which is incorporated herein by reference. In some embodiments, the compositions and methods of the present invention include a cationic lipid having the compound structure:

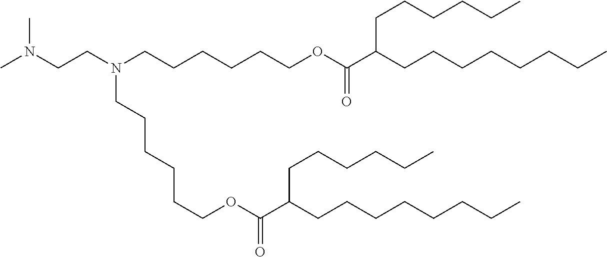 [SMILES: CCCCCCCCC(CCCCCC)C(=O)OCCCCCCN(CCCCCCOC(=O)C(CCCCCC)CCCCCCCC)CCN(C)C]

and pharmaceutically acceptable salts thereof. In some embodiments, the compositions and methods of the present invention include a cationic lipid having the compound structure:

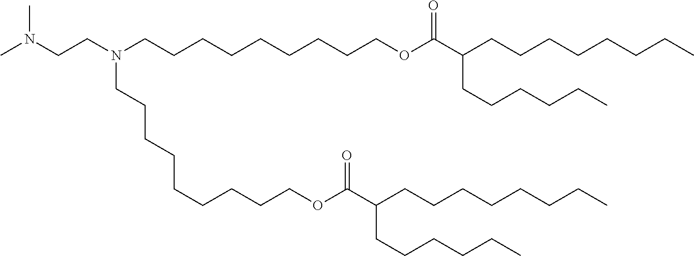 [SMILES: CCCCCCCCC(CCCCCC)C(=O)OCCCCCCCCCN(CCCCCCCCCOC(=O)C(CCCCCC)CCCCCCCC)CCN(C)C]

and pharmaceutically acceptable salts thereof. In some embodiments, the compositions and methods of the present invention include a cationic lipid having the compound structure:

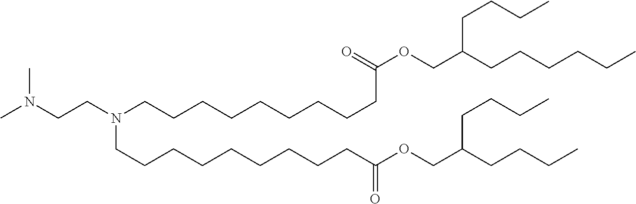 [SMILES: CCCCCCC(CCCC)COC(=O)CCCCCCCCCN(CCCCCCCCCC(=O)OCC(CCCC)CCCC)CCN(C)C]

and pharmaceutically acceptable salts thereof. In some embodiments, the compositions and methods of the present invention include a cationic lipid having the compound structure:

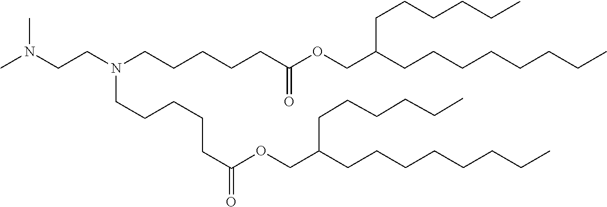 [SMILES: CCCCCCCCC(CCCCCC)COC(=O)CCCCCN(CCCCCC(=O)OCC(CCCCCC)CCCCCCCC)CCN(C)C]

and pharmaceutically acceptable salts thereof. In some embodiments, the compositions and methods of the present invention include a cationic lipid having the compound structure:

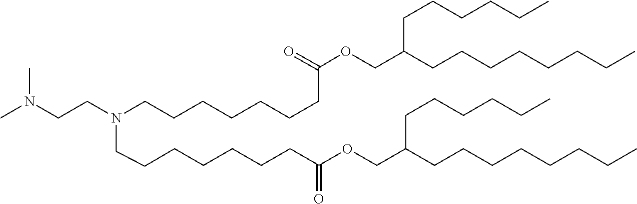 [SMILES: CCCCCCCCC(CCCCCC)COC(=O)CCCCCCCN(CCCCCCCC(=O)OCC(CCCCCC)CCCCCCCC)CCN(C)C]

and pharmaceutically acceptable salts thereof. In some embodiments, the compositions and methods of the present invention include a cationic lipid having the compound structure:

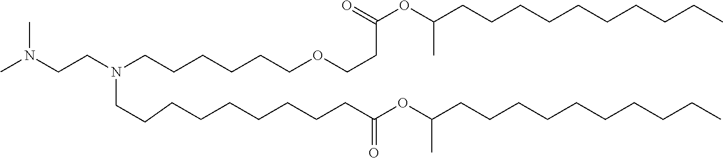 [SMILES: CCCCCCCCCCC(C)OC(=O)CCCCCCCCCN(CCCCCCOCCC(=O)OC(C)CCCCCCCCCC)CCN(C)C]

and pharmaceutically acceptable salts thereof. In some embodiments, the compositions and methods of the present invention include a cationic lipid having the compound structure:

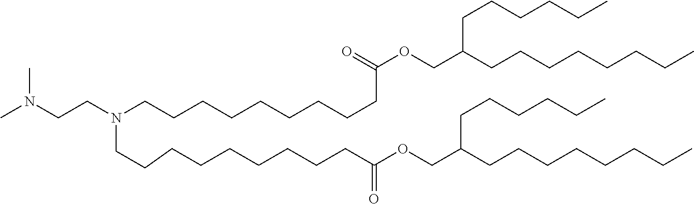 [SMILES: CCCCCCCCC(CCCCCC)COC(=O)CCCCCCCCCN(CCCCCCCCCC(=O)OCC(CCCCCC)CCCCCCCC)CCN(C)C]

and pharmaceutically acceptable salts thereof. In some embodiments, the compositions and methods of the present invention include a cationic lipid having the compound structure:

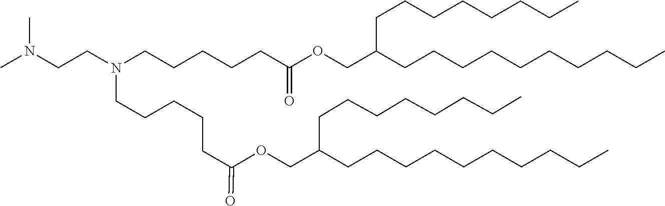 [SMILES: CCCCCCCCCCC(CCCCCCCC)COC(=O)CCCCCN(CCCCCC(=O)OCC(CCCCCCCC)CCCCCCCCCC)CCN(C)C]

and pharmaceutically acceptable salts thereof. In some embodiments, the compositions and methods of the present invention include a cationic lipid having the compound structure:

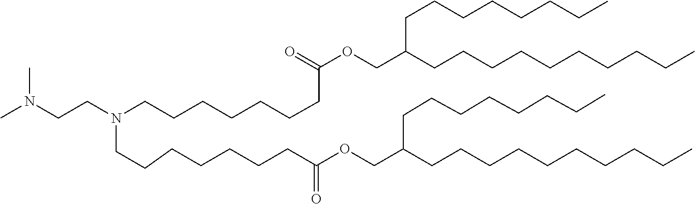 [SMILES: CCCCCCCCCCC(CCCCCCCC)COC(=O)CCCCCCCN(CCCCCCCC(=O)OCC(CCCCCCCC)CCCCCCCCCC)CCN(C)C]

and pharmaceutically acceptable salts thereof. In some embodiments, the compositions and methods of the present invention include a cationic lipid having the compound structure:

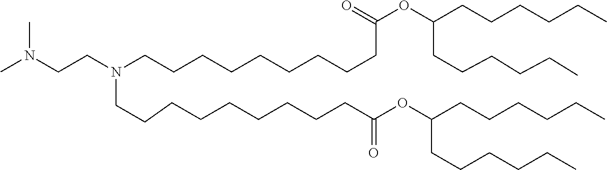 [SMILES: CCCCCCC(CCCCCC)OC(=O)CCCCCCCCCN(CCCCCCCCCC(=O)OC(CCCCCC)CCCCCC)CCN(C)C]

and pharmaceutically acceptable salts thereof. In some embodiments, the compositions and methods of the present invention include a cationic lipid having the compound structure:

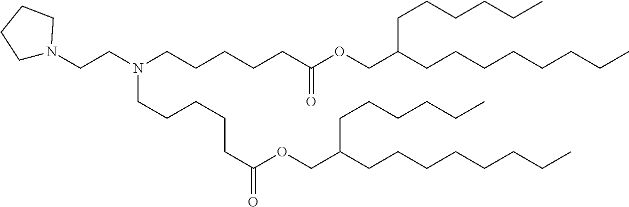 [SMILES: CCCCCCCCC(CCCCCC)COC(=O)CCCCCN(CCCCCC(=O)OCC(CCCCCC)CCCCCCCC)CCN1CCCC1]

and pharmaceutically acceptable salts thereof. In some embodiments, the compositions and methods of the present invention include a cationic lipid having the compound structure:

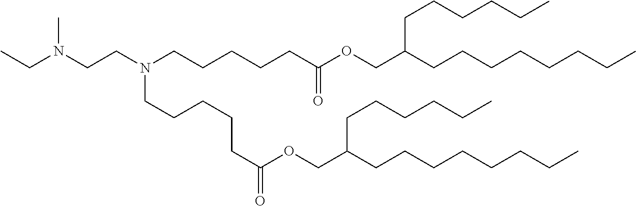 [SMILES: CCCCCCCCC(CCCCCC)COC(=O)CCCCCN(CCCCCC(=O)OCC(CCCCCC)CCCCCCCC)CCN(C)CC]

and pharmaceutically acceptable salts thereof. In some embodiments, the compositions and methods of the present invention include a cationic lipid having the compound structure:

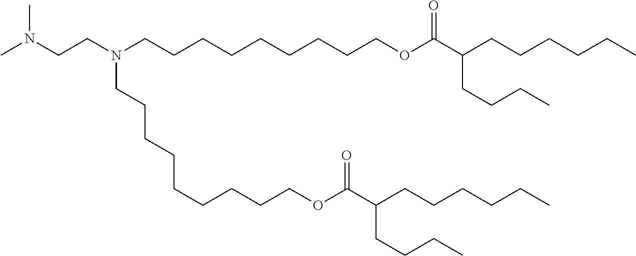 [SMILES: CCCCCCC(CCCC)C(=O)OCCCCCCCCCN(CCCCCCCCCOC(=O)C(CCCC)CCCCCC)CCN(C)C]

and pharmaceutically acceptable salts thereof.

Other suitable cationic lipids for use in the compositions and methods of the invention include the cationic lipids as described in International Patent Publication WO 2017/004143, which is incorporated herein by reference. In some embodiments, the compositions and methods of the present invention include a cationic lipid having the compound structure:

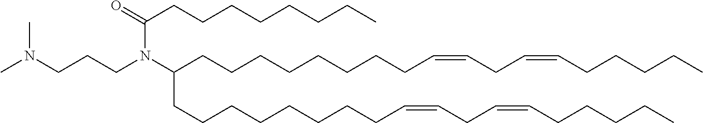 [SMILES: CCCCC/C=C\C/C=C\CCCCCCCCC(CCCCCCCC/C=C\C/C=C\CCCCC)N(CCCN(C)C)C(=O)CCCCCCCC]

and pharmaceutically acceptable salts thereof. In some embodiments, the compositions and methods of the present invention include a cationic lipid having the compound structure:

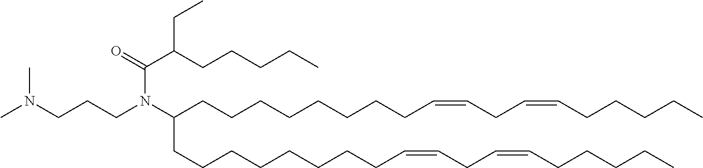 [SMILES: CCCCC/C=C\C/C=C\CCCCCCCCC(CCCCCCCC/C=C\C/C=C\CCCCC)N(CCCN(C)C)C(=O)C(CC)CCCCC]

and pharmaceutically acceptable salts thereof. In some embodiments, the compositions and methods of the present invention include a cationic lipid having the compound structure:

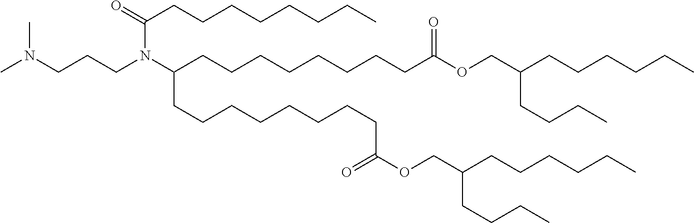 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present invention include a cationic lipid having the compound structure:

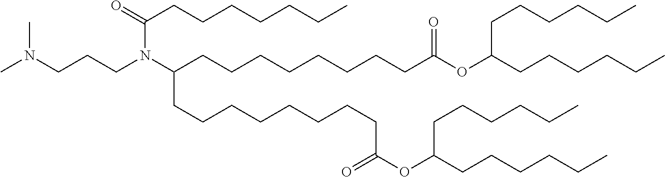 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof. In some embodiments, the compositions and methods of the present invention include a cationic lipid having the compound structure:

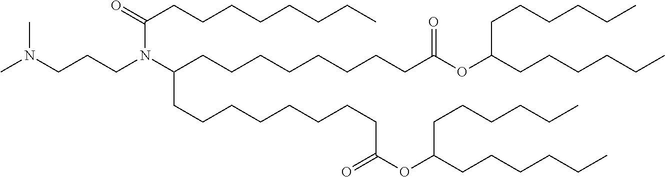 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof. In some embodiments, the compositions and methods of the present invention include a cationic lipid having the compound structure:

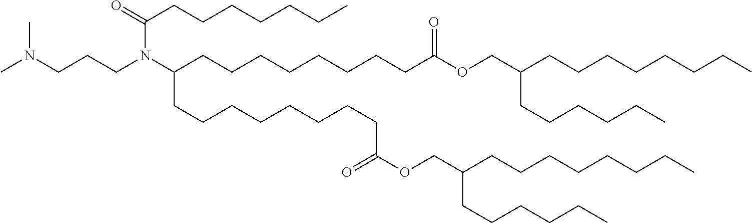 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC]

and pharmaceutically acceptable salts thereof. In some embodiments, the compositions and methods of the present invention include a cationic lipid having the compound structure:

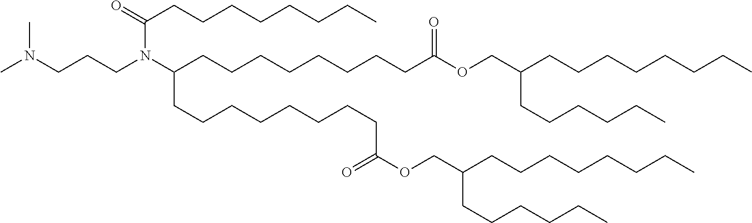 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC]

and pharmaceutically acceptable salts thereof. In some embodiments, the compositions and methods of the present invention include a cationic lipid having the compound structure:

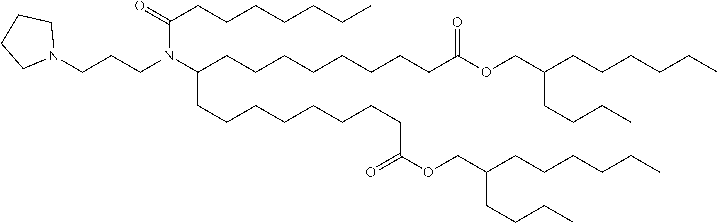 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present invention include a cationic lipid having the compound structure:

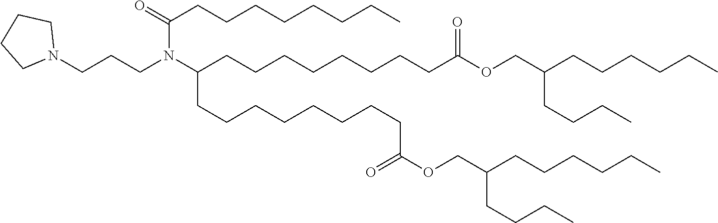 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present invention include a cationic lipid having the compound structure:

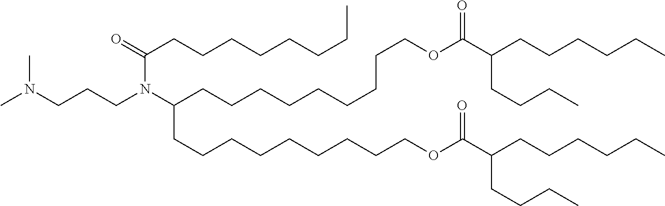 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC]

and pharmaceutically acceptable salts thereof. In some embodiments, the compositions and methods of the present invention include a cationic lipid having the compound structure:

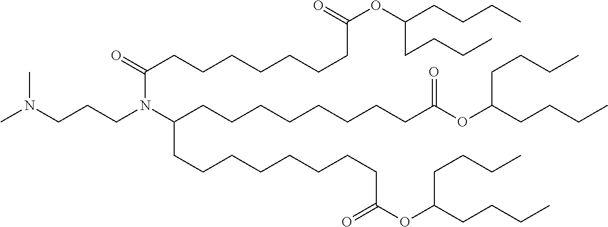 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC]

and pharmaceutically acceptable salts thereof. In some embodiments, the compositions and methods of the present invention include a cationic lipid having the compound structure:

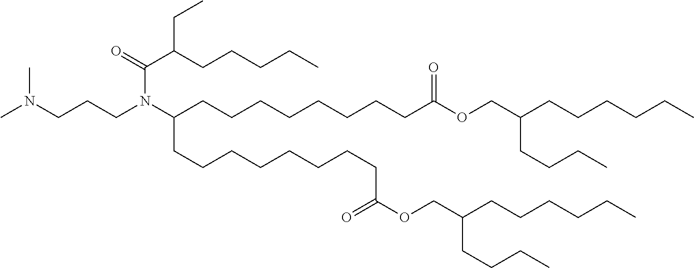 [SMILES: CCCCCCC(CCCC)COC(=O)CCCCCCCCC(CCCCCCCCC(=O)OCC(CCCC)CCCCCC)N(CCCN(C)C)C(=O)C(CC)CCCCC]

and pharmaceutically acceptable salts thereof. In some embodiments, the compositions and methods of the present invention include a cationic lipid having the compound structure:

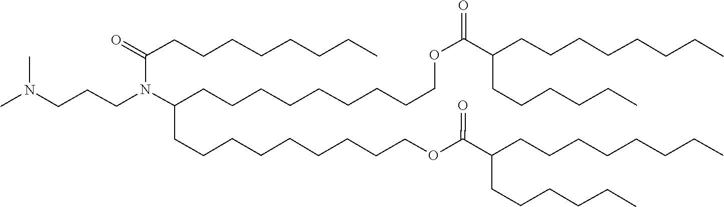 [SMILES: CCCCCCCCC(=O)N(CCCN(C)C)C(CCCCCCCCCOC(=O)C(CCCCCC)CCCCCCCC)CCCCCCCCCOC(=O)C(CCCCCC)CCCCCCCC]

and pharmaceutically acceptable salts thereof. In some embodiments, the compositions and methods of the present invention include a cationic lipid having the compound structure:

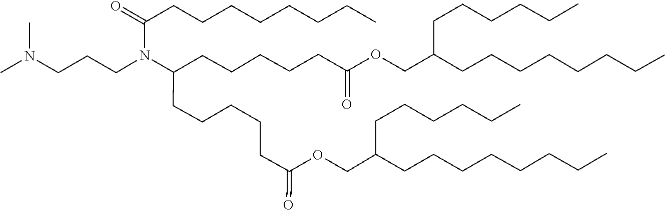 [SMILES: CCCCCCCCC(=O)N(CCCN(C)C)C(CCCCCC(=O)OCC(CCCCCC)CCCCCCCC)CCCCCC(=O)OCC(CCCCCC)CCCCCCCC]

and pharmaceutically acceptable salts thereof. In some embodiments, the compositions and methods of the present invention include a cationic lipid having the compound structure:

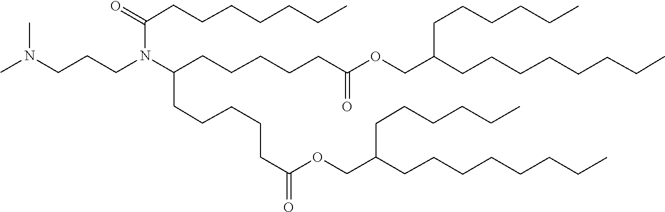 [SMILES: CCCCCCCCC(CCCCCC)COC(=O)CCCCCC(CCCCCC(=O)OCC(CCCCCC)CCCCCCCC)N(CCCN(C)C)C(=O)CCCCCCC]

and pharmaceutically acceptable salts thereof. In some embodiments, the compositions and methods of the present invention include a cationic lipid having the compound structure:

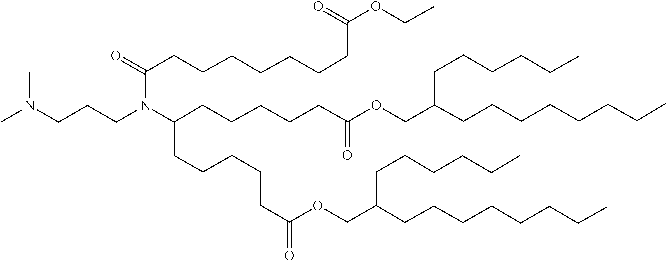 [SMILES: CCCCCCCCC(CCCCCC)COC(=O)CCCCCC(CCCCCC(=O)OCC(CCCCCC)CCCCCCCC)N(CCCN(C)C)C(=O)CCCCCCCC(=O)OCC]

and pharmaceutically acceptable salts thereof. In some embodiments, the compositions and methods of the present invention include a cationic lipid having the compound structure:

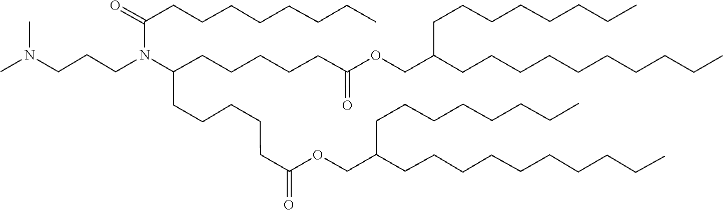 [SMILES: CCCCCCCCCCC(CCCCCCCC)COC(=O)CCCCCC(CCCCCC(=O)OCC(CCCCCCCC)CCCCCCCCCC)N(CCCN(C)C)C(=O)CCCCCCCC]

and pharmaceutically acceptable salts thereof.

Other suitable cationic lipids for use in the compositions and methods of the invention include the cationic lipids as described in International Patent Publication WO 2017/075531, which is incorporated herein by reference. In some embodiments, the compositions and methods of the present invention include a cationic lipid of the following formula:

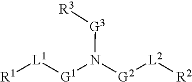

or a pharmaceutically acceptable salt thereof, wherein one of L¹ or L² is —O(C═O)—, —(C═O)O—, —C(═O)—, —O—, —S(O)ₓ, —S—S—, —C(═O)S—, —SC(═O)—, —NR^{a}C(═O)—, —C(═O)NR^{a}—, NR^{a}C(═O)NR^{a}, —OC(═O)NR^{a}—, or —NR^{a}C(═O)O—; and the other of L¹ or L² is —O(C═O)—, —(C═O)O—, —C(═O)—, —O—, —S(O)ₓ, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond; G¹ and G² are each independently unsubstituted C₁-C₁₂ alkylene or C₁-C₁₂ alkenylene; G³ is C₁-C₂₄ alkylene, C₁-C₂₄ alkenylene, C₃-C₈ cycloalkylene, C₃-C₈ cycloalkenylene; R^{a} is H or C₁-C₁₂ alkyl; R¹ and R² are each independently C₆-C₂₄ alkyl or C₆-C₂₄ alkenyl; R³ is H, OR⁵, CN, —C(═O)OR⁴, —OC(═O)R⁴ or —NR⁵C(═O)R⁴; R⁴ is C₁-C₁₂ alkyl; R⁵ is H or C₁-C₆ alkyl; and x is 0, 1 or 2.

Other suitable cationic lipids for use in the compositions and methods of the invention include the cationic lipids as described in International Patent Publication WO 2017/117528, which is incorporated herein by reference. In some embodiments, the compositions and methods of the present invention include a cationic lipid having the compound structure:

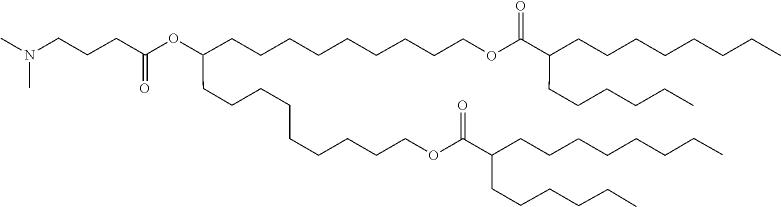 [SMILES: CCCCCCCCC(CCCCCC)C(=O)OCCCCCCCCCC(CCCCCCCCCOC(=O)C(CCCCCC)CCCCCCCC)OC(=O)CCCN(C)C]

and pharmaceutically acceptable salts thereof. In some embodiments, the compositions and methods of the present invention include a cationic lipid having the compound structure:

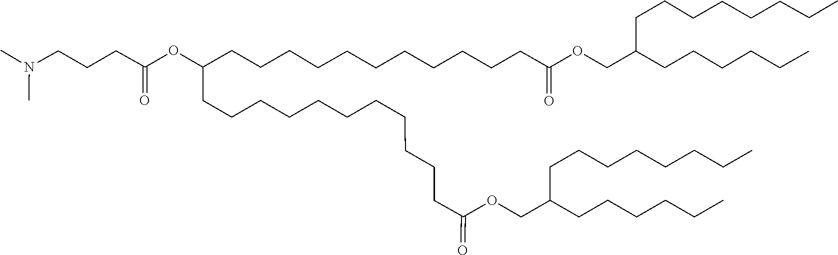 [SMILES: CCCCCCCCC(CCCCCC)COC(=O)CCCCCCCCCCCC(CCCCCCCCCCCC(=O)OCC(CCCCCC)CCCCCCCC)OC(=O)CCCN(C)C]

and pharmaceutically acceptable salts thereof. In some embodiments, the compositions and methods of the present invention include a cationic lipid having the compound structure:

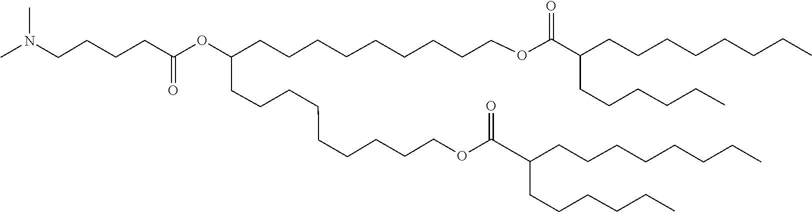 [SMILES: CCCCCCCCCC(CCCCCC)C(=O)OCCCCCCCCCC(CCCCCCCCCOC(=O)CC(CCCCCC)CCCCCCCC)OC(=O)CCCCN(C)C]

and pharmaceutically acceptable salts thereof.

Other suitable cationic lipids for use in the compositions and methods of the invention include the cationic lipids as described in International Patent Publication WO 2017/049245, which is incorporated herein by reference. In some embodiments, the cationic lipids of the compositions and methods of the present invention include a compound of one of the following formulas:

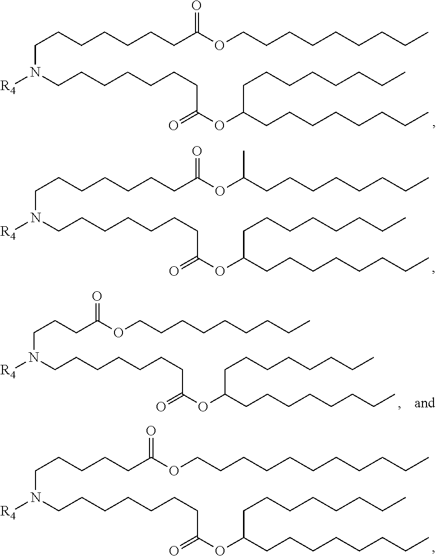

and pharmaceutically acceptable salts thereof. For any one of these four formulas, R₄ is independently selected from —(CH₂)ₙQ and —(CH₂)ₙCHQR; Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), and a heterocycle; and n is 1, 2, or 3. In certain embodiments, the compositions and methods of the present invention include a cationic lipid having a compound structure of:

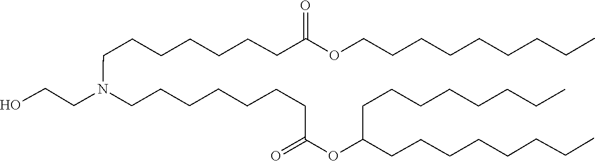 [SMILES: CCCCCCCCCOC(=O)CCCCCCCN(CCO)CCCCCCCC(=O)OC(CCCCCCCC)CCCCCCCC]

and pharmaceutically acceptable salts thereof. In certain embodiments, the compositions and methods of the present invention include a cationic lipid having a compound structure of:

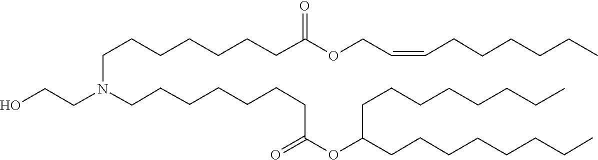 [SMILES: CCCCCC/C=C\COC(=O)CCCCCCCN(CCO)CCCCCCCC(=O)OC(CCCCCCCC)CCCCCCCC]

and pharmaceutically acceptable salts thereof. In certain embodiments, the compositions and methods of the present invention include a cationic lipid having a compound structure of

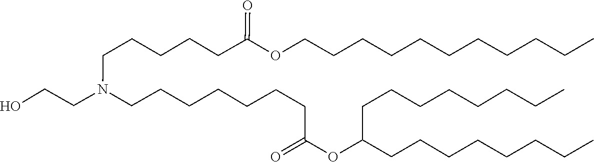 [SMILES: CCCCCCCCCCCOC(=O)CCCCCN(CCO)CCCCCCCC(=O)OC(CCCCCCCC)CCCCCCCC]

and pharmaceutically acceptable salts thereof. In certain embodiments, the compositions and methods of the present invention include a cationic lipid having a compound structure of

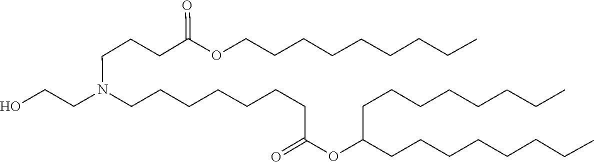 [SMILES: CCCCCCCCCOC(=O)CCCN(CCO)CCCCCCCC(=O)OC(CCCCCCCC)CCCCCCCC]

and pharmaceutically acceptable salts thereof.

Other suitable cationic lipids for use in the compositions and methods of the invention include the cationic lipids as described in International Patent Publication WO 2017/173054 and WO 2015/095340, each of which is incorporated herein by reference. In certain embodiments, the compositions and methods of the present invention include a cationic lipid having a compound structure of

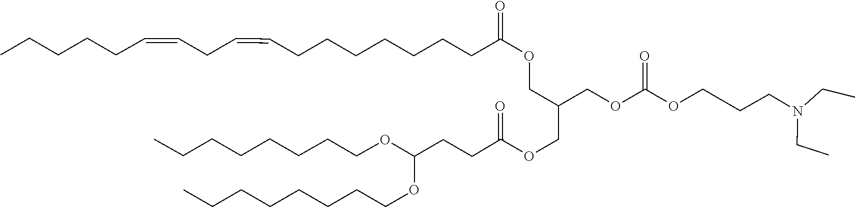 [SMILES: CCCCC/C=C\C/C=C\CCCCCCCC(=O)OCC(COC(=O)CCC(OCCCCCCCC)OCCCCCCCC)COC(=O)OCCCCN(CC)CC]

and pharmaceutically acceptable salts thereof. In certain embodiments, the compositions and methods of the present invention include a cationic lipid having a compound structure of

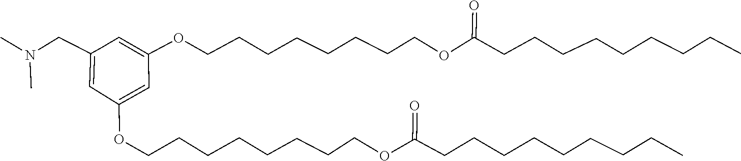 [SMILES: CCCCCCCCCC(=O)OCCCCCCCCOC1=CCC(CN(C)C)=CC(OCCCCCCCCOC(=O)CCCCCCCCC)=C1]

and pharmaceutically acceptable salts thereof. In certain embodiments, the compositions and methods of the present invention include a cationic lipid having a compound structure of

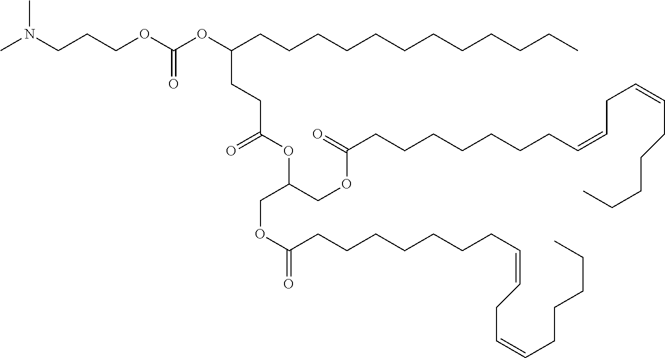 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)OCCCN(C)C]

and pharmaceutically acceptable salts thereof. In certain embodiments, the compositions and methods of the present invention include a cationic lipid having a compound structure of

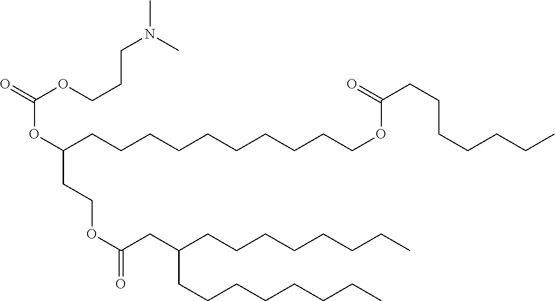 [SMILES: CCCCCCCCC(CCCCCCCC)CC(=O)OCCC(CCCCCCCCCCOC(=O)CCCCCCC)OC(=O)OCCCN(C)C]

and pharmaceutically acceptable salts thereof.

Other suitable cationic lipids for use in the compositions and methods of the present invention include cleavable cationic lipids as described in International Patent Publication WO 2012/170889, which is incorporated herein by reference. In some embodiments, the compositions and methods of the present invention include a cationic lipid of the following formula:

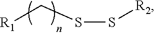

wherein R₁ is selected from the group consisting of imidazole, guanidinium, amino, imine, enamine, an optionally-substituted alkyl amino (e.g., an alkyl amino such as dimethylamino) and pyridyl; wherein R₂ is selected from the group consisting of one of the following two formulas:

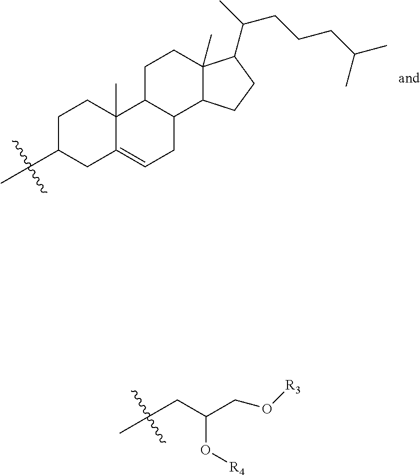

and wherein R₃ and R₄ are each independently selected from the group consisting of an optionally substituted, variably saturated or unsaturated C₆-C₂₀ alkyl and an optionally substituted, variably saturated or unsaturated C₆-C₂₀ acyl; and wherein n is zero or any positive integer (e.g., one, two, three, four, five, six, seven, eight, nine, ten, eleven, twelve, thirteen, fourteen, fifteen, sixteen, seventeen, eighteen, nineteen, twenty or more). In certain embodiments, the compositions and methods of the present invention include a cationic lipid, “HGT4001”, having a compound structure of:

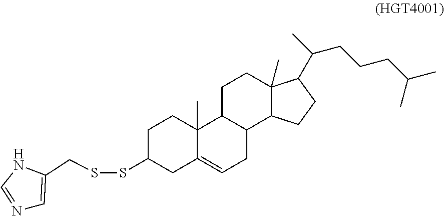 [SMILES: CC(C)CCCC(C)C1CCC2C3CC=C4CC(SSCc5cnc[nH]5)CCC4(C)C3CCC12C]

and pharmaceutically acceptable salts thereof. In certain embodiments, the compositions and methods of the present invention include a cationic lipid, “HGT4002,” (also referred to herein as “Guan-SS-Chol”) having a compound structure of:

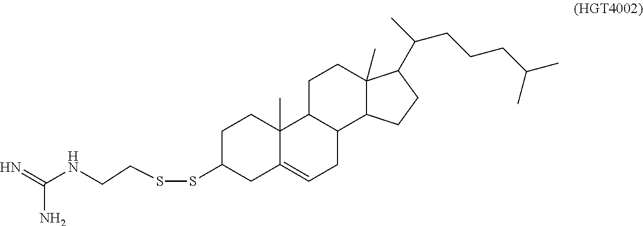 [SMILES: CC(C)CCCC(C)C1CCC2C3CC=C4CC(SSCCNC(=N)N)CCC4(C)C3CCC12C]

and pharmaceutically acceptable salts thereof. In certain embodiments, the compositions and methods of the present invention include a cationic lipid, “HGT4003,” having a compound structure of:

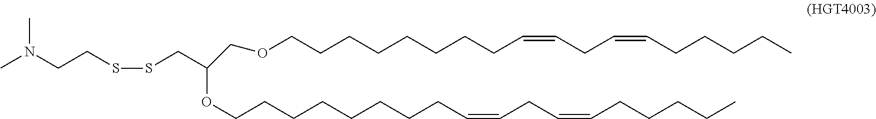 [SMILES: CCCCC/C=C\C/C=C\CCCCCCCCOCC(CSSCCN(C)C)OCCCCCCCC/C=C\C/C=C\CCCCC]

and pharmaceutically acceptable salts thereof. In certain embodiments, the compositions and methods of the present invention include a cationic lipid, “HGT4004,” having a compound structure of:

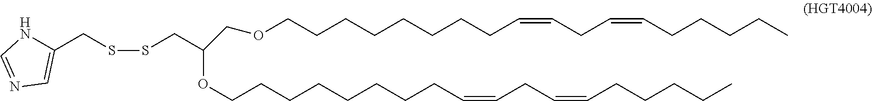 [SMILES: CCCCC/C=C\C/C=C\CCCCCCCCOCC(CSSCc1cnc[nH]1)OCCCCCCCC/C=C\C/C=C\CCCCC]

and pharmaceutically acceptable salts thereof. In certain embodiments, the compositions and methods of the present invention include a cationic lipid “HGT4005,” having a compound structure of:

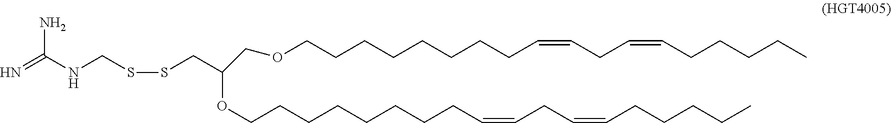 [SMILES: CCCCC/C=C\C/C=C\CCCCCCCCOCC(CSSCNC(=N)N)OCCCCCCCC/C=C\C/C=C\CCCCC]

and pharmaceutically acceptable salts thereof.

Other suitable cationic lipids for use in the compositions and methods of the present invention include cleavable cationic lipids as described in International Application No. PCT/US2019/032522, and incorporated herein by reference. In certain embodiments, the compositions and methods of the present invention include a cationic lipid that is any of general formulas or any of structures (1a)-(21a) and (1b)-(21b) and (22)-(237) described in International Application No. PCT/US2019/032522. In certain embodiments, the compositions and methods of the present invention include a cationic lipid that has a structure according to Formula (I′),

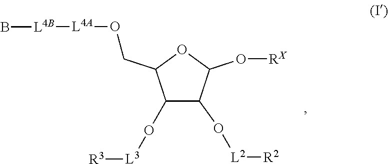

wherein:
R^{X} is independently —H, -L¹-R¹, or -L^{5A}-L^{5B}-B′;
each of L¹, L², and L³ is independently a covalent bond, —C(O)—, —C(O)O—, —C(O)S—, or —C(O)NR^{L}—;
each L^{4A} and L^{5A} is independently —C(O)—, —C(O)O—, or —C(O)NR^{L}—;
each L^{4B} and L^{5B} is independently C₁-C₂₀ alkylene; C₂-C₂₀ alkenylene; or C₂-C₂₀ alkynylene;
each B and B′ is NR⁴R⁵ or a 5- to 10-membered nitrogen-containing heteroaryl;
each R¹, R², and R³ is independently C₆-C₃₀ alkyl, C₆-C₃₀ alkenyl, or C₆-C₃₀ alkynyl;
each R⁴ and R⁵ is independently hydrogen, C₁-C₁₀ alkyl; C₂-C₁₀ alkenyl; or C₂-C₁₀ alkynyl; and
each R^{L} is independently hydrogen, C₁-C₂₀ alkyl, C₂-C₂₀ alkenyl, or C₂-C₂₀ alkynyl.

In certain embodiments, the compositions and methods of the present invention include a cationic lipid that is Compound (139) of International Application No. PCT/US2019/032522, having a compound structure of:

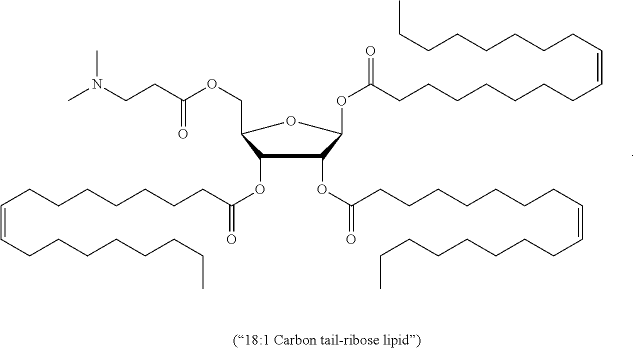

In some embodiments, the compositions and methods of the present invention include a cationic lipid that is TL1-04D-DMA, having a compound structure of:

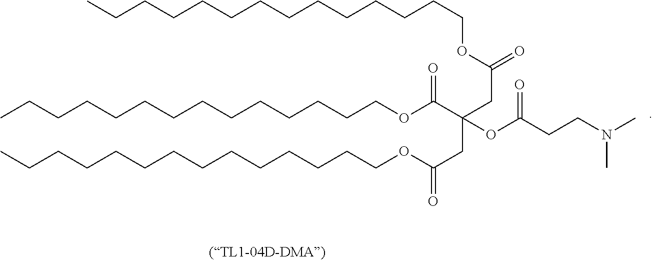 [SMILES: CCCCCCCCCCCCCCOC(=O)CC(CC(=O)OCCCCCCCCCCCCCC)(OC(=O)CCN(C)C)C(=O)OCCCCCCCCCCCCCC]

In some embodiments, the compositions and methods of the present invention include a cationic lipid that is GL-TES-SA-DME-E18-2, having a compound structure of:

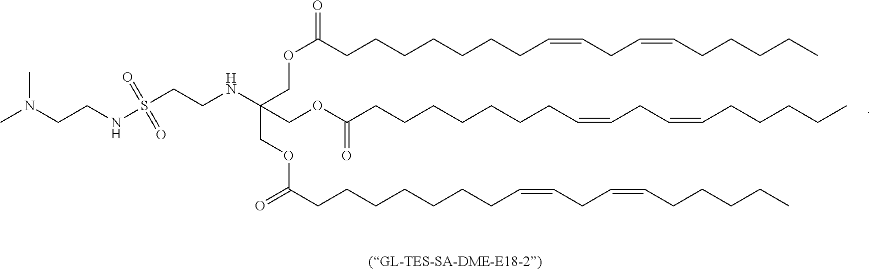 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCC)NCCS(=O)(=O)NCCN(C)C]

In some embodiments, the compositions and methods of the present invention include a cationic lipid that is Guan-SS-Chol, having a compound structure of:

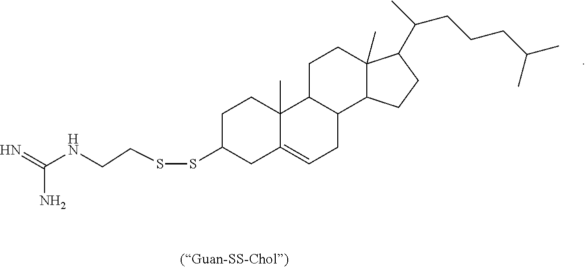 [SMILES: CC(C)CCCC(C)C1CCC2C3CC=C4CC(SSCCNC(=N)N)CCC4(C)C3CCC12C]

In some embodiments, the compositions and methods of the present invention include a cationic lipid that is SY-3-E14-DMAPr, having a compound structure of:

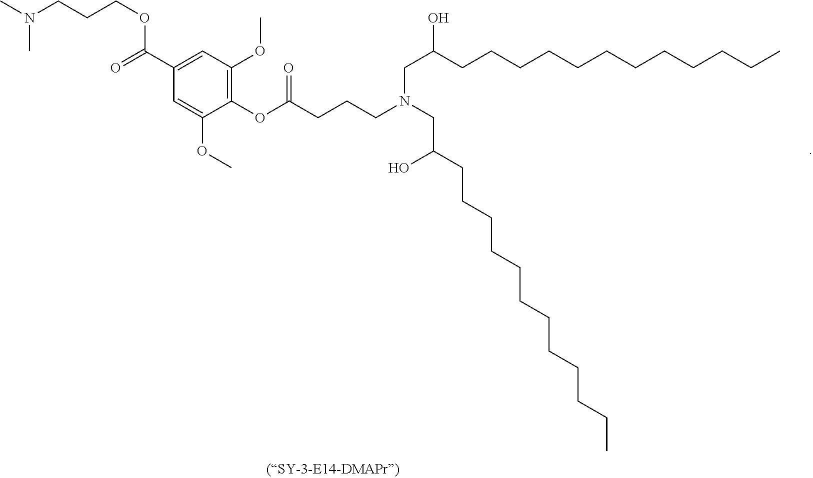 [SMILES: CCCCCCCCCCCCC(O)CN(CCCC(=O)Oc1c(OC)cc(C(=O)OCCCN(C)C)cc1OC)CC(O)CCCCCCCCCCCC]

In some embodiments, the compositions and methods of the present invention include a cationic lipid that is RL3-07D-DMA, having a compound structure of:

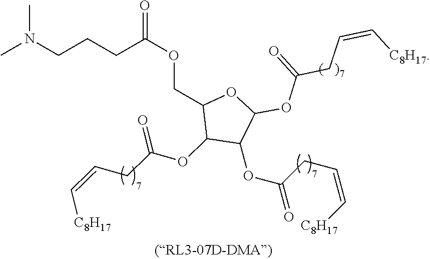

In some embodiments, the compositions and methods of the present invention include a cationic lipid that is TL1-01D-DMA, having a compound structure of:

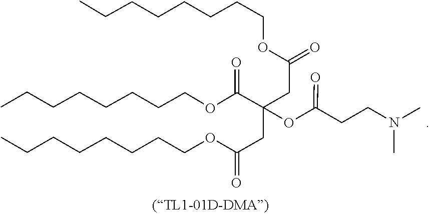 [SMILES: CCCCCCCCOC(=O)CC(CC(=O)OCCCCCCCC)(OC(=O)CCN(C)C)C(=O)OCCCCCCCC]

In some embodiments, the compositions and methods of the present invention include the cationic lipid, N-[1-(2,3-dioleyloxy)propyl]-N,N,N-trimethylammonium chloride (“DOTMA”). (Feigner et al. (Proc. Nat'l Acad. Sci. 84, 7413 (1987); U.S. Pat. No. 4,897,355, which is incorporated herein by reference). Other cationic lipids suitable for the compositions and methods of the present invention include, for example, 5-carboxyspermylglycinedioctadecylamide (“DOGS”); 2,3-dioleyloxy-N-[2(spermine-carboxamido)ethyl]-N,N-dimethyl−1-propanaminium (“DOSPA”) (Behr et al. Proc. Nat.'l Acad. Sci. 86, 6982 (1989), U.S. Pat. Nos. 5,171,678; 5,334,761); 1,2-Dioleoyl-3-Dimethylammonium-Propane (“DODAP”); 1,2-Dioleoyl-3-Trimethylammonium-Propane (“DOTAP”).

Additional exemplary cationic lipids suitable for the compositions and methods of the present invention also include: 1,2-distearyloxy-N,N-dimethyl-3-aminopropane (“DSDMA”); 1,2-dioleyloxy-N,N-dimethyl-3-aminopropane (“DODMA”); 1,2-dilinoleyloxy-N,N-dimethyl-3-aminopropane (“DLinDMA”); 1,2-dilinolenyloxy-N,N-dimethyl-3-aminopropane (“DLenDMA”); N-dioleyl-N,N-dimethylammonium chloride (“DODAC”); N,N-distearyl-N,N-dimethylammonium bromide (“DDAB”); N-(1,2-dimyristyloxyprop-3-yl)-N,N-dimethyl-N-hydroxyethyl ammonium bromide (“DMRIE”); 3-dimethylamino-2-(cholest-5-en-3-beta-oxybutan-4-oxy)-1-(cis,cis-9,12-octadecadienoxy)propane (“CLinDMA”); 2-[5′-(cholest-5-en-3-beta-oxy)-3′-oxapentoxy)-3-dimethy 1-1-(cis,cis-9′,1-2′-octadecadienoxy)propane (“CpLinDMA”); N,N-dimethyl-3,4-dioleyloxybenzylamine (“DMOBA”); 1,2-N,N′-dioleylcarbamyl-3-dimethylaminopropane (“DOcarbDAP”); 2,3-Dilinoleoyloxy-N,N-dimethylpropylamine (“DLinDAP”); 1,2-N,N′-Dilinoleylcarbamyl-3-dimethylaminopropane (“DLincarbDAP”); 1,2-Dilinoleoylcarbamyl-3-dimethylaminopropane (“DLinCDAP”); 2,2-dilinoleyl-4-dimethylaminomethyl-[1,3]-dioxolane (“DLin-K-DMA”); 2-((8-[(3P)-cholest-5-en-3-yloxy]octyl)oxy)-N,N-dimethyl-3-[(9Z,12Z)-octadeca-9,12-dien-1-yloxy]propane-1-amine (“Octyl-CLinDMA”); (2R)-2-((8-[(3beta)-cholest-5-en-3-yloxy]octyl)oxy)-N,N-dimethyl-3-[(9Z,12Z)-octadeca-9,12-dien-1-yloxy]propan-1-amine (“Octyl-CLinDMA (2R)”); (2S)-2-((8-[(3P)-cholest-5-en-3-yloxy]octyl)oxy)-N, fsl-dimethyh3-[(9Z,12Z)-octadeca-9,12-dien-1-yloxy]propan-1-amine (“Octyl-CLinDMA (2S)”); 2,2-dilinoleyl-4-dimethylaminoethyl-[1,3]-dioxolane (“DLin-K-XTC2-DMA”); and 2-(2,2-di((9Z,12Z)-octadeca-9,12-dien-1-yl)-1,3-dioxolan-4-yl)-N,N-dimethylethanamine (“DLin-KC2-DMA”) (see, WO 2010/042877, which is incorporated herein by reference; Semple et al., Nature Biotech. 28: 172-176 (2010)). (Heyes, J., et al., J Controlled Release 107: 276-287 (2005); Morrissey, D V., et al. Nat. Biotechnol. 23(8): 1003-1007 (2005); International Patent Publication WO 2005/121348). In some embodiments, one or more of the cationic lipids comprise at least one of an imidazole, dialkylamino, or guanidinium moiety.

In some embodiments, one or more cationic lipids suitable for the compositions and methods of the present invention include 2,2-Dilinoleyl-4-dimethylaminoethyl-[1,3]-dioxolane (“XTC”); (3aR,5s,6aS)—N,N-dimethyl-2,2-di((9Z,12Z)-octadeca-9,12-dienyl)tetrahydro-3aH-cyclopenta[d][1,3]dioxol-5-amine (“ALNY-100”) and/or 4,7,13-tris(3-oxo-3-(undecylamino)propyl)-N1,N16-diundecyl-4,7,10,13-tetraazahexadecane-1,16-diamide (“NC98-5”).

In some embodiments, one or more cationic lipids suitable for the compositions and methods of the present invention include a cationic lipid that is TL1-04D-DMA, having a compound structure of:

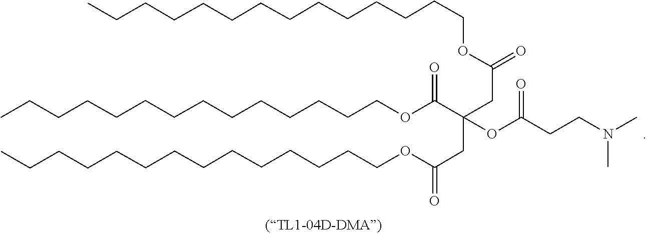 [SMILES: CCCCCCCCCCCCCCOC(=O)CC(CC(=O)OCCCCCCCCCCCCCC)(OC(=O)CCN(C)C)C(=O)OCCCCCCCCCCCCCC]

In some embodiments, one or more cationic lipids suitable for the compositions and methods of the present invention include a cationic lipid that is GL-TES-SA-DME-E18-2, having a compound structure of:

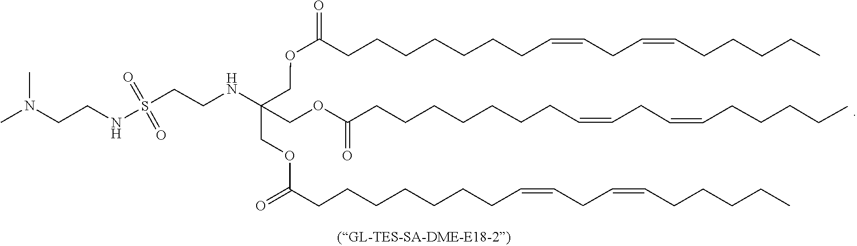 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCC)NCCS(=O)(=O)NCCN(C)C]

In some embodiments, one or more cationic lipids suitable for the compositions and methods of the present invention include a cationic lipid that is SY-3-E14-DMAPr, having a compound structure of:

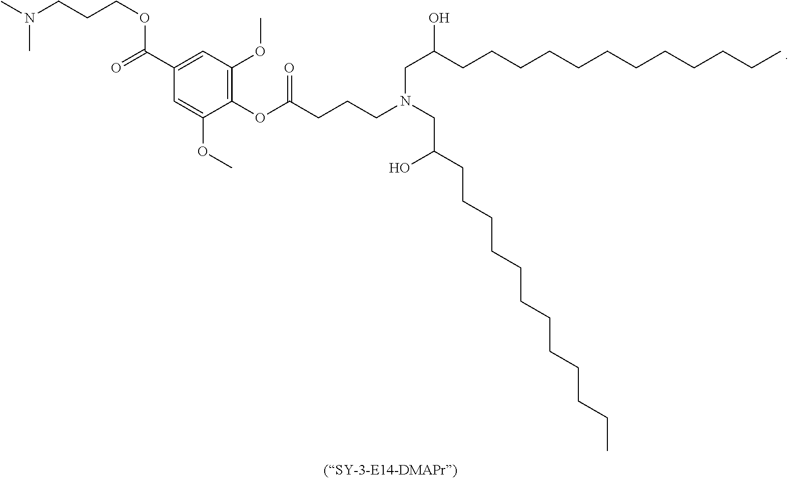 [SMILES: CCCCCCCCCCCCC(O)CN(CCCC(=O)Oc1c(OC)cc(C(=O)OCCCN(C)C)cc1OC)CC(O)CCCCCCCCCCCC]

In some embodiments, one or more cationic lipids suitable for the compositions and methods of the present invention include a cationic lipid that is TL1-01D-DMA, having a compound structure of:

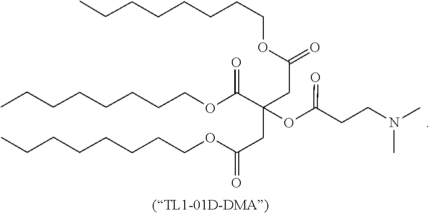 [SMILES: CCCCCCCCOC(=O)CC(CC(=O)OCCCCCCCC)(OC(=O)CCN(C)C)C(=O)OCCCCCCCC]

In some embodiments, one or more cationic lipids suitable for the compositions and methods of the present invention include a cationic lipid that is TL1-TOD-DMA, having a compound structure of:

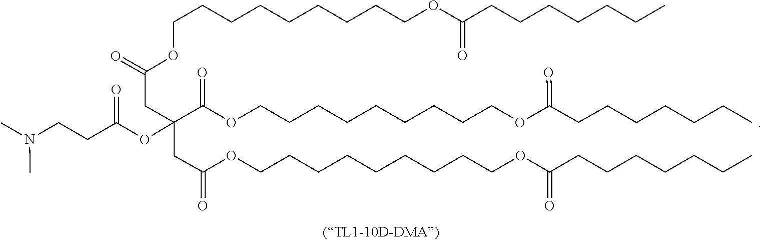 [SMILES: CCCCCCCC(=O)OCCCCCCCCCOC(=O)CC(CC(=O)OCCCCCCCCCOC(=O)CCCCCCC)(OC(=O)CCN(C)C)C(=O)OCCCCCCCCCOC(=O)CCCCCCC]

In some embodiments, one or more cationic lipids suitable for the compositions and methods of the present invention include a cationic lipid that is GL-TES-SA-DMP-E18-2, having a compound structure of:

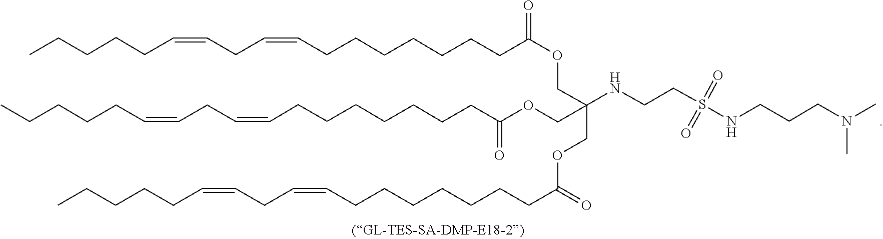 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCC)NCCS(=O)(=O)NCCCN(C)C]

In some embodiments, one or more cationic lipids suitable for the compositions and methods of the present invention include a cationic lipid that is HEP-E4-E10, having a compound structure of:

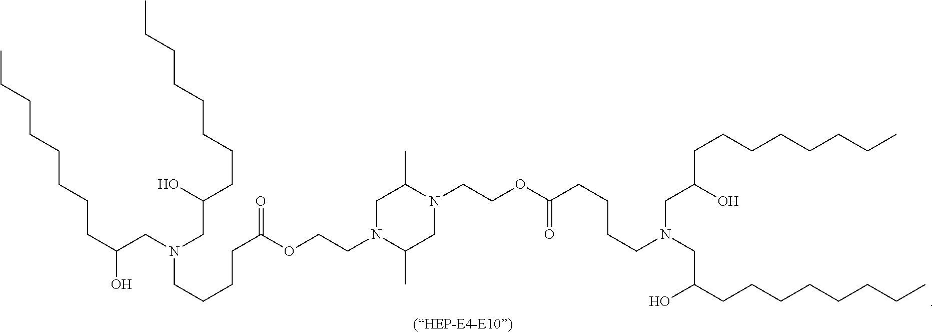 [SMILES: CCCCCCCCC(O)CN(CCCCC(=O)OCCN1CC(C)N(CCOC(=O)CCCCN(CC(O)CCCCCCCC)CC(O)CCCCCCCC)CC1C)CC(O)CCCCCCCC]

In some embodiments, one or more cationic lipids suitable for the compositions and methods of the present invention include a cationic lipid that is HEP-E3-E10, having a compound structure of:

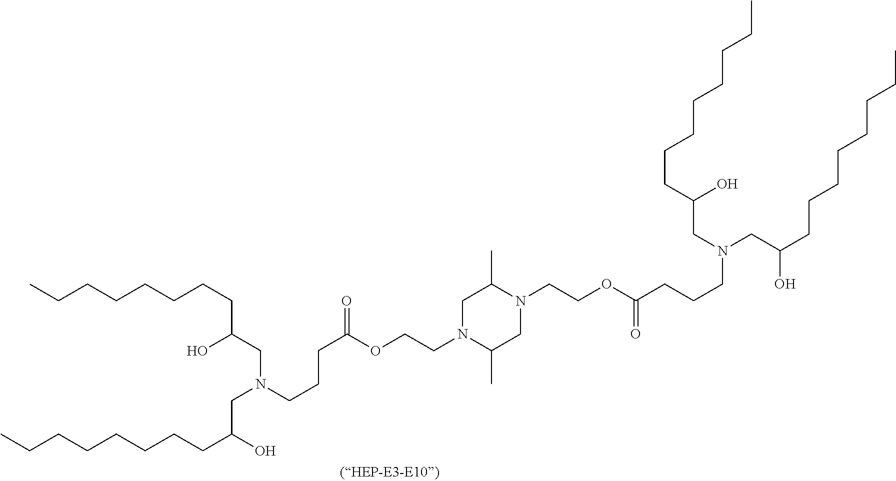 [SMILES: CCCCCCCCC(O)CN(CCCC(=O)OCCN1CC(C)N(CCOC(=O)CCCN(CC(O)CCCCCCCC)CC(O)CCCCCCCC)CC1C)CC(O)CCCCCCCC]

In some embodiments, the compositions of the present invention include one or more cationic lipids that constitute at least about 5%, 10%, 20%, 30%, 35%, 40%, 45%, 50%, 55%, 60%, 65%, or 70%, measured by weight, of the total lipid content in the composition, e.g., a lipid nanoparticle. In some embodiments, the compositions of the present invention include one or more cationic lipids that constitute at least about 5%, 10%, 20%, 30%, 35%, 40%, 45%, 50%, 55%, 60%, 65%, or 70%, measured as a mol %, of the total lipid content in the composition, e.g., a lipid nanoparticle. In some embodiments, the compositions of the present invention include one or more cationic lipids that constitute about 30-70% (e.g., about 30-65%, about 30-60%, about 30-55%, about 30-50%, about 30-45%, about 30-40%, about 35-50%, about 35-45%, or about 35-40%), measured by weight, of the total lipid content in the composition, e.g., a lipid nanoparticle. In some embodiments, the compositions of the present invention include one or more cationic lipids that constitute about 30-70% (e.g., about 30-65%, about 30-60%, about 30-55%, about 30-50%, about 30-45%, about 30-40%, about 35-50%, about 35-45%, or about 35-40%), measured as mol %, of the total lipid content in the composition, e.g., a lipid nanoparticle.

### Non-Cationic Helper Lipids

In some embodiments, the liposomes contain one or more non-cationic (“helper”) lipids. As used herein, the phrase “non-cationic lipid” refers to any neutral, zwitterionic or anionic lipid. As used herein, the phrase “anionic lipid” refers to any of a number of lipid species that carry a net negative charge at a selected pH, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), phosphatidylserine, sphingolipids, cerebrosides, gangliosides, 16-O-monomethyl PE, 16-O-dimethyl PE, 18-1-trans PE, 1-stearoyl-2-oleoyl-phosphatidyethanolamine (SOPE), or a mixture thereof.

In some embodiments, a non-cationic lipid is a neutral lipid, i.e., a lipid that does not carry a net charge in the conditions under which the composition is formulated and/or administered.

In some embodiments, such non-cationic lipids may be used alone, but are preferably used in combination with other lipids, for example, cationic lipids.

In some embodiments, a non-cationic lipid may be present in a molar ratio (mol %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, total non-cationic lipids may be present in a molar ratio (mol %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, the percentage of non-cationic lipid in a liposome may be greater than about 5 mol %, greater than about 10 mol %, greater than about 20 mol %, greater than about 30 mol %, or greater than about 40 mol %. In some embodiments, the percentage total non-cationic lipids in a liposome may be greater than about 5 mol %, greater than about 10 mol %, greater than about 20 mol %, greater than about 30 mol %, or greater than about 40 mol %. In some embodiments, the percentage of non-cationic lipid in a liposome is no more than about 5 mol %, no more than about 10 mol %, no more than about 20 mol %, no more than about 30 mol %, or no more than about 40 mol %. In some embodiments, the percentage total non-cationic lipids in a liposome may be no more than about 5 mol %, no more than about 10 mol %, no more than about 20 mol %, no more than about 30 mol %, or no more than about 40 mol %.

In some embodiments, a non-cationic lipid may be present in a weight ratio (wt %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, total non-cationic lipids may be present in a weight ratio (wt %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, the percentage of non-cationic lipid in a liposome may be greater than about 5 wt %, greater than about 10 wt %, greater than about 20 wt %, greater than about 30 wt %, or greater than about 40 wt %. In some embodiments, the percentage total non-cationic lipids in a liposome may be greater than about 5 wt %, greater than about 10 wt %, greater than about 20 wt %, greater than about 30 wt %, or greater than about 40 wt %. In some embodiments, the percentage of non-cationic lipid in a liposome is no more than about 5 wt %, no more than about 10 wt %, no more than about 20 wt %, no more than about 30 wt %, or no more than about 40 wt %. In some embodiments, the percentage total non-cationic lipids in a liposome may be no more than about 5 wt %, no more than about 10 wt %, no more than about 20 wt %, no more than about 30 wt %, or no more than about 40 wt %.

### Cholesterol-Based Lipids

In some embodiments, the liposomes comprise one or more cholesterol-based lipids. For example, suitable cholesterol-based cationic lipids include, for example, DC-Choi (N,N-dimethyl-N-ethylcarboxamidocholesterol), 1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), or imidazole cholesterol ester (ICE), which has the following structure,

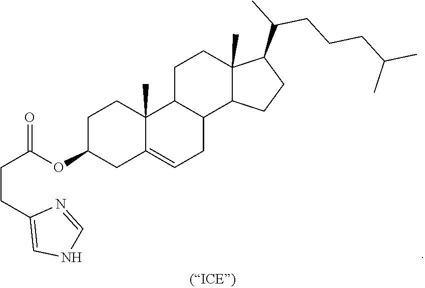 [SMILES: CC(C)CCCC(C)[C@H]1CCC2C3CC=C4C[C@@H](OC(=O)CCc5c[nH]cn5)CC[C@]4(C)C3CC[C@@]21C]

In embodiments, a cholesterol-based lipid is cholesterol.

In some embodiments, the cholesterol-based lipid may comprise a molar ratio (mol %) of about 1% to about 30%, or about 5% to about 20% of the total lipids present in a liposome. In some embodiments, the percentage of cholesterol-based lipid in the lipid nanoparticle may be greater than about 5 mol %, greater than about 10 mol %, greater than about 20 mol %, greater than about 30 mol %, or greater than about 40 mol %. In some embodiments, the percentage of cholesterol-based lipid in the lipid nanoparticle may be no more than about 5 mol %, no more than about 10 mol %, no more than about 20 mol %, no more than about 30 mol %, or no more than about 40 mol %.

In some embodiments, a cholesterol-based lipid may be present in a weight ratio (wt %) of about 1% to about 30%, or about 5% to about 20% of the total lipids present in a liposome. In some embodiments, the percentage of cholesterol-based lipid in the lipid nanoparticle may be greater than about 5 wt %, greater than about 10 wt %, greater than about 20 wt %, greater than about 30 wt %, or greater than about 40 wt %. In some embodiments, the percentage of cholesterol-based lipid in the lipid nanoparticle may be no more than about 5 wt %, no more than about 10 wt %, no more than about 20 wt %, no more than about 30 wt %, or no more than about 40 wt %.

### PEG-Modified Lipids

In some embodiments, the liposome comprises one or more PEGylated lipids.

For example, the use of polyethylene glycol (PEG)-modified phospholipids and derivatized lipids such as derivatized ceramides (PEG-CER), including N-Octanoyl-Sphingosine-1-[Succinyl(Methoxy Polyethylene Glycol)-2000] (C8 PEG-2000 ceramide) is also contemplated by the present invention, either alone or preferably in combination with other lipid formulations together which comprise the transfer vehicle (e.g., a lipid nanoparticle).

Contemplated PEG-modified lipids include, but are not limited to, a polyethylene glycol chain of up to 5 kDa in length covalently attached to a lipid with alkyl chain(s) of C₆-C₂₀ length. In some embodiments, a PEG-modified or PEGylated lipid is PEGylated cholesterol or PEG-2K. The addition of such components may prevent complex aggregation and may also provide a means for increasing circulation lifetime and increasing the delivery of the lipid-nucleic acid composition to the target tissues, (Klibanov et al. (1990) FEBS Letters, 268 (1): 235-237), or they may be selected to rapidly exchange out of the formulation in vivo (see U.S. Pat. No. 5,885,613). Particularly useful exchangeable lipids are PEG-ceramides having shorter acyl chains (e.g., C₁₄ or C₁₈).

The PEG-modified phospholipid and derivitized lipids of the present invention may comprise a molar ratio from about 0% to about 20%, about 0.5% to about 20%, about 1% to about 15%, about 4% to about 10%, or about 2% of the total lipid present in the liposomal transfer vehicle. In some embodiments, one or more PEG-modified lipids constitute about 4% of the total lipids by molar ratio. In some embodiments, one or more PEG-modified lipids constitute about 5% of the total lipids by molar ratio. In some embodiments, one or more PEG-modified lipids constitute about 6% of the total lipids by molar ratio.

### Amphiphilic Block Copolymers

In some embodiments, a suitable delivery vehicle contains amphiphilic block copolymers (e.g., poloxamers).

Various amphiphilic block copolymers may be used to practice the present invention. In some embodiments, an amphiphilic block copolymer is also referred to as a surfactant or a non-ionic surfactant.

In some embodiments, an amphiphilic polymer suitable for the invention is selected from poloxamers (Pluronic®), poloxamines (Tetronic®), polyoxyethylene glycol sorbitan alkyl esters (polysorbates) and polyvinyl pyrrolidones (PVPs).

### Poloxamers

In some embodiments, a suitable amphiphilic polymer is a poloxamer. For example, a suitable poloxamer is of the following structure:

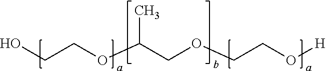

wherein a is an integer between 10 and 150 and b is an integer between 20 and 60. For example, a is about 12 and b is about 20, or a is about 80 and b is about 27, or a is about 64 and b is about 37, or a is about 141 and b is about 44, or a is about 101 and b is about 56.

In some embodiments, a poloxamer suitable for the invention has ethylene oxide units from about 10 to about 150. In some embodiments, a poloxamer has ethylene oxide units from about 10 to about 100.

In some embodiments, a suitable poloxamer is poloxamer 84. In some embodiments, a suitable poloxamer is poloxamer 101. In some embodiments, a suitable poloxamer is poloxamer 105. In some embodiments, a suitable poloxamer is poloxamer 108. In some embodiments, a suitable poloxamer is poloxamer 122. In some embodiments, t a suitable poloxamer is poloxamer 123. In some embodiments, a suitable poloxamer is poloxamer 124. In some embodiments, a suitable poloxamer is poloxamer 181. In some embodiments, a suitable poloxamer is poloxamer 182. In some embodiments, a suitable poloxamer is poloxamer 183. In some embodiments, a suitable poloxamer is poloxamer 184. In some embodiments, a suitable poloxamer is poloxamer 185. In some embodiments, a suitable poloxamer is poloxamer 188. In some embodiments, a suitable poloxamer is poloxamer 212. In some embodiments, a suitable poloxamer is poloxamer 215. In some embodiments, a suitable poloxamer is poloxamer 217. In some embodiments, a suitable poloxamer is poloxamer 231. In some embodiments, a suitable poloxamer is poloxamer 234. In some embodiments, a suitable poloxamer is poloxamer 235. In some embodiments, a suitable poloxamer is poloxamer 237. In some embodiments, a suitable poloxamer is poloxamer 238. In some embodiments, a suitable poloxamer is poloxamer 282. In some embodiments, a suitable poloxamer is poloxamer 284. In some embodiments, a suitable poloxamer is poloxamer 288. In some embodiments, a suitable poloxamer is poloxamer 304. In some embodiments, a suitable poloxamer is poloxamer 331. In some embodiments, a suitable poloxamer is poloxamer 333. In some embodiments, a suitable poloxamer is poloxamer 334. In some embodiments, a suitable poloxamer is poloxamer 335. In some embodiments, a suitable poloxamer is poloxamer 338. In some embodiments, a suitable poloxamer is poloxamer 401. In some embodiments, a suitable poloxamer is poloxamer 402. In some embodiments, a suitable poloxamer is poloxamer 403. In some embodiments, a suitable poloxamer is poloxamer 407. In some embodiments, a suitable poloxamer is a combination thereof.

In some embodiments, a suitable poloxamer has an average molecular weight of about 4,000 g/mol to about 20,000 g/mol. In some embodiments, a suitable poloxamer has an average molecular weight of about 1,000 g/mol to about 50,000 g/mol. In some embodiments, a suitable poloxamer has an average molecular weight of about 1,000 g/mol. In some embodiments, a suitable poloxamer has an average molecular weight of about 2,000 g/mol. In some embodiments, a suitable poloxamer has an average molecular weight of about 3,000 g/mol. In some embodiments, a suitable poloxamer has an average molecular weight of about 4,000 g/mol. In some embodiments, a suitable poloxamer has an average molecular weight of about 5,000 g/mol. In some embodiments, a suitable poloxamer has an average molecular weight of about 6,000 g/mol. In some embodiments, a suitable poloxamer has an average molecular weight of about 7,000 g/mol. In some embodiments, a suitable poloxamer has an average molecular weight of about 8,000 g/mol. In some embodiments, a suitable poloxamer has an average molecular weight of about 9,000 g/mol. In some embodiments, a suitable poloxamer has an average molecular weight of about 10,000 g/mol. In some embodiments, a suitable poloxamer has an average molecular weight of about 20,000 g/mol. In some embodiments, a suitable poloxamer has an average molecular weight of about 25,000 g/mol. In some embodiments, a suitable poloxamer has an average molecular weight of about 30,000 g/mol. In some embodiments, a suitable poloxamer has an average molecular weight of about 40,000 g/mol. In some embodiments, a suitable poloxamer has an average molecular weight of about 50,000 g/mol.

### Other Amphiphilic Polymers

In some embodiments, an amphiphilic polymer is a poloxamine, e.g., tetronic 304 or tetronic 904.

In some embodiments, an amphiphilic polymer is a polyvinylpyrrolidone (PVP), such as PVP with molecular weight of 3 kDa, 10 kDa, or 29 kDa.

In some embodiments, an amphiphilic polymer is a polyethylene glycol ether (Brij), polysorbate, sorbitan, and derivatives thereof. In some embodiments, an amphiphilic polymer is a polysorbate, such as PS 20.

In some embodiments, an amphiphilic polymer is polyethylene glycol ether (Brij), poloxamer, polysorbate, sorbitan, or derivatives thereof.

In some embodiments, an amphiphilic polymer is a polyethylene glycol ether. In some embodiments, a suitable polyethylene glycol ether is a compound of Formula (S-1):

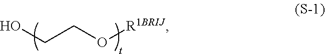

or a salt or isomer thereof, wherein:
t is an integer between 1 and 100;
R^{1BRIJ} independently is C₁₀₋₄₀ alkyl, C₁₀₋₄₀ alkenyl, or C₁₀₋₄₀ alkynyl; and optionally one or more methylene groups of R⁵PEG are independently replaced with C₃₋₁₀ carbocyclylene, 4 to 10 membered heterocyclylene, C₆₋₁₀ arylene, 4 to 10 membered heteroarylene, —N(R^{N})—, —O—, —S—, —C(O)—, —C(O)N(R^{N})—, —NR^{N}C(O)—, —NRC(O)N(R)—, —C(O)O— —OC(O)—, —OC(O)O— —OC(O)N(R^{N})—, —NR^{N}C(O)O— —C(O)S— —SC(O)—, —C(═NR^{N})—, —C(═NR)N(R)—, —NRNC(═NR^{N})— —NR^{N}C(═NR^{N})N(R^{N})—, —C(S)—, —C(S)N(R^{N})—, —NR^{N}C(S)—, —NR^{N}C(S)N(R^{N})—, —(O)—, —S(O)—, —S(O)O—OS(O)O— —OS(O)₂— —S(O)₂O— —OS(O)₂O— —N(R^{N})S(O)—, —S(O)N(R^{N})— —N(R^{N})S(O)N(R^{N})— —OS(O)N(R^{N})— —N(R^{N})S(O)O— —S(O)₂— —N(R^{N})S(O)₂— —S(O)₂N(R^{N})—, —N(R^{N})S(O)₂N(R^{N})— —OS(O)₂N(R^{N})— or —N(R^{N})S(O)₂O—; and
each instance of R^{N} is independently hydrogen, C₁-6 alkyl, or a nitrogen protecting group.

In some embodiment, R^{1BRIJ} is C is alkyl. For example, the polyethylene glycol ether is a compound of Formula (S-la):

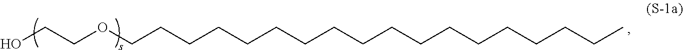

or a salt or isomer thereof, wherein s is an integer between 1 and 100.

In some embodiments, R^{1BRIJ} is C is alkenyl. For example, a suitable polyethylene glycol ether is a compound of Formula (S-1b):

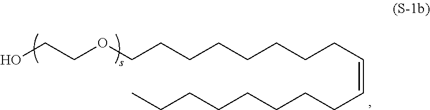

or a salt or isomer thereof, wherein s is an integer between 1 and 100.

Typically, an amphiphilic polymer (e.g., a poloxamer) is present in a formulation at an amount lower than its critical micelle concentration (CMC). In some embodiments, an amphiphilic polymer (e.g., a poloxamer) is present in the mixture at an amount about 1%, about 2%, about 3%, about 4%, about 5%, about 6%, about 7%, about 8%, about 9%, about 10%, about 15%, about 20%, about 25%, about 30%, about 35%, about 40%, about 45%, or about 50% lower than its CMC. In some embodiments, an amphiphilic polymer (e.g., a poloxamer) is present in the mixture at an amount about 0.9%, 0.8%, 0.7%, 0.6%, 0.5%, 0.4%, 0.3%, 0.2%, 0.1% lower than its CMC. In some embodiments, an amphiphilic polymer (e.g., a poloxamer) is present in the mixture at an amount about 55%, 60%, 65%, 70%, 75%, 80%, 90%, or 95% lower than its CMC.

In some embodiments, less than about 0.1%, 0.09%, 0.08%, 0.07%, 0.06%, 0.05%, 0.04%, 0.03%, 0.02%, or 0.01% of the original amount of the amphiphilic polymer (e.g., the poloxamer) present in the formulation remains upon removal. In some embodiments, a residual amount of the amphiphilic polymer (e.g., the poloxamer) remains in a formulation upon removal. As used herein, a residual amount means a remaining amount after substantially all of the substance (an amphiphilic polymer described herein such as a poloxamer) in a composition is removed. A residual amount may be detectable using a known technique qualitatively or quantitatively. A residual amount may not be detectable using a known technique.

In some embodiments, a suitable delivery vehicle comprises less than 5% amphiphilic block copolymers (e.g., poloxamers). In some embodiments, a suitable delivery vehicle comprises less than 3% amphiphilic block copolymers (e.g., poloxamers). In some embodiments, a suitable delivery vehicle comprises less than 2.5% amphiphilic block copolymers (e.g., poloxamers). In some embodiments, suitable delivery vehicle comprises less than 2% amphiphilic block copolymers (e.g., poloxamers). In some embodiments, a suitable delivery vehicle comprises less than 1.5% amphiphilic block copolymers (e.g., poloxamers). In some embodiments, a suitable delivery vehicle comprises less than 1% amphiphilic block copolymers (e.g., poloxamers). In some embodiments, a suitable delivery vehicle comprises less than 0.5% (e.g., less than 0.4%, 0.3%, 0.2%, 0.1%) amphiphilic block copolymers (e.g., poloxamers). In some embodiments, a suitable delivery vehicle comprises less than 0.09%, 0.08%, 0.07%, 0.06%, 0.05%, 0.04%, 0.03%, 0.02%, or 0.01% amphiphilic block copolymers (e.g., poloxamers). In some embodiments, a suitable delivery vehicle comprises less than 0.01% amphiphilic block copolymers (e.g., poloxamers). In some embodiments, a suitable delivery vehicle contains a residual amount of amphiphilic polymers (e.g., poloxamers). As used herein, a residual amount means a remaining amount after substantially all of the substance (an amphiphilic polymer described herein such as a poloxamer) in a composition is removed. A residual amount may be detectable using a known technique qualitatively or quantitatively. A residual amount may not be detectable using a known technique.

### Polymers

In some embodiments, a suitable delivery vehicle is formulated using a polymer as a carrier, alone or in combination with other carriers including various lipids described herein. Thus, in some embodiments, liposomal delivery vehicles, as used herein, also encompass nanoparticles comprising polymers. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, protamine, PEGylated protamine, PLL, PEGylated PLL and polyethylenimine (PEI). When PEI is present, it may be branched PEI of a molecular weight ranging from 10 to 40 kDa, e.g., 25 kDa branched PEI (Sigma #408727).

According to various embodiments, the selection of cationic lipids, non-cationic lipids, PEG-modified lipids, cholesterol-based lipids, and/or amphiphilic block copolymers which comprise the lipid nanoparticle, as well as the relative molar ratio of such components (lipids) to each other, is based upon the characteristics of the selected lipid(s), the nature of the intended target cells, the characteristics of the nucleic acid to be delivered. Additional considerations include, for example, the saturation of the alkyl chain, as well as the size, charge, pH, pKa, fusogenicity and toxicity of the selected lipid(s). Thus the molar ratios may be adjusted accordingly.

Liposomes Suitable for Use with the Present Invention

A suitable liposome for the present invention may include one or more of any of the cationic lipids, non-cationic lipids, cholesterol lipids, PEG-modified lipids, amphiphilic block copolymers and/or polymers described herein at various ratios. In some embodiments, a lipid nanoparticle comprises five and no more than five distinct components of nanoparticle. In some embodiments, a lipid nanoparticle comprises four and no more than four distinct components of nanoparticle. In some embodiments, a lipid nanoparticle comprises three and no more than three distinct components of nanoparticle. As non-limiting examples, a suitable liposome formulation may include a combination selected from cKK-E12, DOPE, cholesterol and DMG-PEG2K; C12-200, DOPE, cholesterol and DMG-PEG2K; HGT4003, DOPE, cholesterol and DMG-PEG2K; ICE, DOPE, cholesterol and DMG-PEG2K; or ICE, DOPE, and DMG-PEG2K.

In particular embodiments, a liposome for use with this invention comprises a lipid component consisting of a cationic lipid, a non-cationic lipid (e.g., DOPE or DEPE), a PEG-modified lipid (e.g., DMG-PEG2K), and optionally cholesterol. Cationic lipids particularly suitable for inclusion in such a liposome include GL-TES-SA-DME-E18-2, TL1-01D-DMA, SY-3-E14-DMAPr, TL1-10D-DMA, HGT4002 (also referred to herein as Guan-SS-Chol), GL-TES-SA-DMP-E18-2, HEP-E4-E10, HEP-E3-E10, and TL1-04D-DMA. These cationic lipids have been found to be particularly suitable for use in liposomes that are administered through pulmonary delivery via nebulization. Amongst these, HEP-E4-E10, HEP-E3-E10, GL-TES-SA-DME-E18-2, GL-TES-SA-DMP-E18-2, TL1-01D-DMA and TL1-04D-DMA performed particularly well.

In some embodiments, an HBEC-ALI (Human Bronchial Epithelial Cell-Air Liquid Interface) system can be used to assess mucociliary transport (MCT) using micro-optical coherence tomography (uOCT), e.g., by visualizing MCT using fluorescent microbeads. Accordingly, HBEC-ALI can be used to assess the potency of a liposome encapsulating a codon-optimized DNAI1 mRNA sequence for use in treating primary ciliary dyskinesia (PCD).

Exemplary liposomes for use with the present invention include one of GL-TES-SA-DME-E18-2, TL1-01D-DMA, SY-3-E14-DMAPr, TL1-10D-DMA, GL-TES-SA-DMP-E18-2, HEP-E4-E10, HEP-E3-E10 and TL1-04D-DMA as a cationic lipid component, DOPE as a non-cationic lipid component, cholesterol as a helper lipid component, and DMG-PEG2K as a PEG-modified lipid component. In some embodiments, the molar ratio of the cationic lipid to non-cationic lipid to cholesterol to PEG-modified lipid may be between about 30-60:25-35:20-30:1-15, respectively. In some embodiments, the molar ratio of cationic lipid to non-cationic lipid to cholesterol to PEG-modified lipid is approximately 40:30:20:10, respectively. In some embodiments, the molar ratio of cationic lipid to non-cationic lipid to cholesterol to PEG-modified lipid is approximately 40:30:25:5, respectively. In some embodiments, the molar ratio of cationic lipid to non-cationic lipid to cholesterol to PEG-modified lipid is approximately 40:32:25:3, respectively. In some embodiments, the molar ratio of cationic lipid to non-cationic lipid to cholesterol to PEG-modified lipid is approximately 50:25:20:5.

In some embodiments, the lipid component of a liposome particularly suitable for pulmonary delivery consists of HGT4002 (also referred to herein as Guan-SS-Chol), DOPE and DMG-PEG2K. In some embodiments, the molar ratio of cationic lipid to non-cationic lipid to PEG-modified lipid is approximately 60:35:5.

In various embodiments, cationic lipids (e.g., cKK-E12, C12-200, ICE, and/or HGT4003) constitute about 30-60% (e.g., about 30-55%, about 30-50%, about 30-45%, about 30-40%, about 35-50%, about 35-45%, or about 35-40%) of the liposome by molar ratio. In some embodiments, the percentage of cationic lipids (e.g., cKK-E12, C12-200, ICE, and/or HGT4003) is or greater than about 30%, about 35%, about 40%, about 45%, about 50%, about 55%, or about 60% of the liposome by molar ratio.

In some embodiments, the ratio of cationic lipid(s) to non-cationic lipid(s) to cholesterol-based lipid(s) to PEG-modified lipid(s) may be between about 30-60:25-35:20-30:1-15, respectively. In some embodiments, the ratio of cationic lipid(s) to non-cationic lipid(s) to cholesterol-based lipid(s) to PEG-modified lipid(s) is approximately 40:30:20:10, respectively. In some embodiments, the ratio of cationic lipid(s) to non-cationic lipid(s) to cholesterol-based lipid(s) to PEG-modified lipid(s) is approximately 40:30:25:5, respectively. In some embodiments, the ratio of cationic lipid(s) to non-cationic lipid(s) to cholesterol-based lipid(s) to PEG-modified lipid(s) is approximately 40:32:25:3, respectively. In some embodiments, the ratio of cationic lipid(s) to non-cationic lipid(s) to cholesterol-based lipid(s) to PEG-modified lipid(s) is approximately 50:25:20:5.

### Ratio of Distinct Lipid Components

In embodiments where a lipid nanoparticle comprises three and no more than three distinct components of lipids, the ratio of total lipid content (i.e., the ratio of lipid component (1):lipid component (2):lipid component (3)) can be represented as x:y:z, wherein

$$( y + z ) = 100 - x .$$

In some embodiments, each of “x,” “y,” and “z” represents molar percentages of the three distinct components of lipids, and the ratio is a molar ratio.

In some embodiments, each of “x,” “y,” and “z” represents weight percentages of the three distinct components of lipids, and the ratio is a weight ratio.

In some embodiments, lipid component (1), represented by variable “x,” is a sterol-based cationic lipid.

In some embodiments, lipid component (2), represented by variable “y,” is a helper lipid.

In some embodiments, lipid component (3), represented by variable “z” is a PEG lipid.

In some embodiments, variable “x,” representing the molar percentage of lipid component (1) (e.g., a sterol-based cationic lipid), is at least about 10%, about 20%, about 30%, about 40%, about 50%, about 55%, about 60%, about 65%, about 70%, about 75%, about 80%, about 85%, about 90%, or about 95%.

In some embodiments, variable “x,” representing the molar percentage of lipid component (1) (e.g., a sterol-based cationic lipid), is no more than about 95%, about 90%, about 85%, about 80%, about 75%, about 70%, about 65%, about 60%, about 55%, about 50%, about 40%, about 30%, about 20%, or about 10%. In embodiments, variable “x” is no more than about 65%, about 60%, about 55%, about 50%, about 40%.

In some embodiments, variable “x,” representing the molar percentage of lipid component (1) (e.g., a sterol-based cationic lipid), is: at least about 50% but less than about 95%; at least about 50% but less than about 90%; at least about 50% but less than about 85%; at least about 50% but less than about 80%; at least about 50% but less than about 75%; at least about 50% but less than about 70%; at least about 50% but less than about 65%; or at least about 50% but less than about 60%. In embodiments, variable “x” is at least about 50% but less than about 70%; at least about 50% but less than about 65%; or at least about 50% but less than about 60%.

In some embodiments, variable “x,” representing the weight percentage of lipid component (1) (e.g., a sterol-based cationic lipid), is at least about 10%, about 20%, about 30%, about 40%, about 50%, about 55%, about 60%, about 65%, about 70%, about 75%, about 80%, about 85%, about 90%, or about 95%.

In some embodiments, variable “x,” representing the weight percentage of lipid component (1) (e.g., a sterol-based cationic lipid), is no more than about 95%, about 90%, about 85%, about 80%, about 75%, about 70%, about 65%, about 60%, about 55%, about 50%, about 40%, about 30%, about 20%, or about 10%. In embodiments, variable “x” is no more than about 65%, about 60%, about 55%, about 50%, about 40%.

In some embodiments, variable “x,” representing the weight percentage of lipid component (1) (e.g., a sterol-based cationic lipid), is: at least about 50% but less than about 95%; at least about 50% but less than about 90%; at least about 50% but less than about 85%; at least about 50% but less than about 80%; at least about 50% but less than about 75%; at least about 50% but less than about 70%; at least about 50% but less than about 65%; or at least about 50% but less than about 60%. In embodiments, variable “x” is at least about 50% but less than about 70%; at least about 50% but less than about 65%; or at least about 50% but less than about 60%.

In some embodiments, variable “z,” representing the molar percentage of lipid component (3) (e.g., a PEG lipid) is no more than about 1%, 2%, 3%, 4%, 5%, 6%, 7%, 8%, 9%, 10%, 15%, 20%, or 25%. In embodiments, variable “z,” representing the molar percentage of lipid component (3) (e.g., a PEG lipid) is about 1%, 2%, 3%, 4%, 5%, 6%, 7%, 8%, 9%, 10%. In embodiments, variable “z,” representing the molar percentage of lipid component (3) (e.g., a PEG lipid) is about 1% to about 10%, about 2% to about 10%, about 3% to about 10%, about 4% to about 10%, about 1% to about 7.5%, about 2.5% to about 10%, about 2.5% to about 7.5%, about 2.5% to about 5%, about 5% to about 7.5%, or about 5% to about 10%.

In some embodiments, variable “z,” representing the weight percentage of lipid component (3) (e.g., a PEG lipid) is no more than about 1%, 2%, 3%, 4%, 5%, 6%, 7%, 8%, 9%, 10%, 15%, 20%, or 25%. In embodiments, variable “z,” representing the weight percentage of lipid component (3) (e.g., a PEG lipid) is about 1%, 2%, 3%, 4%, 5%, 6%, 7%, 8%, 9%, 10%. In embodiments, variable “z,” representing the weight percentage of lipid component (3) (e.g., a PEG lipid) is about 10% to about 10%, about 2% to about 10%, about 3% to about 10%, about 4% to about 10%, about 1% to about 7.5%, about 2.5% to about 10%, about 2.5% to about 7.5%, about 2.5% to about 5%, about 5% to about 7.5%, or about 5% to about 10%.

For compositions having three and only three distinct lipid components, variables “x,” “y,” and “z” may be in any combination so long as the total of the three variables sums to 100% of the total lipid content.

Formation of Liposomes Encapsulating mRNA

The liposomal transfer vehicles for use in the compositions of the invention can be prepared by various techniques which are presently known in the art. For example, multilamellar vesicles (MLV) may be prepared according to conventional techniques, such as by depositing a selected lipid on the inside wall of a suitable container or vessel by dissolving the lipid in an appropriate solvent, and then evaporating the solvent to leave a thin film on the inside of the vessel or by spray drying. An aqueous phase may then be added to the vessel with a vortexing motion which results in the formation of MLVs. Unilamellar vesicles (ULV) can then be formed by homogenization, sonication or extrusion of the multilamellar vesicles. In addition, unilamellar vesicles can be formed by detergent removal techniques.

Various methods are described in published U.S. Application No. US 2011/0244026, published U.S. Application No. US 2016/0038432, published U.S. Application No. US 2018/0153822, published U.S. Application No. US 2018/0125989 and U.S. Provisional Application No. 62/877,597, filed Jul. 23, 2019 and can be used to practice the present invention, all of which are incorporated herein by reference. As used herein, Process A refers to a conventional method of encapsulating mRNA by mixing mRNA with a mixture of lipids, without first pre-forming the lipids into lipid nanoparticles, as described in US 2016/0038432. As used herein, Process B refers to a process of encapsulating messenger RNA (mRNA) by mixing pre-formed lipid nanoparticles with mRNA, as described in US 2018/0153822.

Briefly, the process of preparing mRNA- or MCNA-loaded lipid liposomes includes a step of heating one or more of the solutions (i.e., applying heat from a heat source to the solution) to a temperature (or to maintain at a temperature) greater than ambient temperature, the one more solutions being the solution comprising the pre-formed lipid nanoparticles, the solution comprising the mRNA and the mixed solution comprising the lipid nanoparticle encapsulated mRNA. In some embodiments, the process includes the step of heating one or both of the mRNA solution and the pre-formed lipid nanoparticle solution, prior to the mixing step. In some embodiments, the process includes heating one or more one or more of the solution comprising the pre-formed lipid nanoparticles, the solution comprising the mRNA and the solution comprising the lipid nanoparticle encapsulated mRNA, during the mixing step. In some embodiments, the process includes the step of heating the lipid nanoparticle encapsulated mRNA, after the mixing step. In some embodiments, the temperature to which one or more of the solutions is heated (or at which one or more of the solutions is maintained) is or is greater than about 30° C., 37° C., 40° C., 45° C., 50° C., 55° C., 60° C., 65° C., or 70° C. In some embodiments, the temperature to which one or more of the solutions is heated ranges from about 25-70° C., about 30-70° C., about 35-70° C., about 40-70° C., about 45-70° C., about 50-70° C., or about 60-70° C. In some embodiments, the temperature greater than ambient temperature to which one or more of the solutions is heated is about 65° C.

Various methods may be used to prepare an mRNA solution suitable for the present invention. In some embodiments, mRNA may be directly dissolved in a buffer solution described herein. In some embodiments, an mRNA solution may be generated by mixing an mRNA stock solution with a buffer solution prior to mixing with a lipid solution for encapsulation. In some embodiments, an mRNA solution may be generated by mixing an mRNA stock solution with a buffer solution immediately before mixing with a lipid solution for encapsulation. In some embodiments, a suitable mRNA stock solution may contain mRNA in water at a concentration at or greater than about 0.2 mg/ml, 0.4 mg/ml, 0.5 mg/ml, 0.6 mg/ml, 0.8 mg/ml, 1.0 mg/ml, 1.2 mg/ml, 1.4 mg/ml, 1.5 mg/ml, or 1.6 mg/ml, 2.0 mg/ml, 2.5 mg/ml, 3.0 mg/ml, 3.5 mg/ml, 4.0 mg/ml, 4.5 mg/ml, or 5.0 mg/ml.

In some embodiments, an mRNA stock solution is mixed with a buffer solution using a pump. Exemplary pumps include but are not limited to gear pumps, peristaltic pumps and centrifugal pumps.

Typically, the buffer solution is mixed at a rate greater than that of the mRNA stock solution. For example, the buffer solution may be mixed at a rate at least 1×, 2×, 3×, 4×, 5×, 6×, 7×, 8×, 9×, 10×, 15×, or 20× greater than the rate of the mRNA stock solution. In some embodiments, a buffer solution is mixed at a flow rate ranging between about 100-6000 ml/minute (e.g., about 100-300 ml/minute, 300-600 ml/minute, 600-1200 ml/minute, 1200-2400 ml/minute, 2400-3600 ml/minute, 3600-4800 ml/minute, 4800-6000 ml/minute, or 60-420 ml/minute). In some embodiments, a buffer solution is mixed at a flow rate of or greater than about 60 ml/minute, 100 ml/minute, 140 ml/minute, 180 ml/minute, 220 ml/minute, 260 ml/minute, 300 ml/minute, 340 ml/minute, 380 ml/minute, 420 ml/minute, 480 ml/minute, 540 ml/minute, 600 ml/minute, 1200 ml/minute, 2400 ml/minute, 3600 ml/minute, 4800 ml/minute, or 6000 ml/minute.

In some embodiments, an mRNA stock solution is mixed at a flow rate ranging between about 10-600 ml/minute (e.g., about 5-50 ml/minute, about 10-30 ml/minute, about 30-60 ml/minute, about 60-120 ml/minute, about 120-240 ml/minute, about 240-360 ml/minute, about 360-480 ml/minute, or about 480-600 ml/minute). In some embodiments, an mRNA stock solution is mixed at a flow rate of or greater than about 5 ml/minute, 10 ml/minute, 15 ml/minute, 20 ml/minute, 25 ml/minute, 30 ml/minute, 35 ml/minute, 40 ml/minute, 45 ml/minute, 50 ml/minute, 60 ml/minute, 80 ml/minute, 100 ml/minute, 200 ml/minute, 300 ml/minute, 400 ml/minute, 500 ml/minute, or 600 ml/minute.

According to the present invention, a lipid solution contains a mixture of lipids suitable to form lipid nanoparticles for encapsulation of mRNA. In some embodiments, a suitable lipid solution is ethanol based. For example, a suitable lipid solution may contain a mixture of desired lipids dissolved in pure ethanol (i.e., 100% ethanol). In another embodiment, a suitable lipid solution is isopropyl alcohol based. In another embodiment, a suitable lipid solution is dimethylsulfoxide-based. In another embodiment, a suitable lipid solution is a mixture of suitable solvents including, but not limited to, ethanol, isopropyl alcohol and dimethylsulfoxide.

A suitable lipid solution may contain a mixture of desired lipids at various concentrations. For example, a suitable lipid solution may contain a mixture of desired lipids at a total concentration of or greater than about 0.1 mg/ml, 0.5 mg/ml, 1.0 mg/ml, 2.0 mg/ml, 3.0 mg/ml, 4.0 mg/ml, 5.0 mg/ml, 6.0 mg/ml, 7.0 mg/ml, 8.0 mg/ml, 9.0 mg/ml, 10 mg/ml, 15 mg/ml, 20 mg/ml, 30 mg/ml, 40 mg/ml, 50 mg/ml, or 100 mg/ml. In some embodiments, a suitable lipid solution may contain a mixture of desired lipids at a total concentration ranging from about 0.1-100 mg/ml, 0.5-90 mg/ml, 1.0-80 mg/ml, 1.0-70 mg/ml, 1.0-60 mg/ml, 1.0-50 mg/ml, 1.0-40 mg/ml, 1.0-30 mg/ml, 1.0-20 mg/ml, 1.0-15 mg/ml, 1.0-10 mg/ml, 1.0-9 mg/ml, 1.0-8 mg/ml, 1.0-7 mg/ml, 1.0-6 mg/ml, or 1.0-5 mg/ml. In some embodiments, a suitable lipid solution may contain a mixture of desired lipids at a total concentration up to about 100 mg/ml, 90 mg/ml, 80 mg/ml, 70 mg/ml, 60 mg/ml, 50 mg/ml, 40 mg/ml, 30 mg/ml, 20 mg/ml, or 10 mg/ml.

Any desired lipids may be mixed at any ratios suitable for encapsulating mRNAs. In some embodiments, a suitable lipid solution contains a mixture of desired lipids including cationic lipids, helper lipids (e.g. non cationic lipids and/or cholesterol lipids), amphiphilic block copolymers (e.g. poloxamers) and/or PEGylated lipids. In some embodiments, a suitable lipid solution contains a mixture of desired lipids including one or more cationic lipids, one or more helper lipids (e.g. non cationic lipids and/or cholesterol lipids) and one or more PEGylated lipids.

In certain embodiments, provided compositions comprise a liposome wherein the mRNA is associated on both the surface of the liposome and encapsulated within the same liposome. For example, during preparation of the compositions of the present invention, cationic liposomes may associate with the mRNA or MCNA through electrostatic interactions.

In some embodiments, the compositions and methods of the invention comprise mRNA encapsulated in a liposome. In some embodiments, the one or more mRNA species may be encapsulated in the same liposome. In some embodiments, the one or more mRNA species may be encapsulated in different liposomes. In some embodiments, the mRNA is encapsulated in one or more liposomes, which differ in their lipid composition, molar ratio of lipid components, size, charge (zeta potential), targeting ligands and/or combinations thereof. In some embodiments, the one or more liposome may have a different composition of sterol-based cationic lipids, neutral lipid, PEG-modified lipid and/or combinations thereof. In some embodiments the one or more liposomes may have a different molar ratio of cholesterol-based cationic lipid, neutral lipid, and PEG-modified lipid used to create the liposome.

The process of incorporation of a desired nucleic acid (e.g., mRNA or MCNA) into a liposome is often referred to as “loading”. Exemplary methods are described in Lasic, et al. FEBS Lett., 312: 255-258, 1992, which is incorporated herein by reference. The liposome-incorporated nucleic acids may be completely or partially located in the interior space of the liposome, within the bilayer membrane of the liposome, or associated with the exterior surface of the liposome membrane. The incorporation of a nucleic acid into liposomes is also referred to herein as “encapsulation” wherein the nucleic acid is entirely contained within the interior space of the liposome. The purpose of incorporating an mRNA into a transfer vehicle, such as a liposome, is often to protect the nucleic acid from an environment which may contain enzymes or chemicals that degrade nucleic acids and/or systems or receptors that cause the rapid excretion of the nucleic acids. Accordingly, in some embodiments, a suitable delivery vehicle is capable of enhancing the stability of the mRNA contained therein and/or facilitate the delivery of therapeutic agent (e.g., mRNA or MCNA) to the target cell or tissue.

Suitable liposomes in accordance with the present invention may be made in various sizes. In some embodiments, provided liposomes may be made smaller than previously known liposomes. In some embodiments, decreased size of liposomes is associated with more efficient delivery of therapeutic agent (e.g., mRNA or MCNA). Selection of an appropriate liposome size may take into consideration the site of the target cell or tissue and to some extent the application for which the liposome is being made.

In some embodiments, an appropriate size of liposome is selected to facilitate systemic distribution of antibody encoded by the mRNA. In some embodiments, it may be desirable to limit transfection of the mRNA to certain cells or tissues. For example, to target hepatocytes a liposome may be sized such that its dimensions are smaller than the fenestrations of the endothelial layer lining hepatic sinusoids in the liver; in such cases the liposome could readily penetrate such endothelial fenestrations to reach the target hepatocytes.

Alternatively or additionally, a liposome may be sized such that the dimensions of the liposome are of a sufficient diameter to limit or expressly avoid distribution into certain cells or tissues.

A variety of alternative methods known in the art are available for sizing of a population of liposomes. One such sizing method is described in U.S. Pat. No. 4,737,323, incorporated herein by reference. Sonicating a liposome suspension either by bath or probe sonication produces a progressive size reduction down to small ULV less than about 0.05 microns in diameter. Homogenization is another method that relies on shearing energy to fragment large liposomes into smaller ones. In a typical homogenization procedure, MLV are recirculated through a standard emulsion homogenizer until selected liposome sizes, typically between about 0.1 and 0.5 microns, are observed. The size of the liposomes may be determined by quasi-electric light scattering (QELS) as described in Bloomfield, Ann. Rev. Biophys. Bioeng., 10:421-450 (1981), incorporated herein by reference. Average liposome diameter may be reduced by sonication of formed liposomes. Intermittent sonication cycles may be alternated with QELS assessment to guide efficient liposome synthesis.

Provided Nanoparticles Encapsulating mRNA

In some embodiments, majority of purified nanoparticles in a composition, i.e., greater than about 50%, 55%, 60%, 65%, 70%, 75%, 80%, 85%, 90%, 95%, 96%, 97%, 98%, or 99% of the nanoparticles, have a size of about 150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, about 80 nm, about 75 nm, about 70 nm, about 65 nm, about 60 nm, about 55 nm, about 50 nm, about 45 nm, about 40 nm, about 35 nm, or about 30 nm). In some embodiments, substantially all of the purified nanoparticles have a size of about 150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, about 80 nm, about 75 nm, about 70 nm, about 65 nm, about 60 nm, about 55 nm, about 50 nm, about 45 nm, about 40 nm, about 35 nm, or about 30 nm).

In some embodiments, a lipid nanoparticle has an average size of less than 150 nm. In some embodiments, a lipid nanoparticle has an average size of less than 120 nm. In some embodiments, a lipid nanoparticle has an average size of less than 100 nm. In some embodiments, a lipid nanoparticle has an average size of less than 90 nm. In some embodiments, a lipid nanoparticle has an average size of less than 80 nm. In some embodiments, a lipid nanoparticle has an average size of less than 70 nm. In some embodiments, a lipid nanoparticle has an average size of less than 60 nm. In some embodiments, a lipid nanoparticle has an average size of less than 50 nm. In some embodiments, a lipid nanoparticle has an average size of less than 30 nm. In some embodiments, a lipid nanoparticle has an average size of less than 20 nm.

In some embodiments, the dispersity, or measure of heterogeneity in size of molecules (PDI), of nanoparticles in a composition provided by the present invention is less than about 0.5. In some embodiments, a lipid nanoparticle has a PDI of less than about 0.5. In some embodiments, a lipid nanoparticle has a PDI of less than about 0.4. In some embodiments, a lipid nanoparticle has a PDI of less than about 0.3. In some embodiments, a lipid nanoparticle has a PDI of less than about 0.28. In some embodiments, a lipid nanoparticle has a PDI of less than about 0.25. In some embodiments, a lipid nanoparticle has a PDI of less than about 0.23. In some embodiments, a lipid nanoparticle has a PDI of less than about 0.20. In some embodiments, a lipid nanoparticle has a PDI of less than about 0.18. In some embodiments, a lipid nanoparticle has a PDI of less than about 0.16. In some embodiments, a lipid nanoparticle has a PDI of less than about 0.14. In some embodiments, a lipid nanoparticle has a PDI of less than about 0.12. In some embodiments, a lipid nanoparticle has a PDI of less than about 0.10. In some embodiments, a lipid nanoparticle has a PDI of less than about 0.08.

In some embodiments, greater than about 75%, 80%, 85%, 90%, 95%, 96%, 97%, 98%, or 99% of the purified lipid nanoparticles in a composition provided by the present invention encapsulate an mRNA within each individual particle. In some embodiments, substantially all of the purified lipid nanoparticles in a composition encapsulate an mRNA within each individual particle. In some embodiments, a lipid nanoparticle has an encapsulation efficiency of between 50% and 99%. In some embodiments, a lipid nanoparticle has an encapsulation efficiency of greater than about 60%. In some embodiments, a lipid nanoparticle has an encapsulation efficiency of greater than about 65%. In some embodiments, a lipid nanoparticle has an encapsulation efficiency of greater than about 70%. In some embodiments, a lipid nanoparticle has an encapsulation efficiency of greater than about 75%. In some embodiments, a lipid nanoparticle has an encapsulation efficiency of greater than about 80%. In some embodiments, a lipid nanoparticle has an encapsulation efficiency of greater than about 85%. In some embodiments, a lipid nanoparticle has an encapsulation efficiency of greater than about 90%. In some embodiments, a lipid nanoparticle has an encapsulation efficiency of greater than about 92%. In some embodiments, a lipid nanoparticle has an encapsulation efficiency of greater than about 95%. In some embodiments, a lipid nanoparticle has an encapsulation efficiency of greater than about 98%. In some embodiments, a lipid nanoparticle has an encapsulation efficiency of greater than about 99%.

In some embodiments, a lipid nanoparticle has an N/P ratio of between 1 and 10. As used herein, the term “N/P ratio” refers to a molar ratio of positively charged molecular units in the cationic lipids in a lipid nanoparticle relative to negatively charged molecular units in the mRNA encapsulated within that lipid nanoparticle. As such, N/P ratio is typically calculated as the ratio of moles of amine groups in cationic lipids in a lipid nanoparticle relative to moles of phosphate groups in mRNA encapsulated within that lipid nanoparticle. In some embodiments, a lipid nanoparticle has an N/P ratio above 1. In some embodiments, a lipid nanoparticle has an N/P ratio of about 1. In some embodiments, a lipid nanoparticle has an N/P ratio of about 2. In some embodiments, a lipid nanoparticle has an N/P ratio of about 3. In some embodiments, a lipid nanoparticle has an N/P ratio of about 4. In some embodiments, a lipid nanoparticle has an N/P ratio of about 5. In some embodiments, a lipid nanoparticle has an N/P ratio of about 6. In some embodiments, a lipid nanoparticle has an N/P ratio of about 7. In some embodiments, a lipid nanoparticle has an N/P ratio of about 8.

In some embodiments, a composition according to the present invention contains at least about 0.01 mg, 0.05 mg, 0.1 mg, 0.5 mg, 1 mg, 5 mg, 10 mg, 100 mg, 500 mg, or 1000 mg of encapsulated mRNA. In some embodiments, a composition contains about 0.1 mg to 1000 mg of encapsulated mRNA. In some embodiments, a composition contains at least about 0.5 mg of encapsulated mRNA. In some embodiments, a composition contains at least about 0.8 mg of encapsulated mRNA. In some embodiments, a composition contains at least about 1 mg of encapsulated mRNA. In some embodiments, a composition contains at least about 5 mg of encapsulated mRNA. In some embodiments, a composition contains at least about 8 mg of encapsulated mRNA. In some embodiments, a composition contains at least about 10 mg of encapsulated mRNA. In some embodiments, a composition contains at least about 50 mg of encapsulated mRNA. In some embodiments, a composition contains at least about 100 mg of encapsulated mRNA. In some embodiments, a composition contains at least about 500 mg of encapsulated mRNA. In some embodiments, a composition contains at least about 1000 mg of encapsulated mRNA.

### Pharmaceutical Compositions

The present invention provides compositions for use in the treatment of primary ciliary dyskinesia (PCD). The compositions of the present invention are for use in the manufacture of a medicament for the treatment of primary ciliary dyskinesia (PCD).

Provided liposomally-encapsulated or associated mRNAs, and compositions containing the same, may be administered and dosed in accordance with current medical practice, taking into account the clinical condition of the subject, the site and method of administration, the scheduling of administration, the subject's age, sex, body weight and other factors relevant to clinicians of ordinary skill in the art. As used herein, the term “therapeutically effective amount” is largely determined based on the total amount of the therapeutic agent contained in the pharmaceutical compositions of the present invention. Generally, a therapeutically effective amount is sufficient to achieve a meaningful benefit to the subject, the mammal, (e.g., treating, modulating, curing, preventing and/or ameliorating PCD). For example, a therapeutically effective amount may be an amount sufficient to achieve a desired therapeutic and/or prophylactic effect. Generally, the amount of a therapeutic agent (e.g., mRNA encoding a DNAI1 protein) administered to a subject in need thereof will depend upon the characteristics of the subject. Such characteristics include the condition, disease severity, general health, age, sex and body weight of the subject. One of ordinary skill in the art will be readily able to determine appropriate dosages depending on these and other related factors. In addition, both objective and subjective assays may optionally be employed to identify optimal dosage ranges.

In some embodiments, an effective therapeutic dose of the pharmaceutical composition comprising an mRNA encoding dynein axonemal intermediate chain 1 protein is administered to the mammal at a dosing interval sufficient to reduce for the period of the dosing interval or longer the level of at least one symptom or biomarker associated with PCD in the mammal relative to its state prior to the treatment.

In some embodiments the mammal is a human. A suitable therapeutic dose that may be applicable for a human being can be derived based on animal studies. A basic guideline for deriving a human equivalent dose from studies performed in animals can be obtained from the U.S. Food and Drug Administration (FDA) website at https://www.fda.gov/downloads/drugs/guidances/ucm078932.pdf, entitled, “Guidance for Industry Estimating the Maximum Safe Starting Dose in Initial Clinical Trials for Therapeutics in Adult Healthy Volunteers.” Based on the guidelines for allometric scaling, a suitable dose of, for example, 0.6 mg/kg in a mouse, would relate to a human equivalent dose of 0.0048 mg/kg. Thus, considering the derived human equivalent dose, a projected human therapeutic dose can be derived based on studies in other animals.

In some embodiments, the dosing interval is once every 2 days, or longer. In some embodiments, the dosing interval is once every 3 days or longer. In some embodiments, the dosing interval is once every 4 days or longer. In some embodiments, the dosing interval is once every 5 days or longer. In some embodiments, the dosing interval is once every 6 days or longer. In some embodiments, the dosing interval is once every 7 days or longer. In some embodiments, the dosing interval is once every 8 days or longer. In some embodiments, the dosing interval is once every 9 days or longer. In some embodiments, the dosing interval is once every 10 days or longer. In some embodiments, the dosing interval is once every 11 days or longer. In some embodiments, the dosing interval is once every 12 days or longer. In some embodiments, the dosing interval is once every 13 days or longer. In some embodiments, the dosing interval is once every 14 days or longer once every 15 days or longer, or once every 20 days or longer, or once every 21 days, or once every 22 days, or once every 23 days, or once every 24 days, or once every 25 days, once every 26 days, or once every 27 days, or once every 28 days, or once every 29 days or longer, or once every 30 days or longer, or once every 31 days or longer. In some embodiments, the dosing interval is once every 40, 45 or 50 days or 60 days, or any number of days in between. In some embodiments, the dosing interval is once every 80, 90 or 120 days or 150 days, or any number of days in between.

In some embodiments, the therapeutic low dose is administered at a dosing interval of once every 2 weeks or longer, which is sufficient to reduce the level of at least one symptom or biomarker associated with PCD in the mammal relative to the state prior to the treatment. In some embodiments, the therapeutic low dose is administered at a dosing interval of once every 3 weeks or longer, which is sufficient to reduce the level of at least one symptom or biomarker associated with PCD in the mammal relative to the state prior to the treatment. In some embodiments, the dosing interval is once every 4 weeks or longer. In some embodiments, the dosing interval is once every 5 weeks or longer. In some embodiments, the dosing interval is once every 6 weeks or longer. In some embodiments, the dosing interval is once every 8 weeks or longer. In some embodiments, the dosing interval is once every 12 or 15 or 18 weeks or longer.

In some embodiments, the dosing interval is once a month. In some embodiments, the dosing interval is once in every two months. In some embodiments, the dosing interval is once every three months, or once every four months or once every five months or once every six months or anywhere in between.

In some embodiments, administering the provided composition results in an increased DNAI1 mRNA expression level in a biological sample from a subject as compared to a baseline expression level before treatment. Typically, the baseline level is measured immediately before treatment. Biological samples include, for example, whole blood, serum, plasma, urine and tissue samples (e.g., muscle, liver, skin fibroblasts). In some embodiments, administering the provided composition results in an increased DNAI1 mRNA expression level by at least about 10%, 20%, 30%, 40%, 50%, 60%, 70%, 80%, 90%, or 95% as compared to the baseline level immediately before treatment. In some embodiments, administering the provided composition results in an increased DNAI1 mRNA expression level as compared to a DNAI1 mRNA expression level in subjects who are not treated

According to the present invention, a therapeutically effective dose of the provided composition, when administered regularly, results in an increased DNAI1 protein expression or activity level in a subject as compared to a baseline DNAI1 protein expression or activity level before treatment. Typically, the DNAI1 protein expression or activity level is measured in a biological sample obtained from the subject such as blood, plasma or serum, urine, or solid tissue extracts. In some embodiments, the administering of a composition of the invention results in DNAI1 expression detectable in the liver. In some embodiments, administering the provided composition results in an increased DNAI1 protein expression or activity level in a biological sample (e.g., plasma/serum or urine) by at least about 10%, 20%, 30%, 40%, 50%, 60%, 70%, 80%, 90%, or 95% as compared to a baseline level before treatment. In some embodiments, administering the provided composition results in an increased DNAI1 protein expression or activity level in a biological sample (e.g., plasma/serum or urine) by at least about 10%, 20%, 30%, 40%, 50%, 60%, 70%, 80%, 90%, or 95% as compared to a baseline level before treatment for at least 24 hours, at least 48 hours, at least 72 hours, at least 3 days, at least 4 days, at least 5 days, at least 6 days, at least 7 days, at least 8 days, at least 9 days, at least 10 days, at least 11 days, at least 12 days, at least 13 days, at least 14 days, or at least 15 days.

In some embodiments, the therapeutic dose is sufficient to achieve at least some stabilization, improvement or elimination of symptoms and other indicators, such as biomarkers, are selected as appropriate measures of disease progress, disease regression or improvement by those of skill in the art.

Suitable routes of administration include, for example, oral, rectal, vaginal, transmucosal, pulmonary including intratracheal or inhaled, or intestinal administration; parenteral delivery, including intradermal, transdermal (topical), intramuscular, subcutaneous, intramedullary injections, as well as intrathecal, direct intraventricular, intravenous, intraperitoneal, or intranasal.

In some embodiments, the therapeutically effective dose comprising the mRNA encoding DNAI1 protein is administered to the subject by intramuscular administration.

In some embodiments, the therapeutically effective dose comprising the mRNA encoding DNAI1 is administered to the subject by subcutaneous administration.

In particular embodiments, the intramuscular administration is to a muscle selected from the group consisting of skeletal muscle, smooth muscle and cardiac muscle. In some embodiments the administration results in delivery of the mRNA to a muscle cell. In some embodiments the administration results in delivery of the mRNA to a hepatocyte (i.e., liver cell). In a particular embodiment, the intramuscular administration results in delivery of the mRNA to a muscle cell.

Most commonly, the therapeutically effective dose comprising the mRNA encoding dynein axonemal intermediate chain protein 1 is administered to the subject by intravenous administration.

Alternatively or additionally, liposomally encapsulated mRNAs and compositions of the invention may be administered in a local rather than systemic manner, for example, via injection of the pharmaceutical composition directly into a targeted tissue, preferably in a sustained release formulation. Local delivery can be affected in various ways, depending on the tissue to be targeted. For example, aerosols containing compositions of the present invention can be inhaled (for nasal, tracheal, or bronchial delivery); compositions of the present invention can be injected into the site of injury, disease manifestation, or pain, for example; compositions can be provided in lozenges for oral, tracheal, or esophageal application; can be supplied in liquid, tablet or capsule form for administration to the stomach or intestines, can be supplied in suppository form for rectal or vaginal application; or can even be delivered to the eye by use of creams, drops, or even injection. Formulations containing provided compositions complexed with therapeutic molecules or ligands can even be surgically administered, for example in association with a polymer or other structure or substance that can allow the compositions to diffuse from the site of implantation to surrounding cells. Alternatively, they can be applied surgically without the use of polymers or supports.

In particular embodiments, DNAI1 encoding mRNA is administered intravenously, wherein intravenous administration is associated with delivery of the mRNA to hepatocytes.

In some embodiments, the therapeutically effective dose comprising the mRNA encoding dynein axonemal intermediate chain protein is administered for suitable delivery to the mammal's liver. In some embodiments, the therapeutically effective dose comprising the mRNA encoding dynein axonemal intermediate chain protein is administered for suitable expression in hepatocytes of the administered mammal.

Provided methods of the present invention contemplate single as well as multiple administrations of a therapeutically effective amount of the therapeutic agents (e.g., mRNA encoding a DNAI1 protein) described herein. Therapeutic agents can be administered at regular intervals, depending on the nature, severity and extent of the subject's condition (e.g., PCD). In some embodiments, a therapeutically effective amount of the therapeutic agents (e.g., mRNA encoding a DNAI1 protein) of the present invention may be administered intrathecally periodically at regular intervals (e.g., once every year, once every six months, once every five months, once every three months, bimonthly (once every two months), monthly (once every month), biweekly (once every two weeks), twice a month, once every 30 days, once every 28 days, once every 14 days, once every 10 days, once every 7 days, weekly, twice a week, daily or continuously).

In some embodiments, provided liposomes and/or compositions are formulated such that they are suitable for extended-release of the mRNA contained therein. Such extended-release compositions may be conveniently administered to a subject at extended dosing intervals. For example, in one embodiment, the compositions of the present invention are administered to a subject twice a day, daily or every other day. In some embodiments, the compositions of the present invention are administered to a subject twice a week, once a week, once every 7 days, once every 10 days, once every 14 days, once every 28 days, once every 30 days, once every two weeks, once every three weeks, once every four weeks, once a month, twice a month, once every six weeks, once every eight weeks, once every other month, once every three months, once every four months, once every six months, once every eight months, once every nine months or annually.

In a preferred embodiment, the compositions of the present invention are administered to a subject once a week, once every two weeks or once a month. In a more preferred embodiment, the compositions of the present invention are administered to a subject once every two weeks or once every month. In the most preferred embodiment, the compositions of the present invention are administered to a subject once every month.

In some embodiments the mRNA is administered concurrently with an additional therapy.

Also contemplated are compositions and liposomes which are formulated for depot administration (e.g., intramuscularly, subcutaneously, intravitreally) to either deliver or release an mRNA over extended periods of time. Preferably, the extended-release means employed are combined with modifications made to the mRNA to enhance stability.

A therapeutically effective amount is commonly administered in a dosing regimen that may comprise multiple unit doses. For any particular therapeutic protein, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific protein employed; the duration of the treatment; and like factors as is well known in the medical arts. According to the present invention, a therapeutically effective dose of the provided composition, when administered regularly, results in at least one symptom or feature of PCD is reduced in intensity, severity, or frequency or has delayed onset.

Also contemplated herein are lyophilized pharmaceutical compositions comprising one or more of the liposomes disclosed herein and related methods for the use of such compositions as disclosed for example, in International Patent Application PCT/US12/41663, filed Jun. 8, 2012, the teachings of which are incorporated herein by reference in their entirety. For example, lyophilized pharmaceutical compositions according to the invention may be reconstituted prior to administration or can be reconstituted in vivo. For example, a lyophilized pharmaceutical composition can be formulated in an appropriate dosage form (e.g., an intradermal dosage form such as a disk, rod or membrane) and administered such that the dosage form is rehydrated over time in vivo by the individual's bodily fluids.

In some embodiments, the pharmaceutical composition comprises a lyophilized liposomal delivery vehicle that comprises a cationic lipid, a non-cationic lipid, a PEG-modified lipid and cholesterol. In some embodiments, the pharmaceutical composition has a Dv50 of less than 500 nm, 300 nm, 200 nm, 150 nm, 125 nm, 120 nm, 100 nm, 75 nm, 50 nm, 25 nm or smaller upon reconstitution. In some embodiments, the pharmaceutical composition has a Dv90 of less than 750 nm, 700 nm, 500 nm, 300 nm, 200 nm, 150 nm, 125 nm, 100 nm, 75 nm, 50 nm, 25 nm or smaller upon reconstitution. In some embodiments, the pharmaceutical composition has a polydispersity index value of less than 1, 0.95, 0.9, 0.8, 0.75, 0.7, 0.6, 0.5, 0.4, 0.3, 0.25, 0.2, 0.1, 0.05 or less upon reconstitution. In some embodiments, the pharmaceutical composition has an average particle size of less than 500 nm, 400 nm, 300 nm, 200 nm, 175 nm, 150 nm, 125 nm, 100 nm, 75 nm, 50 nm, 25 nm or upon reconstitution.

In some embodiments, the lyophilized pharmaceutical composition further comprises one or more lyoprotectants, such as sucrose, trehalose, dextran or inulin. Typically, the lyoprotectant is sucrose. In some embodiments, the pharmaceutical composition is stable for at least 1 month or at least 6 months upon storage at 4° C., or for at least 6 months upon storage at 25° C. In some embodiments, the biologic activity of the mRNA of the reconstituted lyophilized pharmaceutical composition exceeds 75% of the biological activity observed prior to lyophilization of the composition.

Provided liposomes and compositions may be administered to any desired tissue. In some embodiments, the DNAI1 mRNA delivered by provided liposomes or compositions is expressed in the tissue in which the liposomes and/or compositions were administered. In some embodiments, the mRNA delivered is expressed in a tissue different from the tissue in which the liposomes and/or compositions were administered. Exemplary tissues in which delivered mRNA may be delivered and/or expressed include, but are not limited to the liver, kidney, heart, spleen, serum, brain, skeletal muscle, lymph nodes, skin, and/or cerebrospinal fluid.

According to various embodiments, the timing of expression of delivered mRNAs can be tuned to suit a particular medical need. In some embodiments, the expression of the protein encoded by delivered mRNA is detectable 1, 2, 3, 6, 12, 24, 48, 72, 96 hours, 1 week, 2 weeks, or 1 month after administration of provided liposomes and/or compositions.

In some embodiments, administering the provided composition results in an increased level of DNAI1 protein in a liver cell (e.g., a hepatocyte) of a subject as compared to a baseline level before treatment. Typically, the baseline level is measured immediately before treatment. In some embodiments, administering the provided composition results in an increased DNAI1 protein level in the liver cell by at least about 10%, 20%, 30%, 40%, 50%, 60%, 70%, 80%, 90%, or 95% as compared to a baseline level before treatment. In some embodiments, administering the provided composition results in an increased DNAI1 protein level in a liver cell as compared to the DNAI1 protein level a liver cell of subjects who are not treated.

In some embodiments, administering the provided composition results in an increased DNAI1 protein level in plasma or serum of subject as compared to a baseline level before treatment. Typically, the baseline level is measured immediately before treatment. In some embodiments, administering the provided composition results in an increased DNAI1 protein level in plasma or serum by at least about 10%, 20%, 30%, 40%, 50%, 60%, 70%, 80%, 90%, or 95% as compared to a baseline level before treatment. In some embodiments, administering the provided composition results in an increased DNAI1 protein level in plasma or serum as compared to a DNAI1 protein level in plasma or serum of subjects who are not treated.

In some embodiments, administering the provided composition results in increased DNAI1 enzyme activity in a biological sample from a subject as compared to the baseline level before treatment. Typically, the baseline level is measured immediately before treatment. Biological samples include, for example, whole blood, serum, plasma, urine and tissue samples (e.g., liver). In some embodiments, administering the provided composition results in an increased DNAI1 enzyme activity by at least about 10%, 20%, 30%, 40%, 50%, 60%, 70%, 80%, 90%, or 95% as compared to a baseline level immediately before treatment. In some embodiments, administering the provided composition results in an increased DNAI1 enzyme activity as compared to DNAI1 activity in subjects who are not treated.

In some embodiments the subject is a mammal. In some embodiments, the mammal is an adult. In some embodiments the mammal is an adolescent. In some embodiments the mammal is an infant or a young mammal. In some embodiments, the mammal is a primate. In some embodiments the mammal is a human. In some embodiments the subject is 6 years to 80 years old.

### EXAMPLES

While certain compounds, compositions and methods of the present invention have been described with specificity in accordance with certain embodiments, the following examples serve only to illustrate the compounds of the invention and are not intended to limit the same.

### Example 1. Establishment of In Vitro Disease Model

In this example, pseudo-stratified epithelium was generated from human induced pluiropetent stem cells. The development of this model enables the study of lung diseases that are difficult to study in human models.

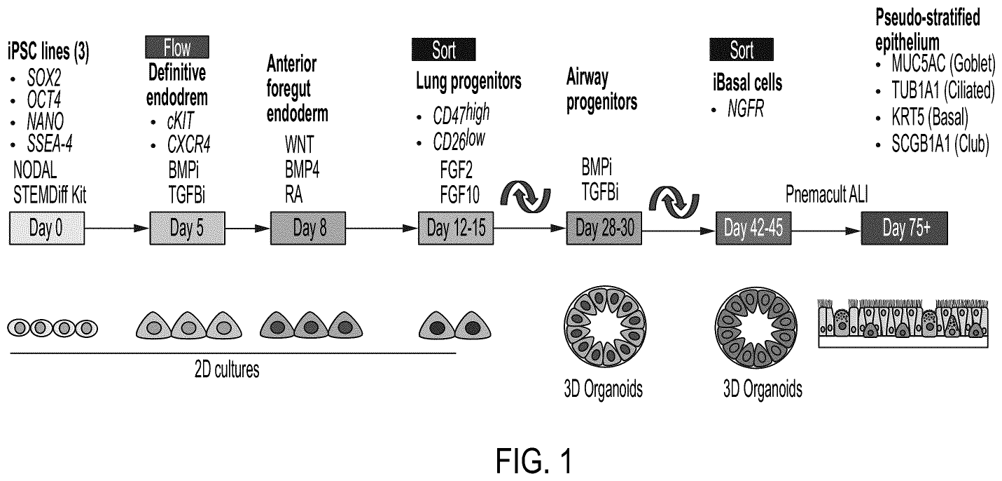

The schematic for in vitro directed differentiation method for generating pseudo-stratified epithelium from human embryonic stem cells is shown in FIG. 1. Timed treatment by exogenous growth factors that mimic endoderm developmental pathways in vivo followed by air-liquid interface culture resulted in maturation of patches of tight junction-coupled differentiated airway epithelial cells that demonstrate stratified epithelium.

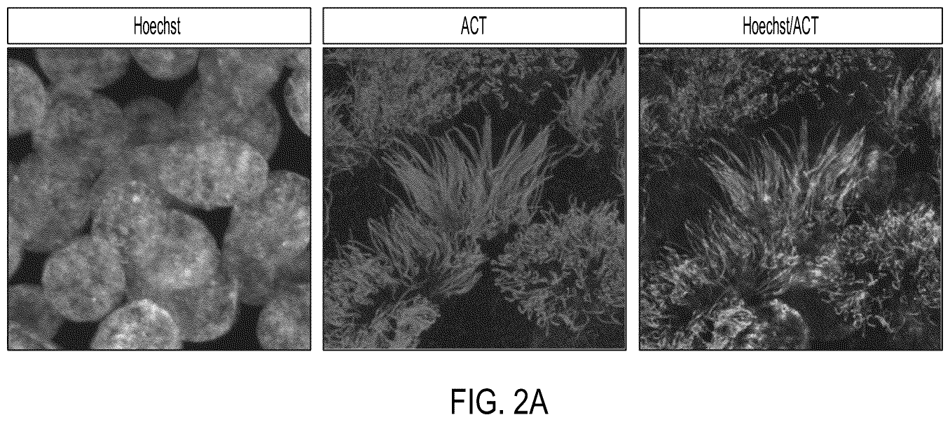

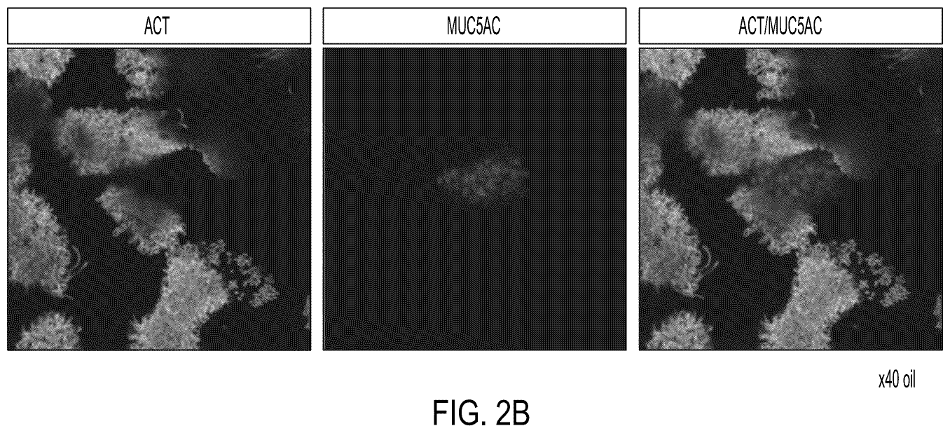

To further assess that the cultured epithelium indeed mimics the epithelium suitable for studying the lung diseases, immunofluorescence was performed. As shown in FIG. 2A, ACT staining showed presence of robust cilia in the epithelium lining. Additionally, FIG. 2B shows the successful expression of MUC5AC at different locations from beta actin. Overall, immunofluorescence data illustrate that the pluripotent cells were differentiated to ciliated, club, globlet and basal cells, mimicking the airway epithelium.

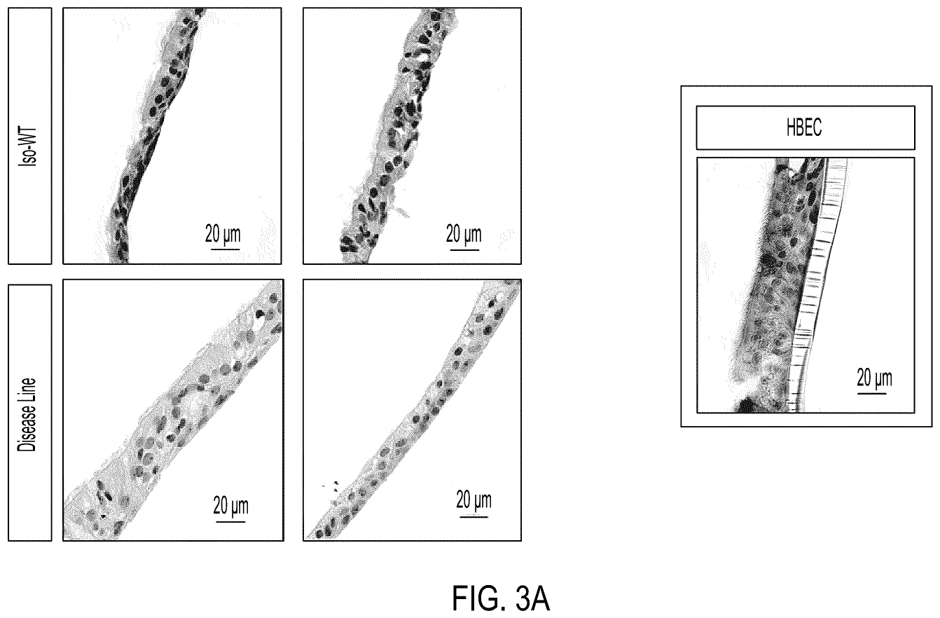

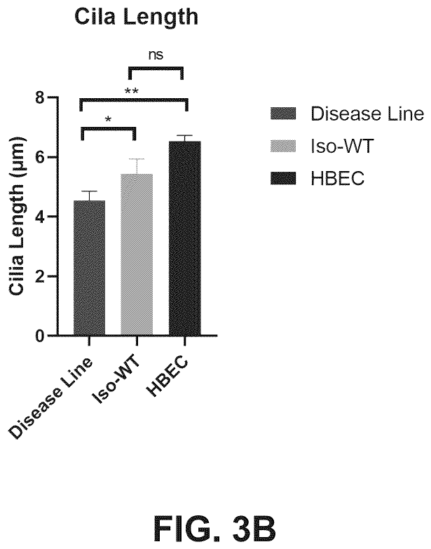

In addition to the “wild-type” model, an “iso-wild-type” (or “iso-WT”) model and a “disease” model were generated in a similar manner from the iPSC lines. For the “iso-wild-type” model, CRISPR-Cas system was used in attempt to knock-down the DNAI1 gene. However, CRISPR-Cas treatment was not successful, and the iPSC lines maintained the DNAI1, and is therefore called “iso-wild-type” model. For the “disease” model, two alleles for DNAI1 were successfully deleted. To validate the “iso-wild-type” and the “disease” models, cilia length of each line was calculated from H&E (Hematoxylin and Eosin) staining samples and compared to that of cilia in HBEC (Human Bronchial Epithelial Cell) sample, a gold standard for airway epithelia. (FIG. 3A). Cilia length is critical for proper cilia movement of mucus, and cilia in PCD patients are too short to function well. FIG. 3B shows that cilia length in Iso-WT model is not statistically different form that in HBEC model, illustrating that Iso-WT is mimicking the non-diseased airway epithelium. Additionally, the data validates that disease model generated in the example has significantly shorter cilia, illustrating the PCD disease model.

### Example 2. Significant Improvement in Ciliary Beat Frequency by Administration and Delivery of hDNAI1 mRNA

This example illustrates that administration of mRNA encoding hDNAI1 protein encapsulated in lipid nanoparticles (LNPs) to disease model resulted in restoration of ciliary beat frequency (CBF).

PCD is a genetically heterogeneous disorder of motile cilia. PCD is characterized by ciliary dysfunction and impaired mucociliary clearance, and it results in an array of clinical manifestations. Ciliary beat frequency (CBF) is typically used as a screening test and is currently associated with the study of ciliary function.

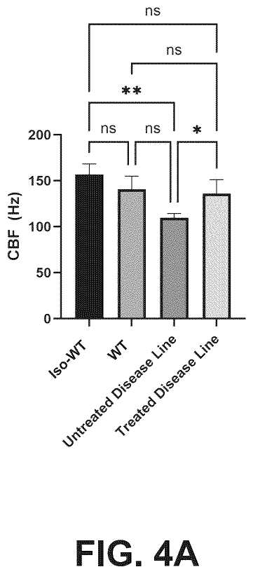

In this study, CBF was used to determine the efficacy of mRNA-LNP in the treatment of PCD, using the disease model described in Example 1. 8 μg of mRNA encoding DNAI1 encapsulated in lipid nanoparticle was administered on the apical side of ALI cultures. As positive controls, Iso-wild-type and Wild-type ALI cultures, described in Example 1, were used. As a negative control, untreated disease model was used, which received 10% trehalose. As shown in FIG. 4A, there was a significant improvement in CBP observed 24 hours post transfection with mRNA-LNP. (CBF values were higher than normal for all samples due to low frame/second capture capability of the camera in this study.)

Overall, the data in this example shows that mRNA-LNPs of the present invention is able to increase the CBF in PCD patients, and restore it to the wild-type, a non-diseased model.

### Example 3. Single Dose Administration of mRNA Encoding DNAI1 In Vivo

In this example, a single dose administration of mRNA encoding DNAI1 encapsulated in LNP was studied.

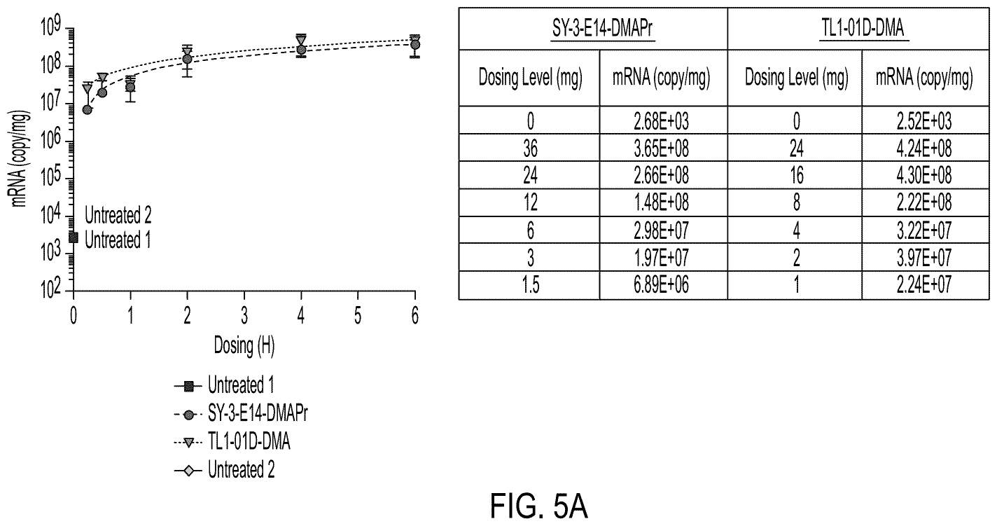

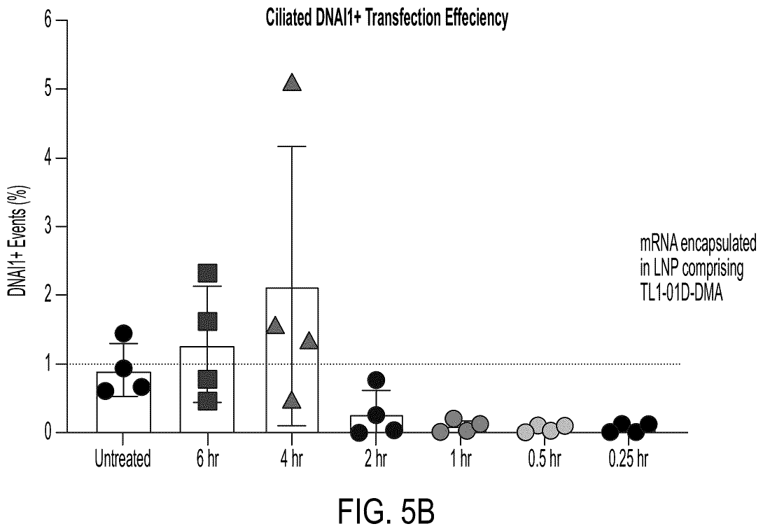

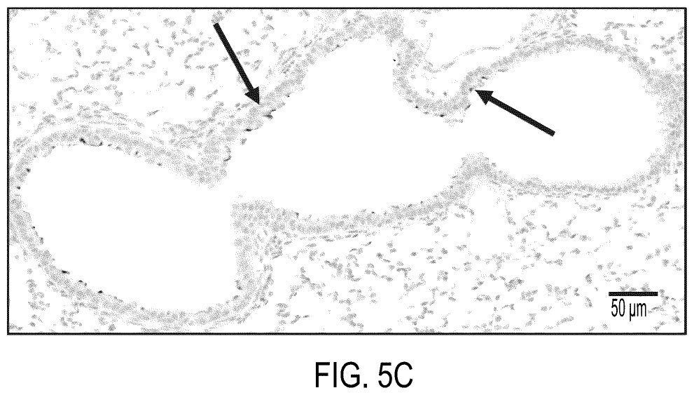

mRNA comprising SEQ ID NO: 10 was encapsulated in LNPs comprising either TL1-01D-DMA or SY-3-E14-DMAPr cationic lipids. Then, the mRNA-LNPs were nebulized once to mice, and the expression of DNAI1 protein was measured in lungs and trachea. As shown in FIG. 5A, dose response was observed with mRNA levels from lungs immediately post-exposure both lipids.

Next, flow cytometry was performed to determine the % of cells that are DNAI1-positive. According to Ostrowski, acquiring 20% DNAI1+ cells can restore functions of cilia, and promote ciliated clearance (Ostrowski, L E et al. “Restoring ciliary function to differentiated primary ciliary dyskinesia cells with a lentiviral vector.” Gene therapy vol. 21, 3 (2014): 253-61). As shown in FIG. 5B, a single administration of the mRNA-LNP comprising TL-1-01D-DMA as a cationic lipid resulted in ciliated DNAI1 positive transfection efficiency of less than 3%, with a peak at 4 hours post administration. FIG. 5C shows the immunohistochemistry data at 6 hours post nebulization, illustrating the DNAI1 expression is localized to airway epithelium.

### Example 4. Dose Response Effect after Nebulized Exposure after Single Administration

In this example, a single dose nebulized administration of mRNA encoding DNAI1 encapsulated in LNP was studied.

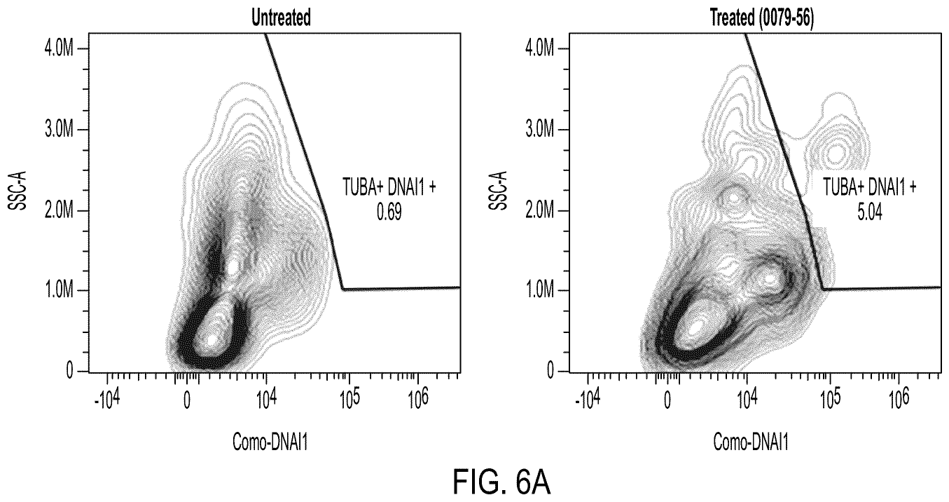

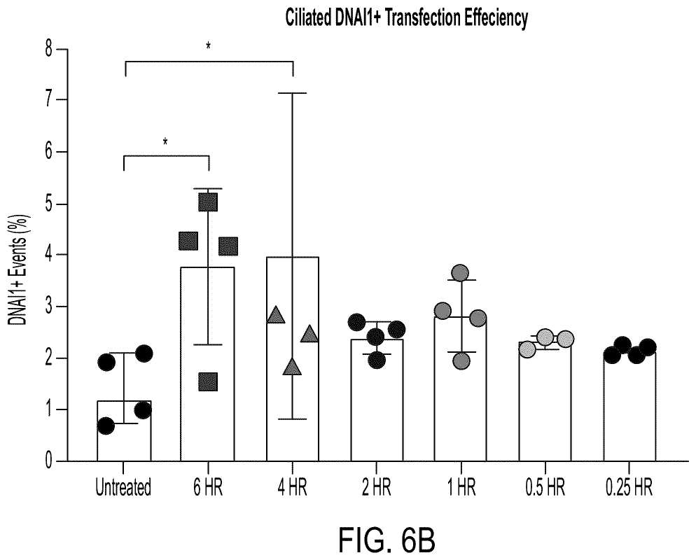

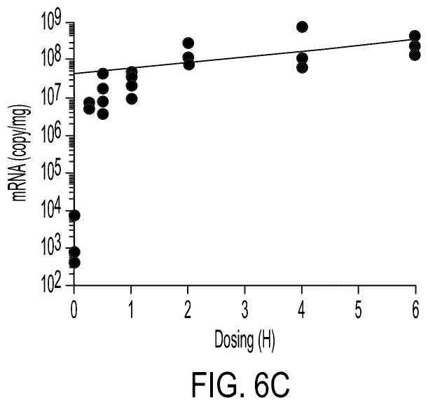

Animals were treated with nebulized MRT-DNAI1-LNP in a DSI inhalation tower so that the route of administration would be relevant for potential human treatment using a nebulizer. This would also enable the determination of dose-exposure relationships. Animals were exposed for different lengths of time while keeping the concentration constant at 0.6 mg/mL of MRT-DNAI1-LNP, ranging from 30 minutes to 6 hours. On day 3, 48-hours post-exposure, lungs were collected and were enzymatically digested with an optimized protocol to obtain single-cell suspensions. Dead cells were excluded using Zombie NIR after which cell markers for CD45 and CD31 were used to segregate leukocytic and endothelial populations respectively. Epithelial cells were then selected by negative selection of CD45 and CD31. After which, multiciliated cells were selected using positive selection for acetylated tubulin (i.e. CD45Neg, CD31Neg, CD326Pos, TUBAPos). Gating live, epithelial, multi-ciliated cells was done on the untreated samples to determine the baseline level of DNAI1 expression (<1%) and compared with the treated samples. Gating parameters were kept constant throughout the experiment. FIG. 6A shows representative density plots of multiciliated epithelial cells that were DNAI1-positive in untreated cells vs treated cells. A new population of cells that are TUBA+/DNAI1+ appear in the gated area, demonstrating protein expression. FIG. 6B displays the flow cytometry results from the lungs from the different exposure times. There is a dose-responsive relationship demonstrating that with longer exposures, there are more TUBA+/DNAI1+ cells. Levels of the mRNA were assessed in the lungs immediately after dosing by RTqPCR. A dose-exposure relationship was observed, suggesting that the animals were properly dosed and correlates with the flow cytometry data showing a dose-response (FIG. 6C).

### Example 5. Duration of DNAI1 Expression in Mouse Lungs

In this example, the duration of DNAI1 expression post-exposure was measured to examine the effect of administering mRNA encoding DNAI1 encapsulated in LNP was studied.

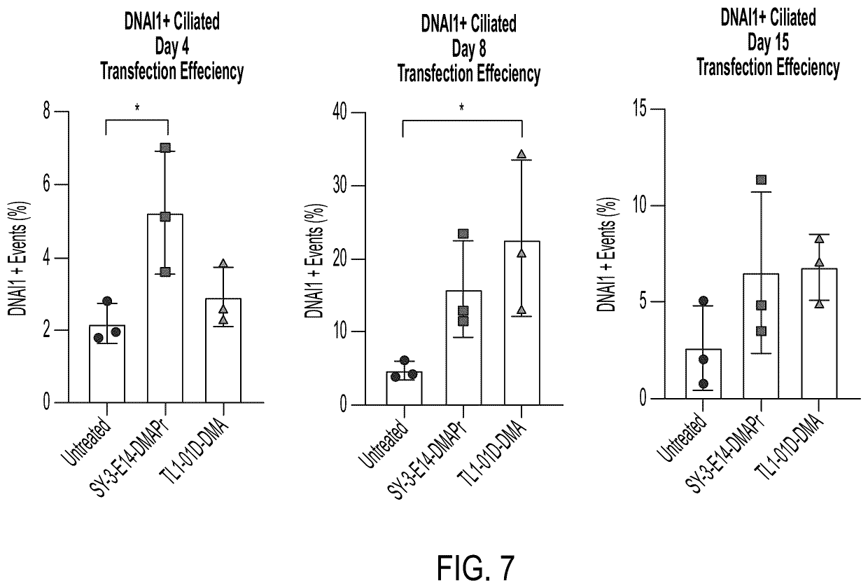

Animals were treated with a single 6-hour exposure with two different LNPs to determine the duration of DNAI1 expression post-exposure. Lungs were harvested either immediately post-dose, day 4, 8, or 15 (FIG. 7). Lungs collected on days 4, 8 and 12 and were enzymatically digested with an optimized protocol to obtain single-cell suspensions. As before, flow cytometry panels were used for gating. Gating live, epithelial, multi-ciliated cells was done on the untreated samples to determine the baseline level of DNAI1 expression (<2%) and compared with the treated samples. The baseline levels may vary from experiment to experiment slightly in a flow cytometry experiment. However, gating parameters were kept constant throughout any one experiment. FIG. 7 shows the percentage of DNAI1-positive events in live multiciliated epithelial cells from the lungs on days 4, 8, and 15 with two different LNPs. Overall, DNAI1 expression was still detectable 15 days after nebulized exposure to LNP-encapsulated MRT-DNAI1.

### Example 6. Repeat Dose Administration of mRNA Encoding DNAI1 In Vivo Restores CBF

In this example, expression after daily repeat dosing regimen of mRNA encoding DNAI1 encapsulated in LNP was studied.

mRNA comprising SEQ ID NO: 10 was encapsulated in LNPs comprising either TL1-01D-DMA (“Lipid 2”) or SY-3-E14-DMAPr (“Lipid 1”) lipids. Animals were exposed for either a single 30-minute exposure or five daily 30-minute exposures. For the animals that had a single exposure, lungs were harvested either immediately post-dose or on day 4, 72 hours post-exposure. For the animals that had multiple exposures, lungs were collected either immediately post-dose on day 5, on day 8, 72 hours after the last exposure, or on day 12, 7 days after the last exposure. For the lungs that were collected immediately post-dose, they were analyzed for mRNA by RTqPCR or parent lipid by mass spectroscopy. For the lungs collected on days 8 and 12, they were enzymatically digested with an optimized protocol to obtain single-cell suspensions. Dead cells were excluded using Zombie NIR after which cell markers for CD45 and CD31 were used to segregate leukocytic and endothelial populations respectively. Epithelial cells were then selected by negative selection of CD45 and CD31. After which, multiciliated cells were selected using positive selection for acetylated tubulin (i.e. CD45Neg, CD31Neg, CD326Pos, TUBAPos). Gating live, epithelial, multi-ciliated cells was done on the untreated samples to determine the baseline level of DNAI1 expression (<1%) and compared with the treated samples. Gating parameters were kept constant throughout the experiment.

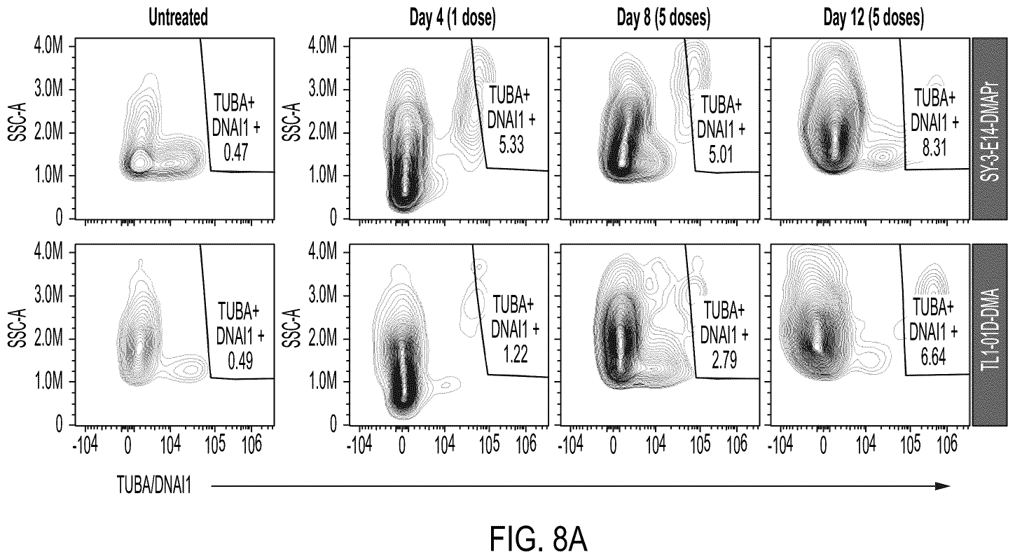

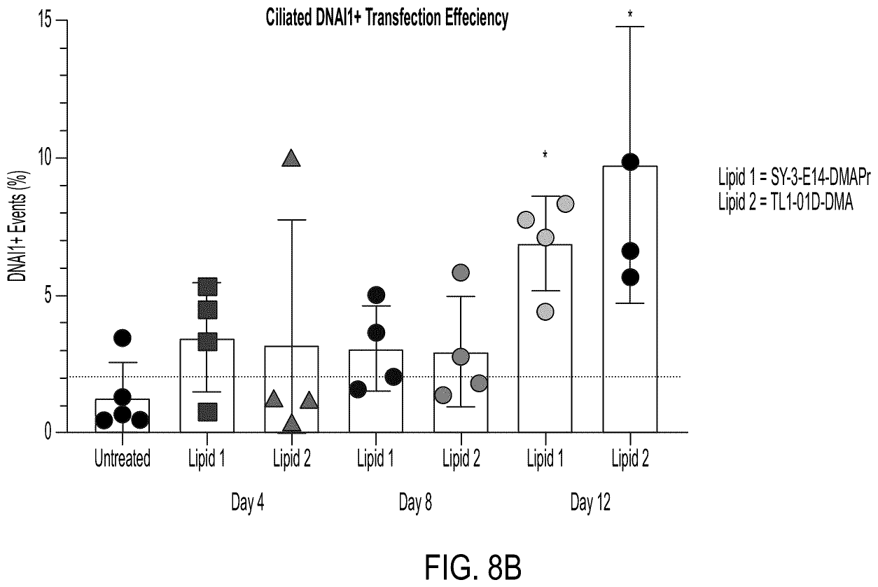

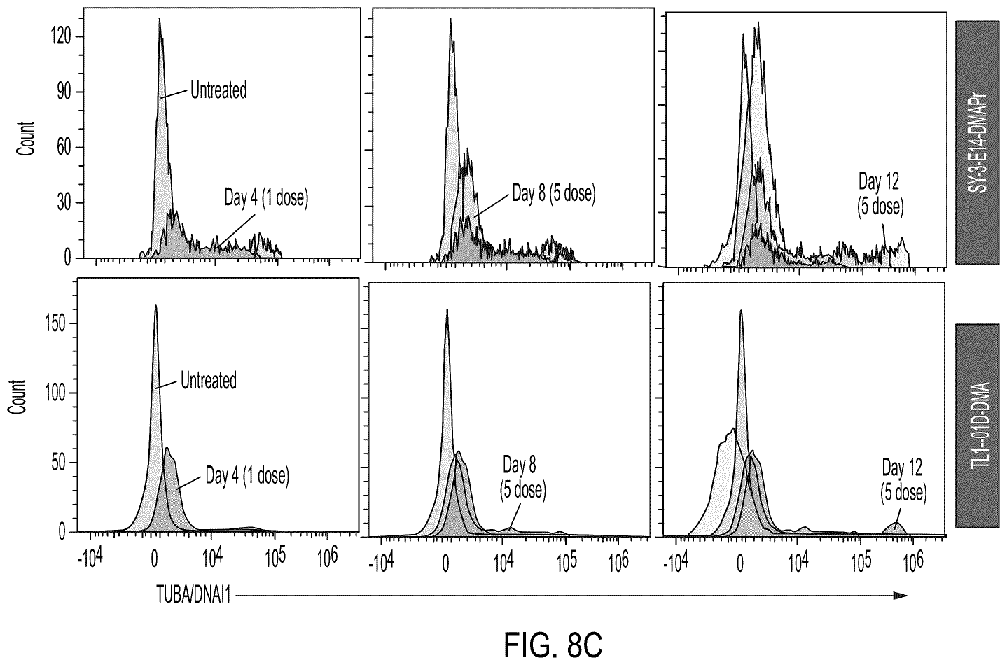

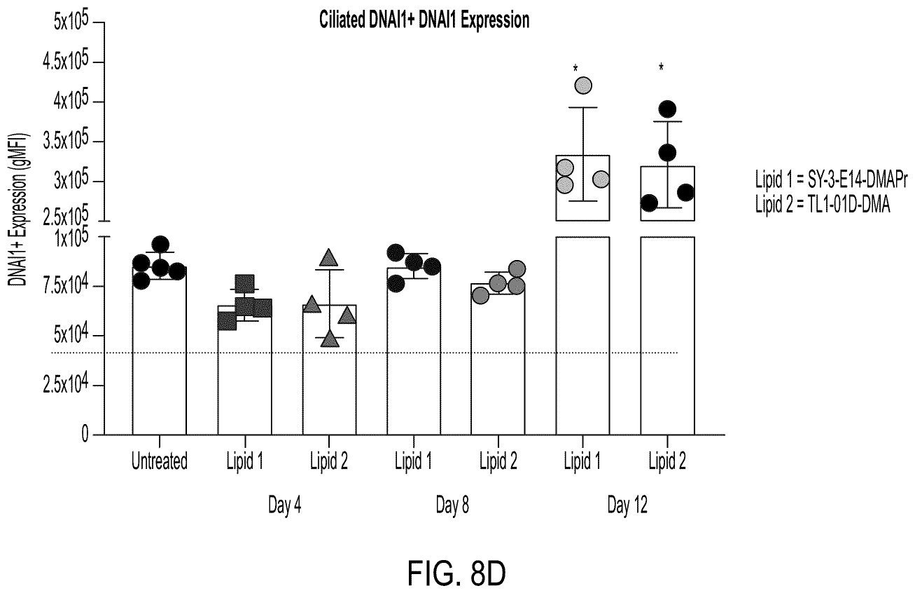

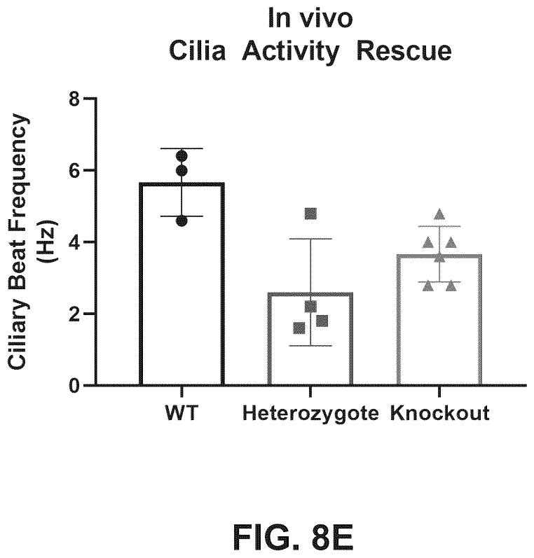

FIG. 8A shows representative density plots of multiciliated epithelial cells that were DNAI1-positive in untreated cells vs treated cells. A new population of cells that are TUBA+/DNAI1+ appear in the gated area, demonstrating protein expression. This study was a head-to-head comparison of the two main cationic lipids, LNP1 and LNP2, in the LNPs encapsulating MRT-DNAI1. The results show that repeat dosing results in a distinct ciliated and DNAI1-positive population for both lipids. Notably, the expression was maintained on day 12.

The ciliated DNAI1-positive transfection efficiency was determined and plotted as shown in FIG. 8B, which shows the flow cytometry results from all animals in the study. After a single nebulized dose there is no significant difference between either untreated or treated animals with the 30-minute exposure. For animals that were treated for multiple days, there was no significant difference between the untreated and treated animals at 72 hours after the last dose. However, by day 12, 7 days after the last dose, there was a significant increase in the number of cells that expressed human DNAI1 in the multi-ciliated cells with both cationic lipids.

FIG. 8C summarizes the gMFI data quantitatively. Analyzing the flow cytometry data for geometric mean fluorescence intensity (gMFI) showed that by day 12, there was continued increase in the DNAI1 fluorescence (FIG. 8C). With the single dose groups (groups 2 and 3), there was a small population of cells that had low DNAI1 signal on day 4 (right panels, red). With the repeat dose group 4, on day 8 there was an increase in the number of cells that expressed low levels of DNAI1 with MRT-DNAI1-LNP1. With the repeat dose group 5, there was no difference in the number of cells that showed DNAI1 expression when compared to group 3 with the single dose group (middle panels, orange). However, by day 12, animals in both groups 4 and 5 had an increased number of cells that expressed high levels of DNAI1 in multi-ciliated cells (left panel, green). The kinetics of expression with LNP2 may have delayed onset of expression but can achieve a high level similar to those dosed with LNP1. DNAI1 expression intensity in the multiciliated cells increases throughout a week past the last dose and the increase achieves statistical significance on day 12 with both treatment groups using 1-way ANOVA *p<0.05, Fisher's LSD Test compared to untreated.

The expression levels are shown in FIG. 8D. Dose-exposure assessments were performed by RT-qPCR from lungs of animals immediately post-dose (FIG. 8D). Overall, there was an increase in mRNA comparing Day 1 (group 2) to Day 5 (group 4) for LNP1. A similar trend was observed in mRNA levels comparing Day 1 (group 3) to Day 5 (group 5) for LNP2. There were higher levels of mRNA observed in animals dosed with LNP2 than LNP1 comparing same day values, but overall, there were no significant differences between groups using 1-way ANOVA/Tukey's multiple comparison test).

Overall, the data shows that the % of DNAI1-positive cells increases significantly on day 12 (about 7 days after the last dose). Additionally, the % of DNAI1-positive cells increased up to about 10%, a significant amount that can restore the ciliary function.

Next, an experiment was performed to assess activity of the expressed DNAI1 by measuring ciliary beat frequency (CBF). Wild-type mice and DNAI1 heterozygote mice were used as a positive control. mRNA encoding DNAI1 encapsulated SY-3-E14-DMAPr containing LNPs were nebulized to DNAI1 knock-out mice. Trachea samples were collected and CBF was imaged at 500 fps at 32° C. As shown in FIG. 8E, administration of the mRNA-LNP to knock-out mice had higher CBF as compared to the heterozygote mice, illustrating that a repeat dosing of DNAI1 encoding mRNA restored the ciliary activity.

### Example 7. Modeling of Weekly Dosing of mRNA Encoding DNAI1 Restores Cilia Activity

This example shows a modelling of weekly dosing regimen in terms of DNAI1 expression.

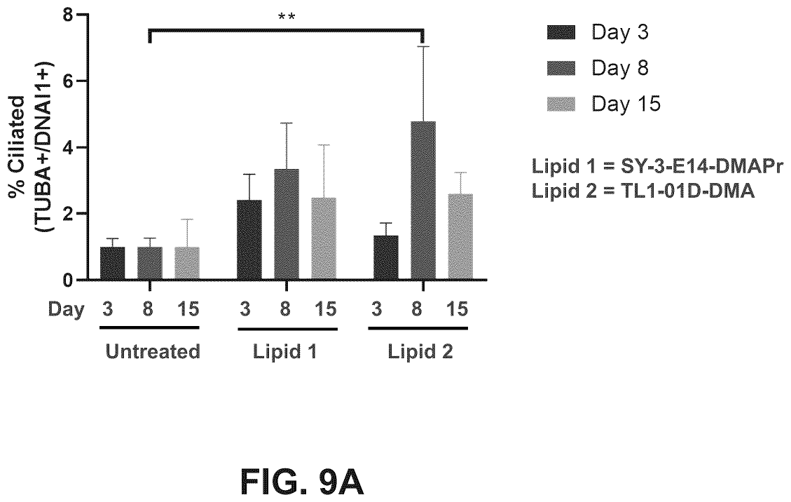

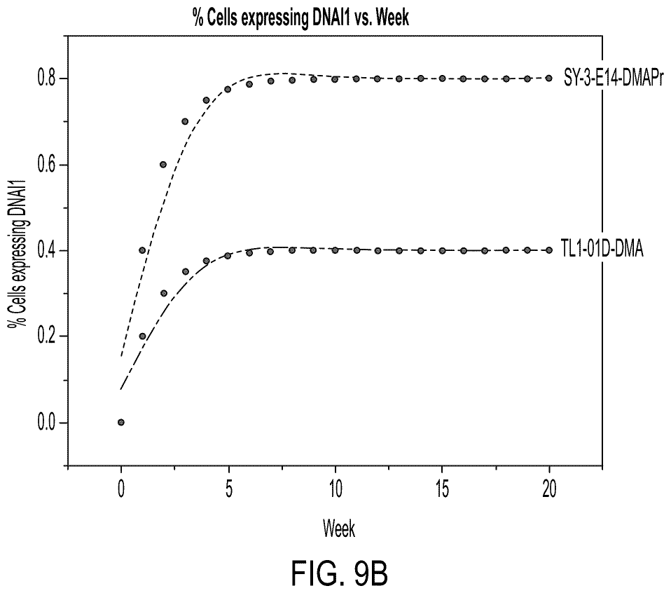

Analysis of % of ciliated and DNAI1-positive cells for up to 15 days showed that protein expression peaked at day 8, and was still present at day 15 (FIG. 9A). Based on this data, the approximate half-life of DNAI1 protein was estimated to be about two weeks. Next, data modelling was performed to examine if a weekly administration would be a viable option for treating PCD, assuming that the half-life of DNAI1 is 2 weeks. As shown in FIG. 9B, the modelling shows that a weekly dosing can possibly achieve a 40% or 80% cells expressing DNAI1. The predicted level of eMax is achieved at least 40% by 5 weeks regardless of the cationic lipids used.

### Example 8. DNAI1 Expression after Weekly Repeat Dosing Paradigm of DNAI1 mRNA-LNP

This example demonstrates an effect of weekly dosing of mRNA encoding DNAI1 encapsulated in LNP.

Given the duration of expression and the ability to detect protein expression with daily repeat dosing, additional work was performed to examine the duration of durability after weekly repeat intratracheal instillation of MRT-DNAI1-LNP1. Animals were dosed weekly for up to six weeks with either saline or MRT-DNAI1-LNP1. A cohort of animals from each group were euthanized and lungs collected seven days after dose 1 through 6. In addition, a cohort of animals were euthanized three days, 7 days, and 15 days after the last dose. Right lungs were snap frozen and analyzed by western blot for overall protein levels and the left lungs were fixed for IHC for spatial distribution.

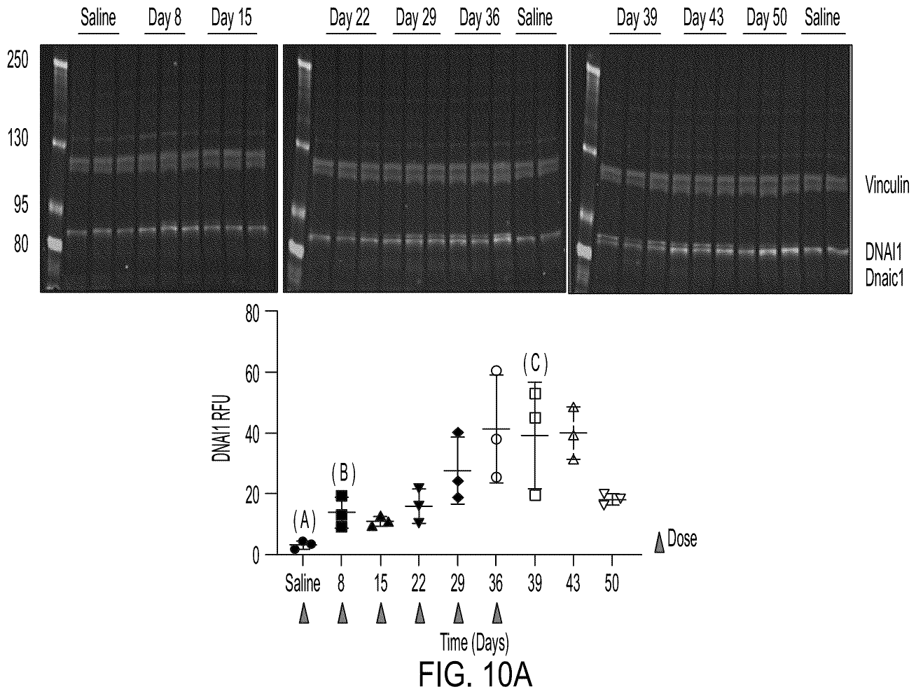

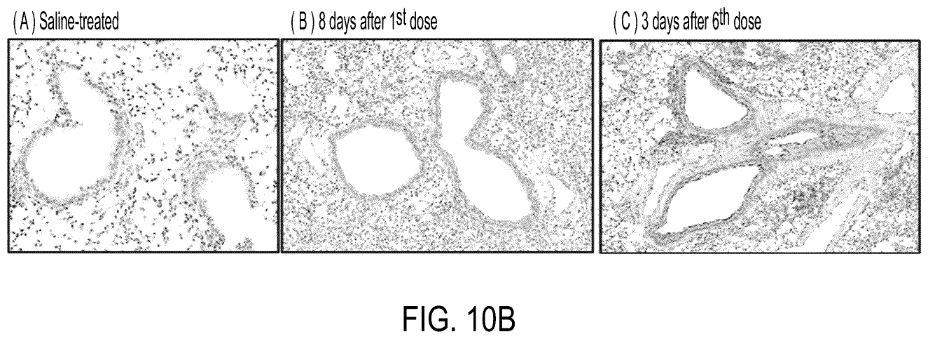

FIG. 10A shows the results from the western blots. For the saline-treated animals, there is a single green band representing the mouse Dnaic1 protein. For the test article-treated animals, the upper green bands represent the human DNAI1 protein while the lower green band represents the mouse Dnaic1 protein. All lanes were loaded with 10 pg total lung homogenate. The loading control protein detected is vinculin (red bands) demonstrating similar amounts of protein added per lane. The graph below summarizes the band intensities of the human DNAI1 protein (DNAI1 relative fluorescence units (RFU) as a function of time.

To determine the spatial distribution of DNAI1 protein expression throughout the lung with weekly dosing, the right lungs were collected for fixation in 10% NBF for overnight and then stored in 70% ethanol until paraffin embedding. The fixed tissues were then sectioned and used for IHC to detect human DNAI1. The IHC protocol was optimized for human-specific DNAI1 detection with minimal background from the mouse Dnaic1 signal. FIG. 10B shows representative IHC results from either saline-treated or test article-treated animals. Overall, there was an increase in the number of DNAI1-positive airway epithelial cells with animals that had undergone increased number of intratracheal instillations. This increase in the IHC staining agrees with the increased DNAI1 signal observed from lung homogenates with the western blot.

### Example 9. Functional Assessment of DNAI1 Expression in a Disease Model

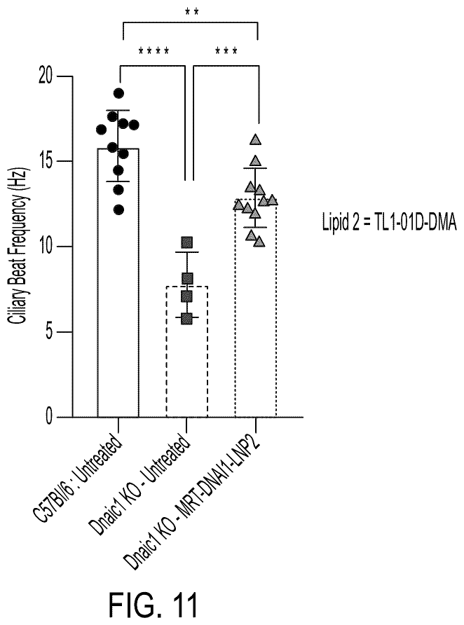

In addition to characterizing the expression of DNAI1 in mouse lungs, additional experiments were performed to determine whether increased ciliary activity was possible with repeat nebulized delivery. While the CD1 mice were used to characterize the expression of MRT-DNAI1, the ciliary function of these mice do not have impaired CBF (data not shown) and correction might not be discernable. An inducible Dnaic1 knockout model (Dnaic1 FL/EX CreERT+/−) demonstrates a PCD phenotype in the upper airways but no increase in pulmonary disease in the lower airways. However, after inducing the loss of endogenous Dnaic1 with tamoxifen treatment, ciliary activity is no longer observed by microscopy. An in vitro air-liquid interface model derived from the trachea tissue demonstrated that lentiviral transduction of the murine Dnaic1 increases the percent ciliated surface area. Here, this PCD disease model was to determine whether exogenous DNAI1 expression can increase ciliary activity in vivo. Knockout of Dnaic1 was induced with tamoxifen treatment by intraperitoneal injections at approximately 8 weeks old. To allow for the turnover of multiciliated cells, the mice were maintained for another 8-10 weeks before using them in an experiment. Based on the expression data generated in the CD1 mice, a repeat dosing regimen was generated that would allow for an accumulation of DNAI1-positive multiciliated population. Animals were dosed utilizing MRT-DNAI1-LNP2 three times weekly for a total of three weeks at 2 hours per exposure. Untreated Dnaic1 knockout animals were used as controls to observe any baseline CBF. Additionally, C57BL/6 control mice were used as a wild-type positive control group because the Dnaic1 disease model has a C75Bl/6/129 background. Animals were taken down 72 hours after the last exposure and the trachea were used to measure ciliary beat frequency using high-speed video microscopy imaged using a 40× water dipping objective by acquiring a 5 second video at 500 frames per second in an environment-control chamber at 37° C. and 5% CO2. Ciliary activity was calculated by generating a kymograph of the ciliary beat frequency in ImageJ and is represented in FIG. 11.

The C57Bl/6 mice showed a frequency of approximately 15 Hz, which served as our control for normal ciliary activity. The untreated disease animals had a significant drop in ciliary beat frequency, presumably due to the loss of ciliated cells after tamoxifen treatment. Notably, with exposure to the MRT-DNAI1-LNP2 treatment, there was an increase in ciliary activity that was significantly different from the untreated mice. While the CBF was still lower than in the C57Bl/6 mice, these data demonstrate that treatment with the exogenous DNAI1 mRNA after inhalation can improve ciliary activity.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A method of treating primary ciliary dyskinesia (PCD) comprising administering to a subject in need of treatment an mRNA encoding a dynein axonemal intermediate chain 1 (DNAI1) protein, wherein the mRNA is administered at a dose ranging from 1 mg to 36 mg daily for 5 consecutive days.

2. The method of claim 1, wherein the administration of the mRNA results in an increase in ciliary beat frequency (CBF) compared to a control.

3. A method of treating primary ciliary dyskinesia (PCD) comprising administering to a subject in need of treatment an mRNA encoding a dynein axonemal intermediate chain 1 (DNAI1) protein, wherein the mRNA is administered at a therapeutically effective dose and interval such that the subject achieves an increase in ciliary beat frequency (CBF) compared to a control.

4. The method of claim 2, wherein the CBF is increased by at least 10% compared to the control.

5. A method of treating primary ciliary dyskinesia (PCD) comprising administering to a subject in need of treatment an mRNA encoding a dynein axonemal intermediate chain 1 (DNAI1) protein, wherein the mRNA is administered at a therapeutically effective dose and interval such that the subject maintains an expression of the DNAI1 protein in airway epithelium at a level at least 10% of the wild type level.

6-8. (canceled)

9. The method of claim 5, wherein the subject maintains an expression of the DNAI1 protein in airway epithelium at a level at least 20%, at least 30%, at least 40%, at least 50%, at least 60%, at least 70%, at least 80%, or at least 90% of the wild type level.

10. The method of claim 5, wherein the subject achieves an increase in ciliary beat frequency (CBF) compared to a control.

11. (canceled)

12. (canceled)

13. The method of claim 3, wherein the control is a CBF in the subject before administration of the mRNA encoding a DNAI1 protein.

14. The method of claim 3, wherein the administration of the mRNA rescues the CBF in the subject to a normal CBF level, optionally wherein the normal CBF level is 6-9 Hz.

15-17. (canceled)

18. The method of claim 1, wherein the administration of the mRNA results in DNAI1-positive transfection efficiency of greater than 5% in ciliated cells.

19. (canceled)

20. The method of claim 1, wherein the DNAI1 mRNA is encapsulated in a liposome, wherein the liposome comprises one or more cationic lipids, one or more non-cationic lipids and one or more PEG-modified lipids.

21. (canceled)

22. The method of claim 20, wherein the one or more cationic lipids are selected from the group consisting of TL1-01D-DMA, TL1-10D-DMA, GL-TES-SA-DMP-E18-2, HEP-E4-E10, HEP-E3-E10 and TL1-04D-DMA, GL-TES-SA-DME-E18-2, Guan-SS-Chol, SY-3-E14-DMAPr, RL3-07D-DMA, cKK-E12, OF-02, ICE (Imidazol-based ester) and combinations thereof.

23. The method of claim 22, wherein the cationic lipid is TL1-01D-DMA or SY-3-E14-DMAPr.

24-37. (canceled)

38. The method of claim 1, wherein the DNAI1 mRNA is codon optimized, optionally wherein the DNAI1 mRNA comprises one or more modified nucleotides.

39. The method of claim 38, wherein the codon-optimized mRNA produces at least 10% more, 15% more, 20% more, 25% more, or at least 30% more DNAI1 protein in comparison to a non-codon-optimized mRNA sequence.

40-45. (canceled)

46. The method of claim 1, wherein the mRNA comprises a coding sequence at least 70%, 75%, 80%, 85%, 90%, or 95% identical to any one of SEQ ID NO: 6 to SEQ ID NO: 10.

47. (canceled)

48. (canceled)

49. The method of claim 1, wherein the mRNA comprises a coding sequence at least 95%, at least 96%, at least 97%, at least 98%, or at least 99% identical to SEQ ID NO: 6 to SEQ ID NO: 10.

50. The method of claim 1, wherein the mRNA comprises a coding sequence set forth in SEQ ID NO: 6 to SEQ ID NO: 10.

51. The method of claim 5, wherein administering the mRNA to the subject is performed by pulmonary, intratracheal, intranasal, intravenous, intramuscular or subcutaneous delivery.

52-63. (canceled)

64. The method of claim 51, wherein the administering the mRNA results in DNAI1 protein expression detectable in one or more internal organs selected from lung, heart, liver, spleen, kidney, brain, stomach, intestines, ovary and testis.

65-88. (canceled)


SEQUENCE LISTING (16 sequences)

SEQ ID NO: 1 - protein, 699 aa, Homo sapiens
MIPASAKAPHKQPHKQSISIGRGTRKRDEDSGTEVGEGTDEWAQSKATVRPPDQLELTDAELKEEFTRILTANNPHAPQNIVRYSFKEGTYKPIGFVNQLAVHYTQVGNLIPKDSDEGRRQHYRDELVAGSQESVKVISETGNLEEDEEPKELETEPGSQTDVPAAGAAEKVTEEELMTPKQPKERKLTNQFNFSERASQTYNNPVRDRECQTEPPPRTNFSATANQWEIYDAYVEELEKQEKTKEKEKAKTPVAKKSGKMAMRKLTSMESQTDDLIKLSQAAKIMERMVNQNTYDDIAQDFKYYDDAADEYRDQVGTLLPLWKFQNDKAKRLSVTALCWNPKYRDLFAVGYGSYDFMKQSRGMLLLYSLKNPSFPEYMFSSNSGVMCLDIHVDHPYLVAVGHYDGNVAIYNLKKPHSQPSFCSSAKSGKHSDPVWQVKWQKDDMDQNLNFFSVSSDGRIVSWTLVKRKLVHIDVIKLKVEGSTTEVPEGLQLHPVGCGTAFDFHKEIDYMFLVGTEEGKIYKCSKSYSSQFLDTYDAHNMSVDTVSWNPYHTKVFMSCSSDWTVKIWDHTIKTPMFIYDLNSAVGDVAWAPYSSTVFAAVTTDGKAHIFDLAINKYEAICNQPVAAKKNRLTHVQFNLIHPIIIVGDDRGHIISLKLSPNLRKMPKEKKGQEVQKGPAVEIAKLDKLLNLVREVKIKT

SEQ ID NO: 2 - RNA, 140 nt, synthetic construct
  source 1..140: mol_type = other RNA
agacagatcgcctggagacgccatccacgctgttttgacctccatagaagacaccgggaccgatccagcctccgcggccgggaacggtgcattggaacgcggattccccgtgccaagagtgactcaccgtccttgacacg

SEQ ID NO: 3 - RNA, 140 nt, synthetic construct
  source 1..140: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacg

SEQ ID NO: 4 - RNA, 105 nt, synthetic construct
  source 1..105: mol_type = other RNA
cgggtggcatccctgtgacccctccccagtgcctctcctggccctggaagttgccactccagtgcccaccagccttgtcctaataaaattaagttgcatcaagct

SEQ ID NO: 5 - RNA, 105 nt, synthetic construct
  source 1..105: mol_type = other RNA
gggtggcatccctgtgacccctccccagtgcctctcctggccctggaagttgccactccagtgcccaccagccttgtcctaataaaattaagttgcatcaaagct

SEQ ID NO: 6 - DNA, 2100 bp, synthetic construct
  source 1..2100: mol_type = other DNA
atgatccccgcctccgccaaagcccctcacaagcaaccgcacaagcaaagcattagcattgggcggggtactcggaagcgcgacgaggactcgggaactgaagtcggagaggggaccgacgaatgggcgcagtcaaaggccaccgtgcgcccaccggaccagctcgagctgaccgatgctgagctgaaggaggagtttacccggatcctgacagccaacaacccacatgcaccgcagaacatcgtgcggtacagcttcaaagagggaacttataagcccattggcttcgtgaaccaactcgcggtgcattacacccaagtcggaaaccttattccgaaggactcggacgaaggcagacgccagcactaccgggacgagctcgtggcaggatcccaggaaagcgtcaaggtcatttccgagactggcaacctcgaggaggacgaagaacctaaggagctggaaaccgaacccggatcccagaccgacgtgccggccgctggggctgccgagaaagtcactgaagaggaactcatgaccccgaagcagccgaaagagagaaagctcaccaaccaattcaacttcagcgagcgcgccagccaaacctacaacaacccagtcagggatcgggaatgtcagaccgaaccgcctccgagaacgaacttctcggcgaccgcgaaccaatgggagatctacgacgcctacgtggaagaactggaaaagcaggaaaagactaaggaaaaggaaaaggccaagactcccgtcgccaagaagtcgggcaaaatggccatgcggaagctcacctccatggaatcacagactgacgacttgatcaagttgagccaggccgcaaagatcatggagcgcatggtcaaccaaaatacttacgacgatatcgcccaagacttcaagtactacgacgacgctgccgatgaataccgagatcaagtcggcaccctactgccgctttggaagttccagaatgacaaggccaagaggctgagcgtgaccgcgctgtgctggaaccccaaataccgcgacctcttcgccgtgggatacggctcctacgatttcatgaagcagagccggggaatgttgctcctttactccctgaagaacccctccttccctgagtacatgttcagctcaaacagcggcgtgatgtgcctcgacattcacgtggaccacccttacctcgtggccgtgggtcactacgacggcaacgtcgcgatctacaacttgaagaagccgcattcacagccctcgttttgctcctcggccaagtccggcaaacattcggacccagtgtggcaagtcaagtggcagaaagatgacatggaccaaaacttgaacttcttcagcgtgtcctccgacggacggatcgtgtcctggaccctcgtgaagcggaagttggtgcatatcgacgtgatcaaattgaaggtcgagggttcgaccaccgaagtgcctgaaggcctgcagcttcaccccgtgggatgcggcactgccttcgacttccacaaggagatcgactacatgttcctcgtgggaaccgaggaagggaagatctacaaatgcagcaagtcctactcatcacaattcctggatacctacgatgcccacaacatgagcgtggataccgtgtcgtggaacccctatcacaccaaggtattcatgtcctgctcctccgactggaccgtcaagatttgggaccacaccatcaagacccccatgttcatctacgacctgaactccgccgtgggggatgtggcctgggccccctactcgtcgaccgtgtttgccgcggtcaccacggacggaaaggcacacattttcgaccttgcgattaacaaatacgaggcgatttgcaaccagcccgtggccgccaaaaagaaccgcctgacccacgttcaattcaacttaatccacccaatcatcatcgtcggcgatgacagaggacacattattagcctgaaacttagccccaacctccgcaagatgcccaaggagaagaagggacaggaagtccagaagggccctgccgtggagattgcaaagctcgataagctcctgaacttagtccgggaagtgaagatcaagacttaa

SEQ ID NO: 7 - DNA, 2100 bp, synthetic construct
  source 1..2100: mol_type = other DNA
atgatccccgctagcgctaaggcccctcacaaacagccccacaagcagagcattagcattggacgcggcacacggaaacgggacgaagattccggaaccgaagtcggggaaggcaccgatgaatgggcccagtccaaggccaccgtgcggcctcctgaccaattggaactgactgacgcggaactgaaggaagaattcaccagaattctaaccgccaacaatcctcatgccccgcaaaacatcgtgcgctactcattcaaggaaggaacctacaagcccattggattcgtgaaccaactcgccgtgcactacactcaagtcggcaacttaattcccaaggattcggacgagggccgccggcagcactacagggatgagcttgtggcaggctcacaggaaagcgtcaaagtcattagcgaaaccggcaaccttgaagaggatgaggagcctaaggagctggaaaccgagccgggttcgcaaaccgacgtgcctgccgccggagccgcagagaaggtcaccgaagaggaactcatgacccccaagcagccgaaggagcgcaaactcactaaccaattcaacttcagcgagagagcgagccagacctacaacaacccagtgcgcgaccgggaatgccagactgagccgcccccgagaactaacttcagcgcgactgccaaccaatgggagatctacgacgcctacgtggaagagttggagaagcaggaaaagaccaaggagaaggaaaaggccaagaccccggtggcgaagaagtccggaaagatggcgatgcggaagttgacgtccatggaatcccagaccgatgacctcatcaagctcagccaggcagcaaagatcatggaaaggatggtgaaccagaacacctacgatgacattgctcaggactttaagtactacgacgatgccgccgacgaataccgcgaccaggtcggaaccctcctcccgctttggaaattccagaacgataaagccaagcgcctcagcgtgactgcgctgtgctggaacccgaagtaccgggaccttttcgccgtgggctatggatcctacgacttcatgaagcaatcgcgggggatgctcctcctgtatagcctgaagaacccgagcttccccgagtacatgttcagctcaaactccggagtgatgtgcctcgacatccacgtggaccacccatacttggtggccgtgggccactacgatggcaatgtcgcgatctacaacctcaagaagccccactcccaaccctcgttctgcagcagcgccaagagcgggaaacactccgacccagtgtggcaagtcaaatggcagaaggacgacatggatcagaacttgaacttcttcagcgtgtcctcagacggacggatcgtgtcctggaccctcgtgaagaggaagttggtccatatcgacgtgatcaagctcaaggtcgagggttcaaccaccgaggtgcccgagggcctgcagctccaccccgtgggctgcggcacggccttcgactttcataaggaaattgactacatgttcctcgtggggactgaagagggaaagatctacaagtgcagcaaatcctactcgtcccaattcctcgacacctacgatgcgcacaacatgagcgtcgacacggtgtcatggaacccctaccatacaaaggtattcatgtcgtgctcctccgactggaccgtgaagatttgggatcacactatcaagaccccgatgttcatatacgacctgaactccgccgtgggagatgtggcttgggcgccctactcatcgaccgtgtttgccgccgtgaccaccgacggaaaggctcacattttcgatcttgccatcaacaagtacgaggcgatttgcaaccagccagtggccgccaagaagaacagactgactcacgtgcagtttaacttaatccaccccattattatcgtgggggacgaccgcggacacattattagcctgaagctcagcccgaacctccggaagatgcctaaggagaagaaggggcaggaagtgcagaaaggcccggccgtggaaattgccaaattggacaagttactgaaccttgtccgcgaagtcaagatcaaaacttaa

SEQ ID NO: 8 - DNA, 2100 bp, synthetic construct
  source 1..2100: mol_type = other DNA
atgatccccgcatccgccaaagcccctcataaacagccccacaaacagtccatctccattggacgggggacccggaaaagggatgaggactctgggacggaagttggagaaggcactgacgaatgggcacagagtaaggctaccgtgagacctcccgaccagctggagctcactgacgcagaactgaaggaggagtttactaggatcctgacagcaaataacccccacgccccacagaatatcgtcagatatagcttcaaagagggcacatacaagcctattgggttcgtgaaccagctggctgtgcattacacacaggtggggaaccttattcctaaagactctgatgaaggccgcagacagcattatagagatgaactggttgcaggatcccaagagtctgtgaaagtgattagcgagaccggcaacctggaagaagatgaggaaccaaaagaactggagacagagcctgggtctcagacagacgtgccagcagctggcgctgccgagaaagtgacagaggaggagctgatgacacctaaacagccaaaagagaggaagctgacaaaccaattcaatttttccgaacgggcatcacagacctacaacaacccagtgcgcgaccgggagtgtcaaaccgaacctcctcctagaacaaacttttctgctactgcaaatcagtgggagatctacgatgcctacgtggaggagctggagaagcaggaaaagactaaggagaaggagaaggcaaagacccccgtggccaaaaaatccggcaaaatggcaatgcggaagctgacttctatggaaagccagactgatgacctgatcaaactgtcccaggcagctaagattatggaaaggatggtcaatcagaatacatatgacgacattgctcaggactttaagtattatgatgatgccgctgacgagtatcgggaccaagtggggacactgctgccactgtggaagtttcaaaacgacaaggctaaaaggctgtccgtgacagcactctgctggaatcccaagtaccgggacctctttgccgtggggtacggatcttacgacttcatgaaacagtccagaggcatgctgctgctgtacagcttgaagaacccctcctttcccgagtacatgttcagctctaattctggagtgatgtgcctggacatccacgtggatcacccttacctcgtggccgttggacactatgacggcaatgtggccatctacaacctgaaaaaaccacactctcagccttccttttgtagctctgcaaagtccggaaagcattccgaccccgtgtggcaagtgaaatggcagaaagacgacatggaccagaatctgaacttcttctccgtctcttcagacggcagaatcgtctcatggactctggtcaaacggaagctggttcacatcgacgtgatcaaactcaaggtcgaaggatcgactactgaggtgccagaaggactgcagctgcacccagtgggatgtggaactgcatttgatttccataaagaaatcgactacatgtttctggtgggaactgaagaggggaagatctataagtgtagcaaatcctattctagccagtttctggatacatacgacgctcacaacatgtccgtggacactgtaagctggaacccctatcataccaaggtgttcatgtcctgcagctccgattggactgttaagatttgggatcacacaatcaagacccctatgtttatctacgatctgaactctgccgtgggggatgtggcctgggcaccatatagctccacagtcttcgcagctgtcactaccgatggaaaggcccacatttttgacctggctatcaacaaatacgaggccatctgcaatcagcctgtggcagcaaagaagaaccgcctgactcacgtgcaattcaacctgattcaccctatcatcattgttggggatgataggggccacattatttctctaaagctgtccccaaatctgcggaaaatgcccaaggagaagaaaggccaggaggtgcagaaaggcccagccgttgaaatcgcaaagctggacaagctgctcaacctcgtccgggaggttaaaatcaaaacctga

SEQ ID NO: 9 - DNA, 2100 bp, synthetic construct
  source 1..2100: mol_type = other DNA
atgatcccagcaagcgccaaggccccacacaaacagccccacaagcagtcgatcagcattggcagggggactcgcaagagagacgaggactccggaacagaagtgggggaggggacagatgaatgggcccagtctaaggccactgttcgccctccggatcagctggaactgacagatgccgagctgaaggaagagttcaccaggattctgactgcaaataatccacacgctccacagaacattgtgagatattcttttaaggagggcacttacaaacccatcgggtttgtgaatcagctggcagtgcattacactcaagtgggcaacctgatccccaaagactctgatgaagggaggcggcagcactatagggacgagctggtcgctgggtcccaagagagcgtgaaagtcatttctgagactggcaacctggaagaggatgaggagccaaaggagctggagactgaaccagggtctcagacagatgtgcccgccgctggagctgctgagaaggtgacagaggaggaactgatgacccctaaacagcctaaggaacggaagctcaccaaccagttcaacttcagcgaaagagctagccagacttataataaccctgtgcgcgaccgggagtgtcagactgagcccccaccaagaaccaatttctccgccactgccaaccagtgggaaatctatgacgcttacgtcgaggagctggagaaacaggagaaaactaaggagaaagaaaaggccaaaacacccgtcgccaaaaagtctggcaagatggccatgagaaaactgacctccatggagtctcagaccgacgacctgatcaaactgtcccaggcagccaagatcatggagaggatggtgaaccagaacacctatgatgacattgcccaggactttaaatactacgatgatgccgctgacgagtatcgggaccaggtggggactctgctgcctctgtggaaattccagaatgataaggctaaacgcctgtccgtgaccgccctctgctggaaccctaagtaccgcgacctctttgctgtggggtacggatcttacgacttcatgaaacagtccagaggcatgctgctgctgtacagcttgaagaacccctcctttcccgagtacatgttcagctctaattctggagtgatgtgcctggacatccacgtggatcacccttacctcgtggccgttggacactatgacggcaatgtggccatctacaacctgaaaaaaccacactctcagccttccttttgtagctctgcaaagtccggaaagcattccgaccccgtgtggcaagtgaaatggcagaaagacgacatggaccagaatctgaacttcttctccgtctcttcagacggcagaatcgtctcatggactctggtcaaacggaagctggttcacatcgacgtgatcaaactcaaggtcgaaggatcgactactgaggtgccagaaggactgcagctgcacccagtgggatgtggaactgcatttgatttccataaagaaatcgactacatgtttctggtgggaactgaagaggggaagatctataagtgtagcaaatcctattctagccagtttctggatacatacgacgctcacaacatgtccgtggacactgtaagctggaacccctatcataccaaggtgttcatgtcctgcagctccgattggactgttaagatttgggatcacacaatcaagacccctatgtttatctacgatctgaactctgccgtgggggatgtggcctgggcaccatatagctccacagtcttcgcagctgtcactaccgatggaaaggcccacatttttgacctggctatcaacaaatacgaggccatctgcaatcagcctgtggcagcaaagaagaaccgcctgactcacgtgcaattcaacctgattcaccctatcatcattgttggggatgataggggccacattatttctctaaagctgtccccaaatctgcggaaaatgcccaaggagaagaaaggccaggaggtgcagaaaggcccagccgttgaaatcgcaaagctggacaagctgctcaacctcgtccgggaggttaaaatcaaaacctga

SEQ ID NO: 10 - DNA, 2100 bp, synthetic construct
  source 1..2100: mol_type = other DNA
atgatcccagcttctgccaaggccccacacaagcagccacacaaacagagcatttccattgggcgcggcacaaggaagagagacgaggactcaggcacagaggtgggcgaaggaaccgacgagtgggctcagagcaaagccacagtgaggcccccagatcagctggagctgacagacgccgagctgaaggaggagtttacccgcatcctgactgccaataacccacacgcaccccagaacatcgtgcgctattcttttaaggaaggaacctataagccaatcggctttgtcaatcagctggctgtgcactacacccaggttgggaacctgatccccaaggatagcgacgagggcaggagacagcattatagagacgagctcgtcgccggaagccaggagtctgtcaaagtgatcagcgaaacaggaaacctggaggaggatgaggagcccaaggaactggaaaccgagcctggcagccagacagatgtgccagccgcaggagccgcagagaaggtgacagaagaggagctcatgacccccaaacagccaaaggagcggaaactgacaaaccagttcaacttcagcgaaagagccagccagacctacaataaccccgtgcgggacagagaatgccagacagagcctccaccacgcaccaacttctccgcaacagctaaccagtgggagatctatgatgcctacgtggaggagctggaaaagcaggagaagaccaaagaaaaggagaaagccaagacccctgtcgccaagaagtccggcaaaatggctatgagaaagctgacatctatggaatcccagactgatgacctgatcaagctgtctcaggcagccaagattatggaaagaatggtgaatcagaacacctatgacgacatcgcccaggattttaagtactatgatgacgctgcagacgagtatagagatcaggtggggaccctgctgccactgtggaagttccagaatgacaaggctaagcgcctgtccgtgacagctctgtgctggaatccaaaatatagggacctcttcgccgtgggctacggctcttatgacttcatgaagcagtcacgcgggatgctgctgctgtacagcctgaaaaatccctcctttcccgagtacatgttcagctctaactccggggtcatgtgtctggatattcatgtggaccatccatacctggtggctgtcgggcactacgatggaaacgtggctatctacaatctgaagaagccacactcccagccctccttttgctcctccgccaagtccggcaagcactccgaccctgtgtggcaggtcaagtggcagaaggacgacatggaccagaacctgaacttcttttctgtgtctagcgatggcaggatcgtgtcctggaccctggtgaagagaaaactggtgcacatcgatgttatcaagctcaaagtcgagggaagcaccaccgaggttcctgagggcctgcagctgcacccagtgggctgcggcacagccttcgactttcataaagagattgactacatgttcctggtgggcacagaggaggggaagatctacaagtgctccaaatcctactccagccagtttctggacacttacgacgctcataatatgagcgtggacaccgtgtcctggaacccttaccacacaaaggtgttcatgagctgcagcagcgactggactgtgaagatttgggaccatactatcaaaaccccaatgtttatctatgatctcaattctgccgtgggcgacgtggcttgggccccctattcctccacagtgttcgcagccgtgactaccgacggaaaagcccacattttcgacctcgctattaacaagtatgaggccatttgtaaccagccagtggctgccaagaagaaccgcctgacccacgtgcagttcaacctgattcacccaattatcattgtgggggacgacagaggacacattatctcactgaagctgtctcctaatctgagaaagatgcctaaggagaagaaaggacaggaggtgcagaagggccctgccgtggaaattgccaaactcgacaagctgctgaacctggtgagggaggtgaagatcaagacatga

SEQ ID NO: 11 - DNA, 2106 bp, synthetic construct
  source 1..2106: mol_type = other DNA
atgccatccaaacaaattagaaagcagagcattagcgtgacccgcggagcccggagaagagatgaggactcgggcaccgatgtcggcgaaggaaccgacgagtggtcgcagtcaaaggctaccgtgcggccgcctgaccagcttgaacttaccgatgccgagctgaaggaagagttcactcgcatcctgaccgccaacaatccccacgcgccgcagaacatcgtgcgctactccttcaaagaaggaacgtacaagctcattggattcgtgaaccaaatggccgtgcacttttcccaagtcggaaacctcattccgaaggattccgacgagggccggcggcagcattaccgcgacgaaatggtggccggatcgcaagaaagcatcaaggtcgtgacttccgaggccgagaacctcgaggaagaagaagaaccgaaagagggcgaaggagaagccgaagccgaggctgaggccggaagccagaccgatatccccgctgccgcagaaactaccgaaaaggtcatcgaagaggagttgatggccccggtgcagcctaaggaacggaagttgactaaccaattcaacttcagcgagagagcctcacagaccttcaacaacccactgagagatagggaatgtcaaatggagcctccgccccggacgaacttctcggccactgcaaaccaatgggagatctacgacgcttacgtggatgaactggagaagcaggaaaagaccaaggaaaaggagaaagccaagaccccggtcgccaaaaagactgagaaaatggccatgcggaagctcacttccatggagagccagtccgacgatattactaaggttacccaggccgccaagatcgtggaaagaatggtgaaccagaatacctacgacgacgtggcacaggattttaagtactatgaggacaccgctgacgaataccgggatcaagaaggcaccctcctgcccttgtggaagttccagaatgacaaggccaagaggctggccgtgactgcgctgtgctggaacccgaagtataaggacctcttcgccgtgggtcatggctcatacgactttatgaagcaatcgcgcgggatgctcctgctctactcgatgaagaacccttccttccccgagtacatgttcagctccgagtcagggatcatgtgcctggatgtccatgtcgaccacccttacctcgtggtcgtgggatattacgacggcaacgtggcgatctacaaccttaagaaaccgcatagccagccctgcttccggagcacctcaaagagcggaaagcacaccgaccccgtgtggcaagtcaagtggcaaaaggacgacatggaccacaacttgaacttcttcagcgtgtcgtccgacggacggatcgtgtcctggaccctcgtcaagtccgagctcgtgcacattgacattatcaagctcaaaaccgagggttccaccactgaaatccccgagggcctgcagctccataccgtgggatgcggcaccgcgttcgacttccataaagagatcgactacatgttcctcgtggggactgaagaggggaagatctacaagtgcagcaagtcgtactcttcccaattcctggacacctacgacgctcataacatggcagtggacgccgtgctgtggaacccataccacactaaggtctttatgtcctgctcctcggactggactgtgaagatttgggaccacaccatcaaaaccccaatgttcatttacgacctgaactccgccgtgggggatgtggcctgggccccgtattcctccaccgtattcgccgccgtgaccaccgacggaaaggcacacgtctttgacctggccgtgaacaaatacgaggcgatttgcaaccagccagtggtggccaagaagaaaaacaagatcactcacgtgcaattcaaccccatccaccccattattatcgtcggcgacgataggggtcacattatatgcctgaagctcagcccgaaccttcgcaagatgcctaaggaaaagaagggccaggaggtgcagaagggtcctgccgtcgaaattgcgaagttggacaagctcctgaacctcgtgcgcgaggtcaagactaagacctaa

SEQ ID NO: 12 - DNA, 2136 bp, synthetic construct
  source 1..2136: mol_type = other DNA
atgatccccgcctccgccaaagcccctcacaagcaaccgcacaagcaaagcattagcattgggcggggtactcggaagcgcgacgaggactcgggaactgaagtcggagaggggaccgacgaatgggcgcagtcaaaggccaccgtgcgcccaccggaccagctcgagctgaccgatgctgagctgaaggaggagtttacccggatcctgacagccaacaacccacatgcaccgcagaacatcgtgcggtacagcttcaaagagggaacttataagcccattggcttcgtgaaccaactcgcggtgcattacacccaagtcggaaaccttattccgaaggactcggacgaaggcagacgccagcactaccgggacgagctcgtggcaggatcccaggaaagcgtcaaggtcatttccgagactggcaacctcgaggaggacgaagaacctaaggagctggaaaccgaacccggatcccagaccgacgtgccggccgctggggctgccgagaaagtcactgaagaggaactcatgaccccgaagcagccgaaagagagaaagctcaccaaccaattcaacttcagcgagcgcgccagccaaacctacaacaacccagtcagggatcgggaatgtcagaccgaaccgcctccgagaacgaacttctcggcgaccgcgaaccaatgggagatctacgacgcctacgtggaagaactggaaaagcaggaaaagactaaggaaaaggaaaaggccaagactcccgtcgccaagaagtcgggcaaaatggccatgcggaagctcacctccatggaatcacagactgacgacttgatcaagttgagccaggccgcaaagatcatggagcgcatggtcaaccaaaatacttacgacgatatcgcccaagacttcaagtactacgacgacgctgccgatgaataccgagatcaagtcggcaccctactgccgctttggaagttccagaatgacaaggccaagaggctgagcgtgaccgcgctgtgctggaaccccaaataccgcgacctcttcgccgtgggatacggctcctacgatttcatgaagcagagccggggaatgttgctcctttactccctgaagaacccctccttccctgagtacatgttcagctcaaacagcggcgtgatgtgcctcgacattcacgtggaccacccttacctcgtggccgtgggtcactacgacggcaacgtcgcgatctacaacttgaagaagccgcattcacagccctcgttttgctcctcggccaagtccggcaaacattcggacccagtgtggcaagtcaagtggcagaaagatgacatggaccaaaacttgaacttcttcagcgtgtcctccgacggacggatcgtgtcctggaccctcgtgaagcggaagttggtgcatatcgacgtgatcaaattgaaggtcgagggttcgaccaccgaagtgcctgaaggcctgcagcttcaccccgtgggatgcggcactgccttcgacttccacaaggagatcgactacatgttcctcgtgggaaccgaggaagggaagatctacaaatgcagcaagtcctactcatcacaattcctggatacctacgatgcccacaacatgagcgtggataccgtgtcgtggaacccctatcacaccaaggtattcatgtcctgctcctccgactggaccgtcaagatttgggaccacaccatcaagacccccatgttcatctacgacctgaactccgccgtgggggatgtggcctgggccccctactcgtcgaccgtgtttgccgcggtcaccacggacggaaaggcacacattttcgaccttgcgattaacaaatacgaggcgatttgcaaccagcccgtggccgccaaaaagaaccgcctgacccacgttcaattcaacttaatccacccaatcatcatcgtcggcgatgacagaggacacattattagcctgaaacttagccccaacctccgcaagatgcccaaggagaagaagggacaggaagtccagaagggccctgccgtggagattgcaaagctcgataagctcctgaacttagtccgggaagtgaagatcaagactgggggcggatccgactataaggatgatgacgataagtaa

SEQ ID NO: 13 - DNA, 2139 bp, synthetic construct
  source 1..2139: mol_type = other DNA
atgatccccgcctccgccaaagcccctcacaagcaaccgcacaagcaaagcattagcattgggcggggtactcggaagcgcgacgaggactcgggaactgaagtcggagaggggaccgacgaatgggcgcagtcaaaggccaccgtgcgcccaccggaccagctcgagctgaccgatgctgagctgaaggaggagtttacccggatcctgacagccaacaacccacatgcaccgcagaacatcgtgcggtacagcttcaaagagggaacttataagcccattggcttcgtgaaccaactcgcggtgcattacacccaagtcggaaaccttattccgaaggactcggacgaaggcagacgccagcactaccgggacgagctcgtggcaggatcccaggaaagcgtcaaggtcatttccgagactggcaacctcgaggaggacgaagaacctaaggagctggaaaccgaacccggatcccagaccgacgtgccggccgctggggctgccgagaaagtcactgaagaggaactcatgaccccgaagcagccgaaagagagaaagctcaccaaccaattcaacttcagcgagcgcgccagccaaacctacaacaacccagtcagggatcgggaatgtcagaccgaaccgcctccgagaacgaacttctcggcgaccgcgaaccaatgggagatctacgacgcctacgtggaagaactggaaaagcaggaaaagactaaggaaaaggaaaaggccaagactcccgtcgccaagaagtcgggcaaaatggccatgcggaagctcacctccatggaatcacagactgacgacttgatcaagttgagccaggccgcaaagatcatggagcgcatggtcaaccaaaatacttacgacgatatcgcccaagacttcaagtactacgacgacgctgccgatgaataccgagatcaagtcggcaccctactgccgctttggaagttccagaatgacaaggccaagaggctgagcgtgaccgcgctgtgctggaaccccaaataccgcgacctcttcgccgtgggatacggctcctacgatttcatgaagcagagccggggaatgttgctcctttactccctgaagaacccctccttccctgagtacatgttcagctcaaacagcggcgtgatgtgcctcgacattcacgtggaccacccttacctcgtggccgtgggtcactacgacggcaacgtcgcgatctacaacttgaagaagccgcattcacagccctcgttttgctcctcggccaagtccggcaaacattcggacccagtgtggcaagtcaagtggcagaaagatgacatggaccaaaacttgaacttcttcagcgtgtcctccgacggacggatcgtgtcctggaccctcgtgaagcggaagttggtgcatatcgacgtgatcaaattgaaggtcgagggttcgaccaccgaagtgcctgaaggcctgcagcttcaccccgtgggatgcggcactgccttcgacttccacaaggagatcgactacatgttcctcgtgggaaccgaggaagggaagatctacaaatgcagcaagtcctactcatcacaattcctggatacctacgatgcccacaacatgagcgtggataccgtgtcgtggaacccctatcacaccaaggtattcatgtcctgctcctccgactggaccgtcaagatttgggaccacaccatcaagacccccatgttcatctacgacctgaactccgccgtgggggatgtggcctgggccccctactcgtcgaccgtgtttgccgcggtcaccacggacggaaaggcacacattttcgaccttgcgattaacaaatacgaggcgatttgcaaccagcccgtggccgccaaaaagaaccgcctgacccacgttcaattcaacttaatccacccaatcatcatcgtcggcgatgacagaggacacattattagcctgaaacttagccccaacctccgcaagatgcccaaggagaagaagggacaggaagtccagaagggccctgccgtggagattgcaaagctcgataagctcctgaacttagtccgggaagtgaagatcaagactgggggcggatcctacccctacgacgtgccagactacgcataa

SEQ ID NO: 14 - DNA, 2100 bp, synthetic construct
  source 1..2100: mol_type = other DNA
atgatccccgcctaagccaaagcccctcataagcaaccgcacaagcaaagcataagctgagggcggggtactcggaagcgtgacgaggactcgggaactgaagtaggagaggggaccgacgaatgggcgcagtcaaaggccaccgtgcgcccaccggaccagctcgagctgaccgatgctgagctgaaggaggagtttacccggatcctgacagccaacaacccacatgcaccgcagaacatcgtgcggtacagcttcaaagagggaacttataagcccattggcttcgtgaaccaactcgcggtgcattacacccaagtcggaaaccttattccgaaggactcggacgaaggcagacgccagcactaccgggacgagctcgtggcaggatcccaggaaagcgtcaaggtcatttccgagactggcaacctcgaggaggacgaagaacctaaggagctggaaaccgaacccggatcccagaccgacgtgccggccgctggggctgccgagaaagtcactgaagaggaactcatgaccccgaagcagccgaaagagagaaagctcaccaaccaattcaacttcagcgagcgcgccagccaaacctacaacaacccagtcagggatcgggaatgtcagaccgaaccgcctccgagaacgaacttctcggcgaccgcgaaccaatgggagatctacgacgcctacgtggaagaactggaaaagcaggaaaagactaaggaaaaggaaaaggccaagactcccgtcgccaagaagtcgggcaaaatggccatgcggaagctcacctccatggaatcacagactgacgacttgatcaagttgagccaggccgcaaagatcatggagcgcatggtcaaccaaaatacttacgacgatatcgcccaagacttcaagtactacgacgacgctgccgatgaataccgagatcaagtcggcaccctactgccgctttggaagttccagaatgacaaggccaagaggctgagcgtgaccgcgctgtgctggaaccccaaataccgcgacctcttcgccgtgggatacggctcctacgatttcatgaagcagagccggggaatgttgctcctttactccctgaagaacccctccttccctgagtacatgttcagctcaaacagcggcgtgatgtgcctcgacattcacgtggaccacccttacctcgtggccgtgggtcactacgacggcaacgtcgcgatctacaacttgaagaagccgcattcacagccctcgttttgctcctcggccaagtccggcaaacattcggacccagtgtggcaagtcaagtggcagaaagatgacatggaccaaaacttgaacttcttcagcgtgtcctccgacggacggatcgtgtcctggaccctcgtgaagcggaagttggtgcatatcgacgtgatcaaattgaaggtcgagggttcgaccaccgaagtgcctgaaggcctgcagcttcaccccgtgggatgcggcactgccttcgacttccacaaggagatcgactacatgttcctcgtgggaaccgaggaagggaagatctacaaatgcagcaagtcctactcatcacaattcctggatacctacgatgcccacaacatgagcgtggataccgtgtcgtggaacccctatcacaccaaggtattcatgtcctgctcctccgactggaccgtcaagatttgggaccacaccatcaagacccccatgttcatctacgacctgaactccgccgtgggggatgtggcctgggccccctactcgtcgaccgtgtttgccgcggtcaccacggacggaaaggcacacattttcgaccttgcgattaacaaatacgaggcgatttgcaaccagcccgtggccgccaaaaagaaccgcctgacccacgttcaattcaacttaatccacccaatcatcatcgtcggcgatgacagaggacacattattagcctgaaacttagccccaacctccgcaagatgcccaaggagaagaagggacaggaagtccagaagggccctgccgtggagattgcaaagctcgataagctcctgaacttagtccgggaagtgaagatcaagacttaa

SEQ ID NO: 15 - DNA, 2138 bp, synthetic construct
  source 1..2138: mol_type = other DNA
atgccatccaaacaaattagaaagcagagcattagcgtgacccgcggagcccggagaagagatgaggactcgggcaccgatgtcggcgaaggaaccgacgagtggtcgcagtcaaaggctaccgtgcggccgcctgaccagcttgaacttaccgatgccgagctgaaggaagagttcactcgcatcctgaccgccaacaatccccacgcgccgcagaacatcgtgcgctactccttcaaagaaggaacgtacaagctcattggattcgtgaaccaaatggccgtgcacttttcccaagtcggaaacctcattccgaaggattccgacgagggccggcggcagcattaccgcgacgaaatggtggccggatcgcaagaaagcatcaaggtcgtgacttccgaggccgagaacctcgaggaagaagaagaaccgaaagagggcgaaggagaagccgaagccgaggctgaggccggaagccagaccgatatccccgctgccgcagaaactaccgaaaaggtcatcgaagaggagttgatggccccggtgcagcctaaggaacggaagttgactaaccaattcaacttcagcgagagagcctcacagaccttcaacaacccactgagagatagggaatgtcaaatggagcctccgccccggacgaacttctcggccactgcaaaccaatgggagatctacgacgcttacgtggatgaactggagaagcaggaaaagaccaaggaaaaggagaaagccaagaccccggtcgccaaaaagactgagaaaatggccatgcggaagctcacttccatggagagccagtccgacgatattactaaggttacccaggccgccaagatcgtggaaagaatggtgaaccagaatacctacgacgacgtggcacaggattttaagtactatgaggacaccgctgacgaataccgggatcaagaaggcaccctcctgcccttgtggaagttccagaatgacaaggccaagaggctggccgtgactgcgctgtgctggaacccgaagtataaggacctcttcgccgtgggtcatggctcatacgactttatgaagcaatcgcgcgggatgctcctgctctactcgatgaagaacccttccttccccgagtacatgttcagctccgagtcagggatcatgtgcctggatgtccatgtcgaccacccttacctcgtggtcgtgggatattacgacggcaacgtggcgatctacaaccttaagaaaccgcatagccagccctgcttccggagcacctcaaagagcggaaagcacaccgaccccgtgtggcaagtcaagtggcaaaaggacgacatggaccacaacttgaacttcttcagcgtgtcgtccgacggacggatcgtgtcctggaccctcgtcaagtccgagctcgtgcacattgacattatcaagctcaaaaccgagggttccaccactgaaatccccgagggcctgcagctccataccgtgggatgcggcaccgcgttcgacttccataaagagatcgactacatgttcctcgtggggactgaagaggggaagatctacaagtgcagcaagtcgtactcttcccaattcctggacacctacgacgctcataacatggcagtggacgccgtgctgtggaacccataccacactaaggtctttatgtcctgctcctcggactggactgtgaagatttgggaccacaccatcaaaaccccaatgttcatttacgacctgaactccgccgtgggggatgtggcctgggccccgtattcctccaccgtattcgccgccgtgaccaccgacggaaaggcacacgtctttgacctggccgtgaacaaatacgaggcgatttgcaaccagccagtggtggccaagaagaaaaacaagatcactcacgtgcaattcaaccccatccaccccattattatcgtcggcgacgataggggtcacattatatgcctgaagctcagcccgaaccttcgcaagatgcctaaggaaaagaagggccaggaggtgcagaagggtcctgccgtcgaaattgcgaagttggacaagctcctgaacctcgtgcgcgaggtcaagactaagaccgggggtggttccgactacaaggatgatgacgataa

SEQ ID NO: 16 - DNA, 2144 bp, synthetic construct
  source 1..2144: mol_type = other DNA
atgccatccaaacaaattagaaagcagagcattagcgtgacccgcggagcccggagaagagatgaggactcgggcaccgatgtcggcgaaggaaccgacgagtggtcgcagtcaaaggctaccgtgcggccgcctgaccagcttgaacttaccgatgccgagctgaaggaagagttcactcgcatcctgaccgccaacaatccccacgcgccgcagaacatcgtgcgctactccttcaaagaaggaacgtacaagctcattggattcgtgaaccaaatggccgtgcacttttcccaagtcggaaacctcattccgaaggattccgacgagggccggcggcagcattaccgcgacgaaatggtggccggatcgcaagaaagcatcaaggtcgtgacttccgaggccgagaacctcgaggaagaagaagaaccgaaagagggcgaaggagaagccgaagccgaggctgaggccggaagccagaccgatatccccgctgccgcagaaactaccgaaaaggtcatcgaagaggagttgatggccccggtgcagcctaaggaacggaagttgactaaccaattcaacttcagcgagagagcctcacagaccttcaacaacccactgagagatagggaatgtcaaatggagcctccgccccggacgaacttctcggccactgcaaaccaatgggagatctacgacgcttacgtggatgaactggagaagcaggaaaagaccaaggaaaaggagaaagccaagaccccggtcgccaaaaagactgagaaaatggccatgcggaagctcacttccatggagagccagtccgacgatattactaaggttacccaggccgccaagatcgtggaaagaatggtgaaccagaatacctacgacgacgtggcacaggattttaagtactatgaggacaccgctgacgaataccgggatcaagaaggcaccctcctgcccttgtggaagttccagaatgacaaggccaagaggctggccgtgactgcgctgtgctggaacccgaagtataaggacctcttcgccgtgggtcatggctcatacgactttatgaagcaatcgcgcgggatgctcctgctctactcgatgaagaacccttccttccccgagtacatgttcagctccgagtcagggatcatgtgcctggatgtccatgtcgaccacccttacctcgtggtcgtgggatattacgacggcaacgtggcgatctacaaccttaagaaaccgcatagccagccctgcttccggagcacctcaaagagcggaaagcacaccgaccccgtgtggcaagtcaagtggcaaaaggacgacatggaccacaacttgaacttcttcagcgtgtcgtccgacggacggatcgtgtcctggaccctcgtcaagtccgagctcgtgcacattgacattatcaagctcaaaaccgagggttccaccactgaaatccccgagggcctgcagctccataccgtgggatgcggcaccgcgttcgacttccataaagagatcgactacatgttcctcgtggggactgaagaggggaagatctacaagtgcagcaagtcgtactcttcccaattcctggacacctacgacgctcataacatggcagtggacgccgtgctgtggaacccataccacactaaggtctttatgtcctgctcctcggactggactgtgaagatttgggaccacaccatcaaaaccccaatgttcatttacgacctgaactccgccgtgggggatgtggcctgggccccgtattcctccaccgtattcgccgccgtgaccaccgacggaaaggcacacgtctttgacctggccgtgaacaaatacgaggcgatttgcaaccagccagtggtggccaagaagaaaaacaagatcactcacgtgcaattcaaccccatccaccccattattatcgtcggcgacgataggggtcacattatatgcctgaagctcagcccgaaccttcgcaagatgcctaaggaaaagaagggccaggaggtgcagaagggtcctgccgtcgaaattgcgaagttggacaagctcctgaacctcgtgcgcgaggtcaagactaagaccgggggtggttcctacccatacgatgtgcctgactacgcgta